(19) United States
(12) Patent Application Publication
(10) Pub. No.: US 2016/0214996 A1
(43) Pub. Date: Jul. 28, 2016

(54) MACROCYCLIC COMPOUNDS FOR THE TREATMENT OF PROLIFERATIVE DISEASES

(71) Applicant: CS Pharmasciences, Inc., Palo Alto, CA (US)

(72) Inventors: Yuntao Song, Palo Alto, CA (US); Alexander James Bridges, Saline, MI (US)

(21) Appl. No.: 15/088,485

(22) Filed: Apr. 1, 2016

Related U.S. Application Data
(63) Continuation of application No. PCT/US2014/058623, filed on Oct. 1, 2014.
(60) Provisional application No. 61/885,347, filed on Oct. 1, 2013.

(51) Int. Cl. C07D 498/22; C07D 491/18; C07D 497/18; C07D 487/22; C07D 498/08; C07D 498/18

(52) U.S. Cl. CPC C07D 498/22; C07D 498/08; C07D 498/18; C07D 497/18; C07D 487/22; C07D 491/18

(57) ABSTRACT

The compounds and salts of the present invention inhibit kinases, especially the anaplastic lymphoma kinase (ALK) and the HGF receptor tyrosine kinase (RTK) c-Met, and are useful for treating or ameliorating abnormal cell proliferative disorders, such as cancer.

### BACKGROUND OF THE INVENTION

1. Field of the Invention

The present invention relates to compounds of formula 1 and their pharmaceutically acceptable salts, to pharmaceutical compositions comprising such compounds and salts, and to the uses thereof. The compounds and salts of the present invention inhibit kinases, especially the anaplastic lymphoma kinase (ALK) and the HGF receptor tyrosine kinase (RTK) c-Met, and are useful for treating or ameliorating abnormal cell proliferative disorders, such as cancer.

2. Background Information

Hyperphosphorylation of proteins is one of the hallmarks of cancer cells, and virtually all cancers looked at will show this phenomenon, often to a very marked extent. Protein hyperphosphorylation is usually mainly caused by overactivity of the enzymes which catalyze phosphotransfers from ATP to hydroxyls in protein side chains (protein kinases PKs), although over-inactivation of the enzymes which cleave these phosphate esters from proteins (protein phosphatases) can also occur. Protein phosphorylations are usually the end product of signaling cascades, whereby one kinase is activated, and it in turn phosphorylates other kinases, which causes them to switch from an inactive state to an active one, and in turn they phosphorylate further proteins including hitherto inactive kinases, leading overall to both signal amplification, and changed function for many proteins, not just the PKs.

PKs usually transfer the phosphate group to a serine or threonine hydroxyl group on the protein, in which case they are serine/threonine kinases (S/TKs). The next most common class of kinases transfer the phosphate onto the phenolic hydroxyl of tyrosine side chains on the target protein and are protein tyrosine kinases (PTKs). A rarer group target hydroxyls associated with various lipids such as phosphatidylinositol and sphingosine, and are called lipid kinases. Some of these are closely structurally related to certain PKs, and because of this, and the frequent required activity of lipid kinases in the same pathways as PKs, they are usually considered along with the PKs. The rarest group are the dual specificity kinases (DSKs), which can phosphorylate both serine/threonine and tyrosine hydroxyls. Generally speaking, none of the PK classes in cells in normal amounts appears to phosphorylate other classes of substrate, although some loss of specificity can be induced under laboratory conditions.

Structurally, the kinases are quite well understood. There is a kinase domain, which may be the whole protein, or only a small part of a much larger modular protein, and this domain has a basic conserved structure of about 35 kD, consisting of two lobes, the N-terminal one being mainly made up of beta-sheets, and the larger C-terminal domain mainly of alpha-helices. There is a deep cleft between the two lobes which binds both ATP and the substrate. The substrate binding domain is quite large, and rather variable, and is used to discriminate between different protein substrates, and maintain specificity of phosphorylation. This specificity can be very variable, with some enzymes such as MEK having only one known substrate, and others being able to phosphorylate hundreds of distinct hydroxyls in proteins.

Phosphorylation can change the conformation of the protein, often converting enzymes from an inactive form to an active form, or vice versa, or causing the protein to associate closely with binding partners, leading to changes in cellular localization, or assembly, or disassembly, of functioning multi-protein complexes. Many of the transducers of signals into cells, and from the cell surface into the nucleus are either PKs, or controlled by PKs. Therefore, inhibitors of the kinase activity of PKs can have very drastic effects on cellular signaling, damping down both normal responses to external signals, and inappropriate over-responses, usually caused by mutations in the signaling molecules themselves. Although such pathways are very widespread in the body, and are involved in one way or another in most bodily functions, and the diseases that can arise from their malfunction, inhibitors of PKs are particularly useful in treating cancer and immunological disorders, both disease classes where over-activity of PKs has been widely documented, and where they often play crucial roles in driving the disease process itself.

Crizotinib is a potent kinase inhibitor (TKI) initially developed as a c-Met inhibitor, and subsequently was approved for inhibition of ALK which is useful in the treatment of NSCLC patients harboring the EML4-ALK fusion protein (Kwak et al., New Eng. J. of Med., 2010, 363, 18, 1693-1703). Crizotinib is disclosed in PCT Publication No. WO 2006/021884 and U.S. Pat. No. 7,858,643. Acquired resistance to crizotinib therapy has be reported and attributed to a L1196M and a C1156Y mutation in the EL4-ALK fusion protein (Choi Y. L. et al., N. Engl. J. Med., 2010, 363, 18, 17341739). As crizotinib therapy becomes more widely available to patients harbouring the EML4ALK gene fusion event, it is likely that the L1196M and C1156Y mutations and possibly other mutations will play a more prevalent role in acquired resistance to crizotinib therapy. See, e.g., Morris et al. United States Patent Publication Number 2011/0256546 describing other ALK inhibitor resistance mutations occurring in the ALK kinase domain of the related gene fusion NPM-ALK). Accordingly, there is a need for ALK inhibitors and EML4-ALK inhibitors that have an appropriate pharmacological profile, for example in terms of potency, selectivity, pharmacokinetics, ability to cross the blood brain barrier and duration of action. More specifically, there is a need for ALK inhibitors that inhibit the EML4-ALK fusion protein having a L1196M and/or C1156Y mutation. Inhibitors with this kind of profile are reported in WO 2013/132376 where the three concatenated aromatic rings which make up crizotinib are constrained into a macrocyclic ring. This constraint offers several advantages, such as the use of the ring to restrict the free rotation about several of the linking bonds in crizotinib, and constrain the molecule into a conformation which closely resembles the optimal enzyme-binding conformation. Such molecules use internal energy to replace the organizational enthalpy and entropy which are normally subtracted from the free energy of binding, leading to higher binding free energies, and consequently higher binding efficacies. Furthermore such constraints into a ring appear to facilitate absorbance relative to uncyclized congeners, and reduce susceptibility to a lot of normal degradative metabolic process, leading to better overall drug exposures for a given quantity of drug dosed. The present invention relates to novel macrocyclic ALK inhibitors which offer superior binding and efficacy profiles, making them suitable for treating both initial EML4-ALK fusion-containing NSCLCs, and those which have acquired resistance to crizotinib via point mutations.

Crizotinib is a more potent inhibitor of c-Met than it is of ALK, and it also potently inhibits a number of other kinases, including STE20-like kinase SLK, which is important in microtubule organization and cellular movement, ROS1, which like ALK is implicated in NSCLC as a fusion protein, RON, which is implicated in gastric and pancreatic cancers, LTK, which is implicated in leukemias caused by common point mutations, EPHB6, which has possibly transforming and tumor-suppressive effects in different tissues, and may be involved with blood pressure regulation, and AXL which has been implicated as a causative mutation in a number of cancers, and which may be an important initiator of resistance to therapy in others. It is also a potent inhibitor of several common resistance mutants of the BCR-ABL oncogene of chronic myelogenous leukemia. Crizotinib has moderate, but potentially developable inhibitory, potency against a variety of other kinases, including Aurora kinases A and B, the B cell kinase BLK, which has potential use in autoimmune inflammatory conditions, HPK1, which has potential use in cancer immunotherapy, IRAK1 & 3, which also have potential use in autoimmune inflammatory conditions, the Src-family kinase LCK, STK10, another immune-associated kinase, several of the immune MAPK cascade activating MEKKs, TIE 1 & 2, angiogenic kinases, and the three neuronal TRK receptor tyrosine kinases. As described in the previous paragraph, the use of cyclization to lock in conformations can be used to produce conformations which favor binding to some kinases over others that a less constrained acyclic congener would have bound to. Although WO 2013/132376 teaches towards compounds with conformations which now bind preferentially to ALK over c-Met, other methods of cyclizing the crizotinib-like pharmacophore will stabilize different conformations of the pharmacophore leading to preferential binding to kinases other than ALK. Therefore, although many compounds of the current invention will be potent and selective ALK inhibitors, other compounds will show enhanced potency and selectivity over crizotinib for other kinases, with the kinases named in the above paragraph being those most likely to be inhibited in a therapeutically useful manner.

Anaplastic lymphoma kinase (ALK) is a member of the receptor tyrosine kinase Superfamily (RTKs), and at an amino acid sequence level is in a subfamily most closely related to Ros-1, leucocyte tyrosine kinase, the insulin receptor, insulin-like growth factor 1 receptor IGF1R and cMet (hepatic growth factor receptor) (Kostich M et al, Genome Biology, 2002, 3, 1-12). As with all members of this gene family, it possesses an extracellular ligand binding domain, a transmembrane spanning sequence, and an intracellular kinase catalytic region/signaling domain. The identity of the signaling ligand for ALK is not yet elucidated and different mechanisms have been proposed in the literature (Stoica G. E. et al, J. Biol. Chem., 2001, 276, 16772-16779; Stoica G. E. et al, J. Biol. Chem., 2002, 277, 35990-35999; Mewng K. et al, PNAS, 2000, 97, 2603-2608; Perez-Pinera P. et al, J. Biol. Chem., 2007, 282, 28683-28690). Stimulation of ALK leads to the kinase becoming activated, and an intracellular signaling cascade via phopholipase-C, PI3Kinase and STAT3 (amongst other signaling proteins) (Turner S. D. et al, Cell Signal, 2007, 19, 740-747).

ALK is largely expressed in the developing nervous system (Iwahara T. et al, Oncogene, 1997, 14, 439-449). Its relative abundance does tend to decrease in the adult animal, though its expression is maintained in certain regions of the brain, spinal cord and the eye (Vernersson E. et al, Gene Expression Patterns, 2006, 6, 448-461).

ALK has an important role in oncology (Webb T. R. et al, Expert Reviews in Anticancer Therapy, 2009, 9, 331-355). Point mutations in the full length ALK enzyme that lead to activation of the enzyme, and also an increase in expression of the full length enzyme, have both been shown to lead to neuroblastoma. In addition, the fusion of ALK with other proteins due to genetic translocation events has also been shown to lead to spontaneous activation of the kinase domain, which has been associated with cancer. A number of such ALK translocations leading to gene fusions are seen in lymphomas, the most prevalent being the nucleophosmin NPM-ALK fusion seen in anaplastic large cell lymphomas. ALK fusion with EML4 leads to a chimeric protein (EML4-ALK) thought to be responsible for transformation in 3-5% of non-small cell lung adenocarcinomas (NSCLC) (Soda M. et aL, Nature, 2007, 448, 561-567).

The cellular Met protein is a heterodimeric transmembrane protein synthesized as a single chain 190 kd precursor which is proteolytically cleaved [G. A. Rodrigues et al., Mol. Cell Biol. 11: 2962-70 (1991)]. The intracellular domain contains a juxtamembrane domain, the kinase domain and a C-terminal domain, which mediates the downstream signalling. c-Met is uniquely activated by hepatocyte growth factor (HGF), also known as scatter factor, and its splice variants, which is its only known biologically active ligand [L. Naldini et al., Oncogene 6: 501-4 (1991)]. HGF is expressed by mesenchymal cells, and its binding to c-Met, which is widely expressed in particular in epithelial cells, results in pleiotropic effects in a variety of tissues including epithelial, endothelial, neuronal and hematopoetic cells. The effects generally include one or all of the following phenomena: i) stimulation of mitogenesis; ii) stimulation of invasion and migration; and iii) stimulation of morphogenesis (tubulogenesis). Furthermore, evidence from genetically modified mice and from cell culture experiments indicate that c-Met acts as a survival receptor and protects cells from apoptosis [N. Tomita et al., Circulation 107: 1411-1417 (2003); S. Ding et al., Blood 101: 4816-4822 (2003); Q. Zeng et al., J. Biol. Chem. 277: 25203-25208 (2002); N. Horiguchi et al., Oncogene 21: 1791-1799 (2002); A. Bardelli et al., Embo J 15: 6205-6212 (1996); P. Longati et al., Cell Death Differ. 3: 23-28 (1996); E. M. Rosen, Symp. Soc. Exp. Biol. 47: 227-234 (1993)]. The coordinated execution of these biological processes by HGF results in a specific genetic program which is termed as “invasive growth”. Under normal conditions, c-Met and HGF are essential for embryonic development in mice, but the physiological role of c-Met/HGF in the adult organism is less well understood, but experimental evidence suggests that they are involved in wound healing, tissue regeneration, hemopoiesis and tissue homeostasis.

The identification of the oncoprotein TPR-MET was a first hint that c-Met may play a role in tumourigenesis. Additional substantial evidence is derived from a number of different experimental approaches. Overexpression of c-Met or HGF in human and murine cell lines induces tumourigenicity and a metastatic phenotype when expressed in nude mice. Transgenic overexpression of c-Met or HGF induces tumourigenesis in mice. Most intriguingly, missense mutations of c-Met or mutations which activate the receptor have been identified in sporadic and hereditary papillary kidney carcinomas (HPRC) as well as in other cancer types like lung, gastric, liver, head and neck, ovarian and brain cancers. Significantly, specific c-Met mutations in HPRC families segregate with disease, forming a causal link between c-Met activation and human cancer [L. Schmidt et a I., Nat. Genet. 16: 68-73 (1997); B. Zbar et al., Adv. Cancer Res. 75: 163-201 (1998)]. Activation mutations with the strongest transforming activities are located in the activation loop (D1228N/H and YI230H/D/C) and in the adjacent P+1 loop (M1250T). Additional weaker mutations have been found near the catalytic loop and within the A lobe of the kinase domain. Furthermore, some mutations in the juxtamembrane domain of c-Met have been observed in lung tumors which do not directly activate the kinase, but rather stabilize the protein by rendering it resistant to ubiquitination and subsequent degradation [M. Kong-Beltran et al., Cancer Res. 66: 283-9 (2006); T. E. Taher et al., J Immunol. 169: 3793-800 (2002); P. Peschard et al., Mol. Cell 8: 995-1004 (2001)]. All of this evidence has made inhibition of the kinase activity of the c-Met receptor a high priority as an anti-cancer target.

The abl gene and the bcr gene are normal genes located on chromosome 9 and 22, respectively. Two fusion genes are created by the reciprocal translocation between these two genes: the bcr-abl gene located on chromosome 22q- and the abl-bcr gene located on chromosome 9q+. The protein of 210 kD (p210Bcr-Abl) is encoded by the bcr-abl gene on the Philadelphia chromosome. The Abl part of the Bcr-Abl protein comprising the Abl tyrosine kinase is strictly regulated in the prototype c-Abl but continuously activated in the Bcr-Abl fusion protein, which results in cell growth disorder. The Bcr-Abl protein can be found in 95% of the patients with Chronic Myelogenous Leukemia (CML) and in 10-25% of the patients with Acute Lymphoblastic Leukemia (ALL). Imatinib, brand-named as Gleevec, is a Bcr-Abl tyrosine kinase inhibitor and has been clinically proven to be an effective formulation for the treatment of CML. (Druker et al. N. Engl. J. Med. 2006, 355, 2408). However, despite continuous treatment with Imatinib, some patients with initially responsive CMLs become resistant to the drug, this being much more common if treatment is initiated in the terminal phase or the blast crisis phase. The molecular basis of drug resistance in almost all of these cases is due to acquisition of new mutations in the kinase domain of the Bcr-Abl protein, which reduce the ability of Imatinib to compete with ATP in the active site of the enzyme. To date, more than 22 mutants have been reported and the most common ones are M244V, G250E, Q252H, Y253H, E255K, E255V, F311L, T351I, F317L, F359V, V379I, L387M, H396P, H396R and etc. (Nardi, et al. Curr. Opin. Hematol. 2004, 11, 35). Other inhibitors, such as Dasatinib, usually produce a strong therapeutic response when given to Imatinib-failure patients, demonstrating that inhibitors of these further mutated Bcr-Abl kinases are themselves valuable potential therapeutics, and there has been a major effort to find such inhibitors.

Trk's are the high affinity receptor tyrosine kinases activated by a group of soluble growth factors called neurotrophins (NT). The Trk receptor family has three members—TrkA, TrkB and TrkC. Among the neurotrophins are (i) nerve growth factor (NGF) which activates TrkA, (ii) brain-derived neurotrophic factor (BDNF) and NT-4/5 which activate TrkB and (iii) NT3 which activates TrkC. Trk's are widely expressed in neuronal tissue and are implicated in the maintenance, signaling and survival of neuronal cells (Patapoutian, A. et al., Current Opinion in Neurobiology, 2001, 11, 272-280). Recent literature has also shown that overexpression, activation, amplification and/or mutation of Trks are associated with many cancers including neuroblastoma (Brodeur, G. M., Nat. Rev. Cancer 2003, 3, 203-216), ovarian cancer (Davidson. B., et al., Clin. Cancer Res. 2003, 9, 2248-2259), breast cancer (Kruettgen et al, Brain Pathology 2006, 16: 304-310), prostate cancer (Dionne et al, Clin. Cancer Res. 1998, 4(8): 1887-1898), pancreatic cancer (Dang et al, Journal of Gastroenterology and Hepatology 2006, 21(5): 850-858), multiple myeloma (Hu et al, Cancer Genetics and Cytogenetics 2007, 178: 1-10), astrocytoma amd medulloblastoma (Kruettgen et al, Brain Pathology 2006, 16: 304-310) glioma (Hansen et al, Journal of Neurochemistry 2007, 103: 259-275), melanoma (Truzzi et al, Journal of Investigative Dermatology 2008, 128(8): 2031-2040, thyroid carcinoma (Brzezianska et al, Neuroendocrinology Letters 2007, 28(3), 221-229.), lung adenocarcinoma (Perez-Pinera et al, Molecular and Cellular Biochemistry 2007, 295(1&2), 19-26), large cell neuroendocrine tumors (Marchetti et al, Human Mutation 2008, 29(5), 609-616), and colorectal cancer (Bardelli, A., Science 2003, 300, 949). In preclinical models of cancer, non-selective small molecule inhibitors of Trk A, B and C and Trk/Fc chimeras were efficacious in both inhibiting tumor growth and stopping tumor metastasis (Nakagawara, A. (2001) Cancer Letters 169:107-114; Meyer, J. et al. (2007) Leukemia, 1-10; Pierottia, M. A. and Greco A., (2006) Cancer Letters 232:90-98; Eric Adriaenssens, E. et al. Cancer Res (2008) 68:(2) 346-351) (Truzzi et al, Journal of Investigative Dermatology 2008, 128(8): 2031-2040. Because of these strong associations, Trk inhibitors have been examined in the clinic as anti-cancer agents, and there is interest in finding new and better Trk inhibitors.

The TIE1 & 2 RTKs are primarily expressed on vascular epithelial cells, where they are receptors for the Angiopoietins, and have been shown to be required for both embryonic blood vessel development and tumor vascularization. In this latter role, inhibitors of the TIEs, which usually also hit a wide variety of other vascular system RTKs, such as the PDGFR and VEGFR RTKs, have been shown to have strong antitumoral properties. Therefore there is considerable interest in finding new and potent TIE inhibitors, especially those which show some selectivity over VEGFR, inhibition of which has been associated with hypertensive liabilities.

### SUMMARY OF THE INVENTION

The present invention provides, in part, novel compounds and pharmaceutically acceptable salts thereof that can selectively modulate the enzyme activity of a number of protein kinases, including, but not limited to, ALK and/or EML4-ALK, AXL, Aur B & C, mutant BCR-ABL, BLK, Eph6B, HPK, IRAK1 & 3, LCK, LTK, various MEKKs, RON, ROS1, SLK, STK10, TIE1 & 2, and TRKs1-3 thereby affecting biological functions, including but not limited to inhibiting cell proliferation and cell invasiveness, inhibiting metastasis, inducing apoptosis or inhibiting angiogenesis. Also provided are pharmaceutical compositions and medicaments, comprising the compounds or salts of the invention, alone or in combination with other therapeutic agents or palliative agents.

The present invention relates to a compound of the formula (I)

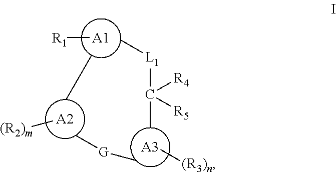

or a pharmaceutically acceptable salt thereof, wherein:

A1 is

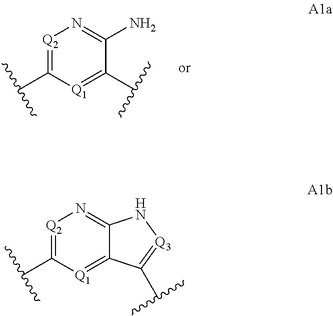

Q₁ is N or CH;

Q₂ is N or CH;

Q₃ is N or C-J-R₁;

A2 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;

A3 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;

J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡O—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;

L₁ is —O—, —S(O)x-, —C(═O)—, —CF₂—, —C(R₄)₂—, —N(R₄)—, or S(O)(═NR₄)— or is a bond;

G is

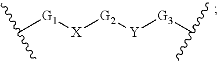

G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, —O(CR₉R₁₀)ₒ—, —S(O)ₓ(CR₉R₁₀)ₒ—, or —NR₈(CR₉R₁₀)ₒ— or a bond;

X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—,

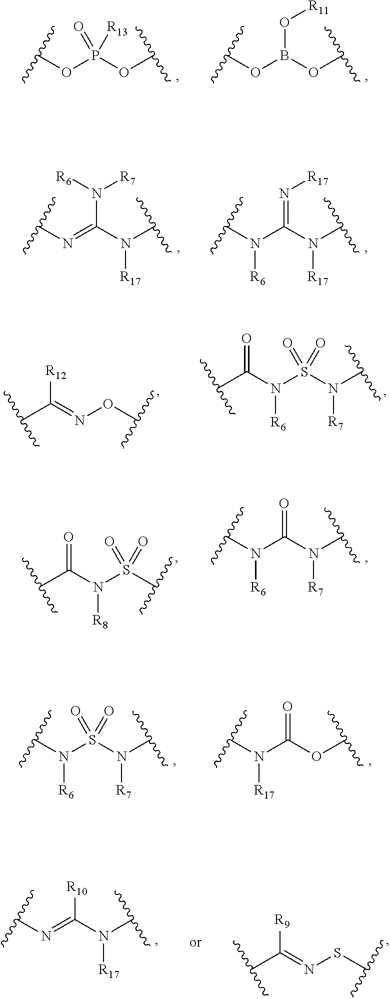

or a divalent ring system selected from the group consisting of:

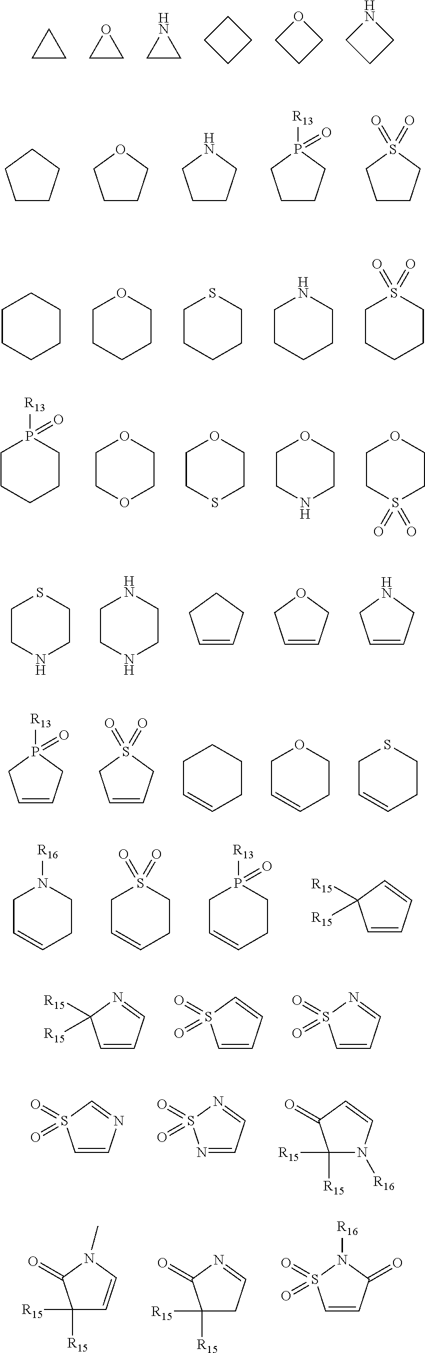

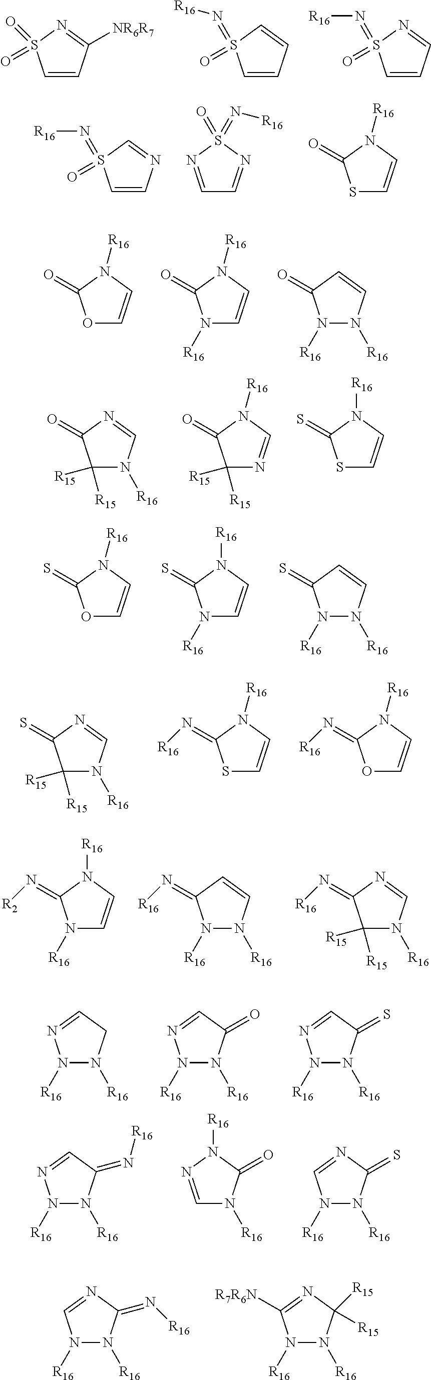

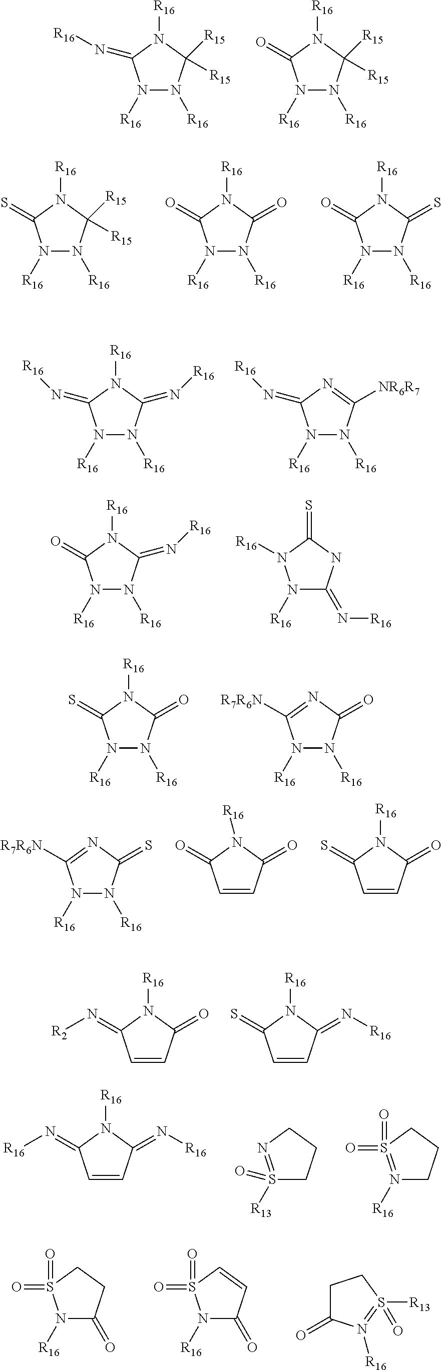

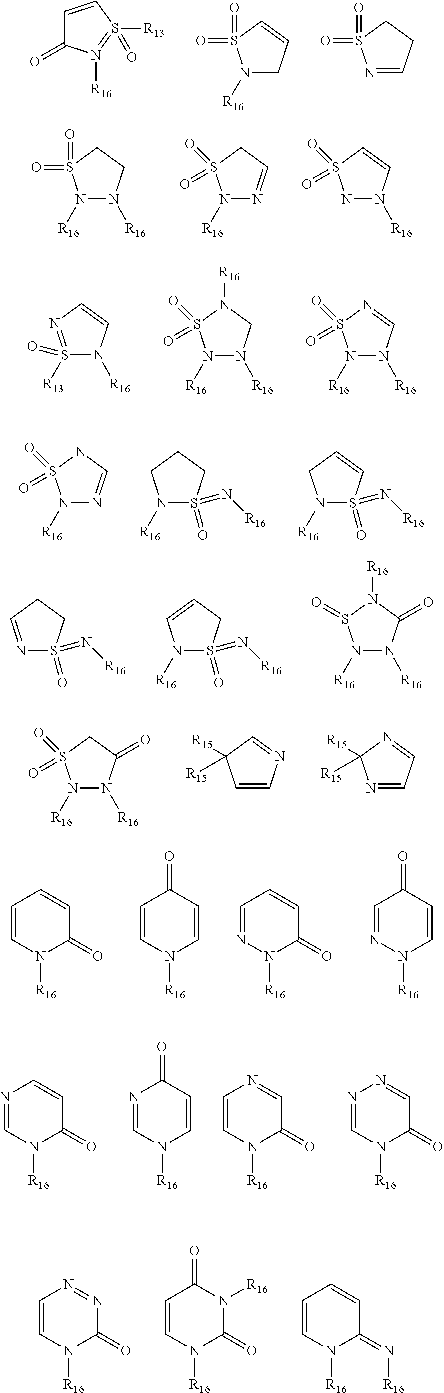

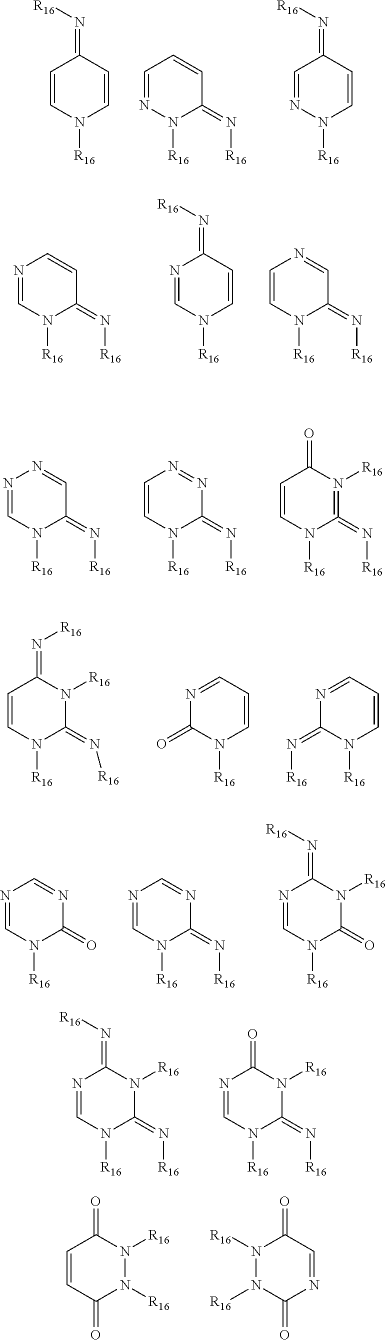

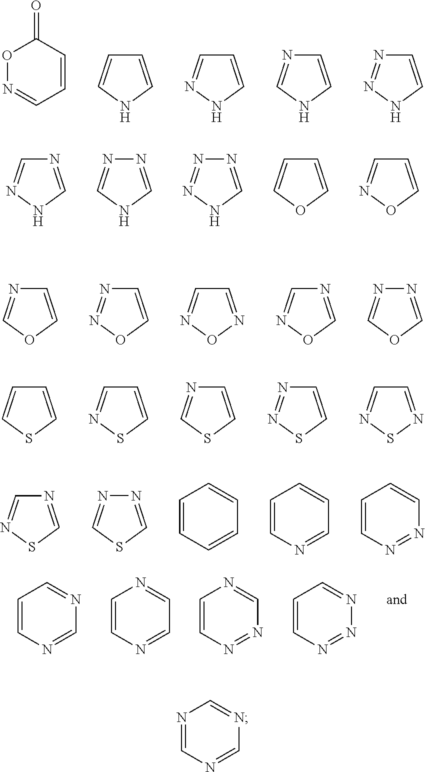

wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;

R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;

each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, —CF₃, —OCF₃, halogen, hydroxy, —NHOH, —NR₈OR₁₁, hydrazino, cyano, nitro, azido, —NR₆R₇, —ONR₆R₇, —O(C₁-C₆ alkyl), —O(C₃-C₆ alkenyl), —O(C₃-C₆ alkynyl), —O(C₃-C₆ cycloalkyl), —O(C₃-C₇ cycloalkenyl), —COR₁₂, —OCOR₁₂, —N(R₈)COR₁₂, —ON(R₈)COR₁₂, —CO₂R₁₁, —CONR₆R₇, —NR₈CONR₆R₇, —NR₈CO₂R₁₁, —ONR₈CO₂R₁₁, —OCO₂R₁₂, —OCONR₆R₇, —S(O)ₓR₁₃, —S(R₁₃)(═O)═NR₈, —S(═O)(═NR₈)NR₆R₇, —SO₂NR₆R₇, —NR₈SO₂R₁₃, —ONR₈SO₂R₁₃, —NR₈SO₂NR₆R₇, —NR₈S(═O)(═NR₈)R₁₃, —N═S(═O)(R₁₃)R₁₃, —N═S(═O)(NR₆R₇)R₁₃, —P(O)(R₁₄)₂, —P(O)(OR₁₁)R₁₃, —P(O)(OR₁₁)₂, —P(O)(NR₆R₇)OR₁₁, —P(O)(NR₆R₇)R₁₃, —P(O)(NR₆R₇)₂, —OP(O)(R₁₄)₂, —OP(O)(OR₁₁)R₁₃, —OP(O)(OR₁₁)₂, —OP(O)(NR₆R₇)OR₁₁, —OP(O)(NR₆R₇)R₁₃, —OP(O)(NR₆R₇)₂, —NR₈P(O)(R₁₄)₂, —NR₈P(O)(OR₁₁)R₁₃, —NR₈P(O)(OR₁₁)₂, —NR₈P(O)(NR₆R₇)OR₁₁, —NR₈P(O)(NR₆R₇)R₁₃, —NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;

each R₃ is independently selected from hydrogen, halogen, C₁-C₆ alkyl, C₂-C₆ alkenyl, C₂-C₆ alkynyl, cyano, nitro, azido, —CHO, —CH₂F, —CHF₂, —CF₃, —O(C₁-C₆ alkyl), and —S(O)ₓ(C₁-C₆ alkyl);

each R₄ and R₅ is independently selected from hydrogen, —CH₂F, —CHF₂, —CF₃, C₁-C₆ alkyl, C₂-C₆ alkenyl, C₂-C₆ alkynyl, and C₃-C₆ cycloalkyl;

each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);

each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;

each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, —OR₁₁, —NR₆R₇, —ONR₆R₇, —O(C₁-C₆ alkyl), —O(C₃-C₆ alkenyl), —O(C₃-C₆ alkynyl), —O(C₃-C₆ cycloalkyl), —O(C₃-C₇ cycloalkenyl), —COR₁₂, —OCOR₁₂, —N(R₈)COR₁₂, —ON(R₈)COR₁₂, —CO₂R₁₁, —CONR₆R₇, —NR₈CONR₆R₇, —ONR₈CONR₆R₇, —NR₈CO₂R₁₁, —ONR₈CO₂R₁₁, —OCO₂R₁₂, —OCONR₆R₇, —S(O)ₓR₁₃, —S(R₁₃)(═O)═NR₈, —S(═O)(═NR₈)NR₆R₇, —SO₂NR₆R₇, —NR₈SO₂R₁₃, —NR₈SO₂NR₆R₇, —ONR₈SO₂R₁₃, —ONR₈SO₂NR₆R₇, —NR₈S(═O)(═NR₈)R₁₃, —N═S(═O)(R₁₃)R₁₃, —N═S(═O)(NR₆R₇)R₁₃, —P(O)(R₁₄)₂, —P(O)(OR₁₁)R₁₃, —P(O)(OR₁₁)₂, —P(O)(NR₆R₇)OR₁₁, —P(O)(NR₆R₇)R₁₃, —P(O)(NR₆R₇)₂, —OP(O)(R₁₄)₂, —OP(O)(OR₁₁)R₁₃, —OP(O)(OR₁₁)₂, —OP(O)(NR₆R₇)OR₁₁, —OP(O)(NR₆R₇)R₁₃, —OP(O)(NR₆R₇)₂, —NR₈P(O)(R₁₄)₂, —NR₈P(O)(OR₁₁)R₁₃, —NR₈P(O)(OR₁₁)₂, —NR₈P(O)(NR₆R₇)OR₁₁, —NR₈P(O)(NR₆R₇)R₁₃, and —NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);

each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;

each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;

each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;

each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);

each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, —NR₆R₇, —O(C₁-C₆ alkyl), —O(C₃-C₆ alkenyl), —O(C₃-C₆ alkynyl), —O(C₃-C₆ cycloalkyl), —O(C₃-C₇ cycloalkenyl), —COR₁₂, —OCOR₁₂, —N(R₈)COR₁₂, —CO₂R₁₁, —CONR₆R₇, —NR₈CONR₆R₇, —NR₈CO₂R₁₁, —OCO₂R₁₂, —OCONR₆R₇, —S(O)ₓR₁₃, —S(R₁₃)(═O)═NR₈, —S(═O)(═NR₈)NR₆R₇, —SO₂NR₆R₇, —NR₈SO₂R₁₃, —NR₈SO₂NR₆R₇, —NR₈S(═O)(═NR₈)R₁₃, —ONR₆R₇, —ON(R₈)COR₁₂, —ONR₈CONR₆R₇, —ONR₈CO₂R₁₁, —ONR₈SO₂R₁₃, —ONR₈SO₂NR₆R₇, —N═S(═O)(R₁₃)R₁₃, —N═S(═O)(NR₆R₇)R₁₃, —P(O)(R₁₄)₂, —P(O)(OR₁₁)R₁₃, —P(O)(OR₁₁)₂, —P(O)(NR₆R₇)OR₁₁, —P(O)(NR₆R₇)R₁₃, —P(O)(NR₆R₇)₂, —OP(O)(R₁₄)₂, —OP(O)(OR₁₁)R₁₃, —OP(O)(OR₁₁)₂, —OP(O)(NR₆R₇)OR₁₁, —OP(O)(NR₆R₇)R₁₃, —OP(O)(NR₆R₇)₂, —NR₈P(O)(R₁₄)₂, —NR₈P(O)(OR₁₁)R₁₃, NR₈P(O)(OR₁₁)₂, —NR₈P(O)(NR₆R₇)OR₁₁, —NR₈P(O)(NR₆R₇)R₁₃, —NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;

each R₁₆ is independently selected from hydrogen, C₁-C₆ alkyl, C₂-C₆

alkenyl, C₂-C₆ alkynyl, C₃-C₆ cycloalkyl, C₃-C₇ cycloalkenyl, CF₃, hydroxy, —O(C₁-C₆ alkyl), —COR₁₂, —CO₂R₁₁, —CONR₆R₇, —S(O)ₓR₁₃, —S(R₁₃)(═O)═NR₈, —SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —P(O)(R₁₄)₂, —P(O)(OR₁₁)R₁₃, —P(O)(OR₁₁)₂, —P(O)(NR₆R₇)OR₁₁, —P(O)(NR₆R₇)R₁₃, and —P(O)(NR₆R₇)₂, or is a bond to A2, A3, X, Y, G₁, G₂ or G₃;

each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, —CF₃, —COR₁₂, —CO₂R₁₁, —CONR₆R₇, —S(O)ₓR₁₃, —S(R₁₃)(═O)═NR₈, —SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —P(O)(R₁₄)₂, —P(O)(OR₁₁)R₁₃, —P(O)(OR₁₁)₂, —P(O)(NR₆R₇)OR₁₁, —P(O)(NR₆R₇)R₁₃, or —P(O)(NR₆R₇)₂, or two R₁₇ groups together with the nitrogen atom to which they are attached form a 4-7 membered heterocyclyl;

m is 0-3;

n is 0-2;

o is 0-3;

p is 0-2; and

x is 0-2;

with the proviso that when A1 is A1a, and only one out of X and Y is present, and G contains one heteroatom selected from N and O within the direct chain that links A2 and A3, and G does not contain a carbon-carbon double bond in the direct chain that links A2 and A3, and R₃ is halogen or C₁₋₆ alkyl, and none of R₂, R₉, R₁₀, R₁₅ and R₁₆ contain phosphorus, a sulfur-nitrogen double bond, sulfur bonded to two nitrogen atoms, nitrogen bonded to one oxygen atom, a carbonate, a carbamate or a urea; then neither X nor Y is selected from the group consisting of: O, NR₈, CONR₈, NR₈CO,

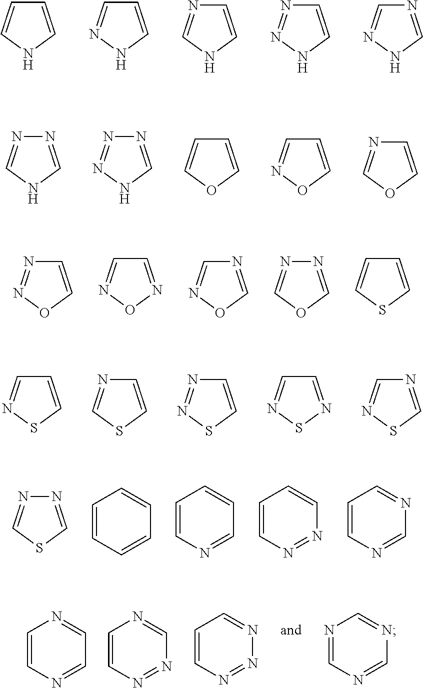

and any R₁₅ or R₁₆ substituent bound to a divalent ring system at a position vicinal to, or vinylogously linked to, C═O, C═N or C═S is not H if tautomerization at such position could lead to aromatization of the divalent ring system.

In one embodiment, the invention relates to a pharmaceutical composition comprising a compound of one of the formulae disclosed herein, or a pharmaceutically acceptable salt thereof, and a pharmaceutically acceptable carrier or excipient.

Another embodiment relates to methods of treating or inhibiting cell proliferation, cell invasiveness, metastases, apoptosis, or angiogenesis in a mammal. In other embodiments, the cell proliferation, cell invasiveness, metastases, apoptosis, or angiogenesis is mediated by ALK, an EML-4 fusion protein, AXL, Aur B & C, mutant BCR-ABL, BLK, Eph6B, HPK, IRAK1 & 3, LCK, LTK, various MEKKs, RON, ROS1, SLK, STK10, TIE1 & 2, and TRKs1-3.

In other embodiments, the compounds of the invention may be combined with other therapeutic or palliative agents. In such embodiments, the amounts of the two or more agents together are effective in treating or inhibiting the cell proliferation, cell invasiveness, metastases, etc. The therapeutic agents include anti-cancer agents such as anti-tumor agents, anti-angiogenesis agents, and antiproliferative agents.

For the purposes of this disclosure, it is understood that each of the embodiments of the compounds of the present invention set forth herein can be combined with any other embodiment describing the compounds provided that such embodiments are not inconsistent with one another.

### DETAILED DESCRIPTION OF THE PREFERRED EMBODIMENTS

As used herein, the term “halogen” or “halo” refers to fluoro, chloro, bromo, or iodo (F, Cl, Br, I). Preferably, halo refers to fluoro or chloro.

The term “alkyl” refers to a saturated, monovalent aliphatic hydrocarbon radical including straight chain and branched chain groups having the specified number of carbon atoms. The term “C₁₋₆ alkyl” or “C₁-C₆ alkyl” refers to a branched or straight chained alkyl radical containing from 1 to 6 carbon atoms, such as methyl, ethyl, n-propyl, isopropyl, n-butyl, isobutyl, sec butyl, t-butyl, pentyl, hexyl, and the like. Similarly, the term ““C₁₋₄ alkyl” or “C₁-C₄ alkyl” refers to a branched or straight chained alkyl radical containing from 1 to 4 carbon atoms, such as methyl, ethyl, n-propyl, isopropyl, n-butyl, isobutyl, sec-butyl, t-butyl, and the like.

In some instances, substituted alkyl groups may be specifically named with reference to the substituent group. For example, “haloalkyl” refers to an alkyl group having the specified number of carbon atoms that is substituted by one or more halo substituents, and typically contain 1-6 carbon atoms and 1, 2 or 3 halo atoms (i.e., “C₁-C₆ haloalkyl”). Thus, a C₁-C₆ haloalkyl group includes trifluoromethyl (—CF₃) and difluoromethyl (—CF₂H).

Similarly, “hydroxyalkyl” refers to an alkyl group having the specified number of carbon atoms that is substituted by one or more hydroxy substituents, and typically contain 1-6 carbon atoms and 1, 2 or 3 hydroxy (i.e., “C₁-C₆ hydroxyalkyl”). Thus, C₁-C₆ hydroxyalkyl includes hydroxymethyl (—CH₂OH) and 2-hydroxyethyl (—CH₂CH₂OH).

The term “C₁₋₆ alkoxy”, “C₁-C₆ alkoxy” or “OC₁₋₆ alkyl” refers to a straight or branched alkoxy group containing from 1 to 6 carbon atoms, such as methoxy, ethoxy, n-propoxy, isopropoxy, n-butoxy, isobutoxy, sec-butoxy, t-butoxy, pentoxy, hexoxy, and the like. The term “C₁₋₄ alkoxy”, “C₁-C₄ alkoxy”, “OC₁₋₄ alkyl” refers to a straight or branched alkoxy group containing from 1 to 4 carbon atoms, such as methoxy, ethoxy, n-propoxy, isopropoxy, n-butoxy, isobutoxy, sec-butoxy, t-butoxy, and the like.

The term “C₃₋₆ cycloalkoxy”, “C₃-C₆ cycloalkoxy”, or “OC₃₋₆ cycloalkyl” refers to a cyclic alkoxy radical containing from 3 to 6 carbon atoms such as cyclopropoxy, cyclobutoxy, cyclopentoxy, and the like.

“Alkoxyalkyl” refers to an alkyl group having the specified number of carbon atoms that is substituted by one or more alkoxy substituents. Alkoxyalkyl groups typically contain 1-6 carbon atoms in the alkyl portion and are substituted by 1, 2 or 3 C₁-C₄ alkyoxy substituents. Such groups are sometimes described herein as C₁-C₄ alkyoxy-C₁-C₆ alkyl. “Aminoalkyl” refers to alkyl group having the specified number of carbon atoms that is substituted by one or more substituted or unsubstituted amino groups, as such groups are further defined herein.

Aminoalkyl groups typically contain 1-6 carbon atoms in the alkyl portion and are substituted by 1, 2 or 3 amino substituents. Thus, a C₁-C₆ aminoalkyl group includes, for example, aminomethyl (—CH₂NH₂), N,N-dimethylamino-ethyl (—CH₂CH₂N(CH₃)₂, 3-(N-cyclopropylamino)propyl (—CH₂CH₂CH₂NH—^{C}Pr) and N-pyrrolidinylethyl (—CH₂CH₂N-pyrrolidinyl).

“Alkenyl” refers to an alkyl group, as defined herein, consisting of at least two carbon atoms and at least one carbon-carbon double bond. Typically, alkenyl groups have 2 to 20 carbon atoms (“C₂-C₂₀ alkenyl”), preferably 2 to 12 carbon atoms (“C₂-C₁₂ alkenyl”), more preferably 2 to 8 carbon atoms (“C₂-C₈ alkenyl”), or 2 to 6 carbon atoms (“C₂-C₆ alkenyl”), or 2 to 4 carbon atoms (“C₂-C₄ alkenyl”). Representative examples include ethenyl, 1-propenyl, 2-propenyl, 1-, 2-, or 3-butenyl, and the like. A “C₂-C₆ alkenyl” denotes a straight-chain or branched group containing 2 to 6 carbon atoms and at least one double bond between two sp² hybridized carbon atoms. This also applies if they carry substituents or occur as substituents of other radicals, for example in O—(C₂-C₆)alkenyl radicals. Examples of suitable C₂-C₆ alkenyl radicals are n-propenyl, iso-propenyl, n-butenyl, iso-butenyl, n-pentenyl, sec-pentenyl, n-hexenyl, sec-hexenyl, and the like. Alkenyl groups may be unsubstituted or substituted by the same groups that are described herein as suitable for alkyl.

“Alkynyl” refers to an alkyl group, as defined herein, consisting of at least two carbon atoms and at least one carbon-carbon triple bond. Alkynyl groups have 2 to 20 carbon atoms (“C₂-C₂₀ alkynyl”), preferably 2 to 12 carbon atoms (“C₂-C₁₂ alkynyl”), more preferably 2 to 8 carbon atoms (“C₂-C₈ alkynyl”), or 2 to 6 carbon atoms (“C₂-C₆ alkynyl”), or 2 to 4 carbon atoms (“C₂-C₄ alkynyl”). Representative examples include, but are not limited to, ethynyl, 1-propynyl, 2-propynyl, 1-, 2-, or 3-butynyl, and the like. Alkynyl groups may be unsubstituted or substituted by the same groups that are described herein as suitable for alkyl. A “C₂-C₆ alkynyl” denotes a straight-chain or branched group containing 2 to 6 carbon atoms and at least one triple bond between two sp hybridized carbon atoms. This also applies if they carry substituents or occur as substituents of other radicals, for example in O—(C₂-C₆)alkynyl radicals. Examples of suitable C₂-C₆ alkynyl radicals are propynyl, butynyl, pentynyl, hexynyl, and the like.

“Alkylene” as used herein refers to a divalent hydrocarbyl group having the specified number of carbon atoms which can link two other groups together. Sometimes it refers to —(CH₂)ₙ— where n is 1-8, and preferably n is 1-4. Where specified, an alkylene can also be substituted by other groups and may include one or more degrees of unsaturation (i.e., an alkenylene or alkynylene moiety) or rings. The open valences of an alkylene need not be at opposite ends of the chain. Thus —CH(Me)- and —C(Me)₂- are also included within the scope of the term ‘alkylenes’, as are cyclic groups such as cyclopropan-1,1-diyl and unsaturated groups such as ethylene (—CH═CH—) or propylene (—CHrCH═CH—). Where an alkylene group is described as optionally substituted, the substituents include those typically present on alkyl groups as described herein.

“Heteroalkylene” refers to an alkylene group as described above, wherein one or more non-contiguous carbon atoms of the alkylene chain are replaced by —N—, —O— —P— or —S—, in manifestations such as —N(R)—, —P(═O)(R)—, —S(O)ₓ— or —S(═O)(═NR)—, where R is H or C₁-C₄ alkyl and x is 0-2. For example, the group —O—(CH₂)₁₋₄— is a ‘C₂-C₅’-heteroalkylene group, where one of the carbon atoms of the corresponding alkylene is replaced by O.

“Aryl” or “aromatic” refers to an all-carbon monocyclic or fused-ring polycyclic having a completely conjugated pi-electron system and possessing aromaticity. The terms “C₆-C₁₂ aryl” and “C₆₋₁₂ aryl” are included within this term and encompass aromatic ring systems of 6 to 12 carbons and containing no heteroatoms within the ring system. Examples of aryl groups are phenyl and naphthalenyl. The aryl group may be substituted or unsubstituted. Substituents on adjacent ring carbon atoms of a C₆-C₁₂ aryl may combine to form a 5- or 6-membered carbocyclic ring optionally substituted by one or more substituents, such as oxo, C₁-C₆ alkyl, hydroxyl, amino and halogen, or a 5- or 6-membered heterocyclic ring containing one, two or three ring heteroatoms selected from N, O and S(O)ₓ (where x is 0, 1 or 2) optionally substituted by one or more substituents, such as oxo, C₁-C₆ alkyl, hydroxyl, amino and halogen. Examples of aryl groups include phenyl, biphenyl, naphthyl, anthracenyl, phenanthrenyl, indanyl, indenyl, and tetrahydronaphthyl. The aryl group may be unsubstituted or substituted as further described herein.

“Heteroaryl” or “heteroaromatic” refers to monocyclic or fused bicyclic or polycyclic ring systems having the well-known characteristics of aromaticity that contain the specified number of ring atoms and include at least one heteroatom selected from N, O, and S as a ring member in an aromatic ring. The inclusion of a heteroatom permits aromaticity in 5-membered rings as well as 6-membered rings. Typically, heteroaryl groups contain 5 to 20 ring atoms (“5-20 membered heteroaryl”), preferably 5 to 14 ring atoms (“5-14 membered heteroaryl”), and more preferably 5 to 12 ring atoms (“5-12 membered heteroaryl”) or 5 to 6 ring atoms (“5-6 membered heteroaryl”). Heteroaryl rings are attached to the base molecule via a ring atom of the heteroaromatic ring, such that aromaticity is maintained. Thus, 6-membered heteroaryl rings may be attached to the base molecule via a ring C atom, while 5-membered heteroaryl rings may be attached to the base molecule via a ring C or N atom. The heteroaryl group may be unsubstituted or substituted as further described herein. As used herein, “5-6 membered heteroaryl” refers to a monocyclic group of 5 or 6 ring atoms containing one, two or three ring heteroatoms selected from N, O, and S, but including tetrazolyl with 4 nitrogens, the remaining ring atoms being C, and, in addition, having a completely conjugated pi-electron system. Substituents on adjacent ring atoms of a 5- or 6-membered heteroaryl may combine to form a fused 5- or 6-membered carbocyclic ring optionally substituted by one or more substituents, such as oxo, C₁-C₆ alkyl, hydroxyl, amino and halogen, or a fused 5- or 6-membered heterocyclic ring containing one, two or three ring heteroatoms selected from N, O, and S(O)ₓ (where x is 0, 1 or 2) optionally substituted by one or more substituents, such as oxo, C₁-C₆ alkyl, hydroxyl, amino and halogen. If said fused ring is itself aromatic, it is referred to as a fused (bicyclic) heteroaromatic species, regardless of whether the second ring contains heteroatoms. A pharmaceutically acceptable heteroaryl is one that is sufficiently stable to be attached to a compound of the invention, formulated into a pharmaceutical composition and subsequently administered to a patient in need thereof.

Examples of 5-membered heteroaryl rings containing 1, 2 or 3 heteroatoms independently selected from O, N, and S, include pyrrolyl, thienyl, furanyl, pyrazolyl, imidazolyl, oxazolyl, isoxazolyl, thiazolyl, isothiazolyl, triazolyl, tetrazolyl, oxadiazolyl and thiadiazolyl. Preferred 6-membered heteroaryl rings contain 1 or 2 nitrogen atoms. Examples of 6-membered heteroaryl are pyridyl, pyridazinyl, pyrimidinyl and pyrazinyl. Examples of fused heteroaryl rings include benzofuran, benzothiophene, indole, benzimidazole, indazole, quinolone, isoquinoline, purine, pyrrolopyrimidine, napthyridine and carbazole.

An “arylene” as used herein refers to a bivalent radical derived from an aromatic hydrocarbon by removal of a hydrogen atom from each of two carbon atoms of the nucleus. In frequent embodiments, the arylene ring is a 1,2-disubstituted or a 1,3-disubstituted arylene. The aryl ring of the arylene moiety may be optionally substituted on open valence positions with groups suitable for an aryl ring, to the extent such substitution is indicated. Preferably, the arylene ring is a C₆-C₁₂ arylene ring, for example a 1,2-phenylene or 1,3-phenylene moiety.

Similarly, a “heteroarylene” as used herein refers to a bivalent radical derived from a heteroaromatic ring by removal of a hydrogen atom from each of two carbon or a carbon atom and a nitrogen atom of the nucleus. In frequent embodiments, the heteroarylene ring is a 1,2-disubstituted or a 1,3-disubstituted heteroarylene. The heteroaryl ring of the heteroarylene moiety is optionally substituted with groups suitable for an heteroaryl ring, to the extent such substitution is indicated. Preferably, the heteroarylene ring is a 5-12 membered, possibly fused, heteroarylene ring, more preferably a 5-6 membered heteroarylene ring, each of which may be optionally substituted.

The terms “heteroalicyclic”, “heterocyclyl”, or “heterocyclic” may be used interchangeably herein to refer to a non-aromatic, saturated or partially unsaturated ring system containing the specified number of ring atoms, including at least one heteroatom selected from N, O, and S as a ring member, wherein the heterocyclic ring is connected to the base molecule via a ring atom, which may be C or N. Heteroalicyclic rings may be fused to one or more other heteroalicyclic or carbocyclic rings, which fused rings may be saturated, partially unsaturated or aromatic. Preferably, heteroalicyclic rings contain 1 to 4 heteroatoms selected from N, O, and S as ring members, and more preferably 1 to 2 ring heteroatoms, provided that such heteroalicyclic rings do not contain two contiguous oxygen atoms. Heteroalicyclic groups may be unsubstituted or substituted by the same groups that are described herein as suitable for alkyl, aryl or heteroaryl.

Preferred heteroalicyclic groups include 3-12 membered heteroalicyclic groups, 5-8 membered heterocyclyl (or heteroalicyclic) groups, 4-12 membered heteroalicyclic monocycles, and 6-12 membered heteroalicyclic bicycles in accordance with the definition herein. As used herein, “3-12 membered heteroalicyclic” refers to a monocyclic or bicyclic group having 3 to 12 ring atoms, in which one, two, three or four ring atoms are heteroatoms selected from N, O, P(O), S(O)ₓ (where x is 0, 1, 2) and S(═O)(═NR) the remaining ring atoms being C. The ring may also have one or more double bonds. However, the ring does not have a completely conjugated pi-electron system. Substituents on two ring carbon atoms may combine to form a 5- or 6-membered bridged ring that is either carbocyclic or heteroalicyclic containing one, two or three ring heteroatoms selected from N, O and S(O)ₓ (where x is 0, 1 or 2). The heteroalicyclic group is optionally substituted by oxo, hydroxyl, amino, C₁-C₆-alkyl and the like.

In frequent embodiments, heteroalicyclic groups contain 3-12 ring members, including both carbon and non-carbon heteroatoms, and preferably 4-6 ring members. In certain preferred embodiments, substituent groups comprising 3-12 membered heteroalicyclic groups are selected from azetidinyl, pyrrolidinyl, piperidinyl, piperazinyl, morpholinyl and thiomorpholinyl rings, each of which may be optionally substituted to the extent such substitution makes chemical sense.

It is understood that no more than two N, O or S atoms are ordinarily connected sequentially, except where an oxo or aza group is attached to N, P or S to form groups such as, but not limited to, nitro, phosphinyl, phosphinamido, sulfoximino and sulfonyl group, or in the case of certain heteroaromatic rings, such as triazine, triazole, tetrazole, oxadiazole, thiadiazole, and the like.

“Cycloalkyl” refers to a non-aromatic, saturated or partially unsaturated carbocyclic ring system containing the specified number of carbon atoms, which may be a monocyclic, bridged or fused bicyclic or polycyclic ring system that is connected to the base molecule through a carbon atom of the cycloalkyl ring. Typically, the cycloalkyl groups of the invention contain 3 to 12 carbon atoms (“C₃-C₁₂ cycloalkyl”), preferably 3 to 8 carbon atoms (“C₃-C₈ cycloalkyl”). Other cycloalkyl groups include partially unsaturated moieties from 4 to 7 carbons (“C₄-C₇ cycloalkenyl”). Representative examples include, e.g., cyclopropane, cyclobutane, cyclopentane, cyclopentene, cyclohexane, cyclohexene, cyclohexadiene, cycloheptane, cycloheptatriene, adamantane, and the like. Cycloalkyl groups may be unsubstituted or substituted by the same groups that are described herein as suitable for alkyl. As used herein, “C₃-C₆ cycloalkyl” refers to an all-carbon, monocyclic or fused-ring polycyclic group of 3 to 6 carbon atoms.

“Cycloalkylalkyl” may be used to describe a cycloalkyl ring, typically a C₃-C₈ cycloalkyl, which is connected to the base molecule through an alkylene linker, typically a C₁-C₄ alkylene. Cycloalkylalkyl groups are described by the total number of carbon atoms in the carbocyclic ring and linker, and typically contain from 4-12 carbon atoms (“C₄-C₁₂ cycloalkylalkyl”). Thus a cyclopropylmethyl group is a C₄-cycloalkylalkyl group and a cyclohexylethyl is a C₈-cycloalkylalkyl. Cycloalkylalkyl groups may be unsubstituted or substituted on the cycloalkyl and/or alkylene portions by the same groups that are described herein as suitable for alkyl groups.

An “arylalkyl” group refers to an aryl group as described herein which is linked to the base molecule through an alkylene or similar linker. Arylalkyl groups are described by the total number of carbon atoms in the ring and linker. Thus a benzyl group is a C₇-arylalkyl group and a phenylethyl is a C₈-arylalkyl. Typically, arylalkyl groups contain 7-16 carbon atoms (“C₇-C₁₆ arylalkyl”), wherein the aryl portion contains 6-12 carbon atoms and the alkylene portion contains 1-4 carbon atoms. Such groups may also be represented as —C₁-C₄ alkylene-C₆-C₁₂ aryl.

“Heteroarylalkyl” refers to a heteroaryl group as described above that is attached to the base molecule through an alkylene linker, and differs from “arylalkyl” in that at least one ring atom of the aromatic moiety is a heteroatom selected from N, O and S. Heteroarylalkyl groups are sometimes described herein according to the total number of non-hydrogen atoms (i.e., C, N, S and O atoms) in the ring and linker combined, excluding substituent groups. Thus, for example, pyridinylmethyl may be referred to as a “C₇”-heteroarylalkyl. Typically, unsubstituted heteroarylalkyl groups contain 6-20 non hydrogen atoms (including C, N, S and O atoms), wherein the heteroaryl portion typically contains 5-12 atoms and the alkylene portion typically contains 1-4 carbon atoms. Such groups may also be represented as —C₁-C₄ alkylene-5-12 membered heteroaryl.

Similarly, “arylalkoxy” and “heteroarylalkoxy” refer to aryl and heteroaryl groups, attached to the base molecule through a heteroalkylene linker (i.e., —O-alkylene-), wherein the groups are described according to the total number of non-hydrogen atoms (i.e., C, N, S and O atoms) in the ring and linker combined. Thus, —O—CH₂-phenyl and —O—CH₂-pyridinyl groups would be referred to as C₈-arylalkoxy and C₈-heteroarylalkoxy groups, respectively.

Where an arylalkyl, arylalkoxy, heteroarylalkyl or heteroarylalkoxy group is described as optionally substituted, the substituents may be on either the divalent linker portion or on the aryl or heteroaryl portion of the group. The substituents optionally present on the alkylene or heteroalkylene portion are the same as those described above for alkyl or alkoxy groups generally, while the substituents optionally present on the aryl or heteroaryl portion are the same as those described above for aryl or heteroaryl groups generally.

“Hydroxy” refers to an —OH group.

“Acyl” refers to a monovalent group —C(O)alkyl wherein the alkyl portion has the specified number of carbon atoms (typically C₁-C₈, preferably C₁-C₆ or C₁-C₄) and may be substituted by groups suitable for alkyl. Thus, C₁-C₄ acyl includes a —C(O)C₁-C₄ alkyl substituent, e.g., —C(O)CH₃. Similarly, “acyloxy” refers to a monovalent group —OC(O)alkyl wherein the alkyl portion has the specified number of carbon atoms (typically C₁-C₈, preferably C₁-C₆ or C₁-C₄) and may be substituted by groups suitable for alkyl. Thus, C₁-C₄ acyloxy includes a —OC(O)C₁-C₄ alkyl substituent, e.g., —OC(O)CH₃.

The term “monocyclic or bicyclic ring system” refers to a an aromatic, saturated or partially unsaturated ring system containing the specified number of ring atoms, and may optionally include one or more heteroatoms selected from N, O, and S as a ring member, wherein the heterocyclic ring is connected to the base molecule via a ring atom, which may be C or N. Included within this term are the terms “cycloalkyl”, “aryl”, “heterocyclyl”, and “heteroaryl”. Typically, the monocyclic or bicyclic ring system of the invention contain 4 to 12 members atoms (“4-12 membered monocyclic or bicyclic ring system”). Bicyclic systems may be connected via a 1,1-fusion (spiro), a 1,2-fusion (fused) or a 1, >2-fusion (bridgehead). Representative examples include cyclopentane, cyclopentene, cyclohexane, norbornyl, spiro[2.3]hexane, phenyl, biphenyl, naphthyl, anthracenyl, phenanthrenyl, pyrrolyl, thienyl, furanyl, pyrazolyl, imidazolyl, oxazolyl, isoxazolyl, thiazolyl, azetidinyl, pyrrolidinyl, piperidinyl, piperazinyl, benzothiophenyl, indolyl, and the like.

All alkyl, alkenyl, alkynyl, cycloalkyl, cycloalkenyl, monocyclic and bicyclic heterocycles, aryl (monocyclic and bicyclic), heteroaryl (monocyclic and bicyclic), cycloalkylalkyl, arylalkyl, arylalkoxy, heteroarylalkyl or heteroarylalkoxy groups (which include any C₁₋₆ alkyl, C₂₋₆ alkenyl, C₂₋₆ alkynyl, C₃₋₆ cycloalkyl, C₄₋₆ cycloalkenyl, C₆₋₁₂ bicycloalkyl, saturated monocyclic heterocycles of 4-12 atoms or saturated bicyclic heterocycles of 6-12 atoms, all C₆₋₁₂ aryl monocycles or bicycles and heteroaryl monocycles or bicycles of 6-12 atoms) can be optionally substituted with multiple substituents independently chosen from halogen, hydroxy, oxo, hydroxylamino, oximino, hydrazino, hydrazono, cyano, nitro, azido, NR₆R₇, OC₁₋₆ alkyl, OC₃₋₆ alkenyl, OC₃₋₆ alkynyl, C₁₋₆ alkyl, OC₃₋₆ cycloalkyl, OC₃₋₇ cycloalkenyl, C₁₋₆ acyl, C₁₋₆ acyloxy, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, —NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, ONR₆R₇, ON(R₈)COR₁₂, ONR₈CONR₆R₇, ONR₈CO₂R₁₁, ONR₈SO₂R₁₃, ONR₈SO₂NR₆R₇.

In a preferred embodiment, all alkyl, alkenyl, alkynyl, cycloalkyl, cycloalkenyl, monocyclic and bicyclic heterocycles, aryl (moncyclic and bicyclic), and heteroaryl (monocyclic and bicyclic) groups (which include any C₁₋₆ alkyl, C₂₋₆ alkenyl, C₂₋₆ alkynyl, C₃₋₆ cycloalkyl, C₄₋₆ cycloalkenyl, C₆₋₁₂ bicycloalkyl, saturated monocyclic heterocycles of 4-12 atoms or saturated bicyclic heterocycles of 6-12 atoms, all C₆₋₁₂ aryl monocycles or bicycles and heteroaryl monocycles or bicycles of 6-12 atoms) can be optionally substituted with from 1 to 3 substituents independently chosen from halogen, hydroxy, oxo, hydroxylamino, oximino, hydrazino, hydrazono, cyano, nitro, azido, NR₆R₇, OC₁₋₆ alkyl, OC₃₋₆ alkenyl, OC₃₋₆ alkynyl, C₁₋₆ alkyl, OC₃₋₆ cycloalkyl, OC₃₋₇ cycloalkenyl, C₁₋₆ acyl, C₁₋₆ acyloxy, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, —NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁O₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, ONR₆R₇, ON(R₈)COR₁₂, ONR₈CONR₆R₇, ONR₈CO₂R₁₁, ONR₈SO₂R₁₃, ONR₈SO₂NR₆R₇.

In preferred embodiment, the term “optionally substituted” means an optional substitution of one to three, preferably one or two groups independently selected from halo, hydroxy, cyano, nitro, C₁-C₄ alkyl, and C₁-C₄ alkoxy. Any combination or subgroupings of these substituents are also specifically contemplated.

Examples of discrete ring systems suitable to be X or Y are set forth below in Lists 1, 2, 3, and 4. When constituting X or Y, these ring systems are referred to as “divalent ring systems.” The ring systems set forth therein may be incorporated into the macrocycle in any orientation which allows for the two ring-incorporating bonds to be vicinal, 1,3-linked, or 1,4-linked.

List 1:

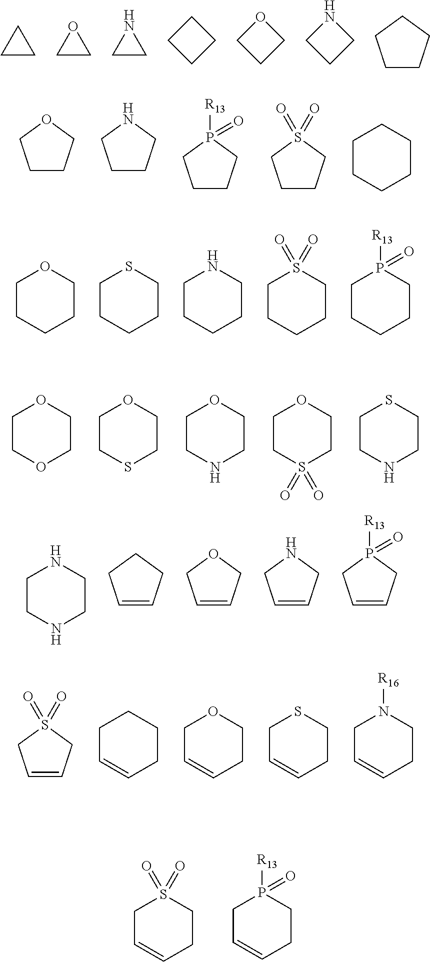

List 2:

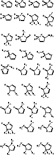

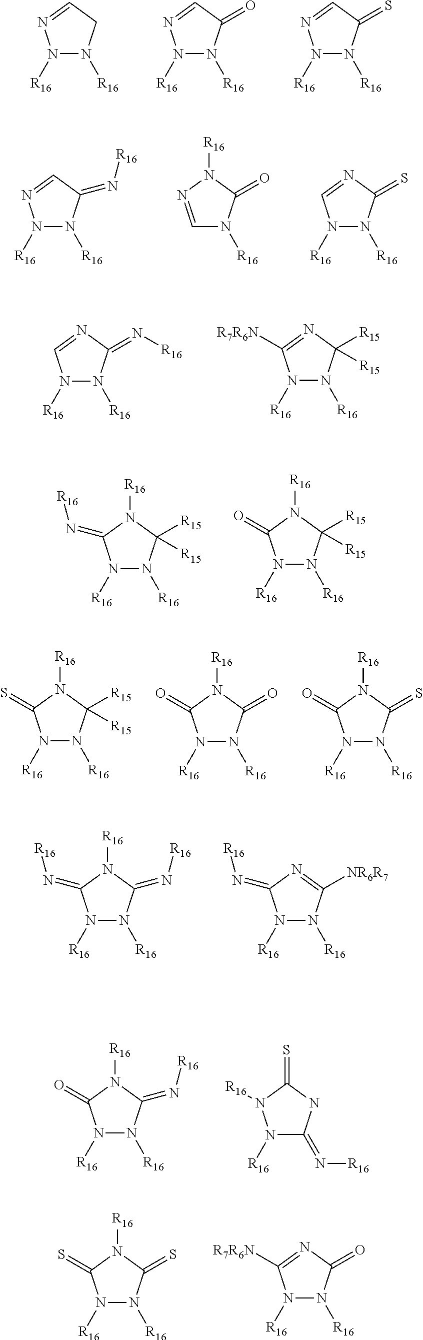

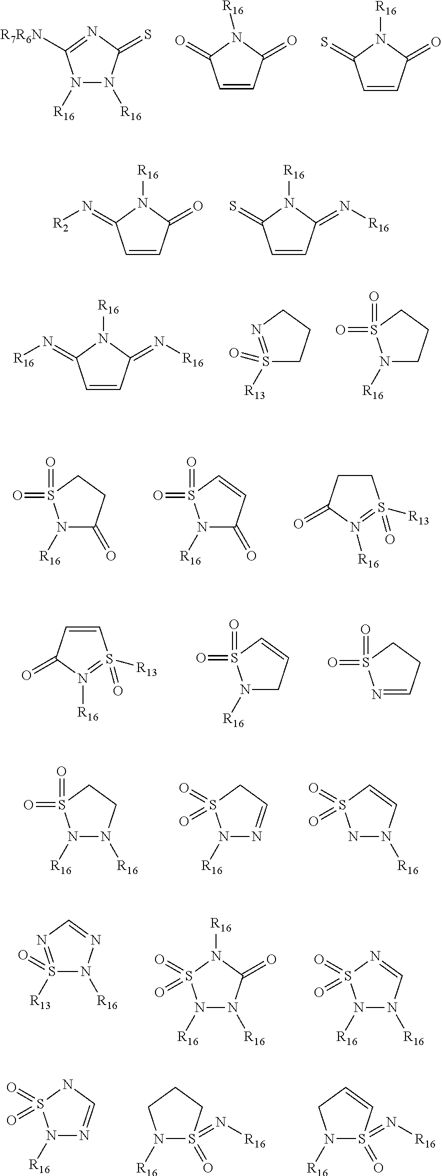

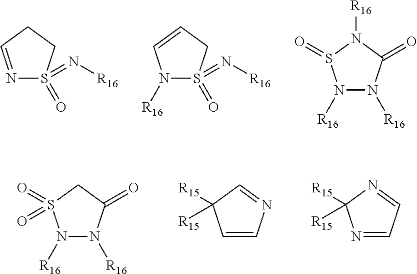

List 3:

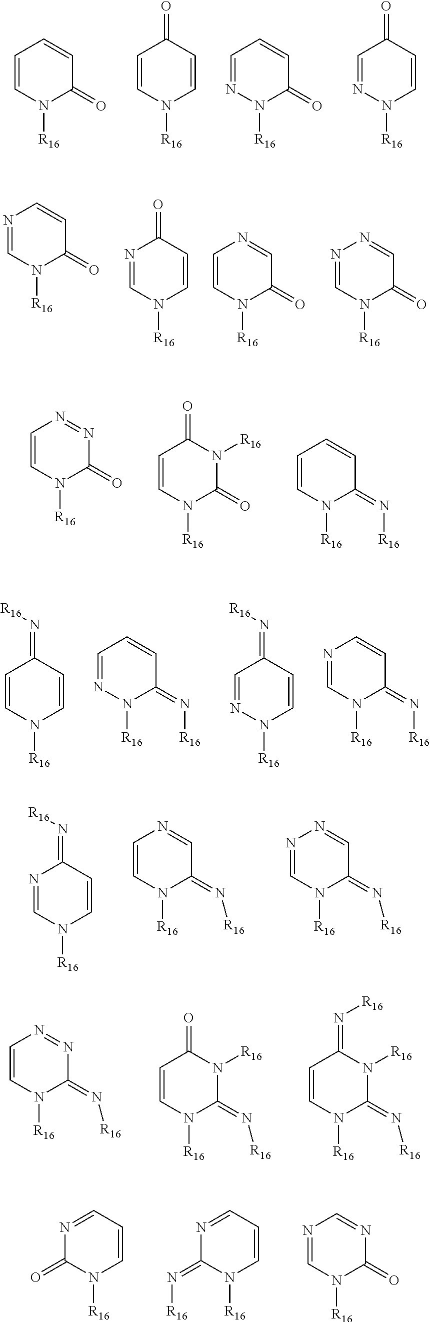

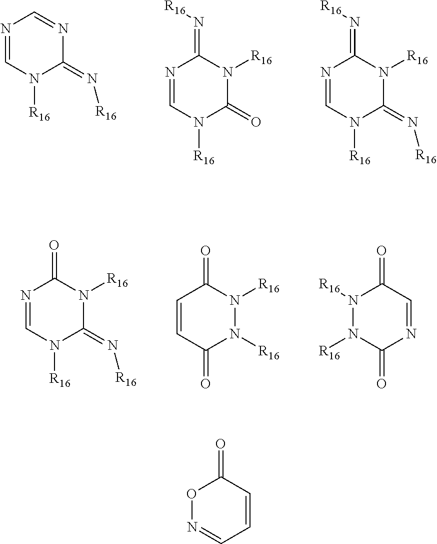

List 4:

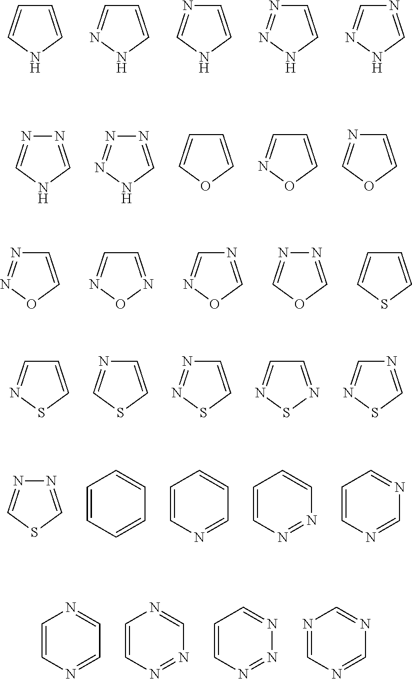

As shown in Lists 1-4, some of the divalent ring systems have one or more R₁₅ and/or R₁₆ substituents. These substituents are shown on the structures in Lists 1-4 in order to demonstrate that the listed ring systems cannot undergo tautomerization to an isomeric aromatic structure. In some instances, the R₁₅ or R₁₆ substituent is defined as “a bond to A2, A3, X, Y, G₁, G₂, or G₃.” This means that the position occupied by the R₁₅ or R₁₆ substituent is bound to one of A2, A3, X, Y, G₁, G₂, and G₃.

The term “optionally substituted” as used herein means an optional substitution of one to three, preferably one or two groups independently selected from halo, hydroxy, cyano, C₁-C₄ alkyl, and C₁-C4 alkoxy. Any combination or subgroupings of these substituents are also specifically contemplated.

The terms “geminal relationship,” “1,1-relationship,” “vicinal relationship,” “1,2-relationship,” “1,3-relationship,” “1,4-relationship,” etc. specify the spacing with which adjoining groups are attached to a ring or chain. A person of ordinary skill would understand that these terms refer to the spacings shown in the following diagram:

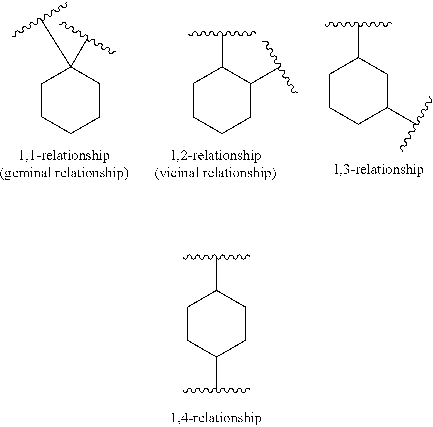 [SMILES: *C1(*)CCCCC1 ; *C1CCCCC1* ; *C1CCCC(*)C1 ; *C1CCC(*)CC1]

Unless otherwise specified, a reference to a divalent substituent or linker group shall be understood to encompass either orientation of the divalent group. For example, a reference to a “—C(═O)CR₉R₁₀—” group linking groups “A” and “B” shall be understood to include both of the following:

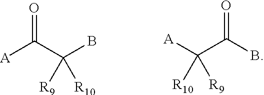

The term “direct chain that links” two groups refers to the shortest chain of atoms linking the two groups. For example, the direct chain that links A2 and A3 contains 3 atoms in the following structure:

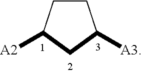

As used herein, a ring position is understood to be “vinylogously linked to C═O, C═N, or C═S” where, as a result of one or more intervening double bonds, the ring could tautomerize to formally transfer a proton from the ring position to the O, N, or S of the C═O, C═N, or C═S. For example, the nitrogen atom in pyridine-4-one is vinylogously linked to the C═O, as shown by the following tautomerization equilibrium:

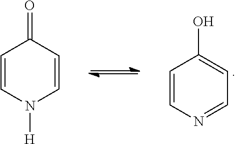 [SMILES: O=c1cc[nH]cc1 ; Oc1ccncc1]

As used in the preparations and examples the following terms have the indicated meanings; “ng” refers to nanograms; “μg” refers to micrograms; “mg” refers to milligrams; “g” refers to grams; “kg” refers to kilograms; “nmole” or “nmol” refers to nanomoles; “mmol” refers to millimoles; “mol” refers to moles; “M” refers to molar, “mM” refers to millimolar, “μM” refers to micromolar, “nM” refers to nanomolar, “L” refers to liters, “mL” refers to milliliters, “μL” refers to microliters.

Pharmaceutically acceptable salts of the compounds of the invention include the acid addition and base salts (including disalts) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of the invention may be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

Compounds of the invention containing one or more asymmetric carbon atoms can exist as two or more stereoisomers. Where a compound of the invention contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds of the present invention are all stereoisomers, geometric isomers and tautomeric forms of the compounds of the invention, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallization.

Compounds of the current invention may also exhibit atropisomerism, where restricted rotation, especially around the bond joining two aryl rings in a biaryl, causes different rotational isomers to be not interconvertible at normal ambient temperatures, and quite possibly not at temperatures where the molecule as a whole remains thermally stable. In such cases distinct stereoisomers due to atropisomerism are also claimed.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of the invention wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴O, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of the invention can generally be prepared by conventional techniques known to those skilled in the art or by processes analogous to those described in the accompanying Examples and Preparations using an appropriate isotopically-labeled reagents in place of the non-labeled reagent previously employed.

The compounds of the present invention may be administered as prodrugs. Thus certain derivatives of compounds of the invention which may have little or no pharmacological activity themselves can, when administered into or onto the body, be converted into compounds of formula 1 (or other formulae disclosed herein) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in the compounds of the invention with certain moieties known to those skilled in the art as ‘pro-moieties’ as described, for example, in “Design of Prodrugs” by H Bundgaard (Elsevier, 1985).

Some examples of such prodrugs include:

where the compound contains a carboxylic acid functionality (—COOH), an ester thereof, for example, replacement of the hydrogen with C₁-C₆ alkyl;

where the compound contains an alcohol functionality (—OH), an ether thereof, for example, replacement of the hydrogen with C₁-C₆ alkanoyloxymethyl (—C₁-C₆ acyloxymethyl); and

where the compound contains a primary or secondary amino functionality (—NH₂ or —NHR where R is not H), an amide thereof, for example, replacement of one or both hydrogens with (C₁-C₁₀)alkanoyl (—C₁-C₁₀ acyl).

Further examples of replacement groups in accordance with the foregoing examples and examples of other prodrug types may be found in the aforementioned references.

Finally, certain compounds of formula (I) may themselves act as prodrugs of other compounds of formula (I).

Additional embodiments of the invention are set forth in formulae (II) through (V) and (XI) through (XX):

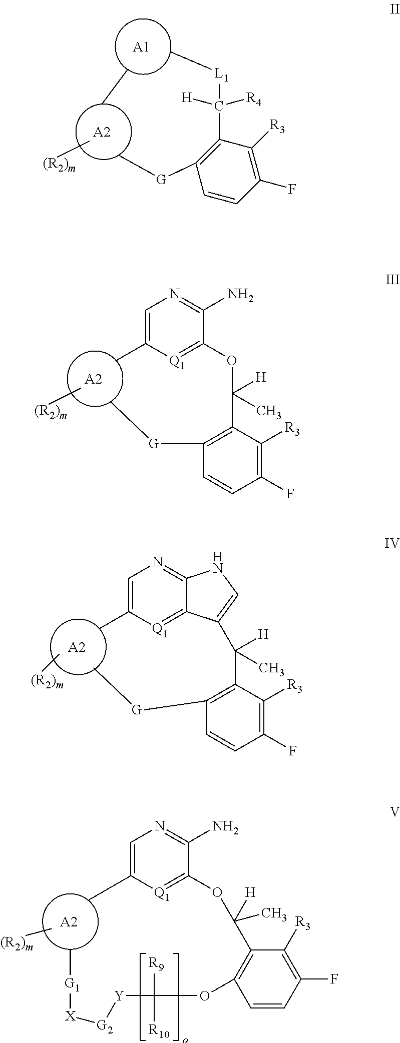

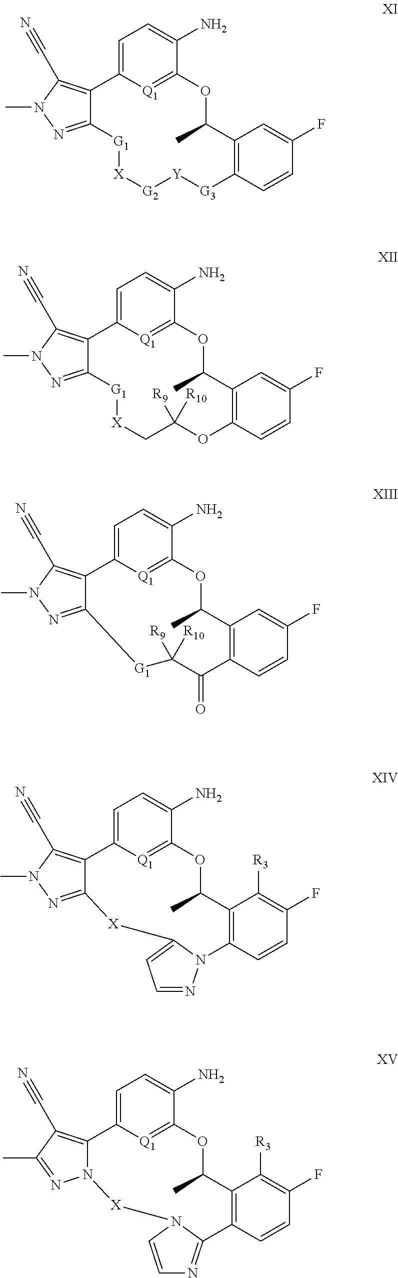

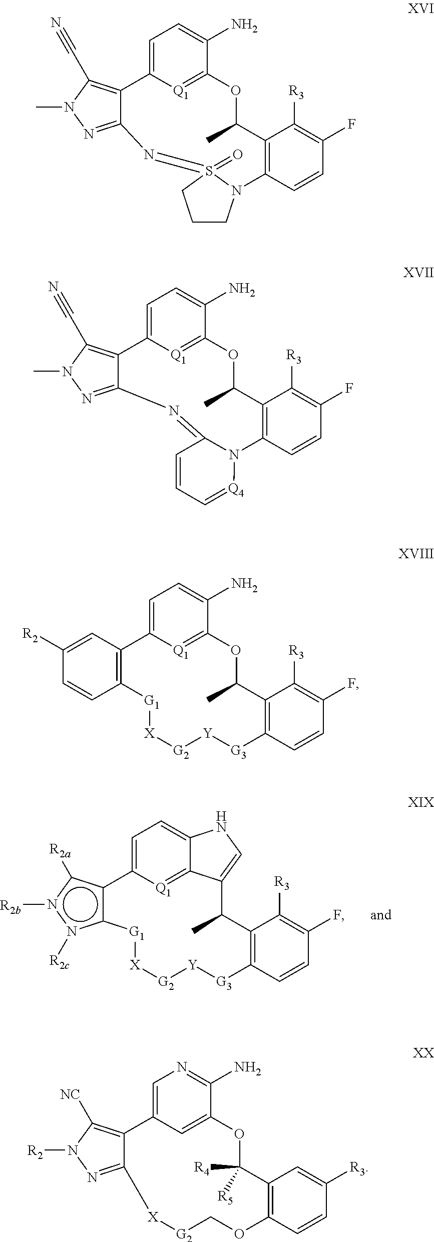

Alternate embodiments of compounds of the invention or stereoisomers or pharmaceutically acceptable salts thereof are given below. Each alternative embodiment set forth below is applicable to the compounds of formula (I) as well as the compounds of formulae (II)-(V) and (XI)-(XX) where appropriate:

(1) Compounds where A1 is

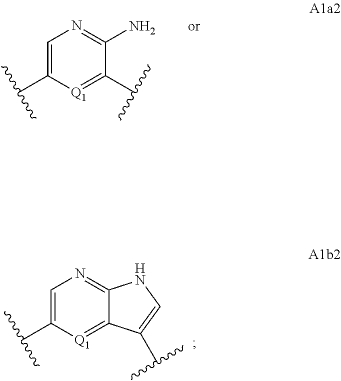

a. Compounds where A1 is A1a2 and Q₁ is CH;
b. Compounds where A1 is A1a2 and Q₁ is N;
c. Compounds where A1 is A1b2 and Q₁ is CH;
d. Compounds where A1 is A1b2 and Q₁ is N.

(2) Compounds where A2 is a benzene ring or a 5- to 6-membered heteroarylene containing up to three heteroatoms selected from S, O, and N, wherein adjacent groups adjoin the benzene ring or heteroarylene in a 1,2- or 1,3-relationship;
a. Compounds where A2 is benzene;
b. Compounds where A2 is a 5- to 6-membered heteroarylene.

(3) Compounds where A3 is optionally substituted benzene.

(4) Compounds where L1 is —O—, —CH₂—, —NH—, or is a bond;
a. Compounds where L1 is —O—;
b. Compounds where L1 is —CH₂—;
c. Compounds where L1 is —NH—;
d. Compounds where L1 is a bond.

(5) Compounds where G is -G₁-X-G₂-Y-G₃-;
a. X and Y are each independently selected from —O—, —NR₈—, —B(OR₁₁)—, —S(O)ₓ—, —S(O)(═NR₈)—, —C(O)NR₈—, —C(R₁₂)═N—, —C(NR₁₇)═N—, —C(OR₁₁)═N—, —C(═NR₆)N(R₇)—, and —C(═NR₁₇)N(R₁₇)—; where x is 0-2;
b. X and Y are each independently selected from —O—, —NR₈—, —S(O)ₓ—, and —S(O)(═NR₈)—;
c. One of X and Y is a divalent ring system selected from List 1, List 2, List 3 or List 4;
   i. One of X and Y is a divalent ring system selected from List 1;
   ii. One of X and Y is a divalent ring system selected from List 2;
   iii. One of X and Y is a divalent ring system selected from List 3;
   iv. One of X and Y is a divalent ring system selected from List 4;
   v. One of X and Y is a heteroarylene selected from the group consisting of pyrrolylene, thienylene, furanylene, imidazylene, pyrazolylene, thiazolylene, oxazolylene, isothiazolylene, isoxazolylene, 1,2,3-triazolylene, 1,2,4-triazolylene, 1,2,3-oxadiazolylene, 1,3,2-oxadiazolylene, 1,2,3-thiadiazolylene, 1,3,2-thiadiazolylene and tetrazolylene, wherein said heteroarylene is optionally substituted with 1-3 R₁₅ or R₁₆ groups;
   vi. One of X and Y is a heteroarylene selected from the group consisting of phenylene, pyridylene, pyrazinylene, pyrimidylene, pyridazinylene, 1,2,4-triazenylene, 1,3,5-triazenylene and 1,2,3-triazenylene, wherein said heteroarylene is optionally substituted with 1-3 R₁₅ or R₁₆ groups;
d. G₂ is —(CR₉R₁₀)ₒ, where o is 1-3;
e. G₂ is —(CR₉R₁₀)ₒ—, —CR₉═CR₁₀—, —C(═O)—, —C(═NR₈)—, —C(═NOR₁₁)—, —C(═NNR₆R₇)—, —CF₂—, or CR₉═CR₁₀CR₉R₁₀—, or a bond; where o is 1-3;
f. Each of G₁, G₂, and G₃ is independently is —(CR₉R₁₀)ₒ—, —CR₉═CR₁₀—, —C(═O)—, —C(═NR₈)—, —C(═NOR₁₁)—, —C(═NNR₆R₇)—, —CF₂—, —S(O)ₓ(CR₉R₁₀)ₒ—, or —CR₉═CR₁₀CR₉R₁₀—, or a bond; wherein o is 1-3 and wherein at least one of G₁, G₂, or G₃ is not a bond; and X and Y are each a bond;
g. Each of G₁, G₂, and G₃ is independently —(CR₉R₁₀)ₒ—, —CR₉═CR₁₀—, —C(═O)—, —C(═NR₈)—, —C(═NOR₁₁)—, —C(═NNR₆R₇)—, —CF₂—, or CR₉═CR₁₀CR₉R₁₀—, or a bond; wherein o is 1-3 and wherein at least one of G₁, G₂, or G₃ is not a bond; and each of X and Y is independently —S(O)ₓ—, —S(O)(═NR₈)—, —P(O)(R₁₃)—, or —P(O)(OR₁₁)—, or a bond; wherein at least one of X and Y is not a bond; and wherein x is 0-2
h. One or two of G₁, G₂, and G₃ is a bond; wherein X and Y are each independently a bond, —S(O)ₓ—, —S(O)(═NR₈)—, —P(R₁₃)—, —P(O)(R₁₃)—, —P(O)(OR₁₁)—, —SO₂NR₈—, —S(O)(R₁₃)═N—, —S(O)(═NR₆)N(R₇)—, —S(O)(N(R₆R₇)═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, C(═NR₆)N(R₇)—, —C(═NR₁₇)N(R₁₇)—,

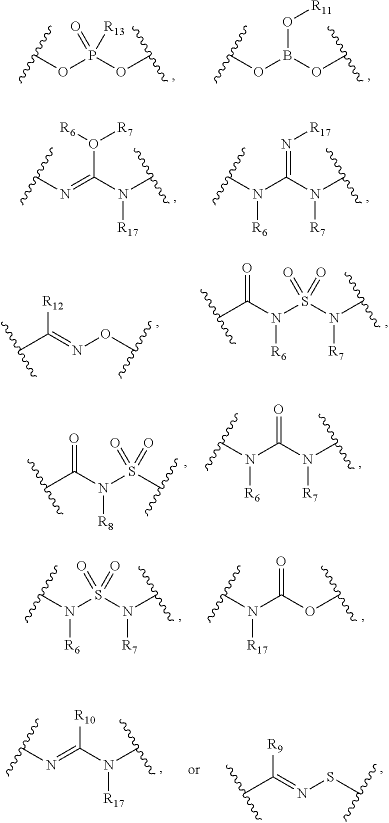

wherein one of X and Y is a bond; and wherein x is 0-2;
i. X and Y are each independently a bond or —S(O)ₓ—, —S(O)(═NR₈)—, —P(R₁₃)—, —P(O)(R₁₃)—, —P(O)(OR₁₁)—, —SO₂NR₈—, —S(O)(R₁₃)═N—, —S(O)(═NR₆)N(R₇)—, —S(O)(N(R₆R₇)═N—, —P(O)(R₁₃)O—, or —P(O)(OR₁₁)O—.
j. G₁ is CR₉R₁₀, —C(═O)—, —C(═NR₈)—, —C(═NOR₁₁)—, —C(═NNR₆R₇)—, —CF₂—, —O(CR₉R₁₀)ₒ—, —S(O)ₓ(CR₉R₁₀)ₒ—, or —NR₈(CR₉R₁₀)ₒ— or a bond; G₂ is a bond; G₃ is CR₉R₁₀, —C(═O)—, —C(═NR₈)—, —C(═NOR₁₁)—, —C(═NNR₆R₇)—, —CF₂—, —O(CR₉R₁₀)—, —S(O)ₓ(CR₉R₁₀)ₒ—, or —NR₈(CR₉R₁₀)ₒ— or a bond; wherein G₁ and G₃ are not both a bond; and one of X and Y is selected from —O—, —NR₈—, —S(O)ₓ— and —S(O)(═NR₈)—.

(6) Compounds where G is —X-G₂-Y—;
a. X and Y are each independently selected from —O—, —NR₈—, —B(OR₁₁)—, —S(O)ₓ—, —S(O)(═NR₈)—, —C(O)NR₈—, —C(R₁₂)═N—, —C(NR₁₇)═N—, —C(OR₁₁)═N—, —C(═NR₆)N(R₇)—, and —C(═NR₁₇)N(R₁₇)—; where x is 0-2;
b. X and Y are each independently selected from —O—, —NR₈—, —S(O)ₓ—, and —S(O)(═NR₈)—;
c. One of X and Y is a divalent ring system selected from List 1, List 2, List 3 or List 4;
   i. One of X and Y is a divalent ring system selected from List 1;
   ii. One of X and Y is a divalent ring system selected from List 2;
   iii. One of X and Y is a divalent ring system selected from List 3;
   iv. One of X and Y is a divalent ring system selected from List 4;
   v. One of X and Y is a heteroarylene selected from the group consisting of pyrrolylene, thienylene, furanylene, imidazylene, pyrazolylene, thiazolylene, oxazolylene, isothiazolylene, isoxazolylene, 1,2,3-triazolylene, 1,2,4-triazolylene, 1,2,3-oxadiazolylene, 1,3,2-oxadiazolylene, 1,2,3-thiadiazolylene, 1,3,2-thiadiazolylene and tetrazolylene, wherein said heteroarylene is optionally substituted with 1-3 R₁₅ or R₁₆ groups;
   vi. One of X and Y is a heteroarylene selected from the group consisting of phenylene, pyridylene, pyrazinylene, pyrimidylene, pyridazinylene, 1,2,4-triazenylene, 1,3,5-triazenylene and 1,2,3-triazenylene, wherein said heteroarylene is optionally substituted with 1-3 R₁₅ or R₁₆ groups;
d. G₂ is —(CR₉R₁₀)ₒ, where o is 1-3;
e. G₂ is —(CR₉R₁₀)ₒ—, —CR₉═CR₁₀—, —C(═O)—, —C(═NR₈)—, —C(═NOR₁₁)—, —C(═NNR₆R₇)—, —CF₂—, or CR₉═CR₁₀CR₉R₁₀—, or a bond; where o is 1-3.

(7) Compounds where G is —X-G₃- or -G₁-Y—;
a. G₁ and G₃ are each —(CR₉R₁₀)ₒ—, —C(═O)—, —C(═NR₈)—, —C(═NOR₁₁)—, —C(═NNR₆R₇)—, —CF₂—, —O(CR₉R₁₀)ₒ—, —S(O)ₓ(CR₉R₁₀)ₒ—, or —NR₈(CR₉R₁₀)ₒ,
b. X and Y are each a divalent ring system selected from Lists 1 through 4.
c. X and Y are each a divalent ring system selected from List 1;
d. X and Y are each a divalent ring system selected from List 2;
e. X and Y are each a divalent ring system selected from List 3;
f. X and Y are each a divalent ring system selected from List 4.

It is understood that further embodiments of the compounds of the invention can be selected by requiring one or more of the preferred embodiments (1) through (6) above of compounds of formulae (I) to (V) and/or (XI) to (XIX) as appropriate, or stereoisomers, pharmaceutically acceptable salts, or prodrugs thereof or by reference to the examples given herein.

For example, further embodiments of the compounds of formula (I) can be obtained by combining (1) and (2); (1) and (2)(a); (1) and (2)(b); (1)(a) and (2); (1)(b) and (2); (1)(c) and (2); (1)(d) and (2); (1)(a) and (2)(a); (1)(b) and (2)(a); (1)(a) and (2)(b); (1) and (3); (2) and (3); (2)(a) and (3); (2)(b) and (3); (1), (2), and (3); (1), (2)(a) and (3); (1), (2)(b), and (3); (1)(a), (2), and (3); (1)(b), (2), and (3); (1)(c), (2), and (3); (1)(d), (2), and (3); (1)(a), (2)(a), and (3); (1)(b), (2)(a), and (3); (1)(a), (2)(b), and (3); (1)(c), (2)(a), and (3); (1)(c), (2)(b), and (3); (1)(d), (2)(b), and (3); (1) and (4); (2) and (4); (3) and (4); (1), (2), (3), and (4); (1)(a), (2), (3), and (4); (1)(b), (2), (3), and (4); (1)(c), (2), (3), and (4); (1)(d), (2), (3), and (4); (1)(a), (2)(a), (3), and (4); (1)(b), (2)(a), (3), and (4); (4)(a); (4)(b); (4)(c); (4)(d); (1), (2) and (4)(a); (1), (2), (3) and (4)(c); (1), (2), (3) and (4)(d); (5); (5)(a); (5)(b); (5)(c), (5)(d); (5)(e); (5)(f); (5)(g); (5)(h); (5)(i); (5)(j); (1) and (5)(a); (1), (2), (3), (4), and (5)(a); (1), (2), (3), (4), and (5)(b); (1), (2), (3), (4), and (5)(c); (1), (2), (3), (4), and (5)(d); (1), (2), (3), (4), and (5)(e); (1), (2), (3), (4), and (5)(f); (1), (2), (3), (4), and (5)(g); (1), (2), (3), (4), and (5)(h); (1), (2), (3), (4), and (5)(i); (1), (2), (3), (4), and (5)(j); (1), (2), (3), (4), and (5)(c)(i); (1), (2), (3), (4), and (5)(c)(ii); (1), (2), (3), (4), and (5)(c)(iii); (1), (2), (3), (4), and (5)(c)(iv); (1), (2), (3), (4), and (5)(c)(v); (1), (2), (3), (4), and (5)(c)(vi); (6); (6)(a); (6)(b); (6)(c), (6)(d); (6)(e); (1) and (6)(a); (1), (2), (3), (4), and (6)(a); (1), (2), (3), (4), and (6)(b); (1), (2), (3), (4), and (6)(c); (1), (2), (3), (4), and (6)(d); (1), (2), (3), (4), and (6)(e); (1), (2), (3), (4), and (6)(c)(i); (1), (2), (3), (4), and (6)(c)(ii); (1), (2), (3), (4), and (6)(c)(iii); (1), (2), (3), (4), and (6)(c)(iv); (1), (2), (3), (4), and (6)(c)(v); (1), (2), (3), (4), and (6)(c)(vi); and the like.

Additional embodiments of the invention are represented by compounds of formula (XI) or pharmaceutically acceptable salts thereof according to any of groups (a) through (e):
a. Q₁ is CH or N; G₁ is —CR₉R₁₀— or a bond; X is a bond; G₂ is —(CR₉R₁₀)₂— or —CR₉═CR₁₀—; Y is a bond; G₃ is —CR₉R₁₀— or a bond; each R₉ is independently H or C₁-C₆ alkyl; and each R₁₀ is independently H or C₁-C₆ alkyl;
b. Q₁ is CH or N; G₁ is —CH₂—, —CH₂CH₂— or a bond; X is a bond; G₂ is a bond; Y is —SO₂—, —S(═O)(═NR₈)—, —SO₂NR₈—, —S(═O)(═NR₆)NR₇—, —N═S(═O)(R₁₃)—, —N═S(═O)(NR₆R₇)—, —N═S(═O)(R₁₃)N(R₇)—, —N(R₈)S(═O₂)N(R₇)—, —N(R₈)C(O)N(R₇)—, —N(R₈)C(O)O—, —N(R₇)C(═NR₆)—, —N═C(N(R₁₇)₂)N(R₈)—, —N═C(N(R₁₇)₂)—, —N(R₈)N(R₇)—, —N(R₈)O—, —N(R₈)S—, —C(O)N(R₈)SO₂—, —C(O)N(R₈)C(O)—, —C(O)N(R₈)N(R₇)—, —C(O)N(R₈)O—, —C(O)N(R₈)S—, —P(═O)(R₁₃)—, —P(═O)(OR₁₁)O—, —OP(═O)(R₁₃)O—, —B(OH)—, or —B(R₁₃)O—; G₃ is —CH₂—, —CH₂CH₂—, or a bond; R₆ is H, C₁-C₆ alkyl, or C₁-C₆ acyl; R₇ is H or C₁-C₆ alkyl; each R₈ is independently H, C₁-C₆ alkyl, —CH₂CH₂OH, —CH₂C(CH₃)₂OH; R₁₁ is H or C₁-C₆ alkyl; R₁₃ is C₁-C₆ alkyl or —CH₂CH₂OH; and each R₁₇ is independently C₁-C₆ alkyl, or two R₁₇ groups together with the nitrogen atom to which they are attached form a 5-6 membered heterocyclyl;
c. Q₁ is CH or N; G₁ is a bond; X is —O— or —NR₈—; G₂ is —(CR₉R₁₀)₂—, —(CR₉R₁₀)₃—, or —CR₉═CR₁₀—; Y is —O— or —NR₈—; G₃ is a bond; each R₈ is independently H or C₁-C₆ alkyl; each R₉ is independently H or C₁-C₆ alkyl; and each R₁₀ is independently H or C₁-C₆ alkyl;
d. Q₁ is CH or N; G₁ is —CH₂— or a bond; X is O, NR₈, or a bond; G₂ is selected from the group consisting of

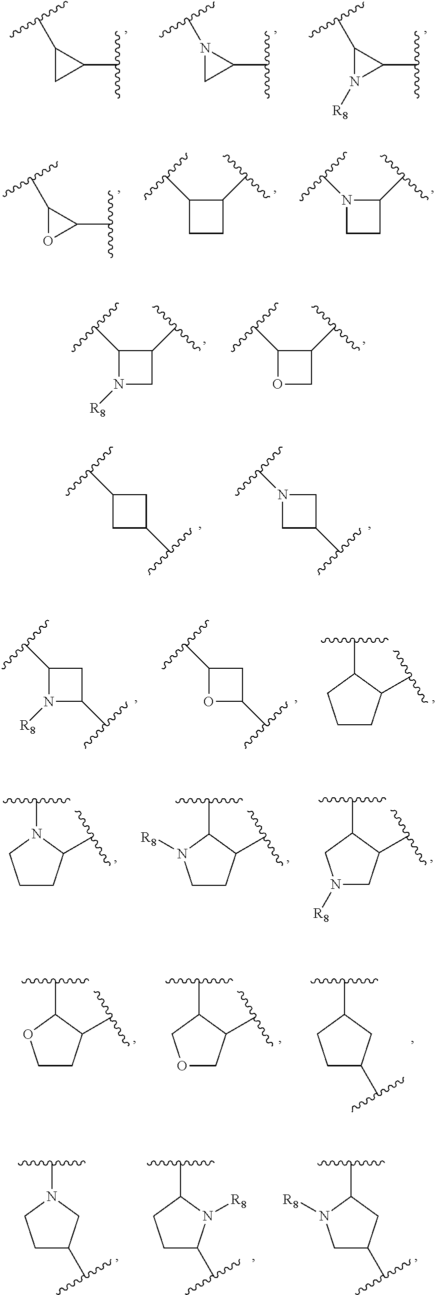

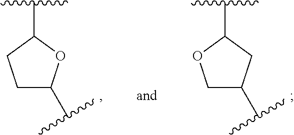 [SMILES: *C1CCC(*)O1 ; *C1COC(*)C1]

Y is O, NR₈, or a bond; G₃ is —CH₂— or a bond; and each R₈ is independently H or C₁-C₆ alkyl;
e. Q₁ is CH or N; G₁ is —CH₂— or —CH₂CH₂—; X is a bond; G₂ is a bond; Y is selected from the group consisting of:

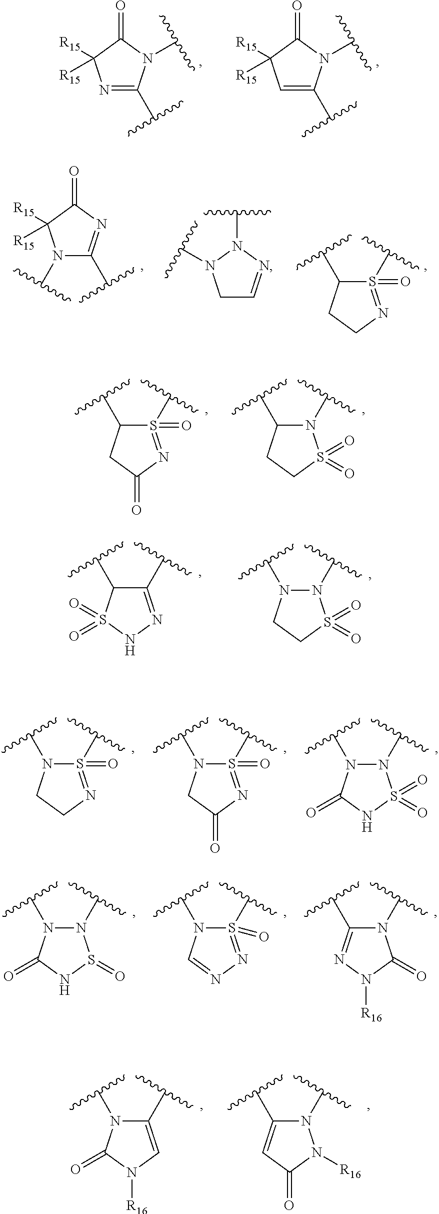

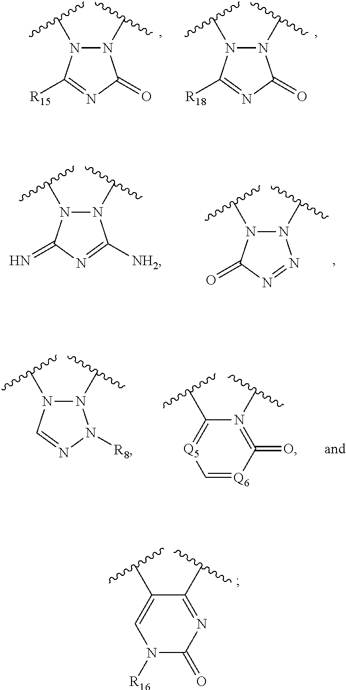

G₃ is a bond; Q₅ is CH or N; Q₆ is CH or N; each R₈ is independently H or C₁-C₆ alkyl; each R₁₅ is —CH₃ or —CH₂OH; each R₁₆ is C₁-C₄ alkyl; and each R₁₈ is —OH or —NH₂.

A further embodiment of the invention is represented by compounds of formula (XII) or pharmaceutically acceptable salts thereof wherein Q₁ is CH or N; G₁ is CH₂ or a bond; X is O, NR₈, or a bond; R₈ is H or C₁-C₆ alkyl; and R₉ and R₁₀ together with the carbon atom to which they are attached form a 3-5 membered cycloalkyl or heterocyclyl.

A further embodiment is represented by compounds of formula (XIII) or pharmaceutically acceptable salts thereof wherein Q₁ is CH or N; G₁ is —CH₂—, —O—, —NR—, —CH₂O—, or —CH₂NR₈—; each R₈ is independently H or C₁-C₆ alkyl; and R₉ and R₁₀ are each independently H, C₁-C₆ alkyl, —CH₂OH, or —CH₂N(R₈), or R₉ and R₁₀ together with the carbon atom to which they are attached form a 3-5 membered cycloalkyl or heterocyclyl.

A further embodiment is represented by compounds of formula (XIV) or pharmaceutically acceptable salts thereof wherein Q₁ is CH or N; R₃ is H or Cl; X is O or NR₈; and R₈ is H or C₁-C₆ alkyl.

Another embodiment is represented by compounds of formula (XV) or pharmaceutically acceptable salts thereof wherein Q₁ is CH or N; R₃ is H or Cl; X is O, NR₈, or SO₂; and R₈ is H or C₁-C₆ alkyl.

Another embodiment is represented by compounds of formula (XVI) or pharmaceutically acceptable salts thereof wherein Q₁ is CH or N; and R₃ is H or Cl.

Another embodiment is represented by compounds of formula (XVII) or pharmaceutically acceptable salts thereof wherein Q₁ is CH or N; Q₄ is CH or N; and R₃ is H or Cl.

Another embodiment is represented by compounds of formula (XVIII) or pharmaceutically acceptable salts thereof wherein Q₁ is CH or N; R₂ is —S(═O)(═NR₈)R₁₃ or —N═S(═O)(R₁₃)₂; R₃ is H or Cl; G₁ is —CH₂— or a bond; X is —O—, —NR—, —C(O)NR₈—, or a bond; G₂ is —CH₂CH₂— or a bond; Y is —O—, —NR₈—, —C(O)NR₈—, a 5-6 membered hetereoarylene, or a bond; G₃ is —CH₂— or a bond; wherein at least two but no more than three of G₁, G₂, G₃, X, and Y is a bond; wherein G₂ is —CH₂CH₂— when neither X nor Y is a bond; R₈ is H or C₁-C₆ alkyl; and R₁₃ is C₁-C₆ alkyl.

Another embodiment is represented by compounds of formula (XIX) or pharmaceutically acceptable salts thereof wherein Q₁ is CH or N; R₂ₐ is H, C₁-C₆ alkyl, or CN; R_{2b} is H or C₁-C₆ alkyl, or is absent; R_{2c} is H, C₁-C₆ alkyl, or —CH₂CH₂OH, or is absent; wherein one of R_{2b} and R_{2c} is absent; R₃ is H or Cl; G₁ is —CH₂— or a bond; X is —O—, —NR₈—, —C(O)NR₈—, or a bond; G₂ is —CH₂CH₂— or a bond; Y is —O—, —NR₈—, —C(O)NR₈—, a 5-6 membered heteroarylene, or a bond; G₃ is —CH₂— or a bond; wherein at least two but no more than three of G₁, G₂, G₃, X, and Y is a bond; wherein G₂ is —CH₂CH₂— when neither X nor Y is a bond; and R₅ is H or C₁-C₆ alkyl.

Another embodiment is represented by compounds of formula (XX) or pharmaceutically acceptable salts thereof wherein G₂ is —CH₂— or —C(O)—; X is —O— or —NR₈—; R₂ is C₁-C₄ alkyl; R₃ is F or Cl; R₄ and R₅ are each independently H, CH₃, CH₂CH₃, CH₂F, CHF₂, or CF₃; and R₅ is H or C₁-C₄ alkyl.

As with any group of structurally related compounds which possesses a particular utility, certain groups and configurations are preferred for the compounds of formula (I) and their end-use application.

The compounds of the invention include:

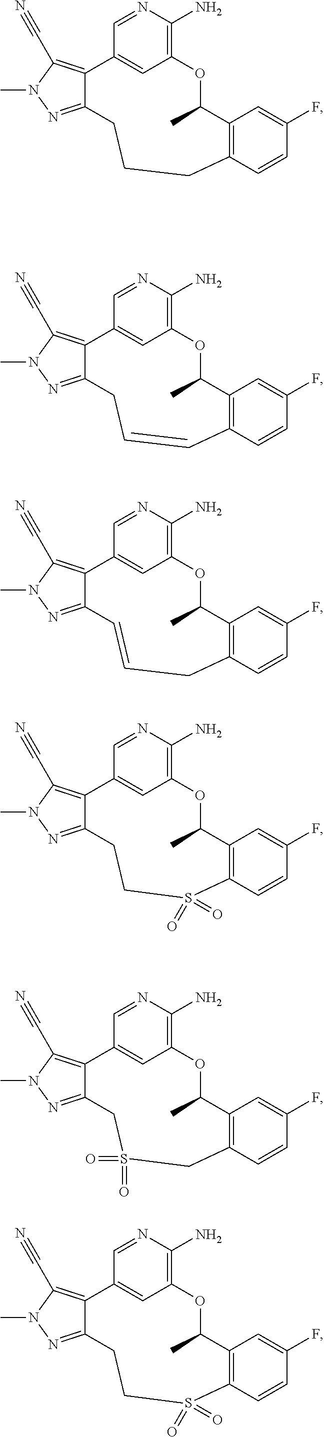

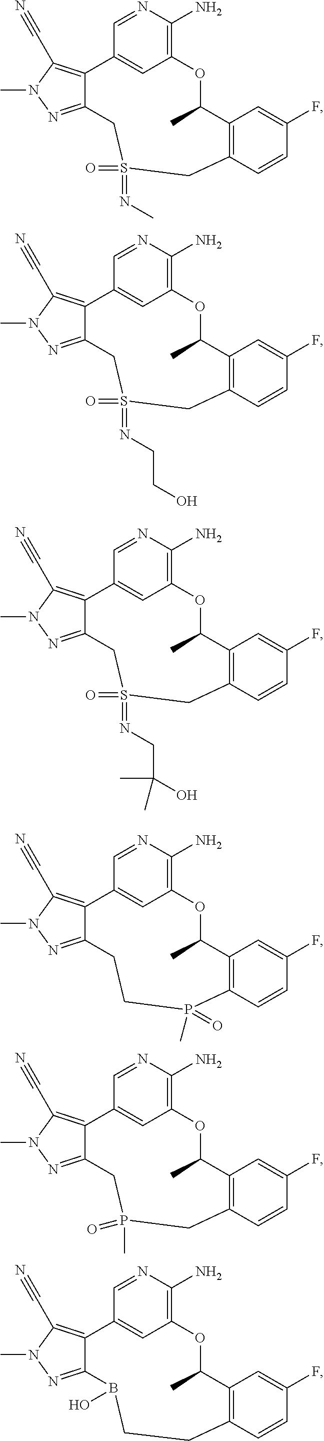

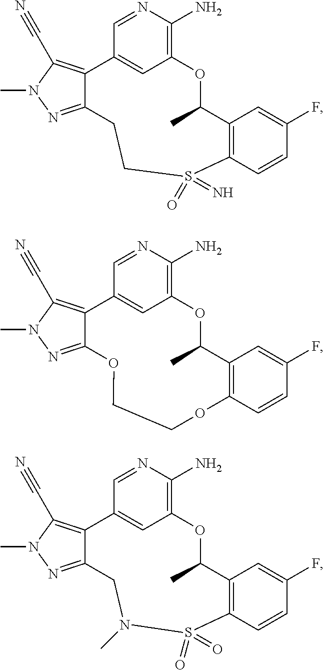

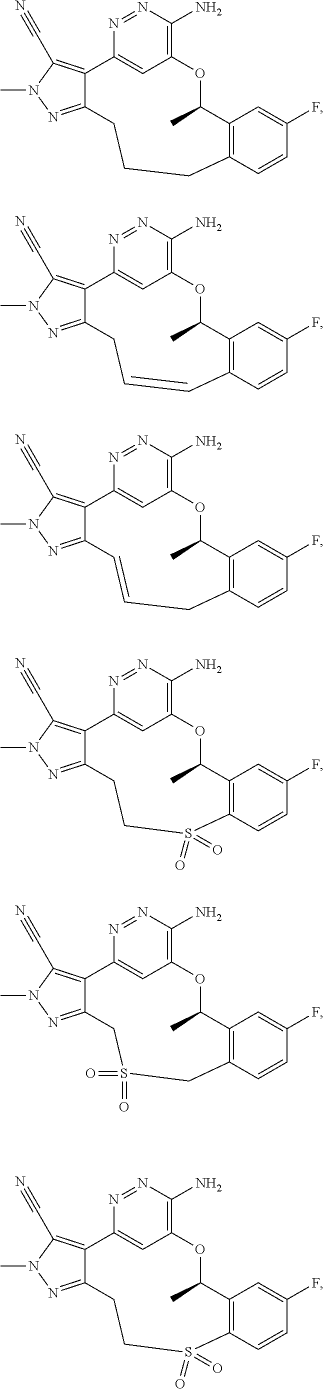 [SMILES: C[C@H]1Oc2cc(nnc2N)-c2c(nn(C)c2C#N)CCCc2ccc(F)cc21 ; C[C@H]1Oc2cc(nnc2N)-c2c(nn(C)c2C#N)C/C=C\c2ccc(F)cc21 ; C[C@H]1Oc2cc(nnc2N)-c2c(nn(C)c2C#N)/C=C/Cc2ccc(F)cc21 ; C[C@H]1Oc2cc(nnc2N)-c2c(nn(C)c2C#N)CCS(=O)(=O)c2ccc(F)cc21 ; C[C@H]1Oc2cc(nnc2N)-c2c(nn(C)c2C#N)CS(=O)(=O)Cc2ccc(F)cc21 ; C[C@H]1Oc2cc(nnc2N)-c2c(nn(C)c2C#N)CCS(=O)(=O)c2ccc(F)cc21]

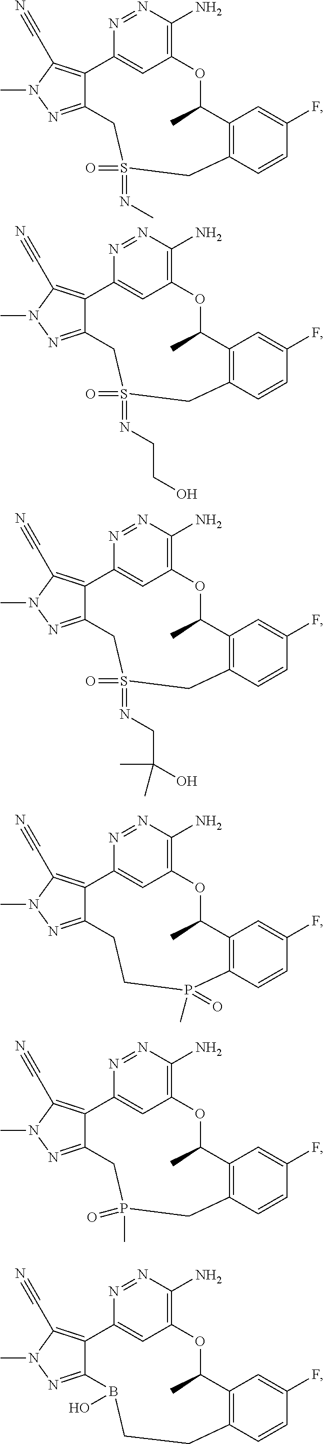

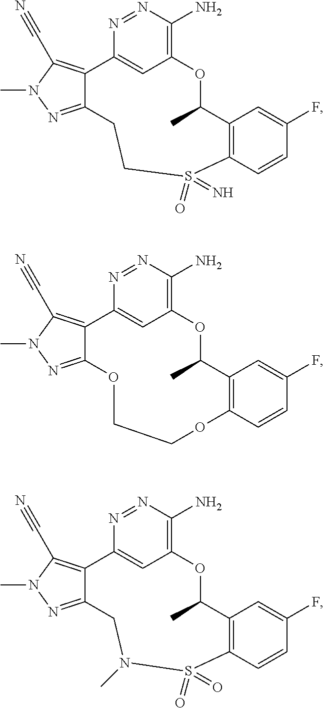 [SMILES: C[C@H]1Oc2cc(nnc2N)-c2c(nn(C)c2C#N)CCS(=N)(=O)c2ccc(F)cc21 ; C[C@H]1Oc2cc(nnc2N)-c2c(nn(C)c2C#N)OCCOc2ccc(F)cc21 ; C[C@H]1Oc2cc(nnc2N)-c2c(nn(C)c2C#N)CN(C)S(=O)(=O)c2ccc(F)cc21]

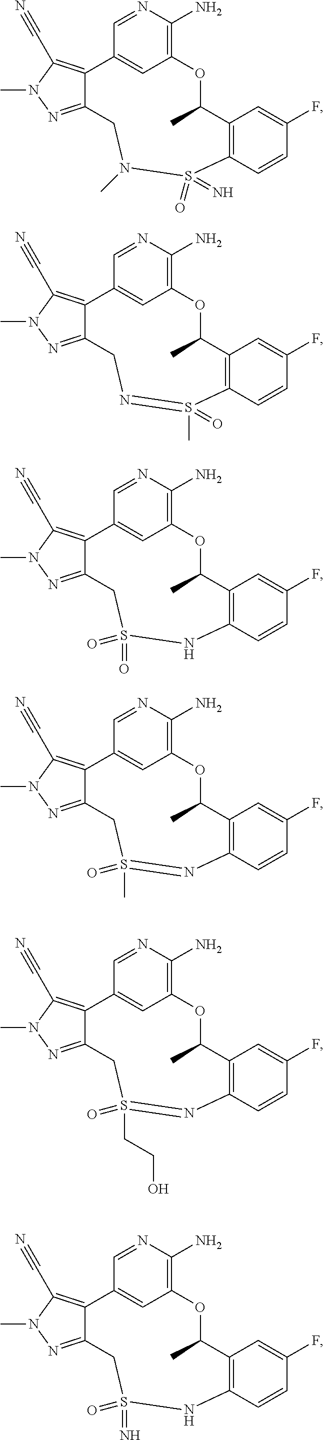

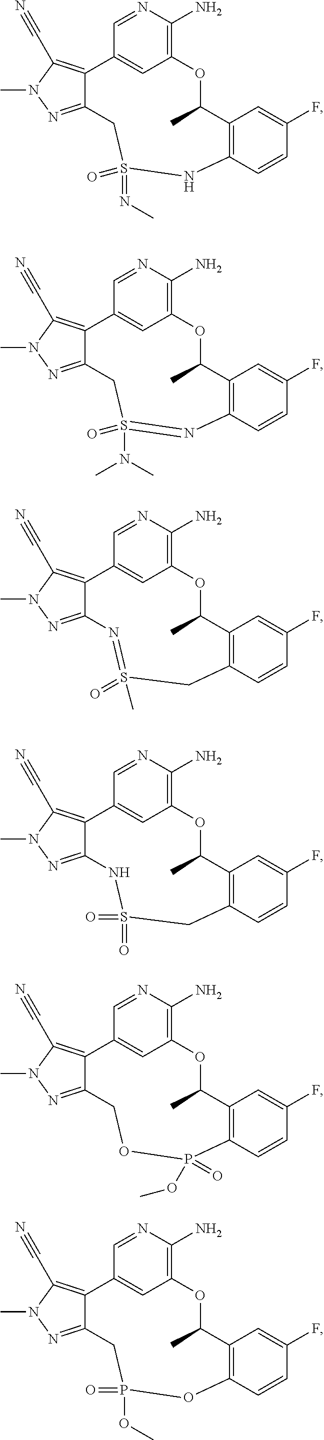

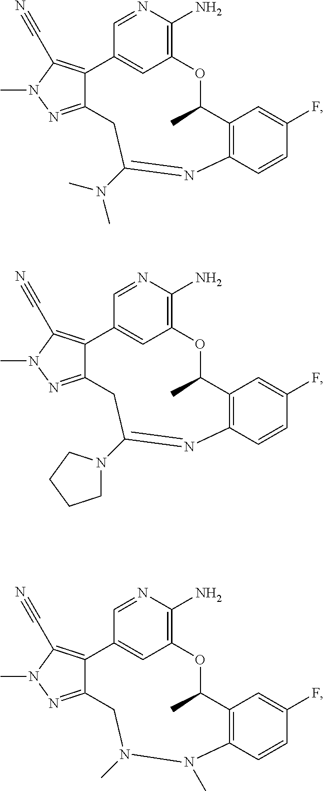 [SMILES: C[C@H]1Oc2cc(cnc2N)-c2c(nn(C)c2C#N)C/C(N(C)C)=N\c2ccc(F)cc21 ; C[C@H]1Oc2cc(cnc2N)-c2c(nn(C)c2C#N)C/C(N2CCCC2)=N\c2ccc(F)cc21 ; C[C@H]1Oc2cc(cnc2N)-c2c(nn(C)c2C#N)CN(C)N(C)c2ccc(F)cc21]

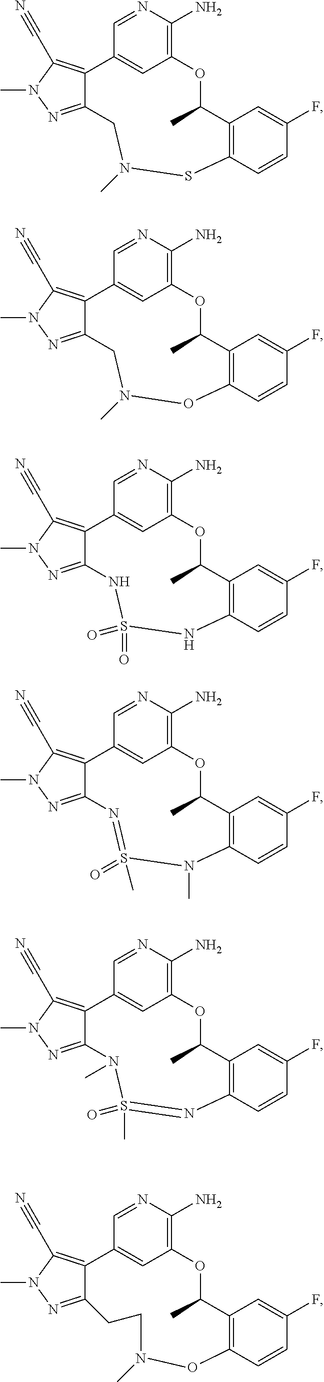

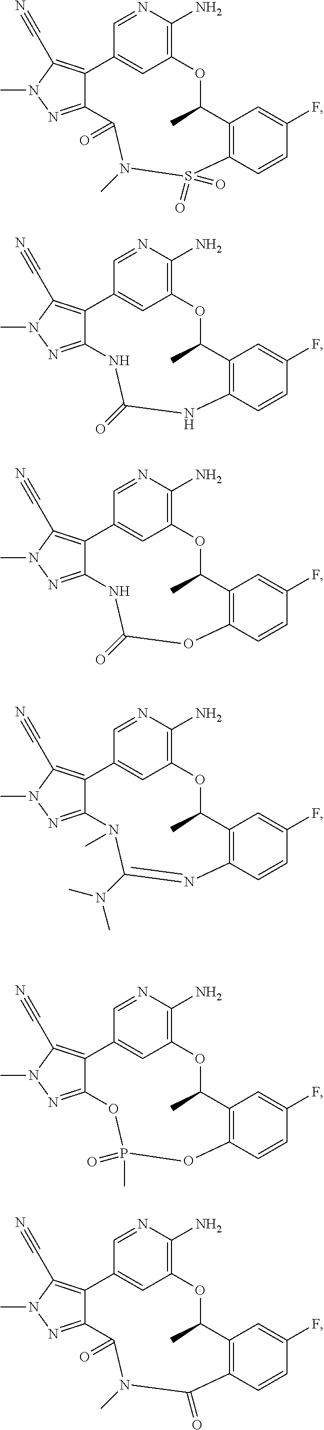

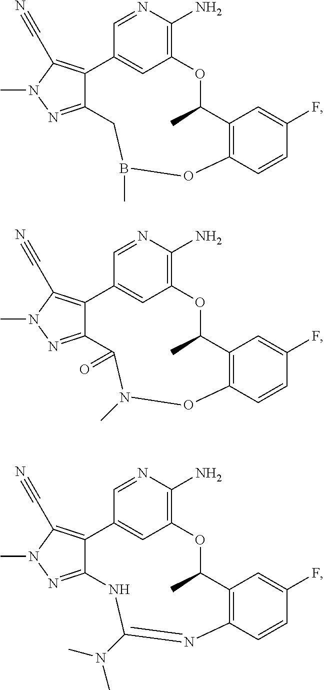

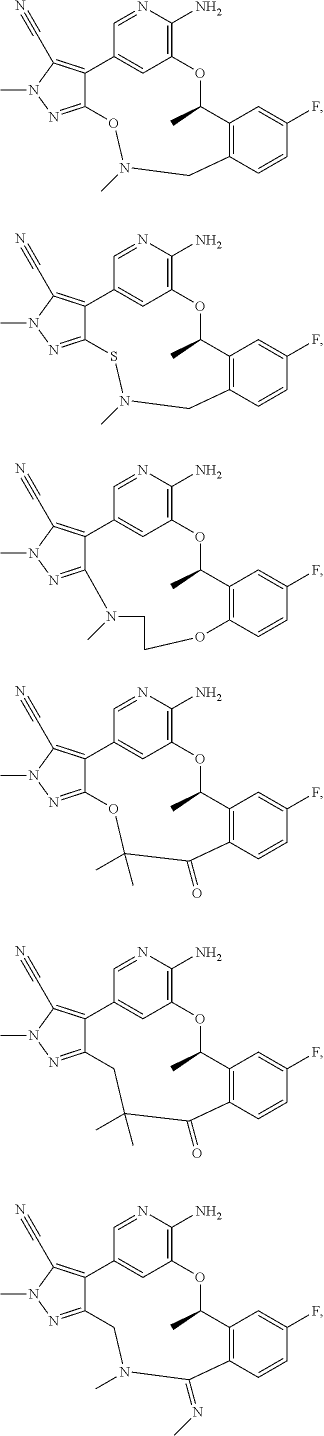

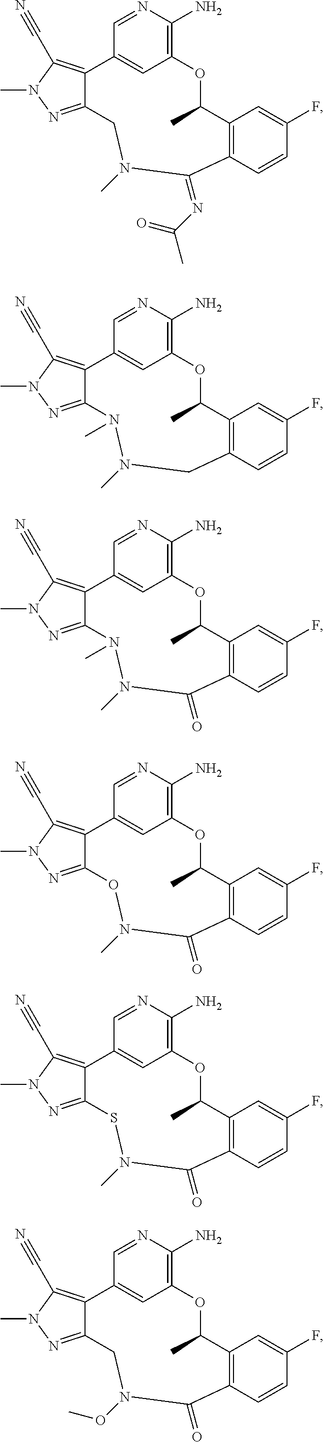

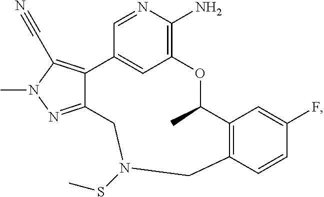 [SMILES: CSN1Cc2ccc(F)cc2[C@@H](C)Oc2cc(cnc2N)-c2c(nn(C)c2C#N)C1]

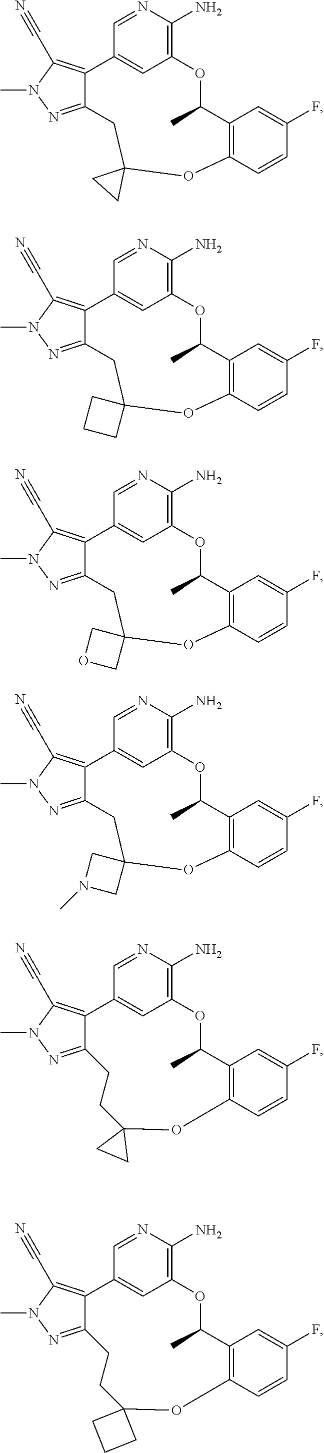

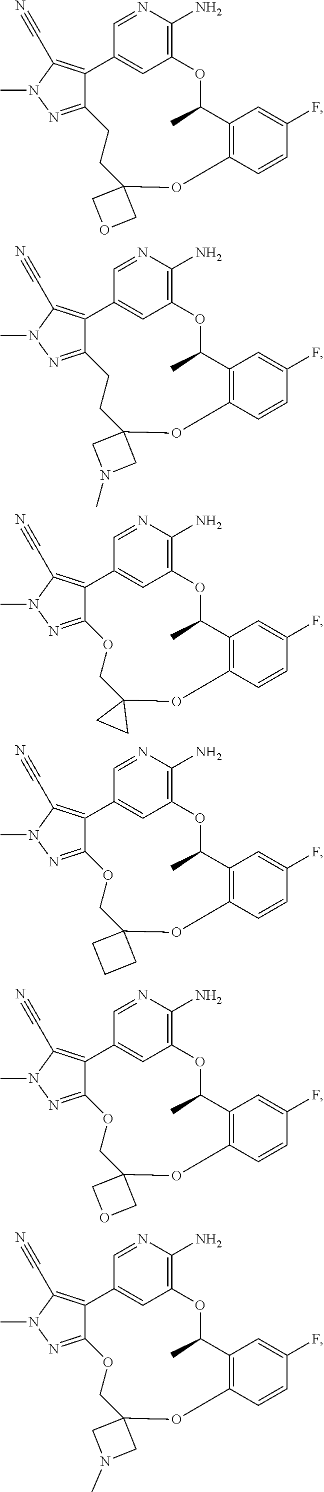

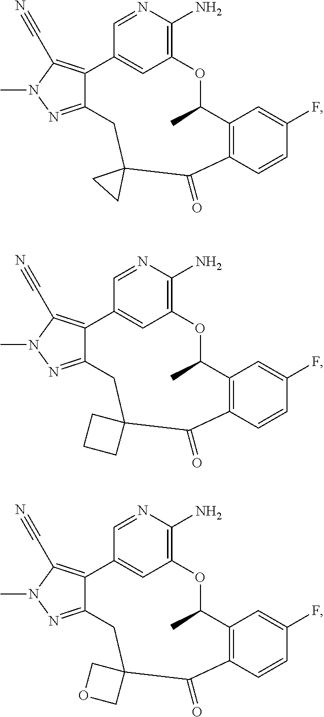 [SMILES: C[C@H]1Oc2cc(cnc2N)-c2c(nn(C)c2C#N)CC2(CC2)C(=O)c2ccc(F)cc21 ; C[C@H]1Oc2cc(cnc2N)-c2c(nn(C)c2C#N)CC2(CCC2)C(=O)c2ccc(F)cc21 ; C[C@H]1Oc2cc(cnc2N)-c2c(nn(C)c2C#N)CC2(COC2)C(=O)c2ccc(F)cc21]

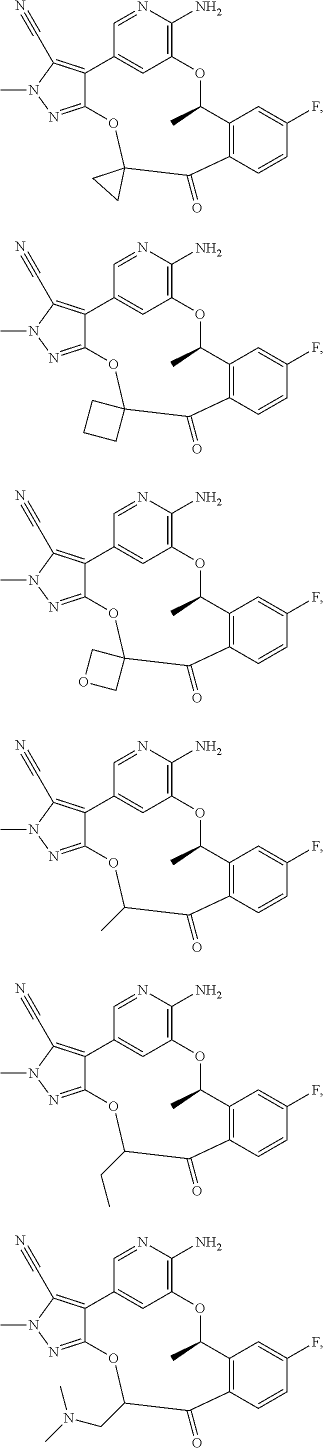

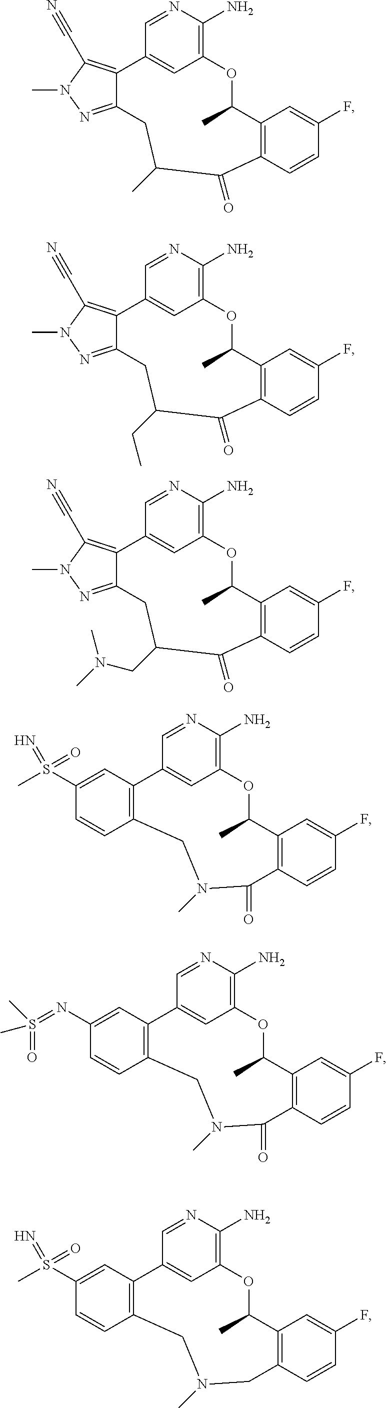

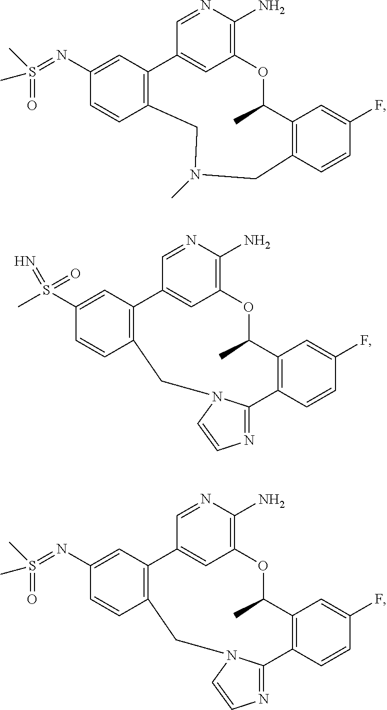

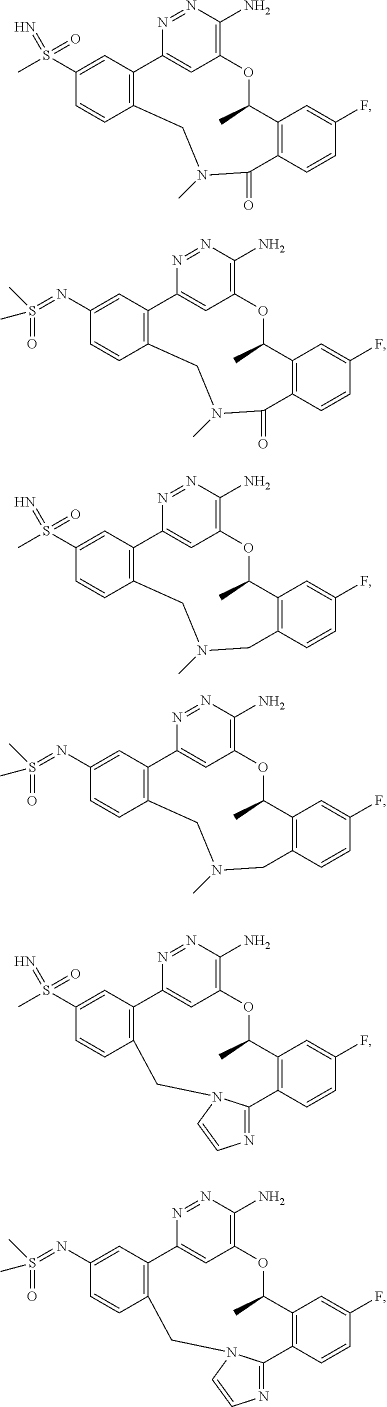

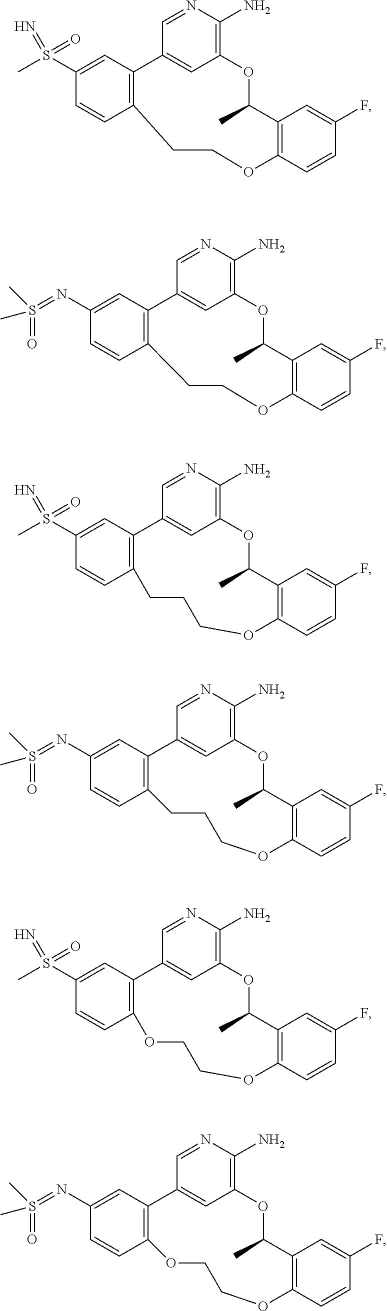 [SMILES: C[C@H]1Oc2cc(cnc2N)-c2cc(S(C)(=N)=O)ccc2CCOc2ccc(F)cc21 ; C[C@H]1Oc2cc(cnc2N)-c2cc(N=S(C)(C)=O)ccc2CCOc2ccc(F)cc21 ; C[C@H]1Oc2cc(cnc2N)-c2cc(S(C)(=N)=O)ccc2CCCOc2ccc(F)cc21 ; C[C@H]1Oc2cc(cnc2N)-c2cc(N=S(C)(C)=O)ccc2CCCOc2ccc(F)cc21 ; C[C@H]1Oc2cc(cnc2N)-c2cc(S(C)(=N)=O)ccc2OCCOc2ccc(F)cc21 ; C[C@H]1Oc2cc(cnc2N)-c2cc(N=S(C)(C)=O)ccc2OCCOc2ccc(F)cc21]

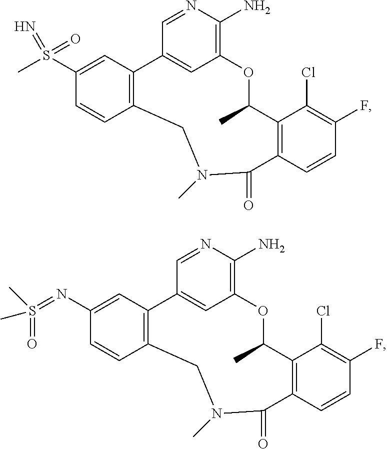

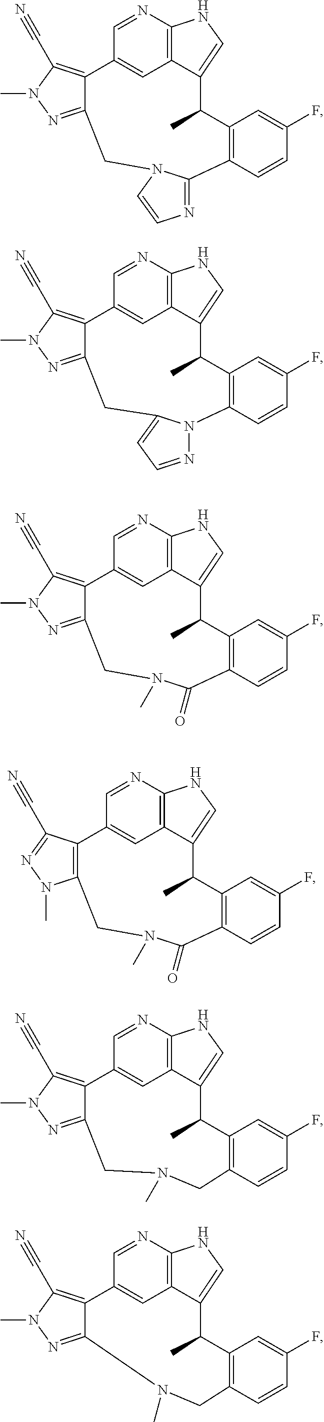

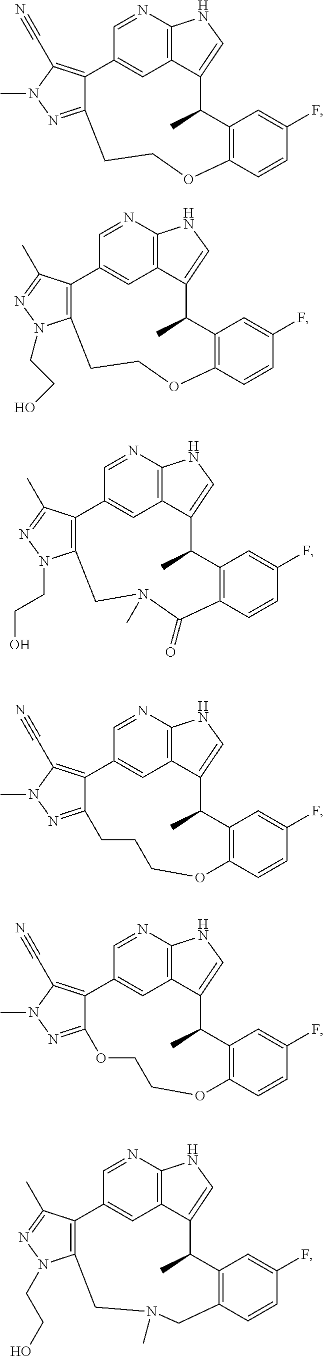

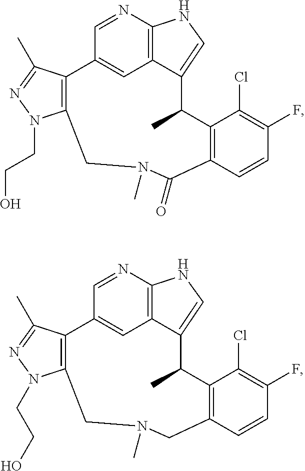 [SMILES: Cc1nn(CCO)c2c1-c1cnc3[nH]cc(c3c1)[C@H](C)c1c(ccc(F)c1Cl)C(=O)N(C)C2 ; Cc1nn(CCO)c2c1-c1cnc3[nH]cc(c3c1)[C@H](C)c1c(ccc(F)c1Cl)CN(C)C2]

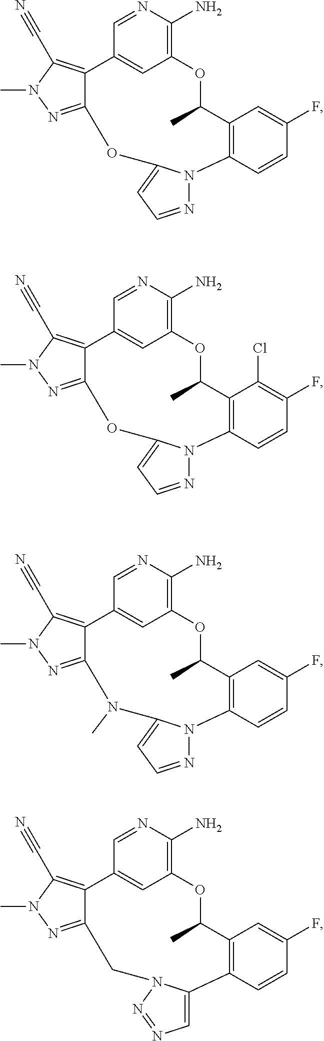 [SMILES: C[C@H]1Oc2cc(cnc2N)-c2c(nn(C)c2C#N)Oc2ccnn2-c2ccc(F)cc21 ; C[C@H]1Oc2cc(cnc2N)-c2c(nn(C)c2C#N)Oc2ccnn2-c2ccc(F)c(Cl)c21 ; C[C@H]1Oc2cc(cnc2N)-c2c(nn(C)c2C#N)N(C)c2ccnn2-c2ccc(F)cc21 ; C[C@H]1Oc2cc(cnc2N)-c2c(nn(C)c2C#N)Cn2nncc2-c2ccc(F)cc21]

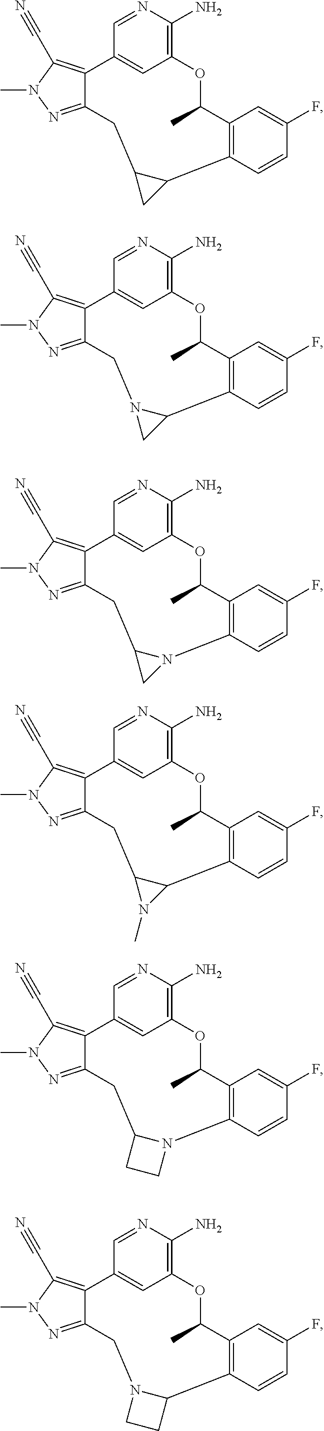

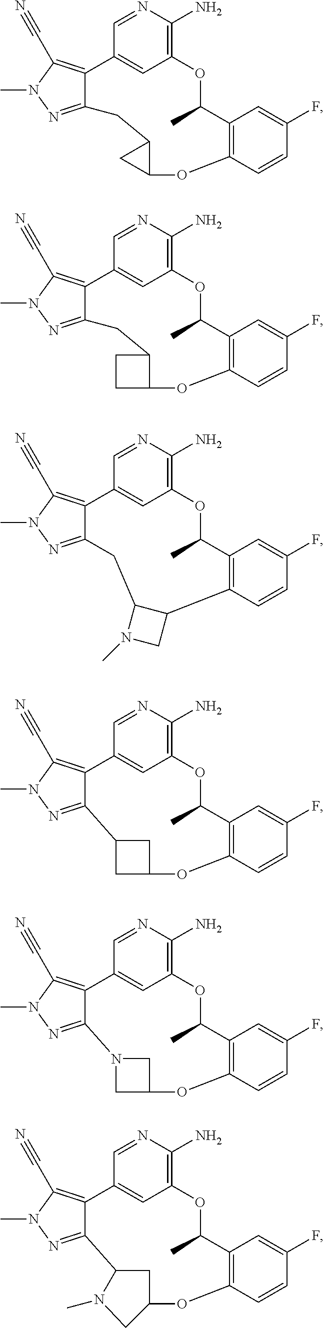

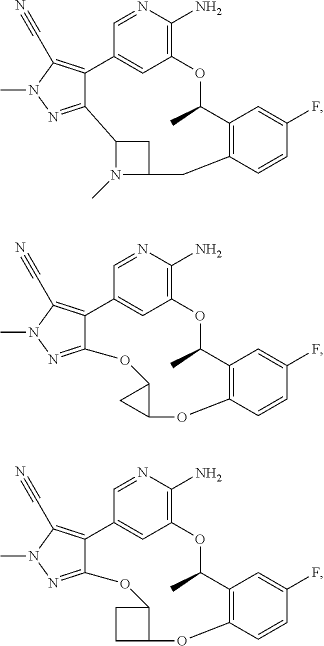 [SMILES: C[C@H]1Oc2cc(cnc2N)-c2c(nn(C)c2C#N)C2CC(Cc3ccc(F)cc31)N2C ; C[C@H]1Oc2cc(cnc2N)-c2c(nn(C)c2C#N)OC2CC2Oc2ccc(F)cc21 ; C[C@H]1Oc2cc(cnc2N)-c2c(nn(C)c2C#N)OC2CCC2Oc2ccc(F)cc21]

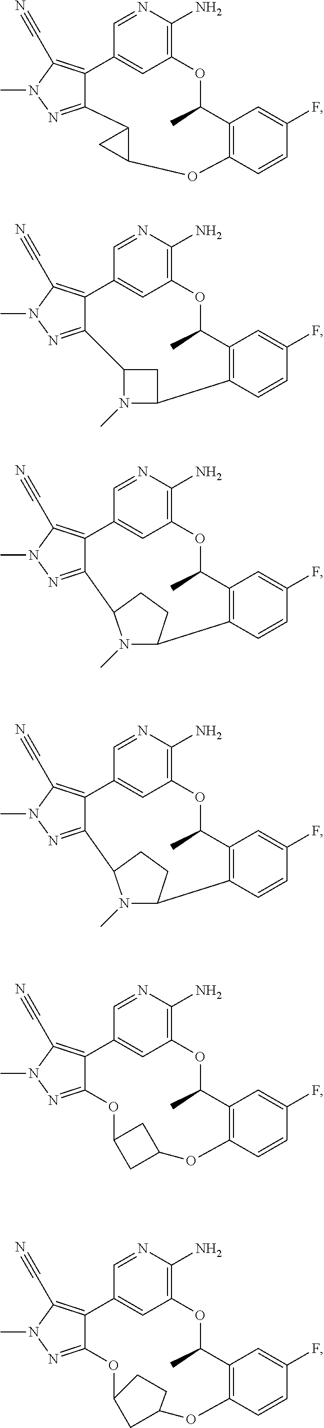 [SMILES: C[C@H]1Oc2cc(cnc2N)-c2c(nn(C)c2C#N)C2CC2Oc2ccc(F)cc21 ; C[C@H]1Oc2cc(cnc2N)-c2c(nn(C)c2C#N)C2CC(c3ccc(F)cc31)N2C ; C[C@H]1Oc2cc(cnc2N)-c2c(nn(C)c2C#N)C2CCC(c3ccc(F)cc31)N2C ; C[C@H]1Oc2cc(cnc2N)-c2c(nn(C)c2C#N)C2CCC(c3ccc(F)cc31)N2C ; C[C@H]1Oc2cc(cnc2N)-c2c(nn(C)c2C#N)OC2CC(C2)Oc2ccc(F)cc21 ; C[C@H]1Oc2cc(cnc2N)-c2c(nn(C)c2C#N)OC2CCC(C2)Oc2ccc(F)cc21]

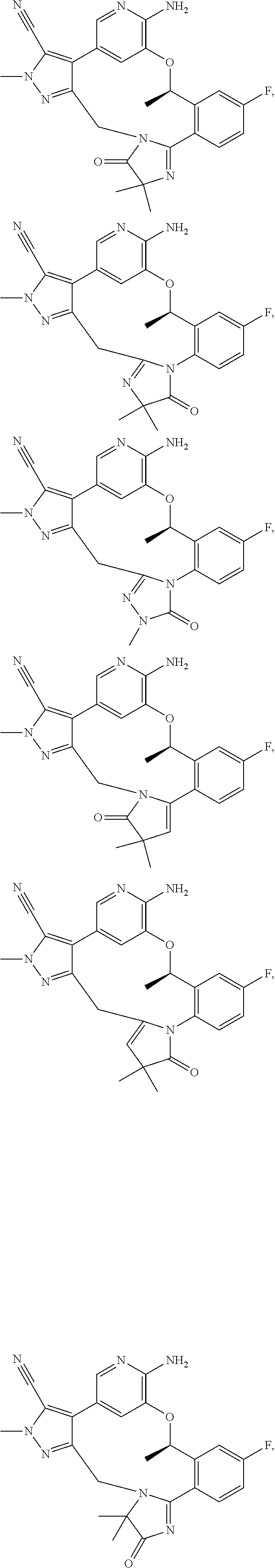

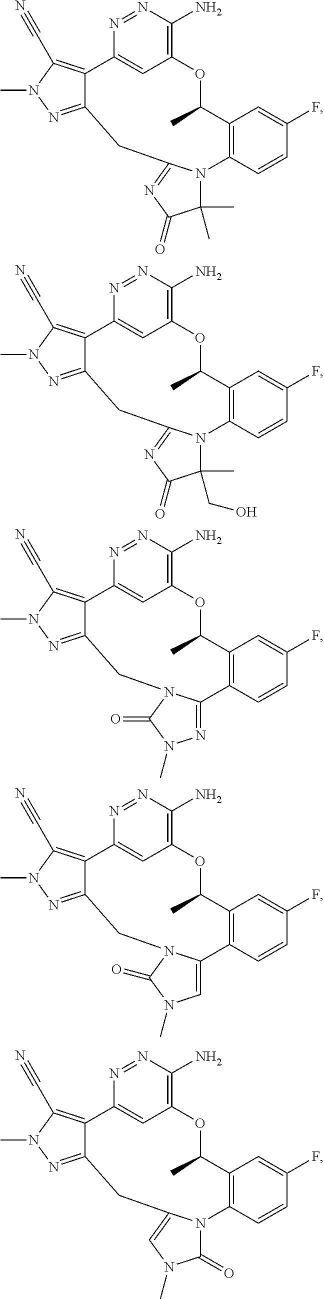

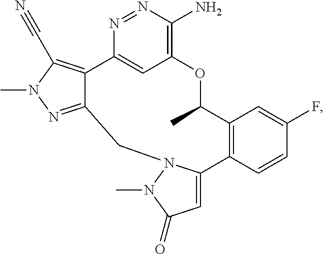 [SMILES: C[C@H]1Oc2cc(nnc2N)-c2c(nn(C)c2C#N)Cn2c(cc(=O)n2C)-c2ccc(F)cc21]

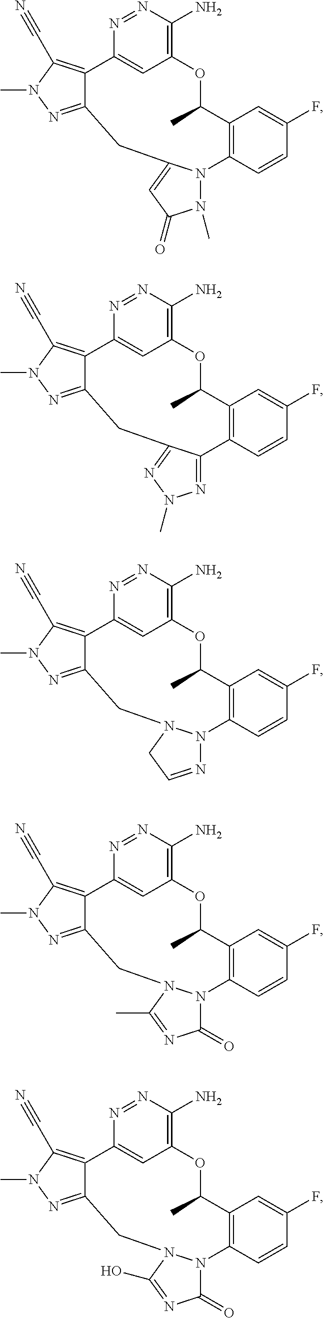

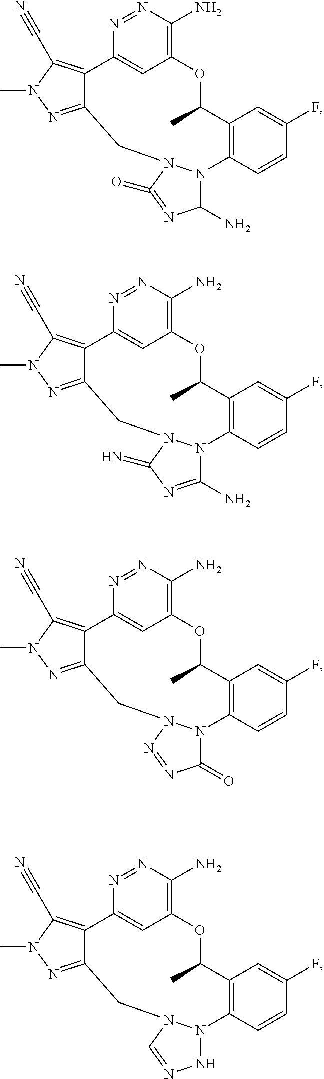

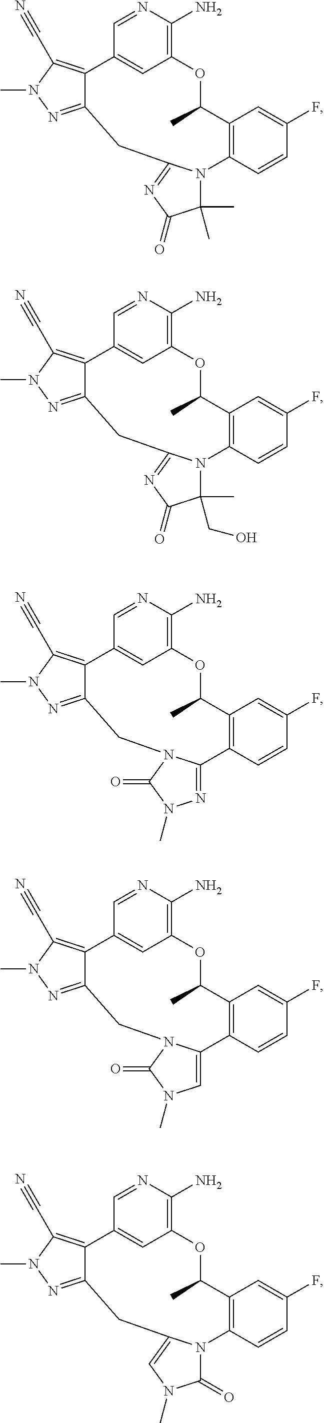 [SMILES: C[C@H]1Oc2cc(cnc2N)-c2c(nn(C)c2C#N)CC2=NC(=O)C(C)(C)N2c2ccc(F)cc21 ; C[C@H]1Oc2cc(cnc2N)-c2c(nn(C)c2C#N)CC2=NC(=O)C(C)(CO)N2c2ccc(F)cc21 ; C[C@H]1Oc2cc(cnc2N)-c2c(nn(C)c2C#N)Cn2c(nn(C)c2=O)-c2ccc(F)cc21 ; C[C@H]1Oc2cc(cnc2N)-c2c(nn(C)c2C#N)Cn2c(cn(C)c2=O)-c2ccc(F)cc21 ; C[C@H]1Oc2cc(cnc2N)-c2c(nn(C)c2C#N)Cc2cn(C)c(=O)n2-c2ccc(F)cc21]

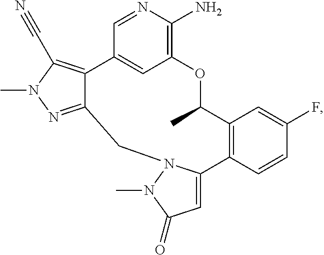 [SMILES: C[C@H]1Oc2cc(cnc2N)-c2c(nn(C)c2C#N)Cn2c(cc(=O)n2C)-c2ccc(F)cc21]

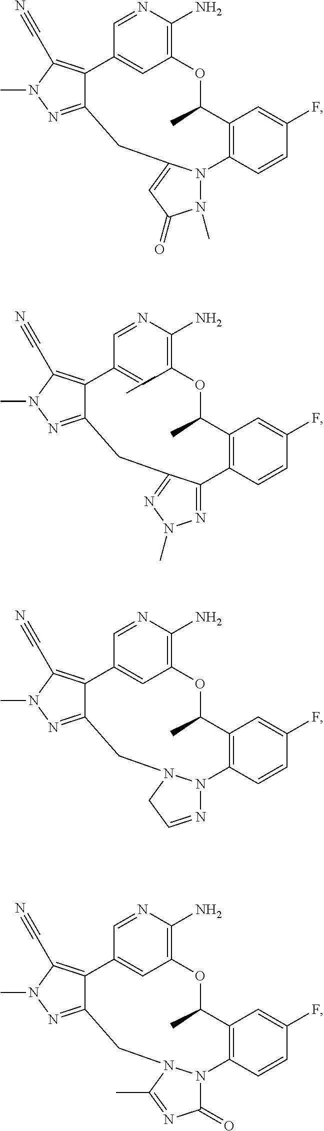

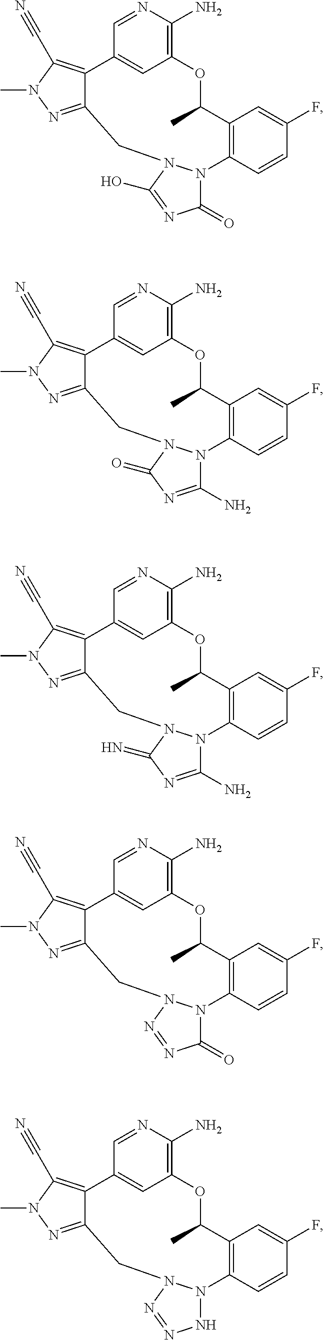 [SMILES: C[C@H]1Oc2cc(cnc2N)-c2c(nn(C)c2C#N)Cn2c(O)nc(=O)n2-c2ccc(F)cc21 ; C[C@H]1Oc2cc(cnc2N)-c2c(nn(C)c2C#N)Cn2c(=O)nc(N)n2-c2ccc(F)cc21 ; C[C@H]1Oc2cc(cnc2N)-c2c(nn(C)c2C#N)Cn2c(=N)nc(N)n2-c2ccc(F)cc21 ; C[C@H]1Oc2cc(cnc2N)-c2c(nn(C)c2C#N)Cn2nnc(=O)n2-c2ccc(F)cc21 ; C[C@H]1Oc2cc(cnc2N)-c2c(nn(C)c2C#N)CN2N=NNN2c2ccc(F)cc21]

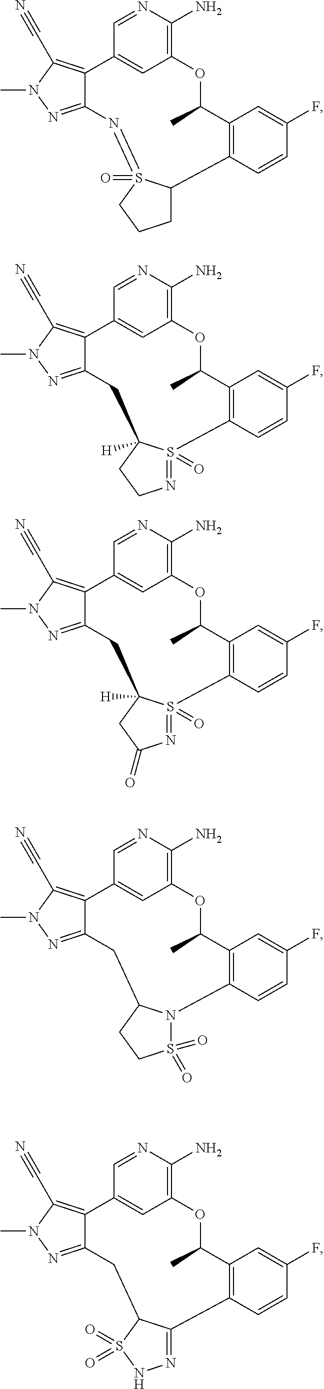

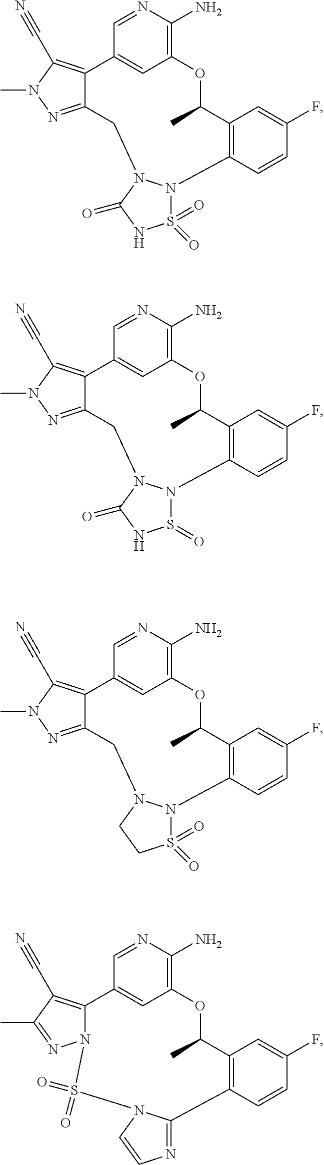 [SMILES: C[C@H]1Oc2cc(cnc2N)-c2c(nn(C)c2C#N)CN2C(=O)NS(=O)(=O)N2c2ccc(F)cc21 ; C[C@H]1Oc2cc(cnc2N)-c2c(nn(C)c2C#N)CN2C(=O)NS(=O)N2c2ccc(F)cc21 ; C[C@H]1Oc2cc(cnc2N)-c2c(nn(C)c2C#N)CN2CCS(=O)(=O)N2c2ccc(F)cc21 ; Cc1nn2c(c1C#N)-c1cnc(N)c(c1)O[C@H](C)c1cc(F)ccc1-c1nccn1S2(=O)=O]

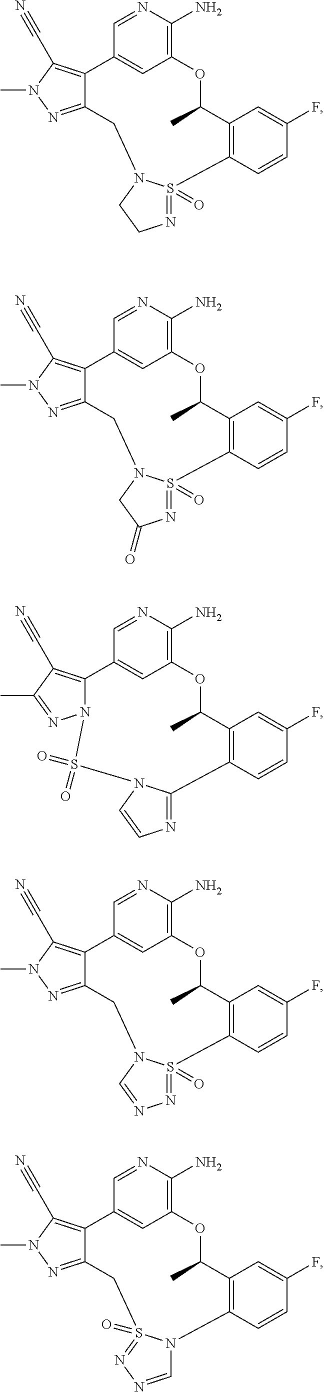 [SMILES: C[C@H]1Oc2cc(cnc2N)-c2c(nn(C)c2C#N)CN2CCN=S2(=O)c2ccc(F)cc21 ; C[C@H]1Oc2cc(cnc2N)-c2c(nn(C)c2C#N)CN2CC(=O)N=S2(=O)c2ccc(F)cc21 ; Cc1nn2c(c1C#N)-c1cnc(N)c(c1)O[C@H](C)c1cc(F)ccc1-c1nccn1S2(=O)=O ; C[C@H]1Oc2cc(cnc2N)-c2c(nn(C)c2C#N)CN2C=NN=S2(=O)c2ccc(F)cc21 ; C[C@H]1Oc2cc(cnc2N)-c2c(nn(C)c2C#N)CS2(=O)=NN=CN2c2ccc(F)cc21]

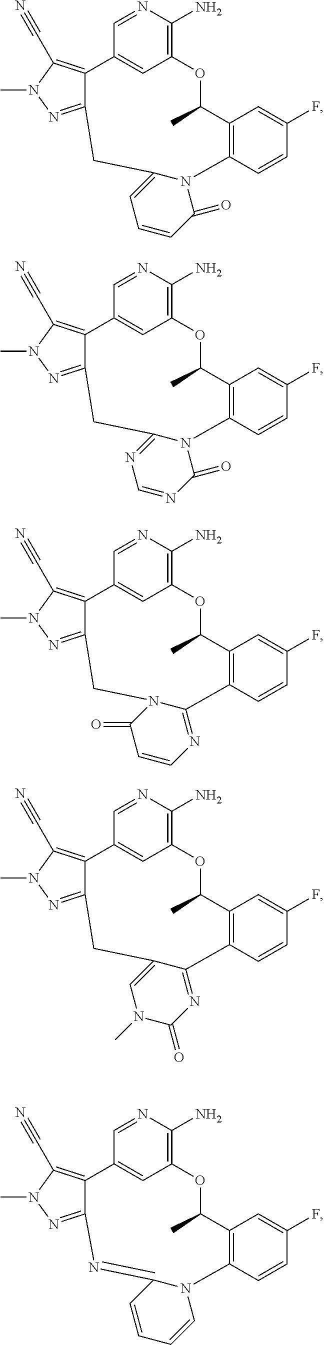

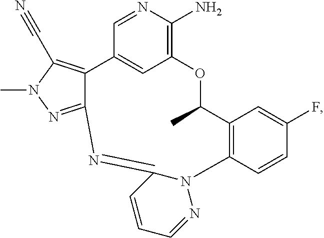 [SMILES: C[C@H]1Oc2cc(cnc2N)-c2c(nn(C)c2C#N)/N=C2\C=CC=NN2c2ccc(F)cc21]

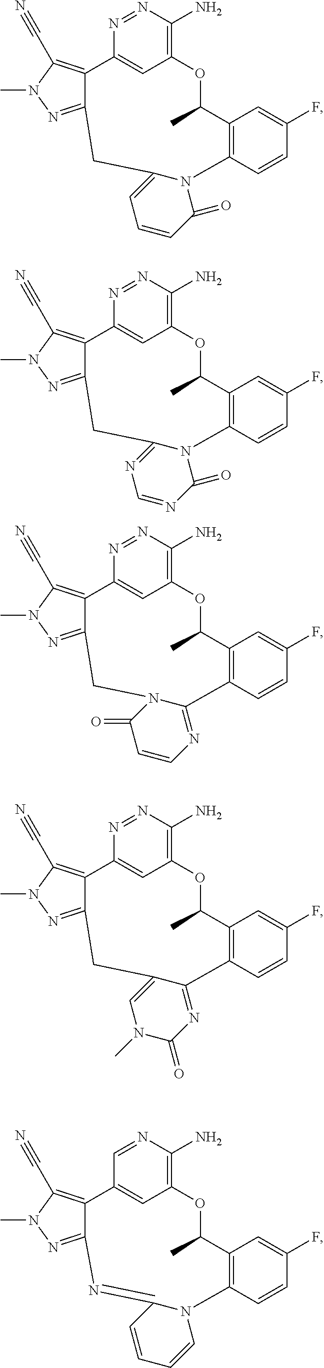

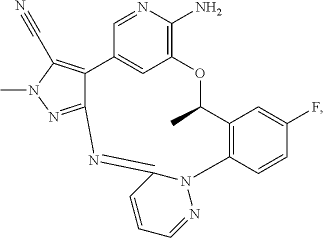 [SMILES: C[C@H]1Oc2cc(cnc2N)-c2c(nn(C)c2C#N)/N=C2\C=CC=NN2c2ccc(F)cc21]

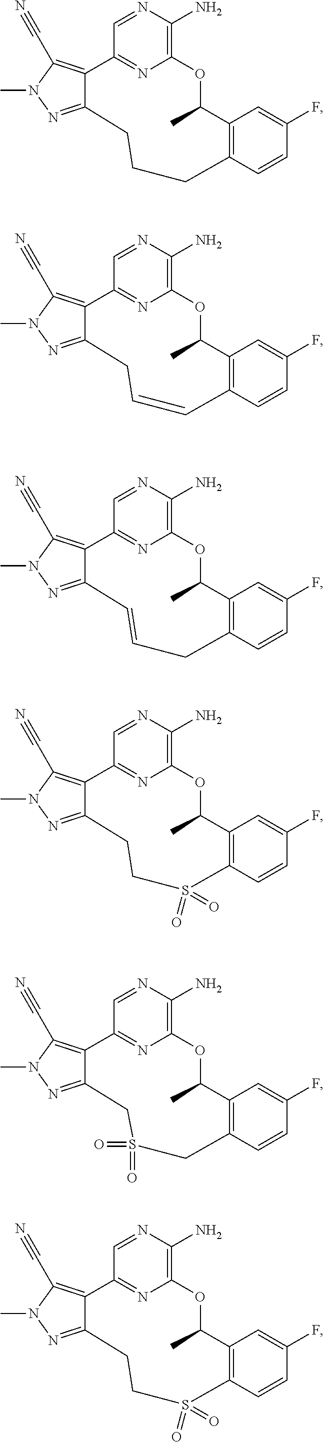 [SMILES: C[C@H]1Oc2nc(cnc2N)-c2c(nn(C)c2C#N)CCCc2ccc(F)cc21 ; C[C@H]1Oc2nc(cnc2N)-c2c(nn(C)c2C#N)C/C=C\c2ccc(F)cc21 ; C[C@H]1Oc2nc(cnc2N)-c2c(nn(C)c2C#N)/C=C/Cc2ccc(F)cc21 ; C[C@H]1Oc2nc(cnc2N)-c2c(nn(C)c2C#N)CCS(=O)(=O)c2ccc(F)cc21 ; C[C@H]1Oc2nc(cnc2N)-c2c(nn(C)c2C#N)CS(=O)(=O)Cc2ccc(F)cc21 ; C[C@H]1Oc2nc(cnc2N)-c2c(nn(C)c2C#N)CCS(=O)(=O)c2ccc(F)cc21]

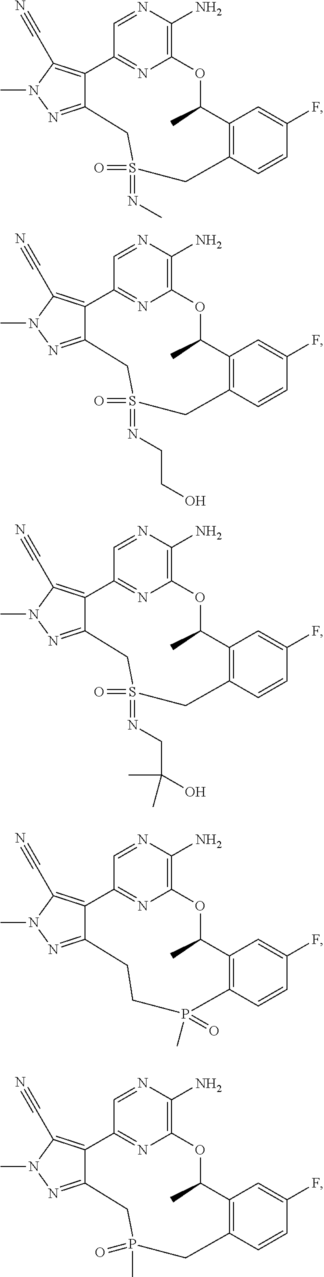

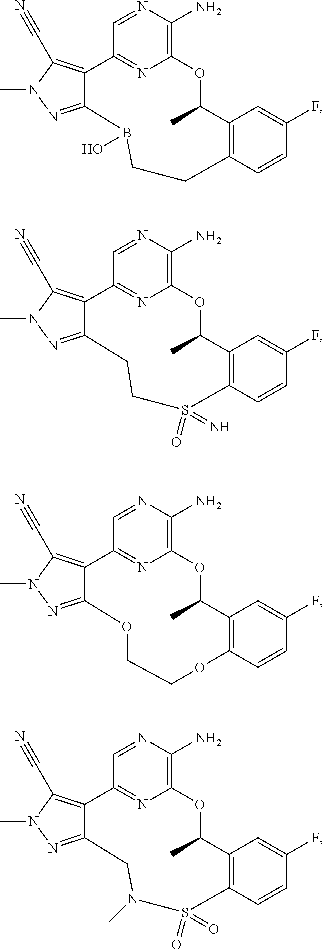 [SMILES: C[C@H]1Oc2nc(cnc2N)-c2c(nn(C)c2C#N)B(O)CCc2ccc(F)cc21 ; C[C@H]1Oc2nc(cnc2N)-c2c(nn(C)c2C#N)CCS(=N)(=O)c2ccc(F)cc21 ; C[C@H]1Oc2nc(cnc2N)-c2c(nn(C)c2C#N)OCCOc2ccc(F)cc21 ; C[C@H]1Oc2nc(cnc2N)-c2c(nn(C)c2C#N)CN(C)S(=O)(=O)c2ccc(F)cc21]

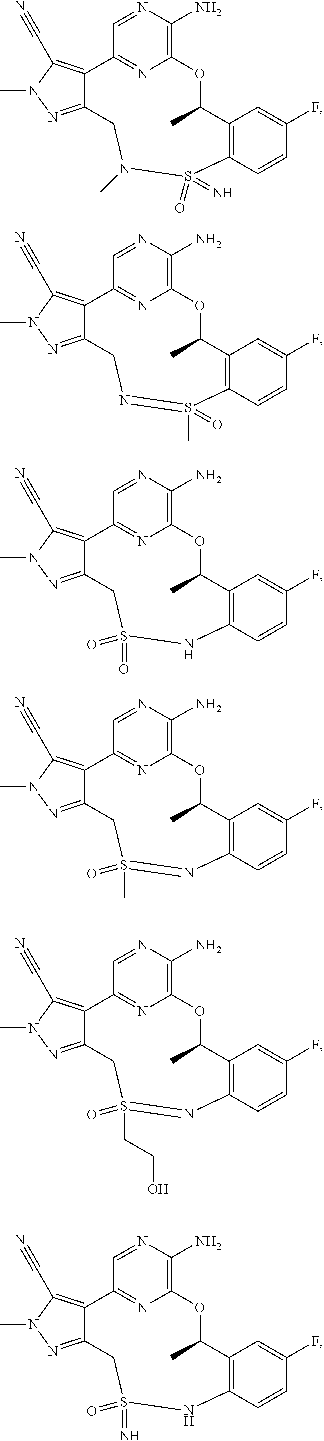

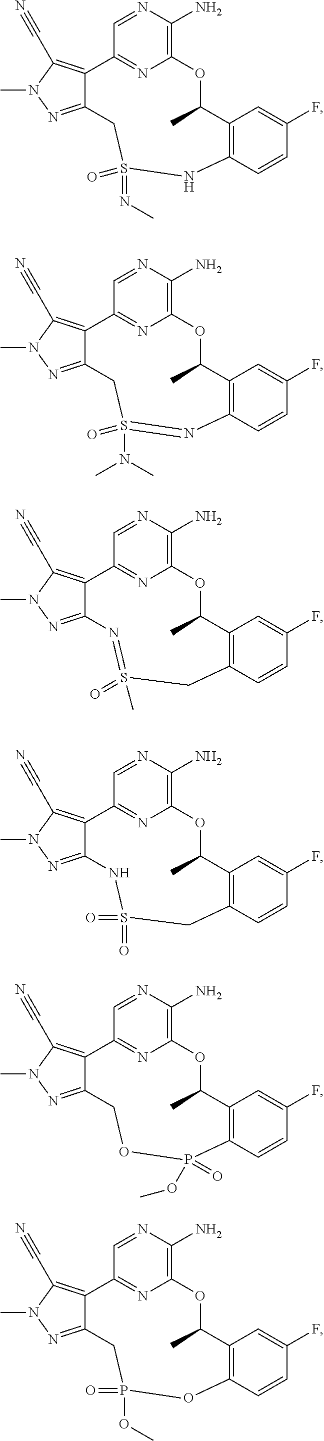

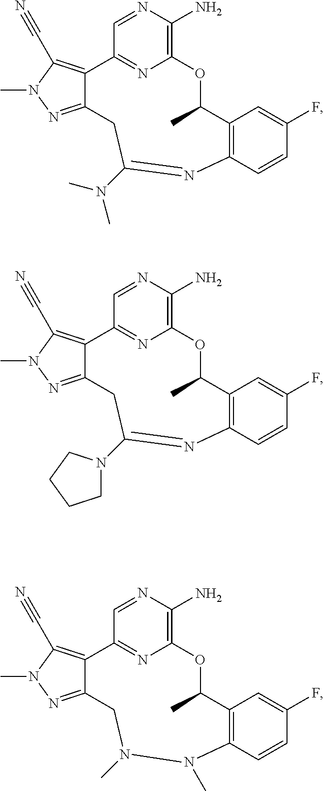 [SMILES: C[C@H]1Oc2nc(cnc2N)-c2c(nn(C)c2C#N)C/C(N(C)C)=N\c2ccc(F)cc21 ; C[C@H]1Oc2nc(cnc2N)-c2c(nn(C)c2C#N)C/C(N2CCCC2)=N\c2ccc(F)cc21 ; C[C@H]1Oc2nc(cnc2N)-c2c(nn(C)c2C#N)CN(C)N(C)c2ccc(F)cc21]

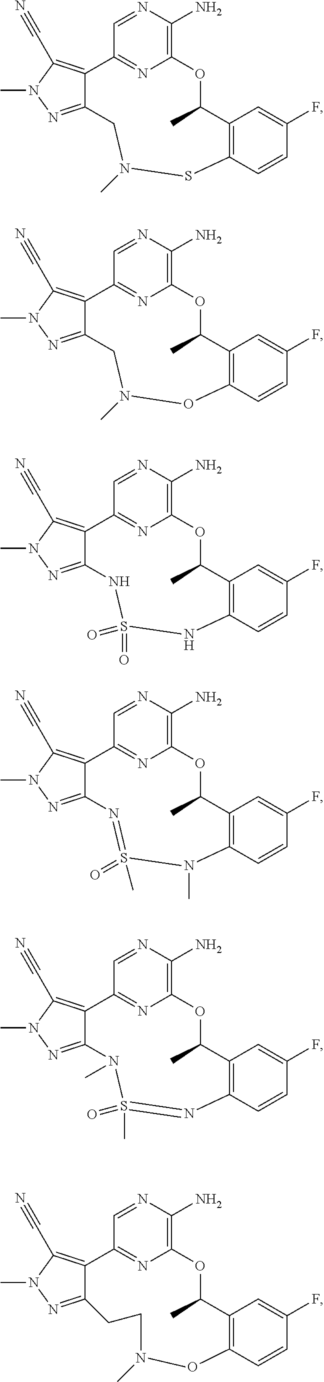

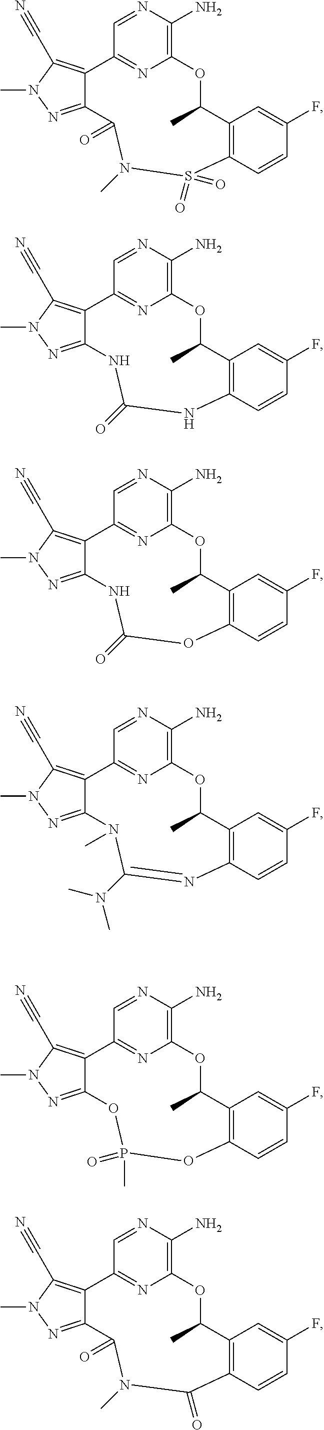

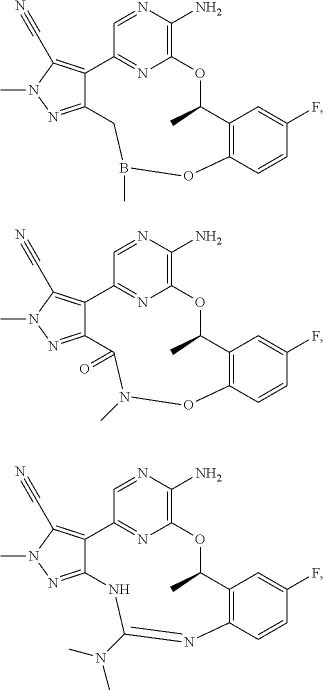

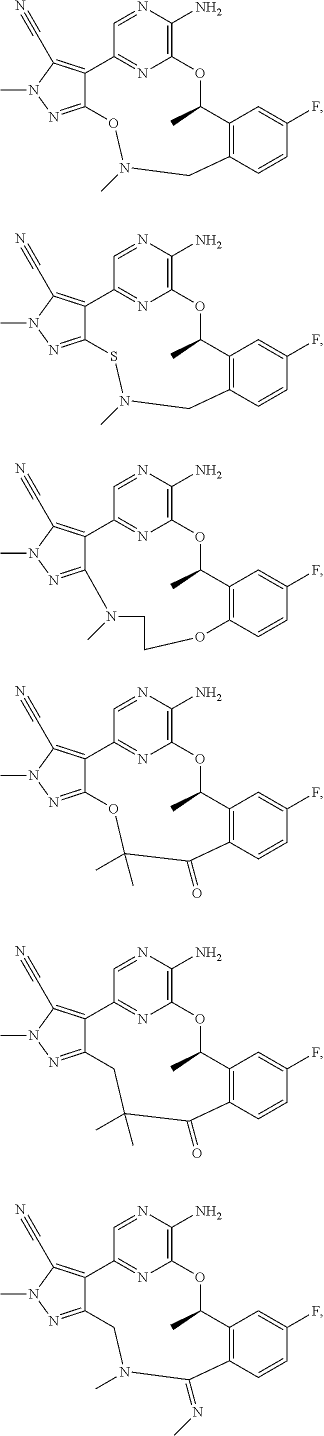

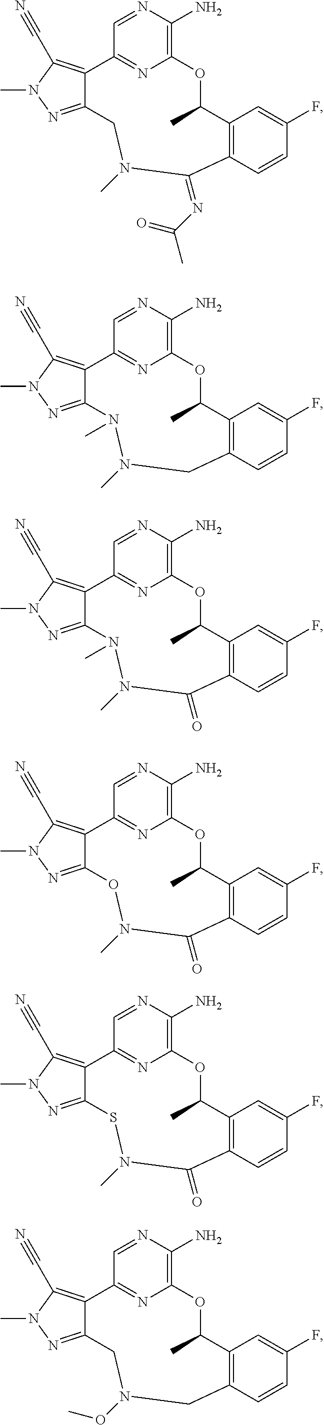

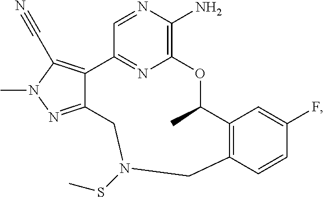 [SMILES: CSN1Cc2ccc(F)cc2[C@@H](C)Oc2nc(cnc2N)-c2c(nn(C)c2C#N)C1]

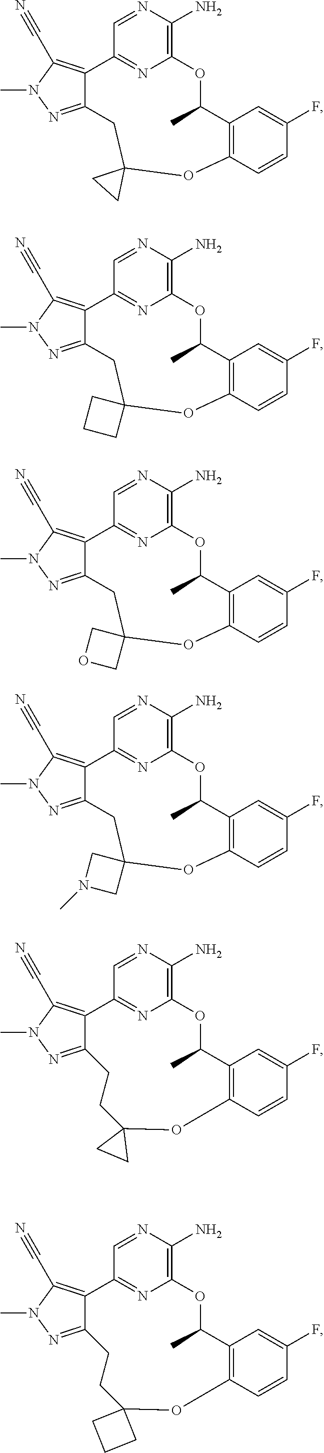

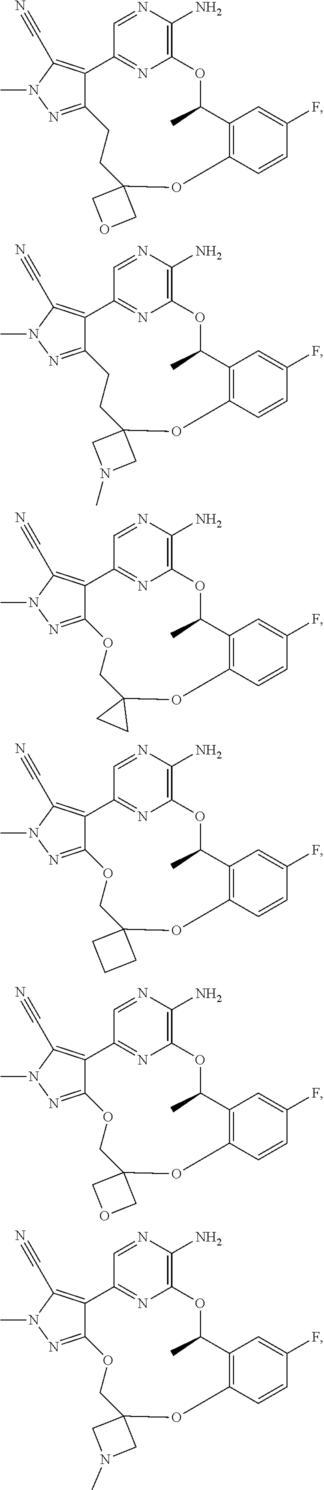

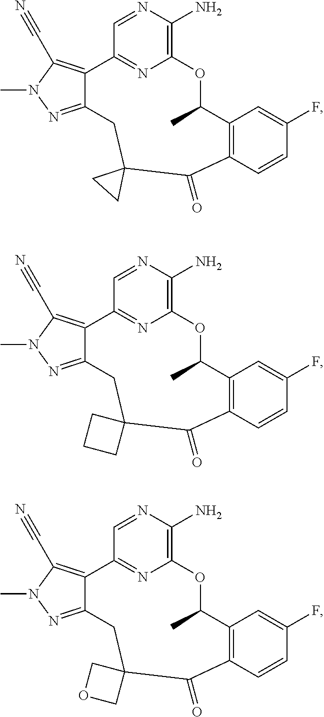 [SMILES: C[C@H]1Oc2nc(cnc2N)-c2c(nn(C)c2C#N)CC2(CC2)C(=O)c2ccc(F)cc21 ; C[C@H]1Oc2nc(cnc2N)-c2c(nn(C)c2C#N)CC2(CCC2)C(=O)c2ccc(F)cc21 ; C[C@H]1Oc2nc(cnc2N)-c2c(nn(C)c2C#N)CC2(COC2)C(=O)c2ccc(F)cc21]

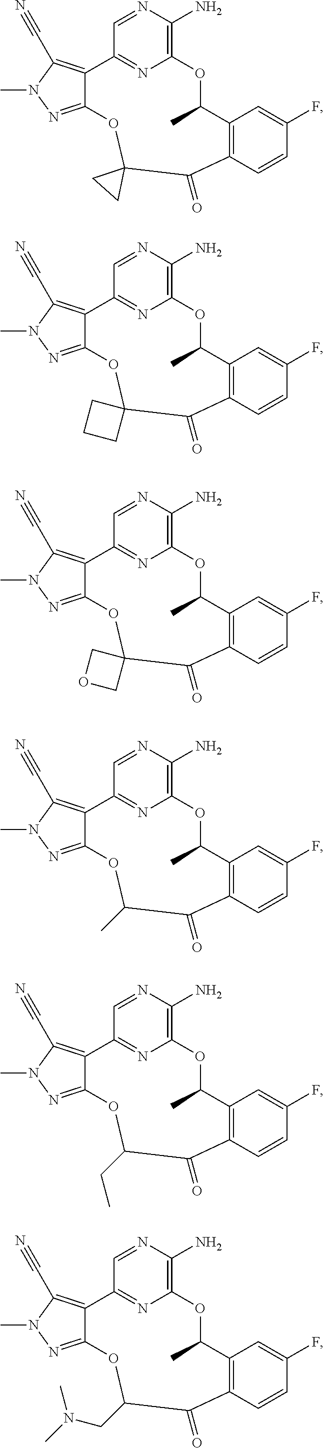

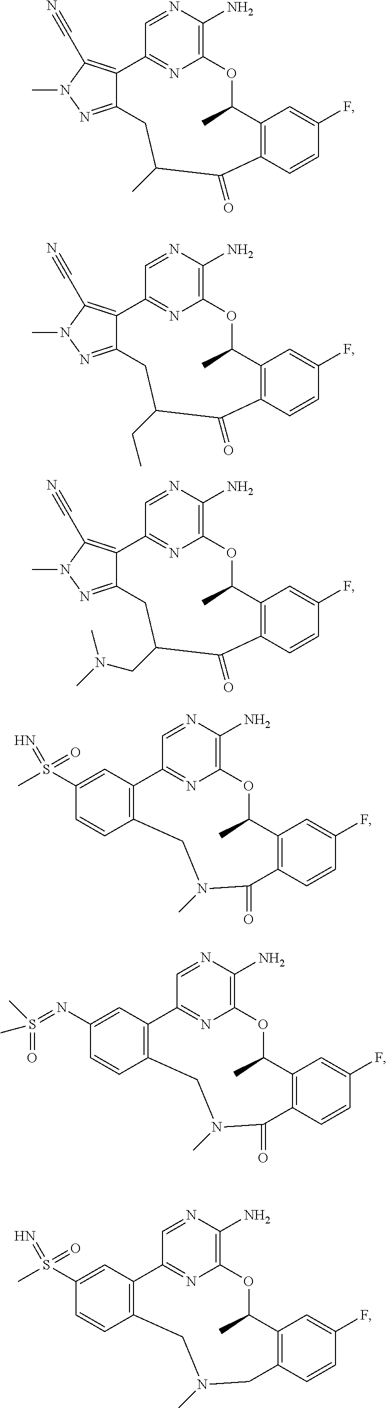

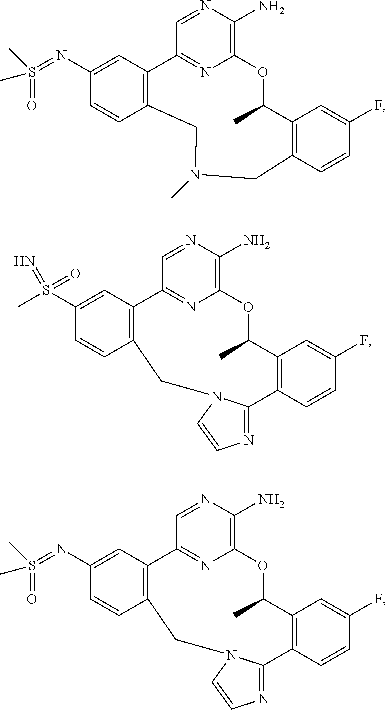

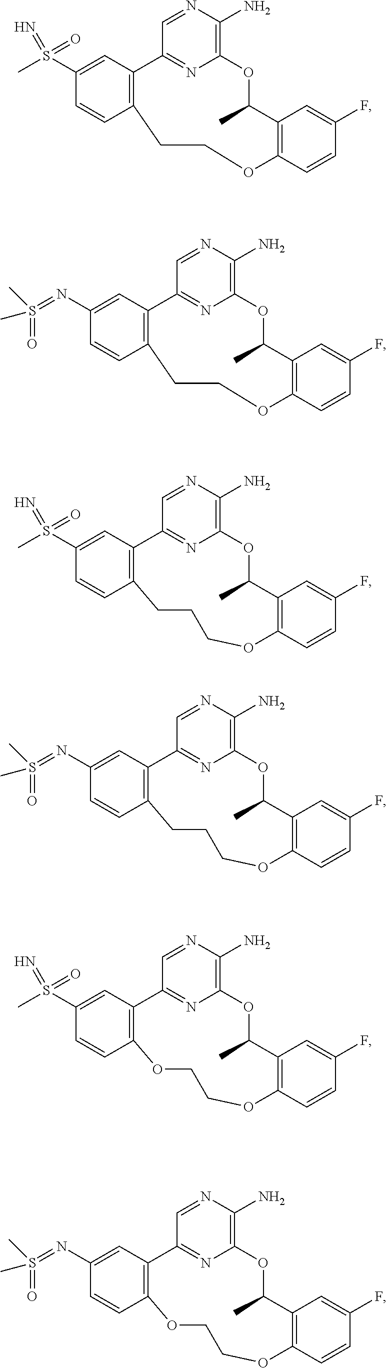 [SMILES: C[C@H]1Oc2nc(cnc2N)-c2cc(S(C)(=N)=O)ccc2CCOc2ccc(F)cc21 ; C[C@H]1Oc2nc(cnc2N)-c2cc(N=S(C)(C)=O)ccc2CCOc2ccc(F)cc21 ; C[C@H]1Oc2nc(cnc2N)-c2cc(S(C)(=N)=O)ccc2CCCOc2ccc(F)cc21 ; C[C@H]1Oc2nc(cnc2N)-c2cc(N=S(C)(C)=O)ccc2CCCOc2ccc(F)cc21 ; C[C@H]1Oc2nc(cnc2N)-c2cc(S(C)(=N)=O)ccc2OCCOc2ccc(F)cc21 ; C[C@H]1Oc2nc(cnc2N)-c2cc(N=S(C)(C)=O)ccc2OCCOc2ccc(F)cc21]

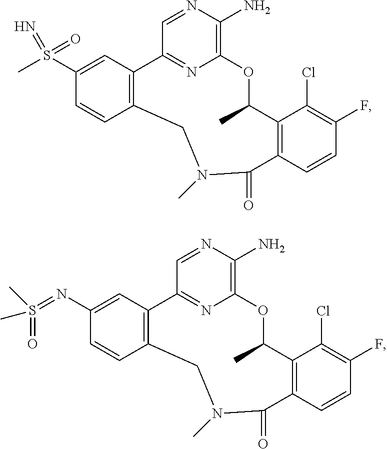

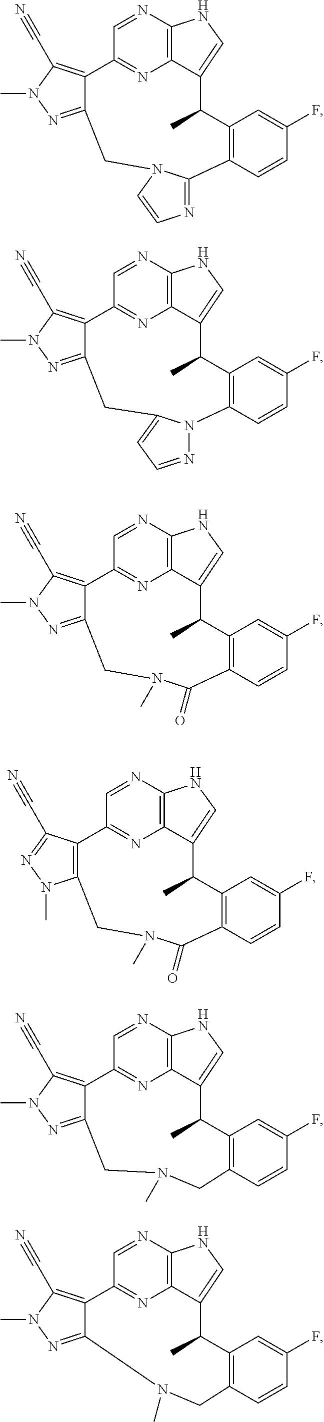

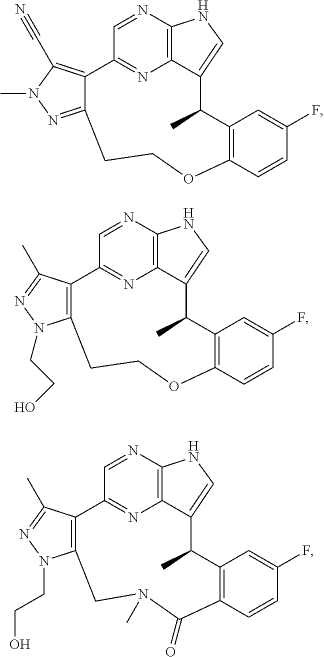

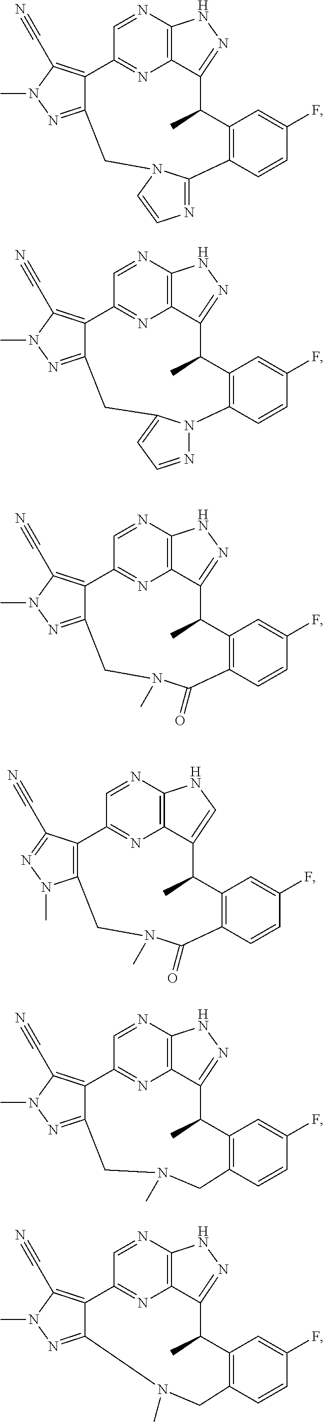

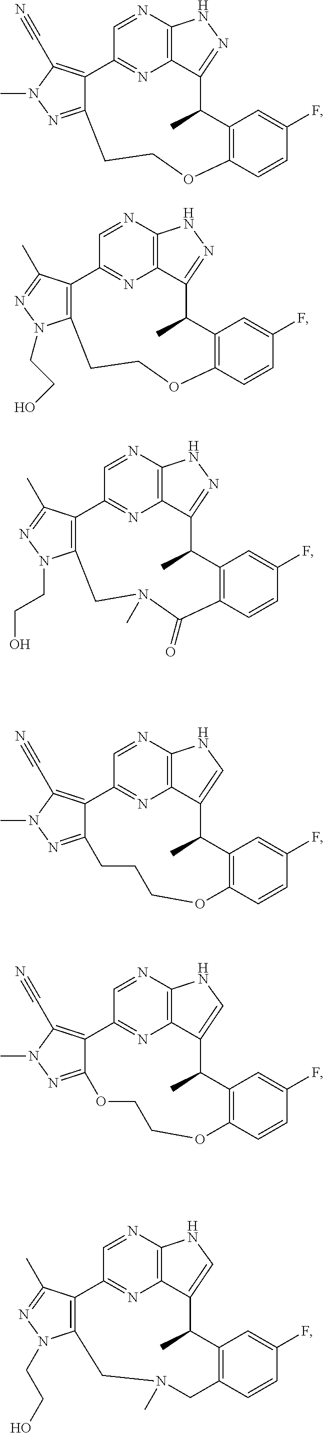

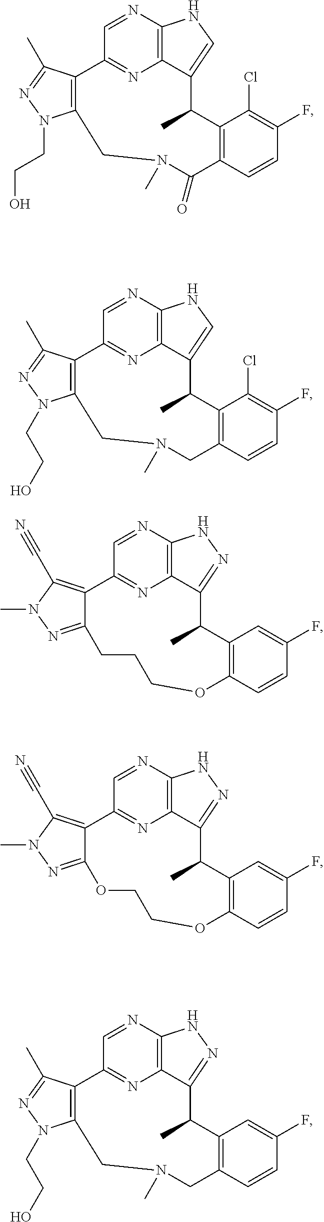

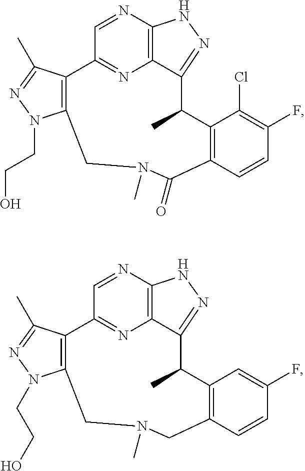 [SMILES: Cc1nn(CCO)c2c1-c1cnc3[nH]nc(c3n1)[C@H](C)c1c(ccc(F)c1Cl)C(=O)N(C)C2 ; Cc1nn(CCO)c2c1-c1cnc3[nH]nc(c3n1)[C@H](C)c1cc(F)ccc1CN(C)C2]

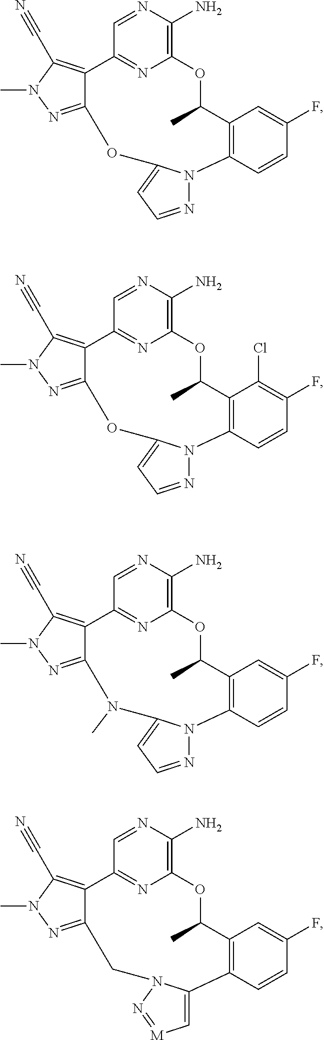

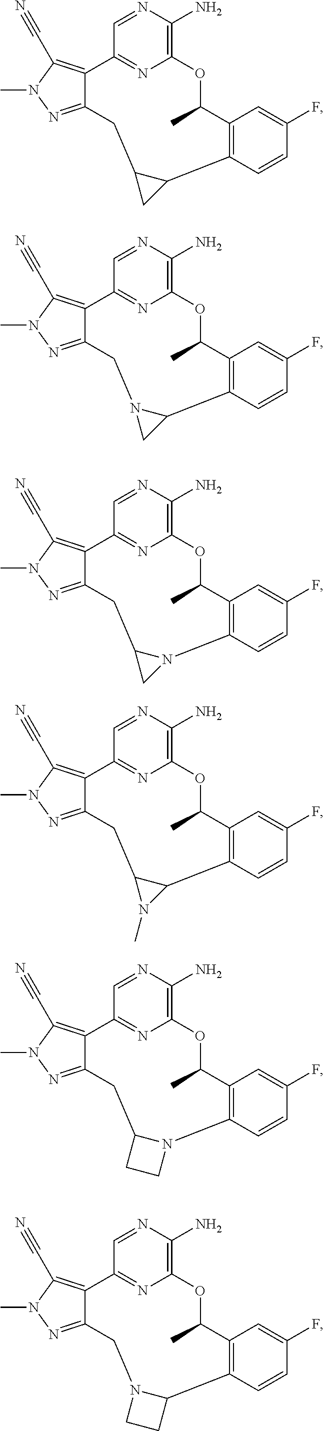

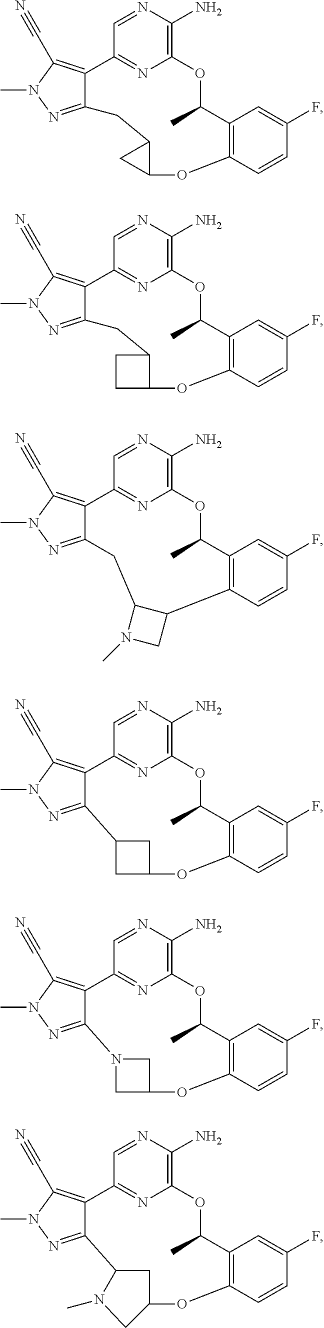

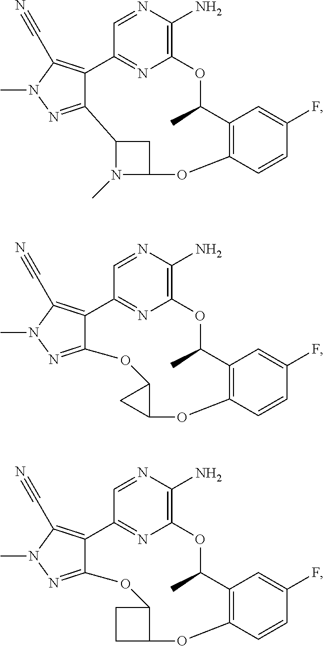 [SMILES: C[C@H]1Oc2nc(cnc2N)-c2c(nn(C)c2C#N)C2CC(Oc3ccc(F)cc31)N2C ; C[C@H]1Oc2nc(cnc2N)-c2c(nn(C)c2C#N)OC2CC2Oc2ccc(F)cc21 ; C[C@H]1Oc2nc(cnc2N)-c2c(nn(C)c2C#N)OC2CCC2Oc2ccc(F)cc21]

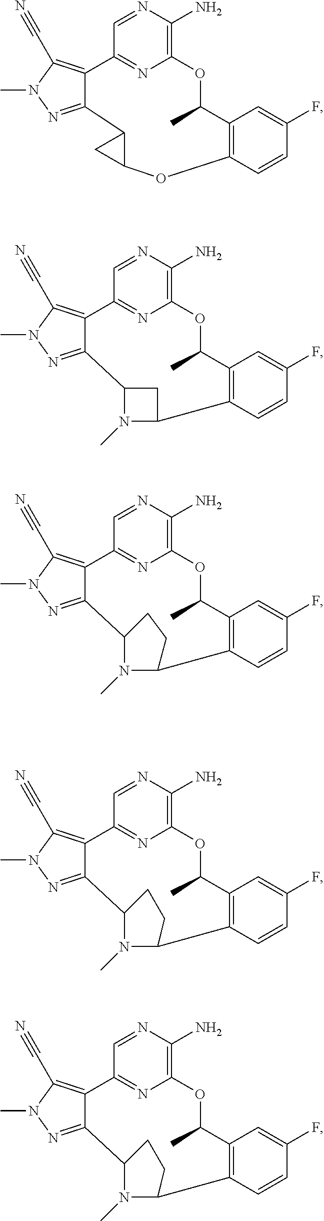 [SMILES: C[C@H]1Oc2nc(cnc2N)-c2c(nn(C)c2C#N)C2CC2Oc2ccc(F)cc21 ; C[C@H]1Oc2nc(cnc2N)-c2c(nn(C)c2C#N)C2CC(c3ccc(F)cc31)N2C ; C[C@H]1Oc2nc(cnc2N)-c2c(nn(C)c2C#N)C2CCC(c3ccc(F)cc31)N2C ; C[C@H]1Oc2nc(cnc2N)-c2c(nn(C)c2C#N)C2CCC(c3ccc(F)cc31)N2C ; C[C@H]1Oc2nc(cnc2N)-c2c(nn(C)c2C#N)C2CCC(c3ccc(F)cc31)N2C]

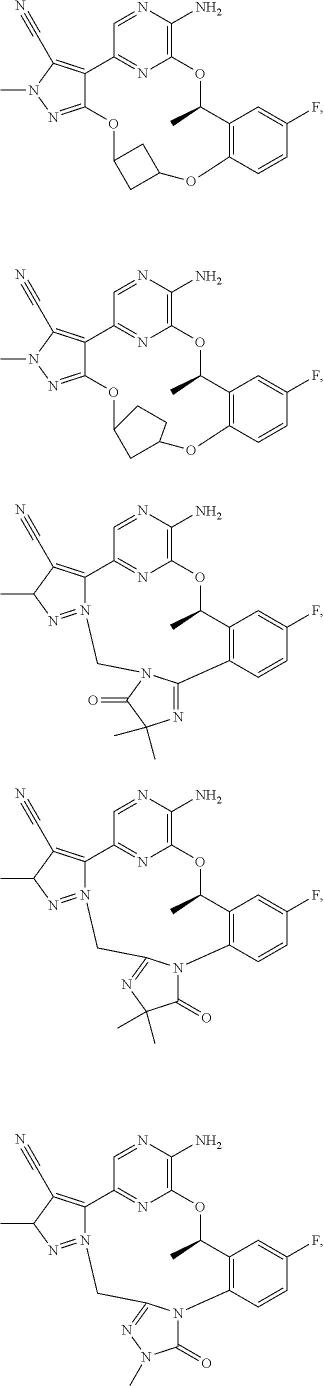

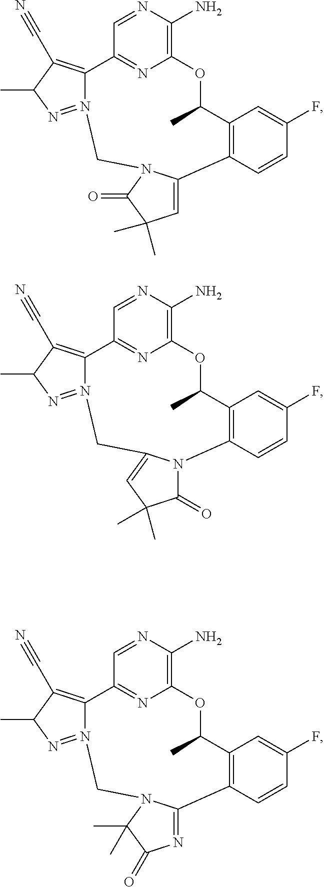

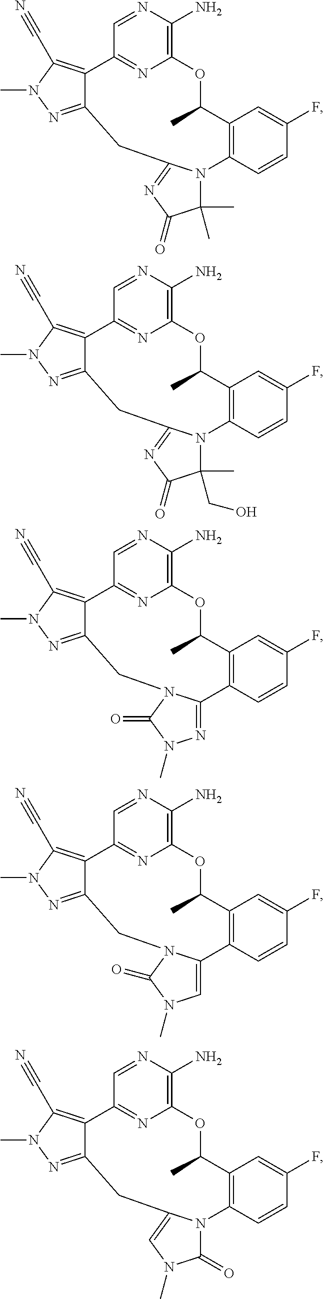

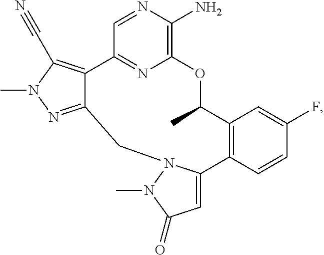 [SMILES: C[C@H]1Oc2nc(cnc2N)-c2c(nn(C)c2C#N)Cn2c(cc(=O)n2C)-c2ccc(F)cc21]

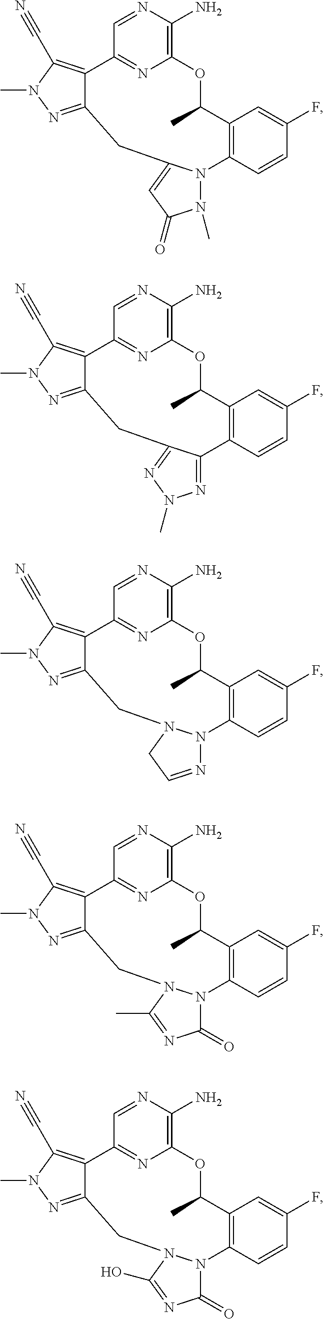

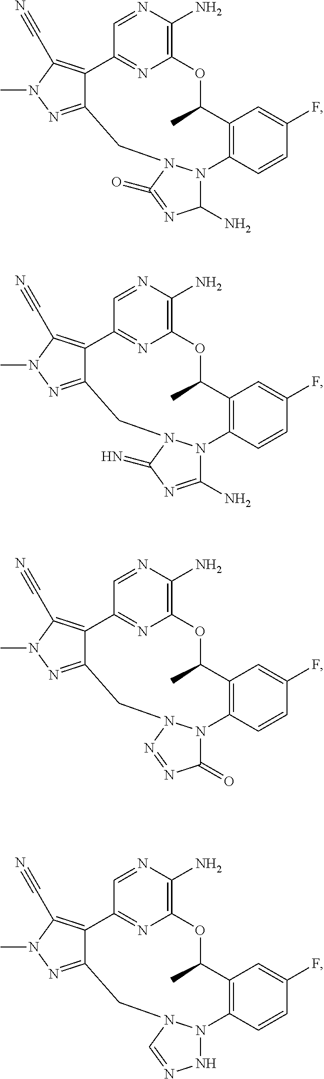

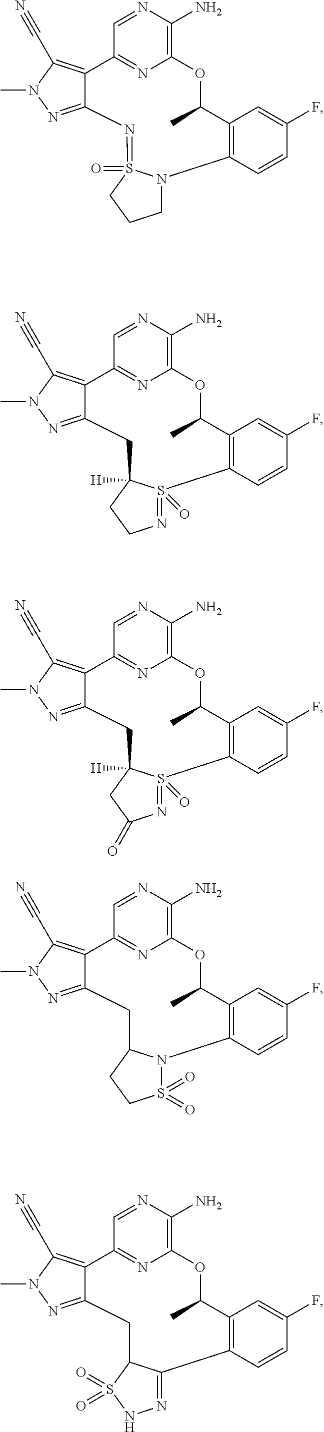 [SMILES: C[C@H]1Oc2nc(cnc2N)-c2c(nn(C)c2C#N)N=S2(=O)CCCN2c2ccc(F)cc21 ; C[C@H]1Oc2nc(cnc2N)-c2c(nn(C)c2C#N)C[C@@H]2CCN=S2(=O)c2ccc(F)cc21 ; C[C@H]1Oc2nc(cnc2N)-c2c(nn(C)c2C#N)C[C@@H]2CC(=O)N=S2(=O)c2ccc(F)cc21 ; C[C@H]1Oc2nc(cnc2N)-c2c(nn(C)c2C#N)CC2CCS(=O)(=O)N2c2ccc(F)cc21 ; C[C@H]1Oc2nc(cnc2N)-c2c(nn(C)c2C#N)CC2C(=NNS2(=O)=O)c2ccc(F)cc21]

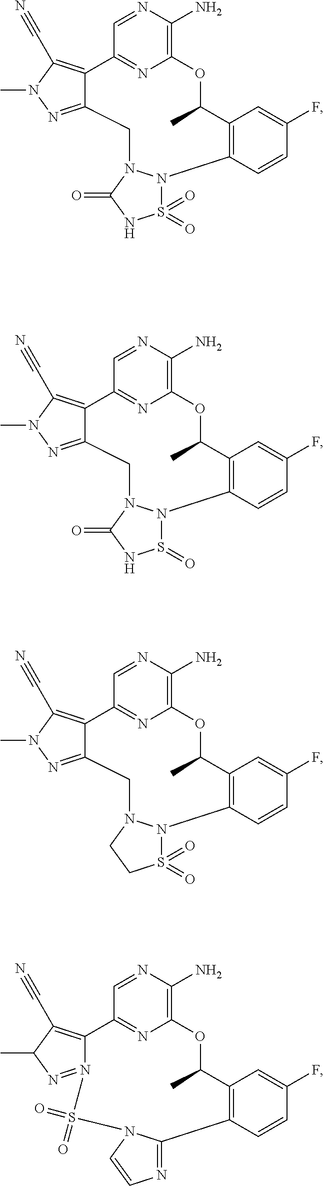

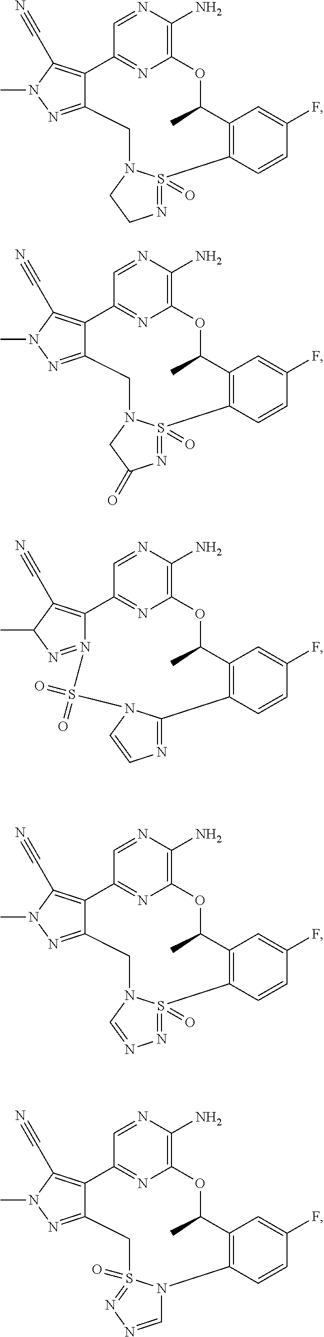

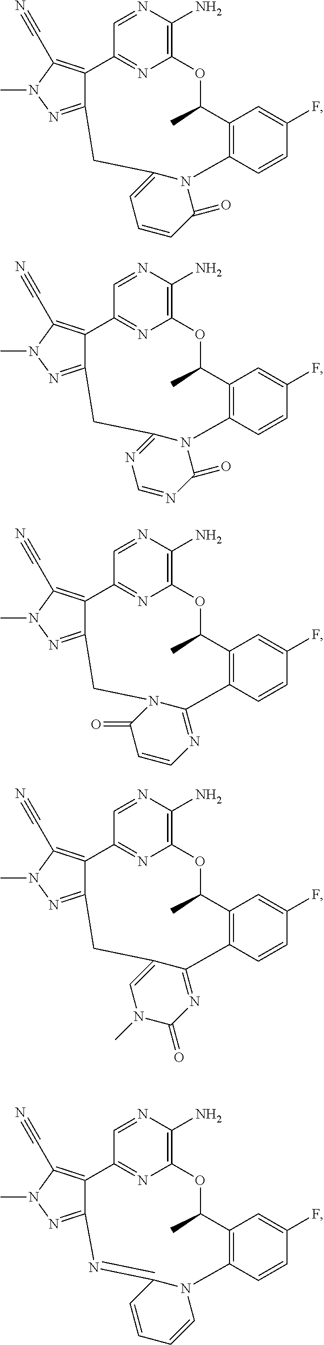

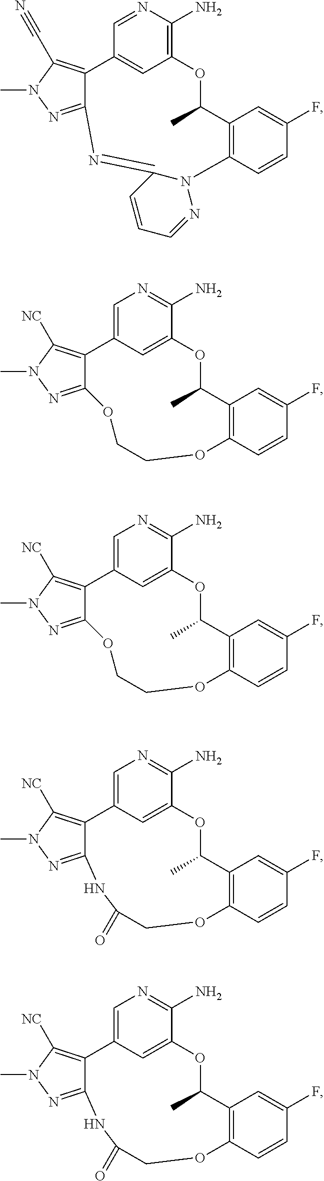 [SMILES: C[C@H]1Oc2cc(cnc2N)-c2c(nn(C)c2C#N)/N=C2\C=CC=NN2c2ccc(F)cc21 ; C[C@H]1Oc2cc(cnc2N)-c2c(nn(C)c2C#N)OCCOc2ccc(F)cc21 ; C[C@@H]1Oc2cc(cnc2N)-c2c(nn(C)c2C#N)OCCOc2ccc(F)cc21 ; C[C@@H]1Oc2cc(cnc2N)-c2c(nn(C)c2C#N)NC(=O)COc2ccc(F)cc21 ; C[C@H]1Oc2cc(cnc2N)-c2c(nn(C)c2C#N)NC(=O)COc2ccc(F)cc21]

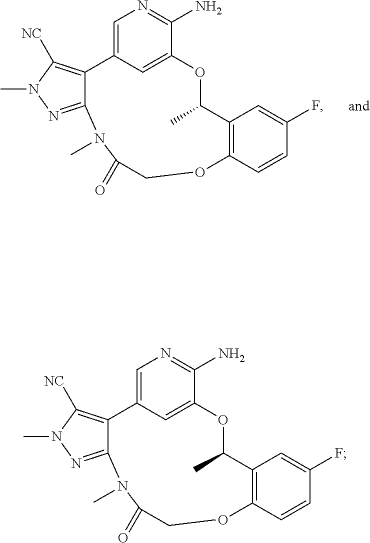 [SMILES: C[C@@H]1Oc2cc(cnc2N)-c2c(nn(C)c2C#N)N(C)C(=O)COc2ccc(F)cc21 ; "and" ; C[C@H]1Oc2cc(cnc2N)-c2c(nn(C)c2C#N)N(C)C(=O)COc2ccc(F)cc21]

or a pharmaceutically acceptable salt thereof.

Synthetic Methods

Compounds of the invention and intermediates thereof can be prepared in a number of ways known to one of ordinary skill in the art of organic synthesis. The reagents and starting materials are readily available to one of ordinary skill in the art.

A particularly useful reference of synthetic methods which may be applicable to preparation of compounds of the present invention may be found in Larock, R. C. Comprehensive Organic Transformations, VCH: New York, 1989, hereby incorporated by reference. Another useful reference for choosing appropriate protecting groups used in the protection of the reactive functional groups present in the compounds described in this invention is Greene and Wuts, Protective Groups In Organic Synthesis, Wiley and Sons, 1991, also incorporated by reference herein as if fully set forth.

Compounds of the present invention may be prepared by a variety of techniques, some of which are illustrated below. It is understood by one skilled in the art that these methods are representative, and are not limiting. Many reactions relevant to building compounds of the current invention are described in WO 2013/132376 and US 2013/0253197, both of which are incorporated by reference in their entirety.

Method A.

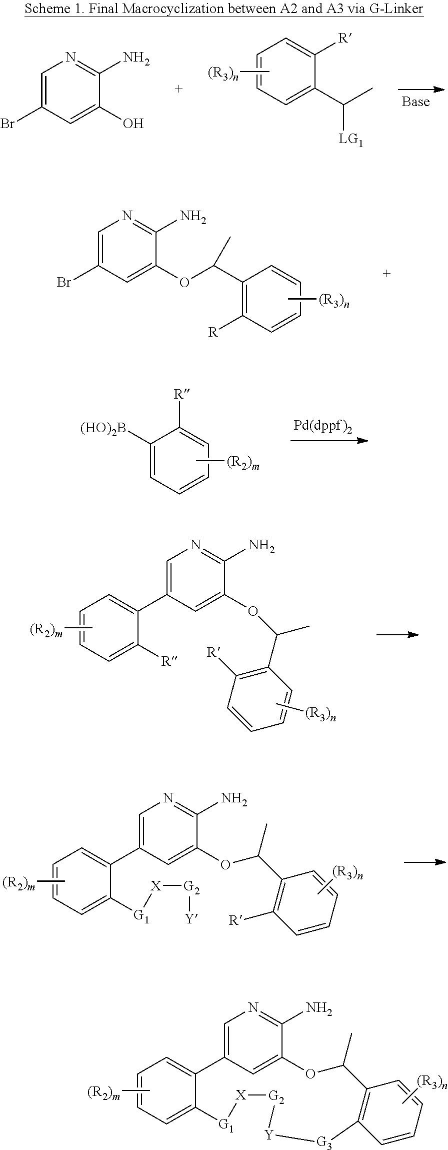

In Scheme 1 illustrated above, the A-ring pyridine is coupled to a benzylic position via nucleophilic displacement of a benzylic leaving group LG₁ under S_{N}2 conditions. LG₁ is most likely to be a halide, preferably a chloride or a bromide, but it could also be a sulfonate ester, or in the presence of appropriate transition metal catalysts an acyloxy group, carbonate, etc. The third aryl group is then introduced by one of many couplings used to make biaryls, illustrated in this case as a Suzuki-Miyauri coupling, between the 5-bromine on the pyridine, and the boronic acid residue on the aryl boronate. Such a compound is then cyclized by building the G-linker between the R′ and R″ groups on the A2 and A3 rings. It is understood that although the linker is drawn out as containing all five possible components G₁, X, G₂, Y and G₃, that the components of these as laid out in the specification are very variable, and that as few as one of these five elements, or as many as all five may be present in any particular compound. As this leads to a very wide array of possible linker termini, this chemistry, and consequently the nature of R′ and R″ can be very varied indeed. R′ and R″ can be many different functional groups, as needed to attach the linker most efficiently to A2 and A3. The chemistry to build the linker, regardless of whether it is built onto one of A2 and A3 from the beginning, or is largely prebuilt, and then attached when near completion to A2 and A3 is very variable in both synthetic techniques used, and number of steps, the latter being very dependent on the number of components present in the linker. As illustrated above the linking may be done first via first coupling R″ to X, or a suitable derivative of it, which may include for instance all or a part of the G₁ moiety, which may then be built up to a moiety containing Y′, which may or may not contain a part or the whole of G₃, which can be coupled to R′ to complete the macrocycle. Alternatively linkage of R′ to Y′, could be the first step, and the final completion of the macrocycle be coupling of R″ to X′, or whatever the left hand terminus of the linker is. Or the macrocyclization, if more convenient may be done at some internal point in the G-linking group. For example, one or both of R′ and R″ can be halogen, which can be chain extended in a very wide variety of ways, involving many forms of coupling reaction familiar to one skilled in the art, introducing carbon fragments of multiple lengths and oxidation levels, or oxygen, nitrogen sulfur or heterocycles, using well established transition metal catalyst systems. The newly introduced groups may then be cyclized directly, or further transformed prior to cyclization, and possibly further modified after the cyclization. In some manifestations one or both of R′ and R″ can have all of the elements required for the linker already built into them, relying on a single chemical transformation to close the ring, which may optionally be followed by further modification of the ring system. Likewise the ring may be cyclized by forming a direct bond to A2 or to A3, or by performing the macrocyclization anywhere along the G-linker group. For example, a G₂ alkenyl linker could be formed by using the Grubbs' catalyst to cyclize terminal double bonds on R′ and R″, or an X peptide linker can be formed by simply coupling a free amine attached by an appropriate chain to A2 to a carboxylic acid attached by an appropriate chain to A3, using standard peptide coupling technology. Or an R″ which had a terminal alcohol could be coupled to an R′ halogen on A3 using Buchwald catalysts optimized for aryl ether formation.

All of this chemistry to build G-linkers, and put them on A2 and A3 can be broken down into a series of simple steps, well known to one of ordinary skill in the art, as it is essentially modular. For example, taking a couple of cases where X and Y are both present, one could have the G-linker from A3 to A2 being —OCH₂CH₂N(Me)- or CH₂SCH₂ (2-pyrrolyl-1-linked) to A2. In the first case the A3 component could be a 3-hydroxy-4-bromopyrazole, which before or after Suzuki coupling to A1, is treated with 2-(N-Boc-N-methylamino)ethanol and PPh3-DEAD, to make the aryl ether. Treatment with TFA to remove the Boc could then be followed by treating the liberated amine with 2-iodo-5-fluoroacetophenone in a Buchwald amination, to complete the G-linker, and leave the compound ready for the A1-A2 attachment reaction. In the second case the A3 ring could be phenyl in which case a 2-bromotoluene could be brominated with NBS (before or after Suzuki coupling to A1) and turned into the benzylic thiol by treatment with thioacetic acid and excess base. This could in turn be reacted via Mitsunobu reaction with pyrrole-2-methanol to form the complete linker, which is now N-arylated on pyrrole using a copper catalyst with the A3 ring which could be the same iodofluoroacetophenone used in the other illustration. If the known 3-ethenyl-1,5-dimethylpyrazole is used in an olefin metathesis reaction with a 2-vinylsulfonyl-5-fluoroacetophenone, a —CH═CHSO₂— linker will be formed directly, and the double bond can be reduced out to give the —CH₂CH₂SO₂— linker.

Method B

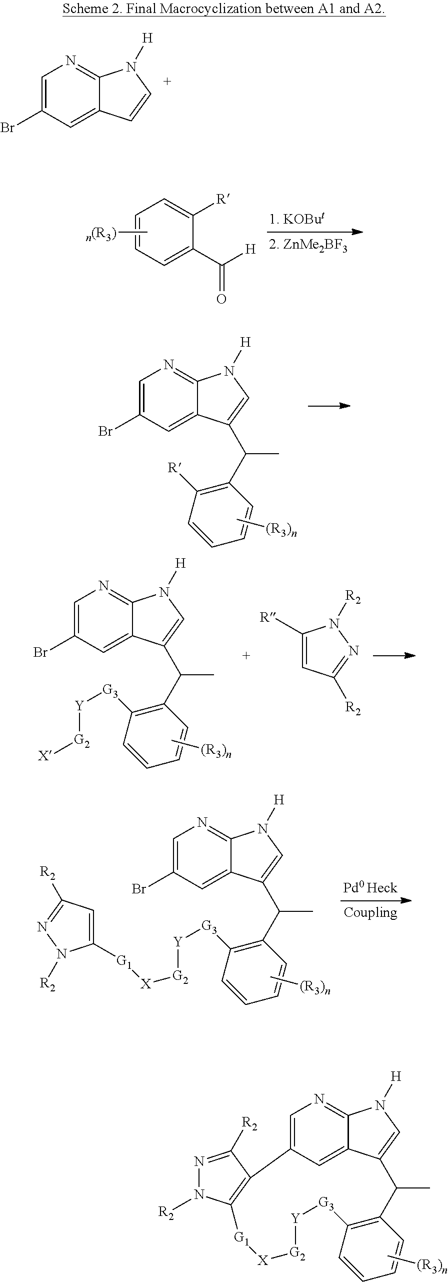

In Scheme 2 above, the first step in the scheme is the base catalyzed enaminoaldol condensation between 5-bromopyrrolo[2,3-b]pyridinine and a suitably substituted benzaldehyde derivative to link the A1 and A3 rings. The resultant benzhydrol derivative is benzylically methylated using a soft cationed metal methyl such as dimethyl zinc in the presence of a Lewis acid such as BF₃. The G-linker is then built on to R′, which as discussed above may require a very wide number of different chemistries, depending on both the nature and the presence of the components of the G-linker. The A2 ring, in this case a pyrazole is then added by coupling it to the linker group, as illustrated above. However, the whole linker group could equally well be attached at the R″ position of the linker, and coupled via X, or a precursor of G3 to R′, or parts of the G-linker could be attached to A3, via R′ and other parts to the R″ position of A2, and the G-linker completed by an internal coupling reaction. The macrocyclization would then be completed by the formation of a biaryl bond between A1 and A2, illustrated in this case as a formal Heck coupling, of which there are several examples in the literature using rhodium, copper and palladium catalysts. However, there are many other technologies available to do this biaryl coupling, and the 4-proton of pyrazole A2 could equally well be replaced by one of a number of substituents, allowing such reactions to be employed in place of the illustrated one.

Method C

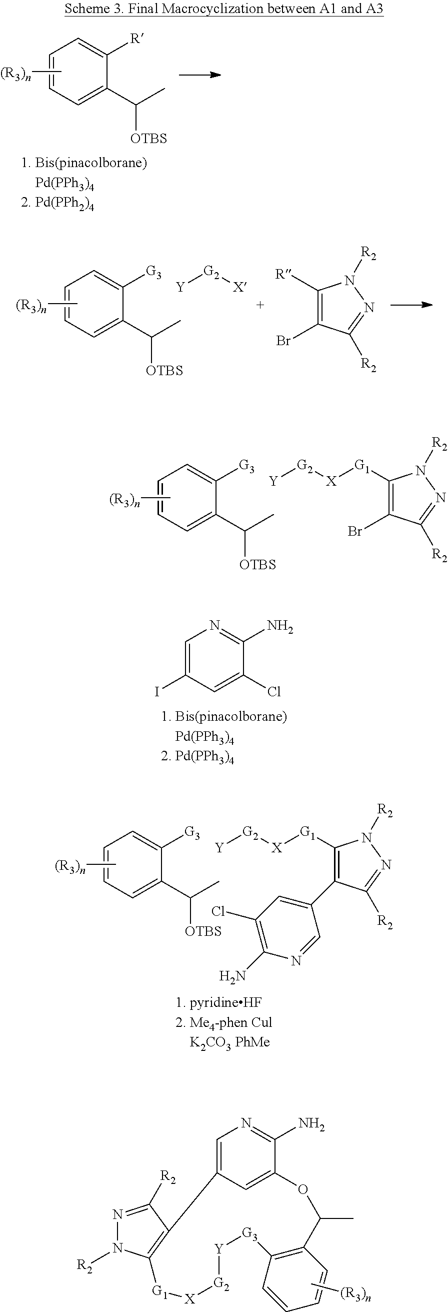

In the synthesis illustrated in Scheme 3, the synthesis starts with the elaboration of the linker group G onto a suitable 2-substituted, silicon-protected 1-phenylethanol derivative. This elaboration is preferably a coupling of the completed precursor of the G-linker, but if that is not possible, the linker can be built up in a number of sequential steps, as has been illustrated above. Once the linker is fully elaborated it is coupled via the R″ group on the 3-position of a suitably substituted 4-bromopyrazole derivative. Alternatively, a part of the linker can already be coupled to the A2 ring, and the linking of the A2 and A3 rings can be carried out using a coupling in the interior part of the G-linker. The product in this illustration is then boronated, allowing for a Suzuki coupling with a chloroiodopyridine derivative to form a biaryl with an A1 aminopyridyl group. Deprotection of the benzyl alcohol is then followed by a macrocyclization reaction using copper catalyzed Buchwald etherification onto the pyridyl chloro substituent.

In the synthetic schemes shown below several of these strategies are employed, and specific examples of chemistries capable of building representative examples of the many possible G-linkers are included. Many other chemistries allowing similar construction of other compounds of the invention will be evident to one skilled in the art, and because the above schemes represent essentially different timing sequences available for constructing the macrocycle, any of the intermediates and chemistries discussed can be applicable to any of the generic schemes.

Synthons for A1

The commercial availability of all of the 3- and 5-halo-2-aminopyridines, and their ready reaction with halogens or halosuccinimides allows one to prepare virtually any of the 3,5-dihalo-2-aminopyridines (Tetrahedron 51, 8649 (1995), WO 2013/029548, WO 2011/022473, WO 2010/083246) allowing for one to choose the halogen at each position optimally suited for the coupling reaction to be run on it. Similarly, 3-hydroxyl-2-nitropyridine can be 5-halogenated with N-halosuccinimides to form 2-nitro-3-hydroxy-5-halopyridines (WO 2004/041210) and he ready 2-nitration of the four commercially available 5-halo-3-hydroxypyridines also allows one access to these compounds. (WO 2012/116050) The 2-nitro-3-hydroxy-5-halopyridines are very versatile synthons for the compounds of the present invention, which in turn one can use as is for making 3-pyridyl ethers, convert into sulfonates, to make for complementary coupling partners to the 2-amino-3,5-dihalopyridines discussed above, or one can reduce them to the corresponding 2-aminopyridines and use those as taught in WO 2013/132376.

In a somewhat more limited fashion, a wide variety of 2-amino-3,5-dihalopyrazines and 2-amino-3-hydroxy-5-halopyrazines are readily available from commercially available 2-aminopyrazine and 2-amino-3-hydroxypyrazine. Treatment of 2-aminopyrazine with N-halosuccinimides under mild conditions leads to 5-halogenation (Chemistry, Eur J 16, 5645 (2010)), and under more forceful conditions to 2-amino-3,5-dihalopyrazines. (Tetrahedron 68, 9713 (2012)) Treatment of 2-amino-3-hydroxypyrazine with POBr₃ gives 2-amino-3-bromopyrazine, which can be halogenated a second time at the 5-position. (J. Praktishe Chemie 311 40 (1969) If a wider variety of halogens is required, displacement on chloro- and nitropyrazines by TBAF is well precedented (J Med Chem 54, 4735 (2011), and the commercial availability of 3,5-diiodopyrazine allows for monodisplacement with either an amine or hydroxy, followed by further halogenation or nitration to make other synthons for the pyrazines.

Both 5-bromopyrrolo[2,3-b]pyridine and 5-bromopyrrolo[2,3-b]pyrazine are commercially available, and under basic conditions will react readily with electrophiles at the 3-position, or can undergo ready transition metal-catalyzed arylations at the 5-position (although they may require N1-protection prior to some coupling reactions.

Thus all of the synthons required for the A1 moiety in the molecule are readily available to one skilled in the art, and in a format which allows them to be readily used in well known organic procedures to be incorporated into the eventual macrocycle.

Synthons for A2

A2 can be chosen from either a benzene/naphthalene derivative or from one of a large number of heteroaromatics, which can be monocyclic or bicyclic, all of which are known. In order to be able to form the two bonds required of it, the biaryl bond to A1 and the highly variable G-linker to A3, A2 requires to have either separated 1,2- or 1,3- a functional group which will support a biaryl coupling reaction, either with an aryl halide on the A1 synthon, or something that aryl halide can be converted into, such as a borate or stannyl derivative. This makes halide (Suzuki, Stille reactions) and hydrogen (Heck-Mizoroki) the best groups to have at these positions. The position which is going to become the anchor point for the G-linker can be much more varied, depending on what the nature of the G-linker is. None of this will involve chemistry unfamiliar to one of ordinary skill in the art. For example, almost any conceivable linker can be made by elaborating on a halide at that position, as they can be precursors to C—C, C—N, C—O, C—P and C—S bonds using processes, especially transition metal-catalyzed processes, familiar to those of ordinary skill in the art. Many of these compounds are available with the linker already partially built into the A2 ring. For example an ether linker can come from the aromatic hydroxyl compound which may be the form the ring is initially synthesized as. Or an aryl nitro compound can be reduced to the corresponding amino group, which can be acylated, phosphorylated or sulfonated by the corresponding acid chlorides. This would allow for compounds where G₁ was absent. Or if it is a C—C bond simple methyl, a carbaldehyde, or a carboxylic acid derivative can be used as the basis to build out the G-linker, and this would be appropriate in cases where G₁ is present. As aryl halides, particularly bromides and iodides are very reactive in a wide array of transition metal catalyzed couplings, they can be precursors for compounds which have G₁ bonded to the ring, or X, regardless of whether X has a heteroatom directly linked to A2, or a carbonyl, or an aromatic or partially saturated ring directly bonded to A2, either through carbon or a heteroatom. Or X can be absent as well, in which case R′ being either carbon or halogen allows for direct bonding to G₂, and in the case of G₂ being absent also, R′ as OH, SH or NH, allows for a bond to be formed directly to Y. Large numbers of suitable compounds for the various A2- to G-linker precursors are commercially available, and there are literature syntheses extant for the majority of these possibilities.

1-Alkyl-5-cyanopyrazoles linked via the 4-position to A1 and the 3-position to the G-linker are a strongly preferred manifestation of A2. Other pyrazoles such as 1,3- and 1,5 dialkyl pyrazoles, linked into the macrocycle by the 3,4 and 4,5-positions respectively are also preferred. Such compounds are readily available, with a major route into them being the addition, cyclization, and consequent N-dealkylation of 1,1-dialkylhydrazines to acetylene dicarboxylate esters. (Chem Ber 111, 780(1978), Angew Chem 87, 551 (1975)). These compounds can readily be functionalized by electrophiles, including halogen at the 4-position, Chem Ber 109, 268 1976), and the carboxylate can be converted into a methyl group or a cyano group as required. Once this is completed, the 3-hydroxy can be converted into a halide or sulfonare leaving group, and used to link into the G-linker. Many such manipulations are disclosed in WO 2013/132376.

Synthons for A3

A3 can be chosen from either a benzene/naphthalene derivative or from one of a large number of heteroaromatics, which can be monocyclic or bicyclic, all of which are known. In order to be able to form the two bonds required of it, the 1-2-atom linker to A1 and the highly variable G-linker to A2, A3 requires to two linker groups placed ortho to one another in the aromatic system. The synthon needs to contain a C—C bond where the linker to A1 is to be built and in most cases this can be provided by having either an aldehyde or hydroxymethyl (which can be converted via halide or sulfonate into many methylene-S, methylene-N or methylene-C) bonds as needed. The best precursor for the G-linker is halogen, as that allows for facile C—C, C—N, C—O, C—P and C—S bonds using processes familiar to those of ordinary skill in the art, as discussed for A2. In this case, many of the preferred compounds will have a concatenated, directly bonded, ring next to A3, and if aromatic, biaryl coupling reactions or Buchwald couplings will be optimal for introducing Y directly bonded to A3. If however Y is a non-aromatic ring directly bonded to A3, other functional groups such as nitro, or a carboxylic acid derivative may be better starting points for the synthesis. The same preferences for whether A3 is directly bonded to G₃, Y, G₂ or even X, which were discussed for the A2 moiety also hold here, with R″ being O, N or S, being preferred to synthesize compounds where G3 is absent, and carbon containing R″ groups being generally more suitable for G3-containing linkers, and once again the R′=halogen giving the greatest flexibility allowing one to form alkyl-type linkers, (G3) heteroatom-type linkers (Y) or directly bonded ring systems (Y).

When A1 is A1a, a preferred linker and A3 ring combination is a phenyl ring substituted with fluorine, making a benzylic ether linkage to A1, and a phenolic ether linkage into the G-linker. This requires the preparation of 2-(1-hydroxyalkyl)-4-fluorophenols or suitable precursors to the phenolic oxygen. Many such precursors are available commercially such as 4-fluoro-2-acylphenols, 4-fluoro-2-acylanilines, 4-fluoro-2-acylbenzoic acids, and 5-fluoro-2-haloacylbenzenes. For one skilled in the art conversion of such compounds into the desired 1-(3-fluorophenyl)1-hydroxyalkyl derivatives is available by many routes, including a variety which allow the chirality of the alcohol (when appropriate) to be controlled. Incorporation of many of these synthons into a macrocyclic ring in the appropriate fashion are described in WO 2013/132376. If the group L₁ is an oxygen atom, Buchwald etherifications or sometimes nucleophilic displacements can be done directly on a 3-halopyridine derivative, or the alcohol can be converted into a leaving group, for instance by conversion into a halide or sulfonate, or via use of the Mitsunobu reaction, and displaced with the hydroxyl of a 3-hydroxypyridine. In cases where the L₁ linker is not oxygen, the alcohol can be dislaced by a suitable nitrogen, sulfur or one carbon nucleophile, and subsequently coupled to the halopyridine by either transition metal catalyzed coupling or nucleophilic displacement.

When A1 is A1 b, the same substitution pattern for the A3 ring also requires formation of 2-hydroxy (or synthetic precursor)-5-fluoroacylbenzenes, especially the benzaldehydes. These are essentially identical precursors to those when A1a is the head group, and can be modified in similar fashion. However, the way that they are incorporated into the final product, and coupled with A1b are fundamentally different to the scenarios described for A1a incorporation. Here a C—C bond must be formed between C3 of the pyrrolopyr(id/az)ine and the carbonyl carbon, which can be done by treating the mixture with base, to make the pyrrole anion, which then adds across the carbonyl double bond, to give a bis-benzylic alcohol. This alcohol can be reduced out directly, for example with BF3/triethylsilane, or it can be eliminated to form a 1,1-diarylethene, which can be subsequently reduced, possibly chirally to the desired product, or if the benzylic alcohol is secondary, it can be displaced with soft cation (Mg, Zn, Cd etc.) organometallic reagent in the presence of a suitable Lewis acid such as BF₃. For many examples of this type of process, see US 2013/0253197.

Joining A1 and A2

Whenever in the synthesis the A1 and A2 rings are joined, whether it be at an early step, or the final macrocyclization, the reactions used are very well precedented, with a large number of possibilities to choose from. In many cases both aromatic partners have to be functionalized, and both of these functional groups are replace by the new carbon-carbon bond, but much emphasis is now placed on having only one of the molecules functionalized, and the other replacing an aromatic C—H bond with the C—C bond of the biaryl, although in this case regiochemical control may be more difficult. This chemistry has been extensively reviewed, and a few of these reviews, and the references they cover are incorporated by reference. “Gold-Catalyzed Direct Arylation” Science 337 1644 (2012), “Rhodium-catalyzed C—H bond arylation of arenes” Topics in Current Chemistry 292, 231 (2010). “Intramolecular oxidative cross-coupling of arenes” Chem Soc Rev 39 540 (2010). “Synthesis of Biaryls” Science of Synthesis 45b 547 (2010). “C(aryl)-O activation of aryl carboxylates in nickel-catalyzed biaryl syntheses” Angew Chem Int Edn 48, 3569 (2009). “Biaryl coupling reaction for natural product synthesis” Heterocycles 75, 1305 (2008).

Constructing the G-Linkers

As the G-linkers are highly variable, a wide variety of chemistries will be required to produce them, and much of that chemistry may be prebuilt into A2 and A3. For example when linkers involve C—C bonds directly to A2 or A3, use of a carboxylic acid, aldehyde or hydroxymethyl group at the attachment point on the A2 or A3 synthon can often facilitate formation of the linker. If the linker is a partially or fully unsaturated ring, having a halogen present on A2 or A3, and using a biaryl type of coupling, of the type refereneced in the previous paragraph, may work better, or one may be able to elaborate the X/Y ring directly onto the carbon anchor point, especially if it a carboxylate derivative.

Because of their modular nature, the more complex G-linkers can be largely concatenated from simple, well known fragments, using simple reactions such as nucleophilic displacements, acylations, sulfonations etc., and these larger pieces then have to be incorporated into the (incipient) macrocycle. This can involve transition metal coupling reactions, where one of the incipient A2-G-linker or A3-G-linker bonds is represented on one fragment by halide/sulfonate, and on the other by boronate/stannane, or it can involve part of the G-linker being formed as it is coupled to A2 or A3, such as for example a Wittig reaction of a G-linker phosphonium salt to an A2-araldehyde, optionally followed by a reduction.

Many of the G-linkers are quite simple such as -oxyethoxy- and in such cases may well be produced very straightforwardly, for instance by having both the A2-ring and A3-ring as aryl alcohols, so that one could do say a Mitsunobu reaction on the A2 fragment with 2-bromoethanol, and then displace the halide with the alkoxide derived from the A3 ring hydroxyl. Other case, involving simple amine or thio derivatives (or their various oxidized sulfur higher homologues, can be prepared by having benzylic or homobenzylic alcohols or halides which can be displaced by nitrogen or sulfur sequentially, and then nave their redox or alkylation status changed appropriately using reactions well known to one of skill in the art. One can also envision joining larger carbon containing fragments for G₁-G₃, where o is 2 or 3, using Wittig/Peterson type olefinations, or olefin metathesis reactions using Grubbs or Hoveyda catalysts. Other linkers can be elaborated one the entire G-linker chain has been concatenated by carrying out straightforward chemical modifications. For example carbonyls, which can be readily introduced into the G-linker chain via a wide variety of techniques including Heck-type carbonylations, umpolung carbonyl anion alkylations, and addition of an organometallic to a carboxylic acid derivative, can be converted into oximes, hydraxzones, difluoromethyl, alkenyl, alkyl, amino and hydroxy (all after a reduction step) etc.

More complex linker groups can be built piece by piece. For example a phosphonamidoate can be incorporated by first adding a phosphonate diester to say G3 already linked to A3 via simple C-alkylation. The esters can be hydrolyzed, and the acid converted to the phosphonyl dichloride with PCI₅, and then treated with one equivalent of amine, which might be linked to A2 to complete the G-linker atoms, followed by a final ethanolysis to make the O-ethylphosphonoamidoate. Similarly a sulfoximine could be added to the chain by taking an already complete chain containing a thioether, and then selectively oxidizing that the sulfoxide, which would then be oxidized with t-butyl hypochlorite, followed by addition of ammonia.

Some general schemes for putting the macrocycle together were shown above, and below are some illustrative examples of synthetic routes to make representative examples of the current invention.

### Methods of Treatment

The invention further relates to therapeutic methods and uses comprising administering the compounds of the invention, or pharmaceutically acceptable salts thereof, alone or in combination with other therapeutic or palliative agents.

In one embodiment, the invention relates to a method for treating or inhibiting cell proliferation, cell invasiveness, metastases, apoptosis, or angiogenesis in a mammal comprising administering to the mammal a therapeutically effective amount of a compound of the invention, or pharmaceutically acceptable salt thereof.

In another embodiment, the invention relates to a method for treating or inhibiting cell proliferation, cell invasiveness, metastases, apoptosis, or angiogenesis in a mammal comprising administering to the mammal a therapeutically effective amount of a compound of the invention, or pharmaceutically acceptable salt thereof, in combination with a with a second therapeutic agent wherein the amounts of the compound of the invention and the second therapeutic agent together are effective in treating or inhibiting said cell proliferation, cell invasiveness, metastases, apoptosis, or angiogenesis.

In one embodiment, the second therapeutic agent is an anti-tumor agent which is selected from the group consisting of mitotic inhibitors, alkylating agents, antimetabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In other embodiments, the cell proliferation, cell invasiveness, metastases, apoptosis, or angiogenesis is mediated by ALK, an ALK-EML-4 fusion protein, AXL, Aur B & C, mutant BCR-ABL, BLK, Eph6B, HPK, IRAK1 & 3, LCK, LTK, various MEKKs, RON, ROS1, SLK, STK10, TIE1 & 2, and TRKs1-3.

In a further embodiment, the cell proliferation, cell invasiveness, metastases, apoptosis, or angiogenesis is associated with a cancer selected from the group consisting of basal cell cancer, medulloblastoma cancer, liver cancer, rhabdomyosarcoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon, cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

A further embodiment of the invention relates to a compound of the invention for use as a medicament, and in particular for use in the treatment of diseases where the inhibition of ALK and/or an ALK fusion protein, e.g., EML4-ALK, activity may induce benefit, such as cancer. A still further embodiment of the present invention relates to the use of the compounds of the invention, or pharmaceutically acceptable salts thereof, for the manufacture of a drug having an ALK inhibitory activity for the treatment of ALK-mediated diseases and/or conditions, in particular the diseases and/or conditions listed above.

In another embodiment, the invention relates to a method for the treatment of treatment of pain, including acute pain; chronic pain; neuropathic pain; inflammatory pain (including e.g. osteoarthritis pain, rheumatoid arthritis pain); visceral pain; nociceptive pain including post-surgical pain; and mixed pain types involving the viscera, gastrointestinal tract, cranial structures, musculoskeletal system, spine, urogenital system, cardiovascular system and CNS, including cancer pain, back and orofacial pain in a mammal comprising administering to the mammal a therapeutically effective amount of a compound of the invention, or a pharmaceutically acceptable salt thereof. As contemplated herein, the term “pain” includes acute pain, chronic pain, neuropathic pain, inflammatory pain, visceral pain, nociceptive pain, and mixed pain types involving the visera, gastrointestinal tract, cranial structures, musculoskeletal system, spine, urogenital system, cardiovascular system, and central nervous system, including cancer pain, back pain and orofacial pain.

The term “therapeutically effective amount” refers to that amount of a compound being administered which will relieve to some extent one or more of the symptoms of the disorder being treated. Regarding the treatment of cancer, a therapeutically effective amount refers to that amount which has the effect of reducing the size of the tumor, inhibiting (i.e., slowing or stopping) tumor metastases, inhibiting (i.e. slowing or stopping) tumor growth or tumor invasiveness, and/or relieving to some extent one or more signs or symptoms related to the cancer.

A therapeutically effective amount can be readily determined by the attending diagnostician, as one skilled in the art, by the use of conventional techniques and by observing results obtained under analogous circumstances. In determining the therapeutically effective amount, the dose, a number of factors are considered by the attending diagnostician, including, but not limited to: the species of mammal; its size, age, and general health; the specific disease involved; the degree of involvement or the severity of the disease; the response of the individual patient; the particular compound administered; the mode of administration; the bioavailability characteristic of the preparation administered; the dose regimen selected; the use of concomitant medication; and other relevant circumstances.

The term “treating”, as used herein, unless otherwise indicated, means reversing, alleviating, inhibiting the progress of, or preventing the disorder or condition to which such term applies, or one or more symptoms of such disorder or condition. The term “treatment” also refers to the act of treating as “treating” is defined immediately above. The term “treating” also includes adjuvant treatment of a mammal.

As used herein “cancer” refers to any malignant and/or invasive growth or tumor caused by abnormal cell growth, including solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type.

In another embodiment, the invention provides a method for inhibiting cell proliferation, comprising contacting cells with a compound of the invention or a pharmaceutically acceptable salt thereof in an amount effective to inhibit proliferation of the cells. In another embodiment, the invention provides methods for inducing cell apoptosis, comprising contacting cells with a compound described herein in an amount effective to induce apoptosis of the cells.

“Contacting” refers to bringing a compound or pharmaceutically acceptable salt of the invention and a cell expressing ALK, or one of the other target kinases which is playing a transforming role in the particular cell type, together in such a manner that the compound can affect the activity of ALK, or the other kinase, either directly or indirectly. Contacting can be accomplished in vitro (i.e., in an artificial environment such as, e.g., without limitation, in a test tube or culture medium) or in vivo (i.e., within a living organism such as, without limitation, a mouse, rat or rabbit.)

In some embodiments, the cells are in a cell line, such as a cancer cell line. In other embodiments, the cells are in a tissue or tumor, and the tissue or tumor may be in a mammal, including a human.

Administration of the compounds of the invention may be effected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage.

Dosage unit form, as used herein, refers to physically discrete units suited as unitary dosages for the mammalian mammals to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Appropriate dosages may vary with the type and severity of the condition to be treated and may include single or multiple doses. An attending diagnostician understands that for any particular mammal, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

Useful dosages of the compounds of the invention can be determined by comparing their in vitro activity, and in vivo activity in animal models. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compounds of the present invention can be administered to a patient at dosage levels in the range of about 0.1 to about 2,000 mg per day. For a normal human adult having a body weight of about 70 kilograms, a dosage in the range of about 0.01 to about 10 mg per kilogram of body weight per day is preferable. However, the specific dosage used can vary. For example, the dosage can depended on a numbers of factors including the requirements of the patient, the severity of the condition being treated, and the pharmacological activity of the compound being used. The determination of optimum dosages for a particular patient is well-known to those skilled in the art. In some instances, dosage levels below the lower limit of the aforesaid range may be more than adequate, while in other cases still larger doses may be employed without causing harmful side effect, provided that such larger doses are first divided into several smaller doses for administration throughout the day.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions suitable for the delivery of compounds of the invention and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid.

The compounds of the invention may also be used in fast-dissolving, fast-disintegrating dosage forms such as those described in Expert Opinion in Therapeutic Patents, 11 (6), 981986 by Liang and Chen (2001), the disclosure of which is incorporated herein by reference in its entirety.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight to 10 weight %, preferably from 0.5 weight % to 3 weight % of the tablet.

Other possible ingredients include anti-oxidants, colorants, flavoring agents, preservatives and taste-masking agents.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration discussed above may be formulated to be immediate and/or modified release. Modified release formulations include delayed-, sustained-, pulsed-, controlled-, targeted and programmed release. Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Thus, compounds of the invention may be formulated as a solid, semi-solid, or thixotropic liquid for administration as an implanted depot providing modified release of the active compound. Examples of such formulations include drug-coated stents and poly(glycolide-co-dl-lactide) or PGLA microspheres.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

The term “combination therapy” refers to the administration of a compound of the invention together with at least one additional pharmaceutical or medicinal agent, either sequentially or simultaneously. Combination therapy encompasses the use of the compounds of the present invention and other therapeutic agents either in discreet dosage forms or in the same pharmaceutical formulation. The compounds of the invention may be used in combination (administered either simultaneously, sequentially, or separately) with one or more therapeutic agents.

In one embodiment of the present invention the anti-cancer agent used in conjunction with a compound of the invention and pharmaceutical compositions described herein is an antiangiogenesis agent (e.g., an agent that stops tumors from developing new blood vessels). Examples of anti-angiogenesis agents include for example VEGF inhibitors, VEGFR inhibitors, TIE-2 inhibitors, PDGFR inhibitors, angiopoetin inhibitors, PKCI3 inhibitors, CQX-2 (cyclooxygenase II) inhibitors, integrins (alpha-v/beta-3), MMP-2 (matrix-metalloprotienase 2) inhibitors, and MMP-9 (matrix-metalloprotienase 9) inhibitors. Preferred anti-angiogenesis agents include sunitinib (SutenFM), bevacizumab (Avastin™), and axitinib (AG 13736).

Additional anti-angiogenesis agents include vatalanib (CGP 79787), Sorafenib (Nexavar™), pegaptanib octasodium (Macugen™), vandetanib (Zactima™) PF-0337210 (Pfizer), SU 14843 (Pfizer), AZD 2171 (AstraZeneca), ranibizumab (Lucentis™), Neovastat™ (AE 941), tetrathiomolybdata (Coprexa™), AMG 706 (Amgen), VEGF Trap (AVE 0005), CEP 7055 (Sanofi-Aventis), XL 880 (Exelixis), telatinib (BAY 57-9352), and CP-868,596 (Pfizer).

Other examples of anti-angiogenesis agents which can be used in conjunction with a compound of the invention and pharmaceutical compositions described herein include celecoxib (Celebrex™), parecoxib (Dynastat™), deracoxib (SC 59046), lumiracoxib (Preige™), valdecoxib (Bextra™), rofecoxib (Vioxx™), iguratimod (Careram™), IP 751 (Invedus), SC-58125 (Pharmacia) and etoricoxib (Arcoxia™). Other anti-angiogenesis agents include exisulind (Aptosyn™), salsalate (Amigesic™), diflunisal (Dolobid™), ibuprofen (Motrin™), ketoprofen (Orudis™) nabumetone (Relafen™), piroxicam (Feldene™), naproxen (Aleve™, Naprosyn™), diclofenac (Voltaren™), indomethacin (Indocin™), sulindac (Clinoril™), tolmetin (Tolectin™), etodolac (Lodine™), ketorolac (Toradol™), and oxaprozin (Daypro™) Other anti-angiogenesis agents include ABT 510 (Abbott), apratastat (TMI 005), AZD 8955 (AstraZeneca), incyclinide (Metastat™), and PCK 3145 (Procyon). Other anti-angiogenesis agents include acitretin (Neotigason™), plitidepsin (Aplidine™), cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™).

In another embodiment the anti-cancer agent is a so called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors. Preferred signal transduction inhibitors include gefitinib (Iressa™), cetuximab (Erbitux™), erlotinib (Tarceva™), trastuzumab (Herceptin™) sunitinib (Sutent™), and imatinib (Gleevec™).

Additional examples of signal transduction inhibitors which may be used in conjunction with a compound of the invention and pharmaceutical compositions described herein include BMS 214662 (Bristol-Myers Squibb), lonafarnib (Sarasar™) pelitrexol (AG 2037), matuzumab (EMO 7200), nimotuzumab (TheraCIM h-R3™) panitumumab (Vectibix™), Vandetanib (Zactima™), pazopanib (SB 786034), ALT 110 (Alteris Therapeutics), BIBW 2992 (Boehringer Ingelheim), and Cervene™ (TP 38). Other examples of signal transduction inhibitor include PF-2341 066 (Pfizer), PF-299804 (Pfizer), canertinib, pertuzumab (Omnitarg™), Lapatinib (Tycerb™), pelitinib (EKB 569), miltefosine (Miltefosin™), BMS 599626 (Bristol-Myers Squibb), Lapuleucel-T (Neuvenge™), NeuVax™ (E75 cancer vaccine), Osidem™, mubritinib (TAK-165), panitumumab (Vectibix™), lapatinib (Tycerb™), pelitinib (EKB 569), and pertuzumab (Omnitarg™). Other examples of signal transduction inhibitors include ARRY 142886 (Array Biopharm), everolimus (Certican™), zotarolimus (Endeavor™), temsirolimus (Torisel™), and AP 23573 (ARIAO). Additionally, other signal transduction inhibitors include XL 647 (Exelixis), sorafenib (Nexavar™), LE-AON (Georgetown University), and GI-4000 (Globelmmune). Other signal transduction inhibitors include ABT 751 (Abbott), alvocidib (flavopiridol), BMS 387032 (Bristol Myers), EM 1421 (Erimos), indisulam (E 7070), seliciclib (CYC 200), BIO 112 (Onc Bio), BMS 387032 (Bristol-Myers Squibb), PO 0332991 (Pfizer), and AG 024322 (Pfizer).

This invention contemplates the use of compounds of the invention together with classical antineoplastic agents. Classical antineoplastic agents include hormonal modulators such as hormonal, anti-hormonal, androgen agonist, androgen antagonist and anti-estrogen therapeutic agents, histone deacetylase (HOAC) inhibitors, gene silencing agents or gene activating agents, ribonucleases, proteosomics, Topoisomerase I inhibitors, Camptothecin derivatives, Topoisomerase II inhibitors, alkylating agents, anti metabolites, poly(AOP-ribose) polymerase-1 (PARP-1) inhibitor, microtubulin inhibitors, antibiotics, plant derived spindle inhibitors, platinum-coordinated compounds, gene therapeutic agents, antisense oligonucleotides, vascular targeting agents (VTAs), and statins.

Examples of antineoplastic agents used in combination with compounds of the invention include Velcade (bortezomib), 9-aminocamptothecin, belotecan, camptothecin, diflomotecan, edotecarin, exatecan (Daiichi), gimatecan, 10-hydroxycamptothecin, irinotecan HCl (Camptosar), lurtotecan, Orathecin (rubitecan, Supergen), topotecan, camptothecin, 10-hydroxycamptothecin, 9-aminocamptothecin, irinotecan, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, altretamine, AP-5280, apaziquone, brostallicin, bendamustine, busulfan, carboquone, carmustine, chlorambucil, dacarbazine, estramustine, fotemustine, glufosfamide, ifosfamide, lomustine, mafosfamide, mechlorethamine, melphalan, mitobronitol, mitolactol, mitomycin C, mitoxatrone, nimustine, ranimustine, temozolomide, thiotepa, and platinumcoordinated alkylating compounds such as cisplatin, Paraplatin (carboplatin), eptaplatin, lobaplatin, nedaplatin, Eloxatin (oxaliplatin, Sanofi), streptozocin, satrplatin, and combinations thereof.

The invention also contemplates the use of the compounds of the invention together with dihydrofolate reductase inhibitors (such as methotrexate and trimetresate glucuronate), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil), Alimta (premetrexed disodium), capecitabine (Xeloda™), cytosine arabinoside, Gemzar™ (gemcitabine), Tegafur, doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, and N-(5-[N-(3,4-dihydro-2-methyl-4-oxoquinazolin-6-ylmethyl)-N-methylamino]-2thenoyl)-L-glutamic acid, and combinations thereof.

Other examples of classical antineoplastic cytotoxic agents used in combination therapy with a compound of the invention, optionally with one or more other agents include Abraxane (Abraxis BioScience, Inc.), Batabulin (Amgen), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paciltaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta™), DMXAA (Antisoma), ibandronic acid, L-asparaginase, pegaspargase (Oncaspar™), Efaproxiral (Efaproxyn™—radiation therapy)), bexarotene (Targretin™), Tesmilifene, Theratope™ (Biomira), Tretinoin (Vesanoid™), tirapazamine (Trizaone™), motexafin gadolinium (Xcytrin™) Cotara™ (mAb), and NBI-3001 (Protox Therapeutics), polyglutamate-paclitaxel (Xyotax™) and combinations thereof.

### EXAMPLES

### Abbreviations

DMSO dimethylsulfoxide

DTT dithiothreitol

ATP adenosine triphosphate

EDTA ethylenediaminetetraacetic acid

Kᵢ enzyme inhibition constant

DMEM Dulbecco's Modified Eagle Medium

NCS newborn calf serum

PBS phosphate buffered saline

PMSF phenylmethanesulfonyl fluoride

ELISA enzyme-linked immunosorbent assay

IgG immunoglobulin G

FBS fetal bovine serum

BDNF brain derived neurotrophic factor

TEA triethylamine

LCMS liquid chromatography-mass spectroscopy

TFAA trifluoroacetic anhydride

THF tetrahydrofuran

EtOAc ethyl acetate

EA ethyl acetate

PE petroleum ether

DMF dimethylformamide

DIAD diisopropylazodicarboxylate

DIP-Cl chloro-bis[(1R,2S,3R,5R)-2,6,6-trimethylnorpinan-3-yl]borane

NBS N-bromosuccinimide

MeCN acetonitrile

Boc₂O di-tert-butyl dicarbonate

DCM dichloromethane

HTRF homogeneous time resolved fluorescence

ATP adenosine triphosphate

HEPES 4-(2-hydroxyethyl)-1-piperazineethanesulfonic acid

BSA bovine serum albumin

DTT dithiothreitol

### Synthesis of Compounds of the Invention

### Example 1

### Synthesis of Compound 1

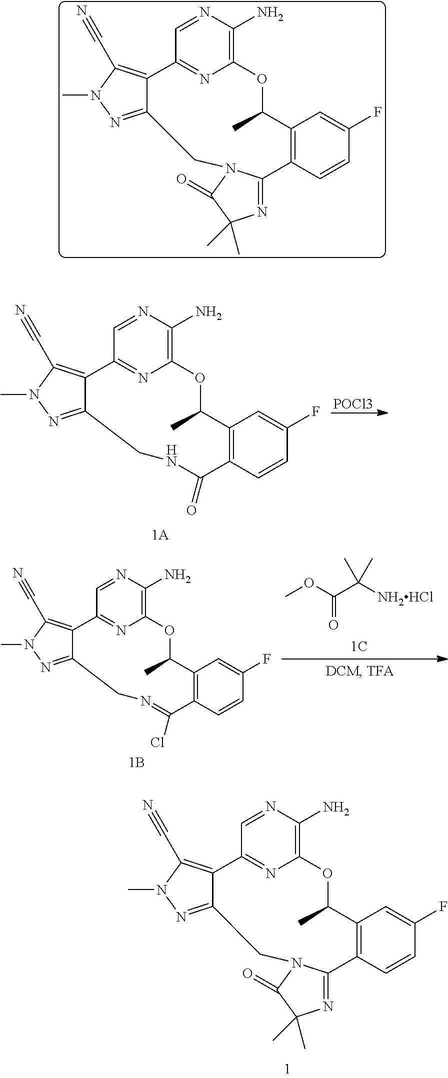

Compound 1A is a known compound, and the synthesis was described in WO2013132376A1, Page 395, as Example 104.

Step 1.

Compound 1A is mixed with neat POCl3 and stirred at room temperature for 30 minutes, then POCl3 is removed in vacuo to give iminochloride 1B.

Step 2.

To a solution of 1B in dichloromethane is added 1 C (1.1 mole equivalent) followed by TEA (1.2 equivalent). The resulting mixture is stirred at room temperature for 8 hours. Saturated NH₄Cl solution is added, the two layers are separated and the aqueous layer is extracted with DCM three times, and the combined organic phases are dried over Na₂SO₄, filtered, and concentrated in vacuo. The residue is further purified by chromatography to give the desired product 1.

### Example 2

### Synthesis of Compound 2

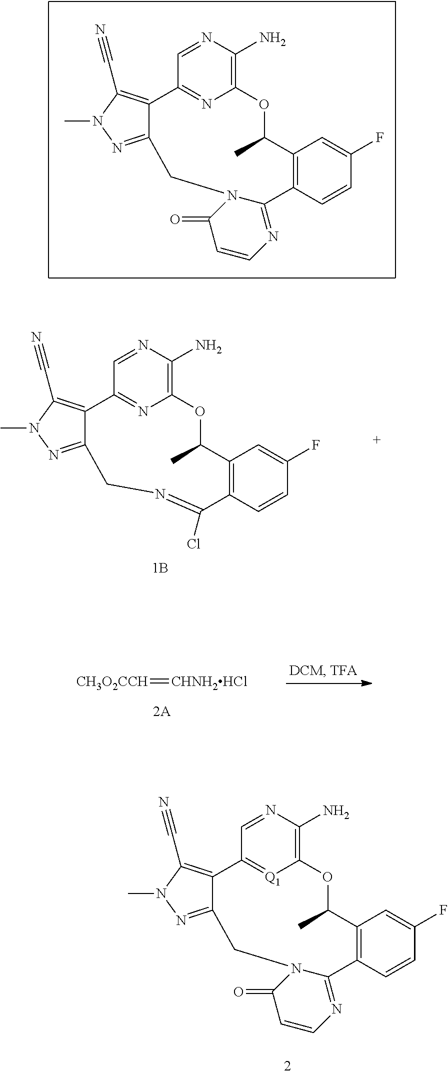

To a solution of 1B in dichloromethane is added 2A (1.1 mole equivalent) followed by TEA (1.2 equivalent). The resulting mixture is stirred at room temperature for 8 hours. Saturated NH₄Cl solution is added, the two layers are separated, the aqueous layer is extracted with DCM three times, and the combined organic phases are dried over Na₂SO₄, filtered, and concentrated in vacuo. The residue is further purified by chromatography to give the desired product 2.

### Example 3

### Synthesis of Compound 3

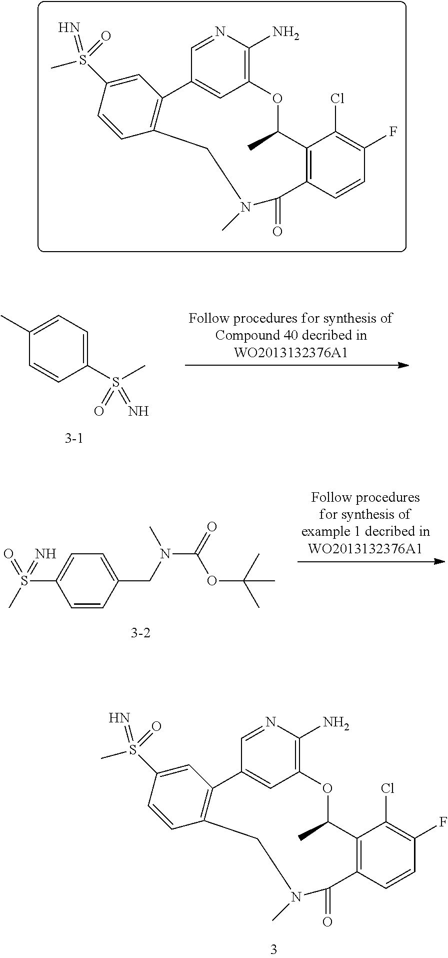

Compound 3-1 is transformed to 3-2 by following the experimental procedures described in patent application WO2013132376A1 for the synthesis of compound 40. Compound 3-2 is transformed to desired product 3 by following the experimental procedures shown for the synthesis of Example 1 in the same patent application.

### Example 4

### Compound 4

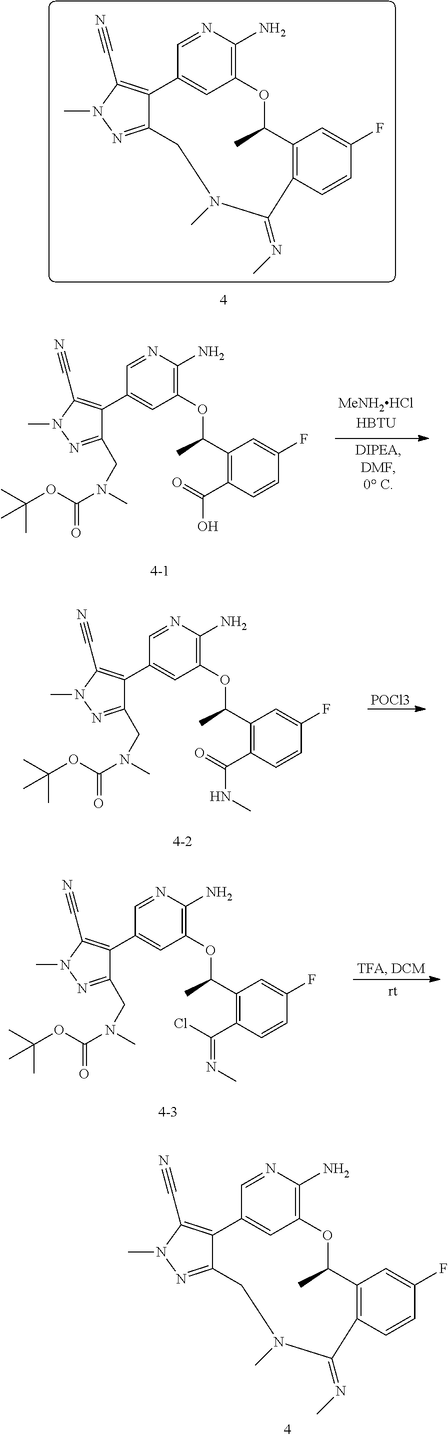

Compound 4-1 is a known compound, and the synthesis was described in WO2013132376A1 as compound 341. Compound 4-1 is treated with a coupling reagent such as HBTU, then reacts with methylamine hydrocholoride in situ in the presence of base such as DIPEA to give compound 4-2. Compound 4-2 reacts with POCl3 to give compound 4-3, which is treated with TFA to remove the BOC protecting group to give the desired product 4.

### Example 5

### Compound 5

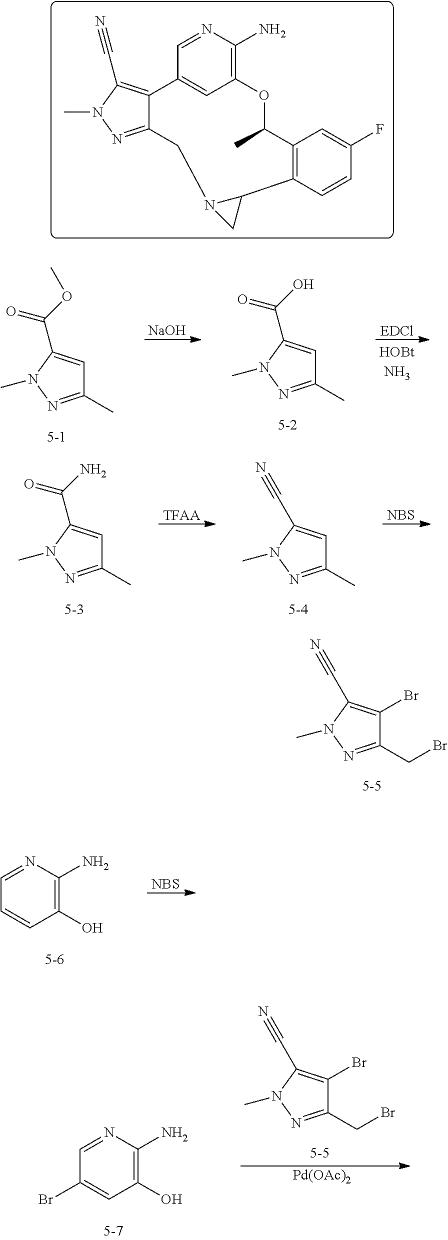

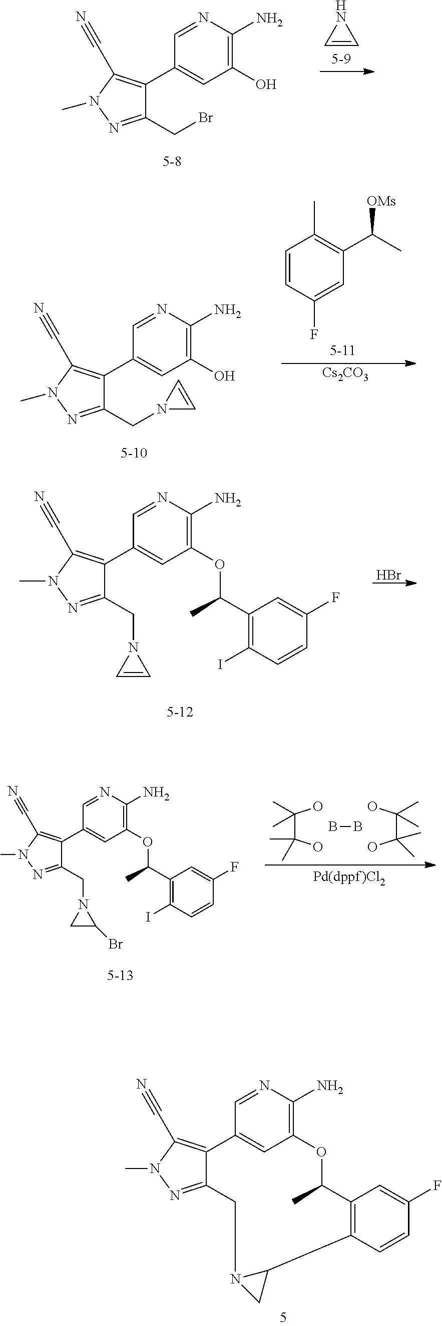

Hydrolysis of 5-1 using NaOH under standard hydrolysis conditions in MeOH can give the acid 5-2, which can be converted to amide 5-3 under standard amide formation conditions using acid activating group such as EDCl and HOBt and reacting with ammonia. Dehydration of the primary amide 5-3 can give nitril 5-4 using TFAA as the dehydration agent. Bromination of compound 5-4 using bromination reagent such as NBS can give di-bromide 5-5. A detailed procedure of this type of transformation can be found in patent application WO2013132376A1 for the synthesis of compound 42.

Compound 5-6 can be brominated by reacting with NBS to give compound 5-7, which can be coupled with 5-5 using Pd(OAc)2 as the catalyst to give compound 5-8. The displacement reaction of 5-8 and 5-9 can give 5-10, which can further react with compound 5-11 to give compound 5-12, a similar experimental procedure can be found in the patent application WO2013132376A1 for this transformation. Bromination reaction with HBr can afford compound 5-13, which can be converted to a bronic ester, then intramolecularly couple with the iodo benzene moiety to give the desired product 5.

### Example 6

### Compound 6

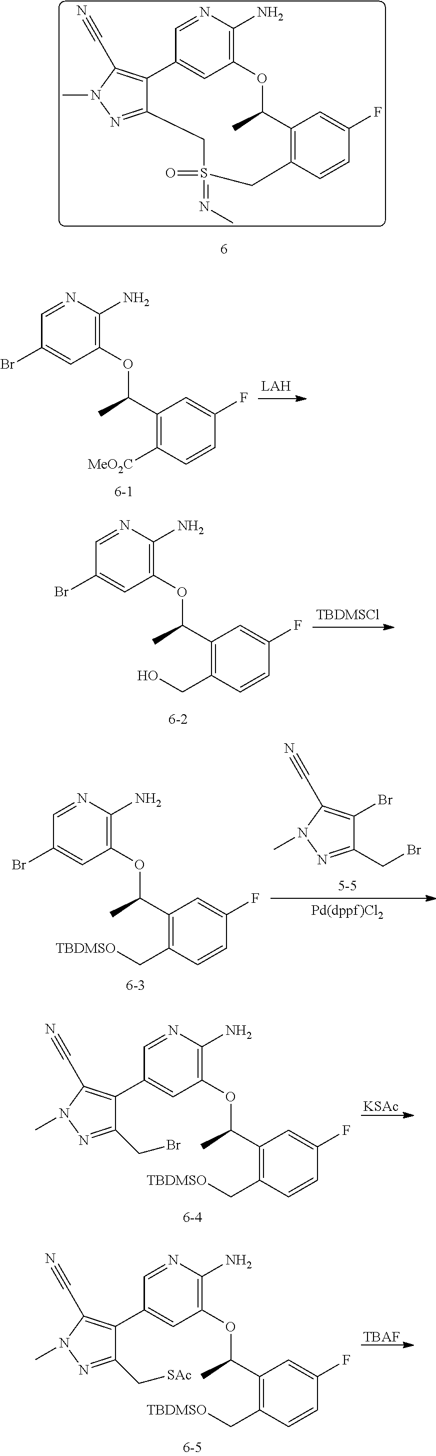

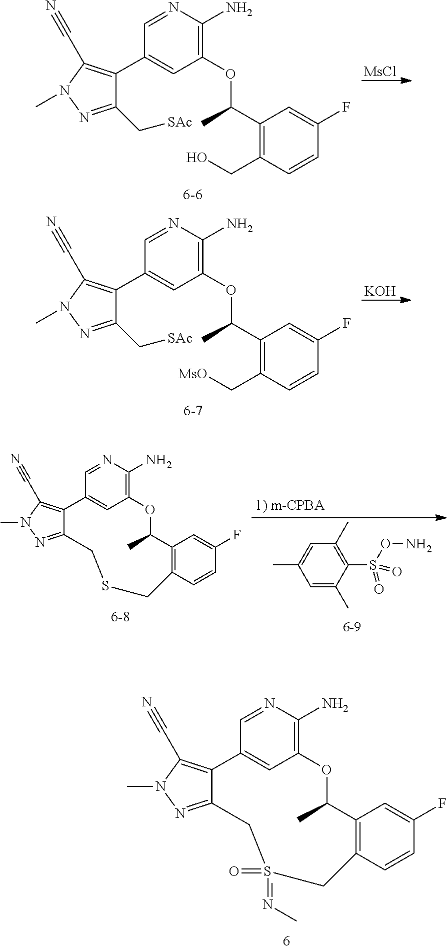

Compound 6-1 is a known compound and the preparation can be found in WO2013132376A1 for the synthesis of compound 35.

Ester 6-1 can be reduced to alcohol 6-2 and protected as silyl ether 6-3. Palladium catalyzed coupling reaction of compound 6-3 and 5-5 (the preparation is shown in the preparation of compound 5 in this patent application) can afford compound 6-4, which can react with KSAc to give compound 6-5. Deprotection of the silyl ether produces alcohol 6-8, which can be converted to compound 6-7, and deprotection of the thiol group can produce the free thiol which can displace the mesylate group intramolecularly to give the thioether 6-8. Oxidation to sulfone and subsequent reaction of sulfone with reagent 6-9 can give the desired product 6.

### Example 7

### Compound 7

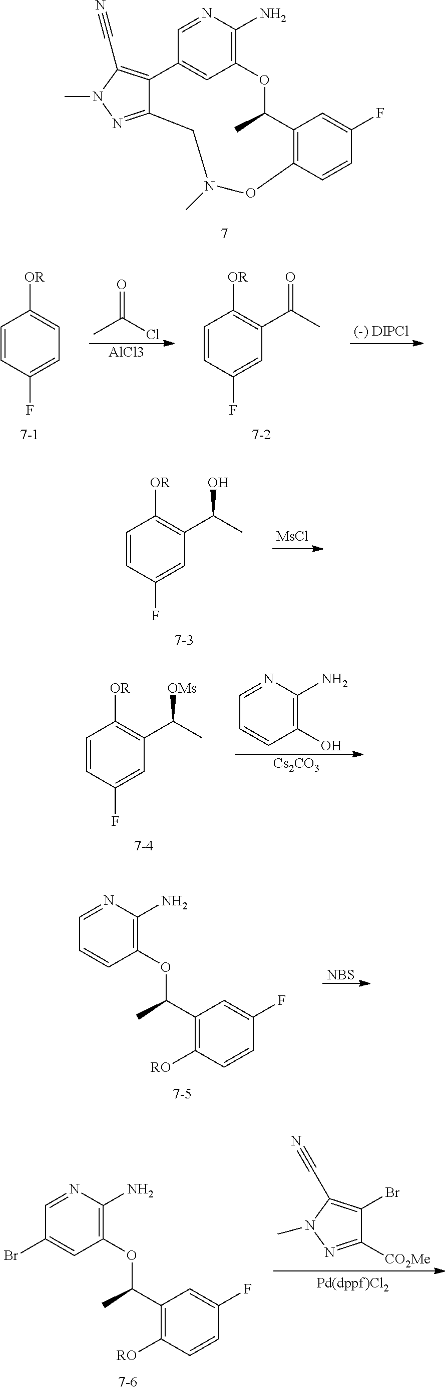

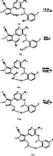

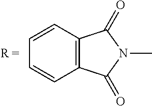

Acylation of compound 7-1 under standard conditions can give compound 7-2, which is enantiomerically reduced to a chiral alcohol 7-3. Mesylation of compound 7-3 gives 7-4, which can react with 5-7 to give compound 7-5. Cross coupling of 7-5 with the corresponding bromopyrazole gives compound 7-7, which can react with hydrazine to remove the protection group on the nitrogen to give compound 7-8. Intramolecular coupling reaction using a coupling reagent such as EDCl can give compound 7-9. The carbonyl group of compound 7-9 can be reduced to give compound 7-10. The secondary amine group of compound 7-10 can be methylated under reductive amination reaction conditions to give desired compound 7.

### Example 8

### Compound 8

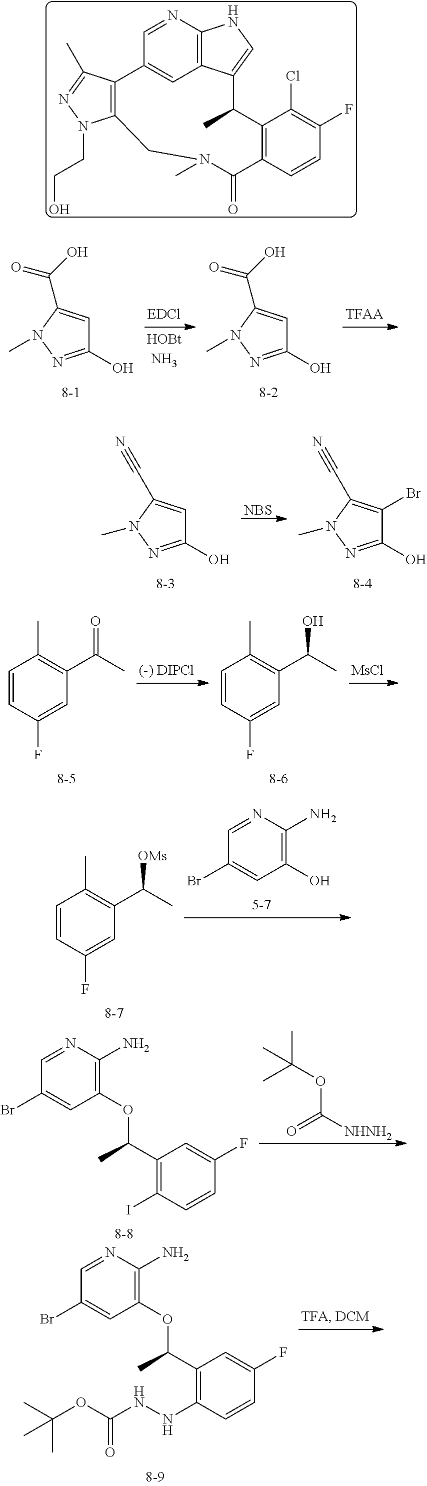

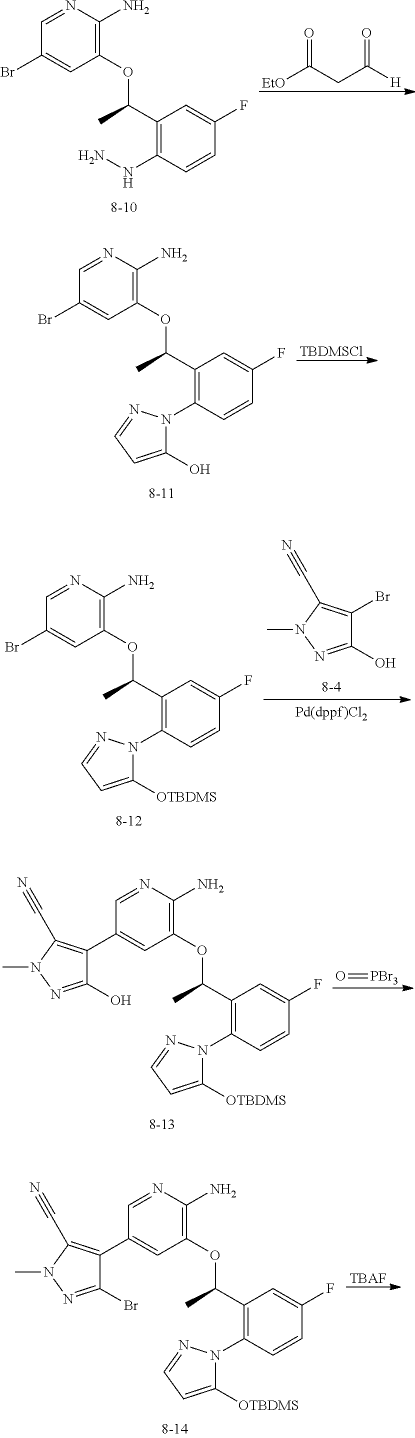

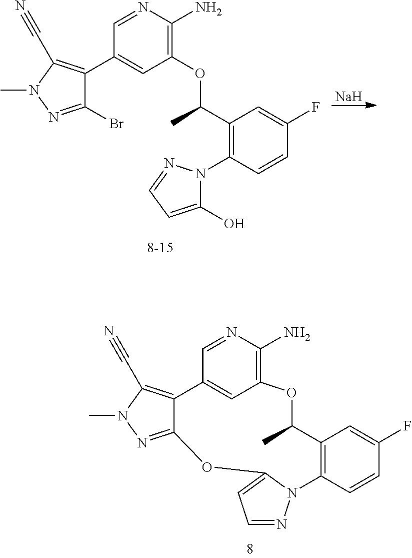

The acid 8-1 can be converted to amide 8-2, which can be further dehydrated to give compound 8-3. Bromination of compound 8-3 can give compound 8-4.

Compound 8-5 can be enantiomerically reduced to the chiral alcohol 8-6, which can be mesylated to give compound 8-7. Compound 8-7 can react with 5-7 to give compound 8-8, which can react with BOC protected hydrazine using a catalyst, such as CuI, to give compound 8-9. The removal of the BOC protecting group under acid conditions can give compound 8-10, which can be converted to compound 8-11. Protection of the OH group as a silyl ether can give compound 8-12, which can be coupled with 8-4 to give compound 8-13. Compound 8-13 can react with POBr3 to give compound 8-14, and deprotection of the hydroxyl group of compound 8-14 can give 8-15, which can be treated with a strong base, such as NaH, to give the desired product 8.

### Example 9

### Compound 9

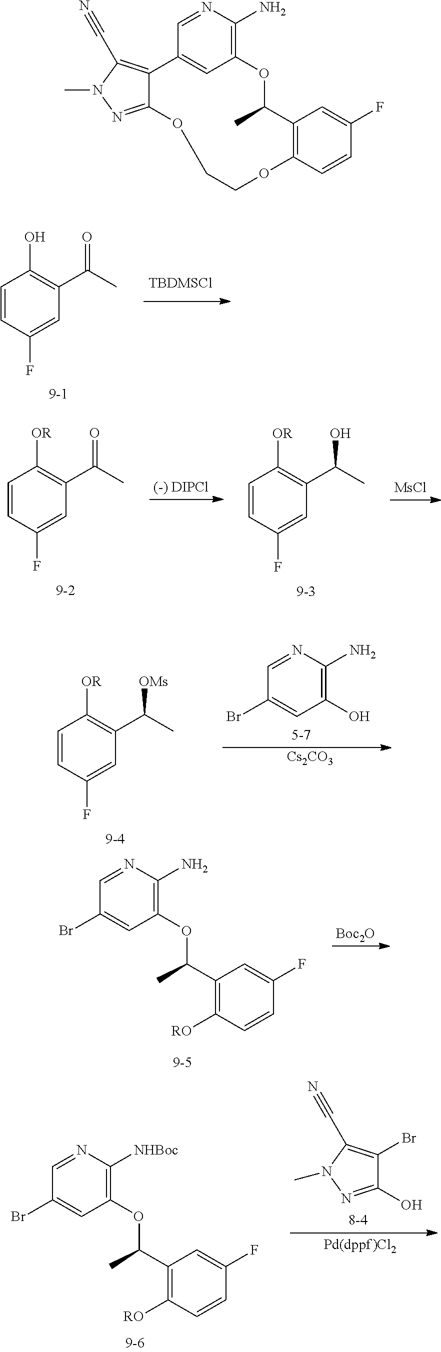

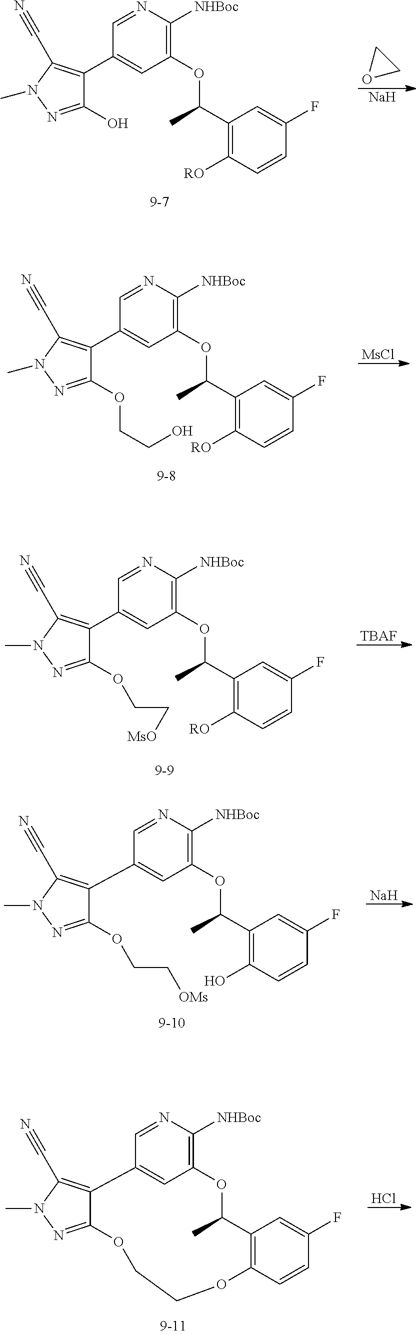

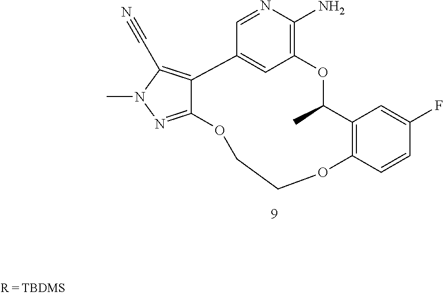 [SMILES: C[C@H]1Oc2cc(cnc2N)-c2c(nn(C)c2C#N)OCCOc2ccc(F)cc21]

Protection of the hydroxyl group of Compound 9-1 as a silyl ether can give compound 9-2, which can be enantiomerically reduced to the chiral alcohol 9-3. Compound 9-3 can be mesylated to give compound 9-4. Compound 9-4 can react with 5-7 to give compound 9-5. BOC protection of the amino group can give compound 9-6, which can couple with 8-4 to give compound 9-7. The hydroxyl group of compound 9-7 can be further alkylated to give compound 9-8, which can be mesylated to give compound 9-9. Removal of the protecting group on the phenolic oxygene can give compound 9-10, which can be further treated with astrong base, such as NaH, to give compound 9-11. Removal of the protecting group on the nitrogen can give the desired product 9.

### Example 10

### Compound 10

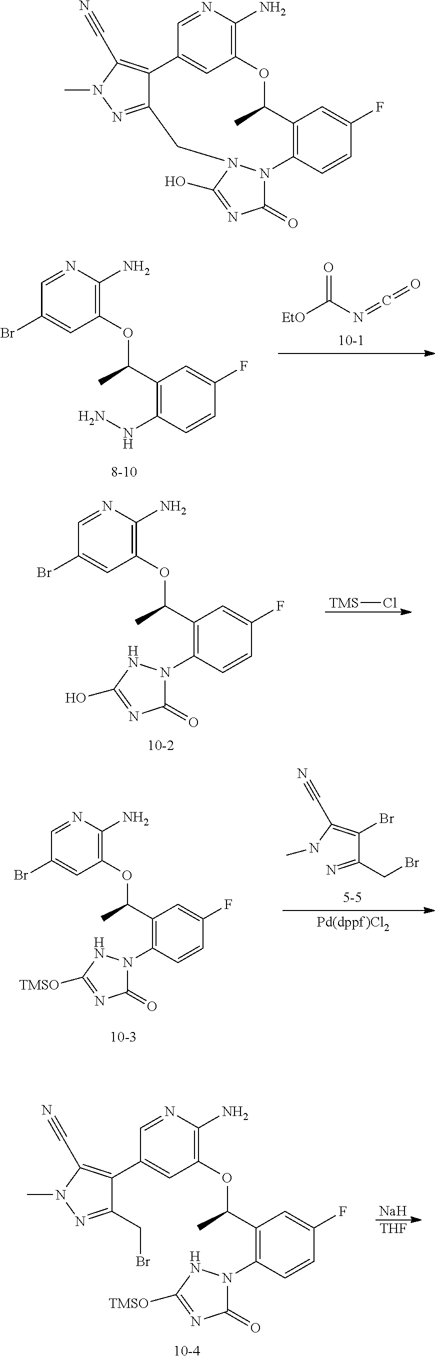

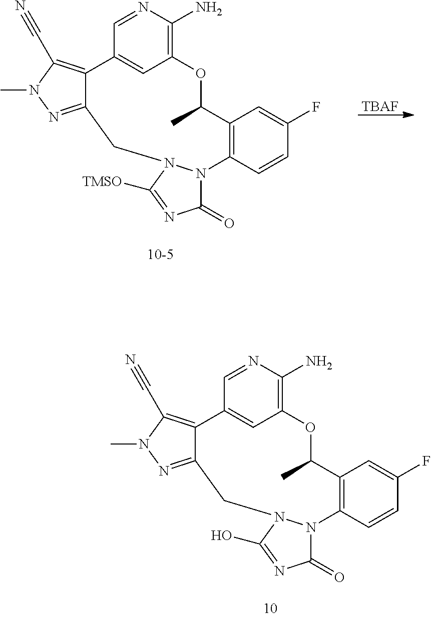

Compound 8-10 can react with compound 10-1 to give compound 10-2. Protection of the hydroxyl group of compound 10-2 can give compound 10-3, which can be coupled with compound 5-5 to give compound 10-4. Treatment of compound 10-4 with a strong base, such as NaH, can give compound 10-5, which can be treated with TBAF to remove the protection group to give desired compound 10.

### Example 11

### Compound 11

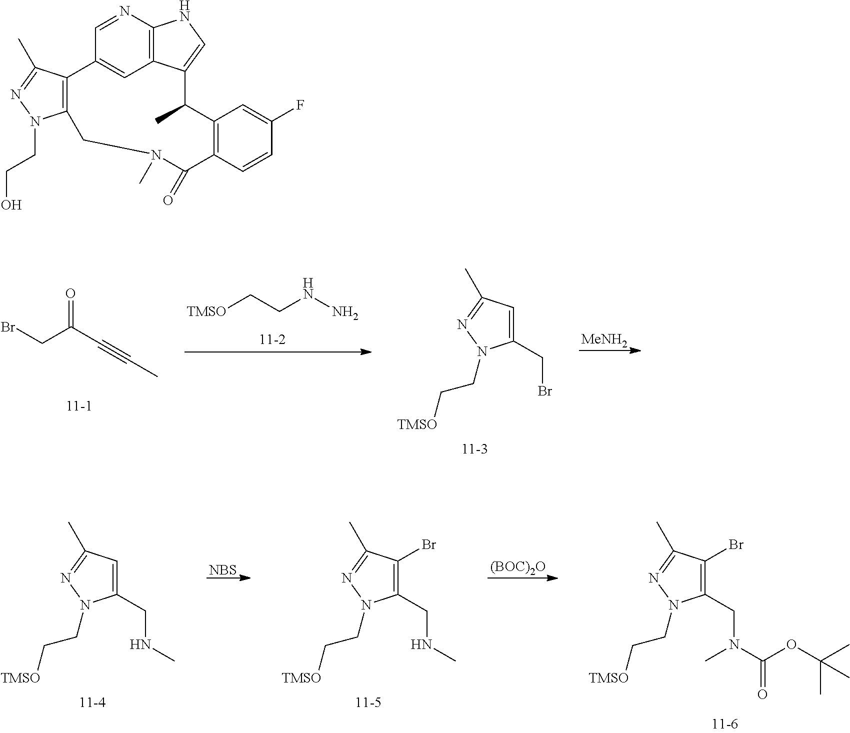

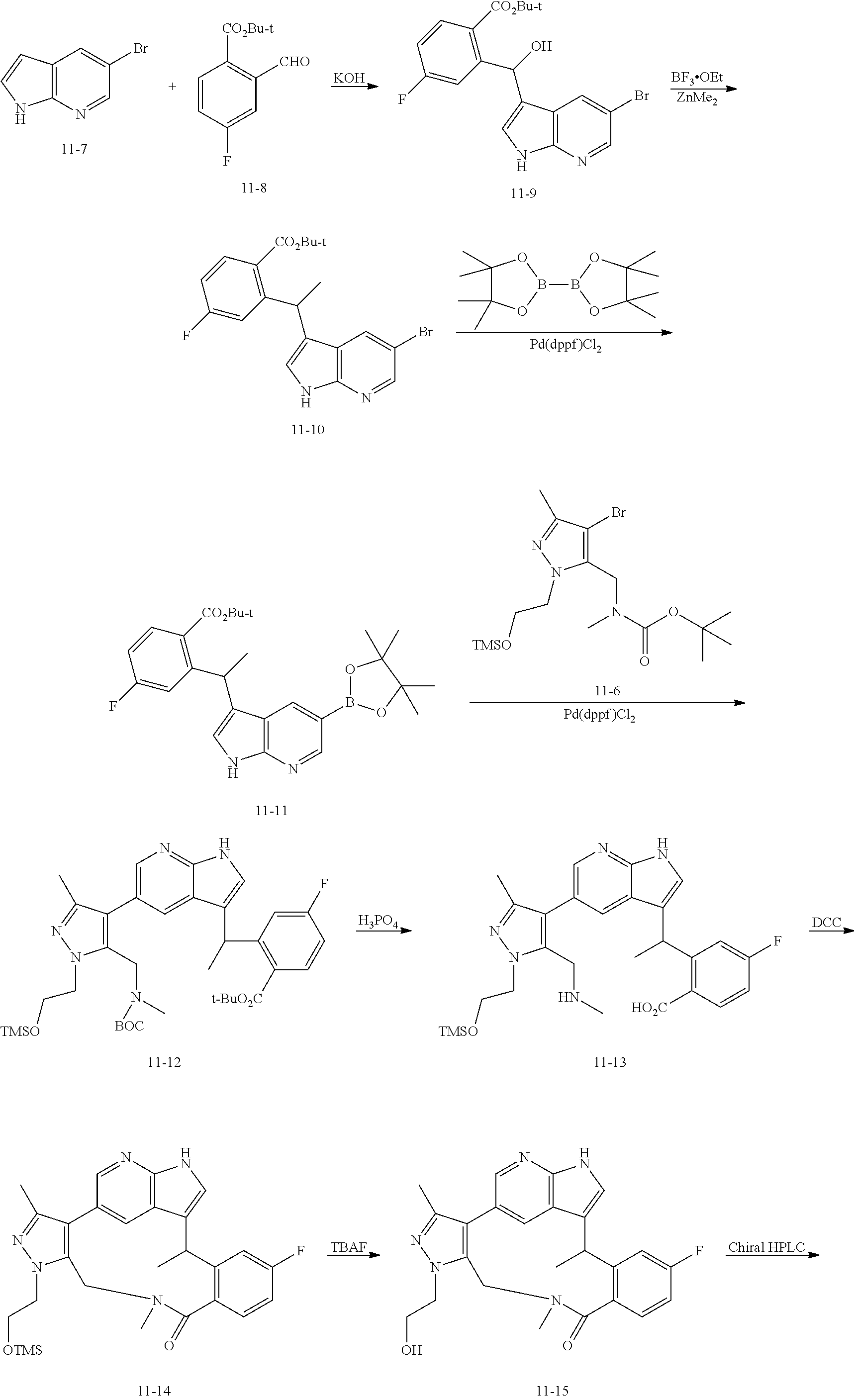

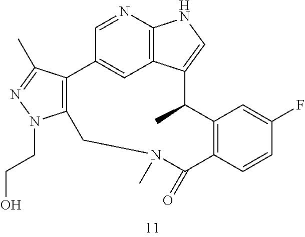 [SMILES: Cc1nn(CCO)c2c1-c1cnc3[nH]cc(c3c1)[C@H](C)c1cc(F)ccc1C(=O)N(C)C2]

Compound 11-1 can react with compound 11-2 to give compound 11-3, which can be converted to compound 11-4. Bromination of compound 11-4 with NBS can give compound 11-5. The nitrogen of compound 11-5 can be protected using a BOC protecting group to give compound 11-6.

Compound 11-7 can react with compound 11-8 in presence of a base, such as KOH, to give compound 11-9. The hydroxyl group of compound 11-9 can be converted to a methyl group to give compound 11-10, which can be converted to a bronic ester compound 11-11. Coupling of compound 11-11 with compound 11-6 can give compound 11-12. Treatment of compound 11-12 with a strong acid, such H₃PO₄, can give compound 11-13, which can be converted to compound 11-14 under standard amide bond formation reaction condition. Removal of the protecting group using TBAF can give compound 11-15. Separation by chial HPLC can give the desired compound 11.

### Example 12

### Compound 12

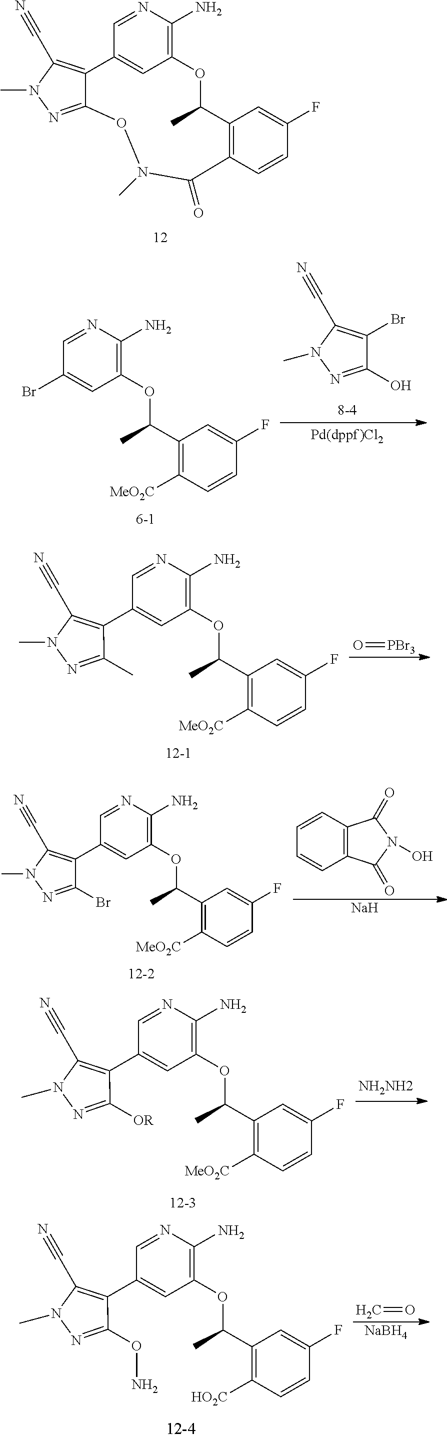

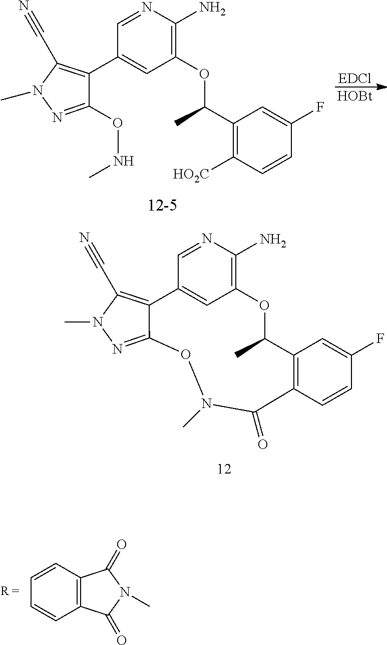

Compound 6-1 can be coupled with compound 8-4 to give 12-1, which can be treated with POBr₃ to give compound 12-2. Compound 12-2 can be converted to compound 12-3. Deprotection of the nitrogen of compound 12-3 can give compound 12-4, which can be methylated under reductive amination reaction conditions, such as using NaBH₄ and formaldehyde, to give compound 12-5. The intramolecular coupling reaction of compound 12-5 under amide bond formation reaction conditions, such as using EDCl as the activating reagent in present of HOBt, to give the desired product 12.

### Example 13

### Synthesis of Compounds 13a and 13b

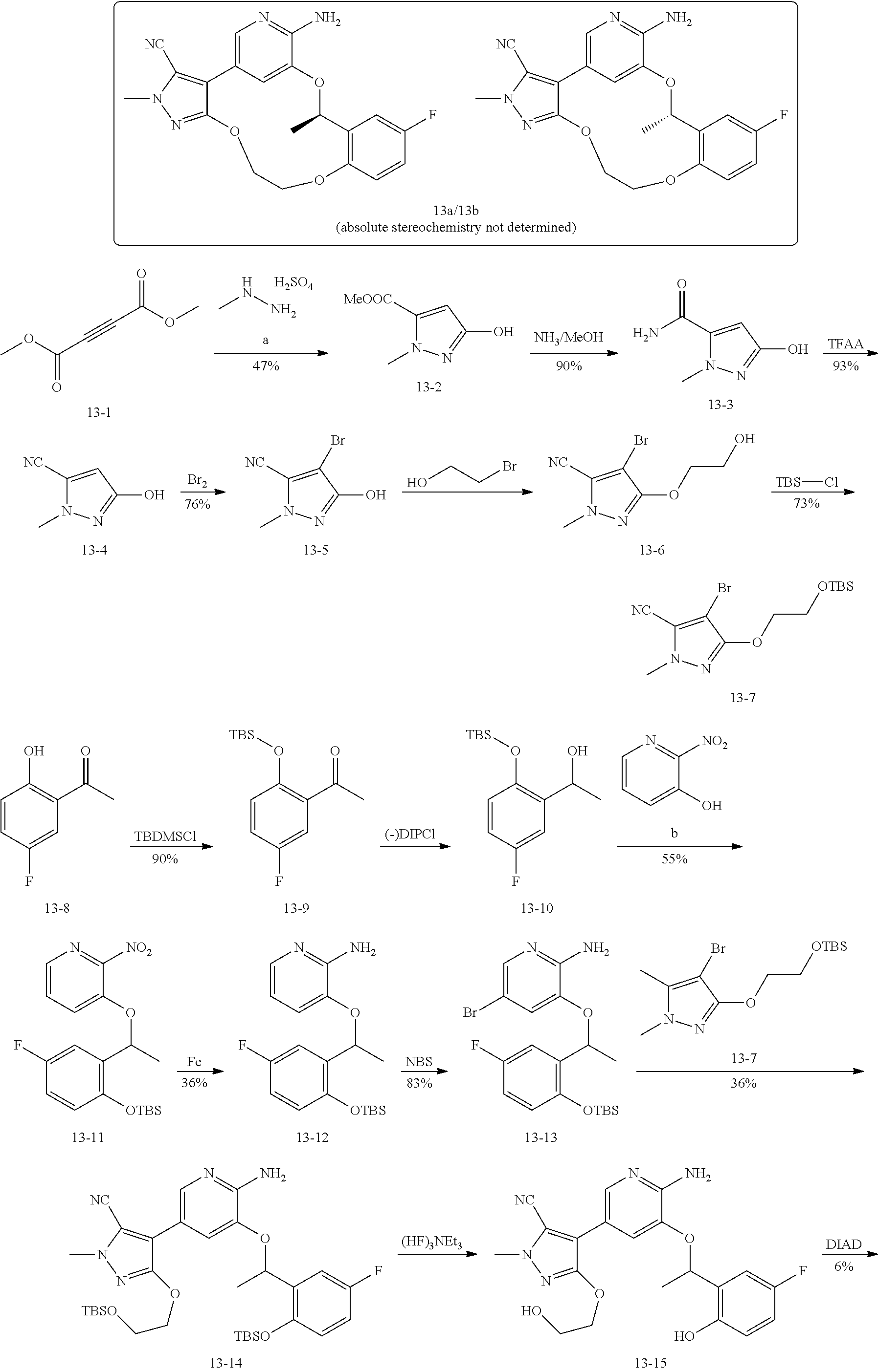

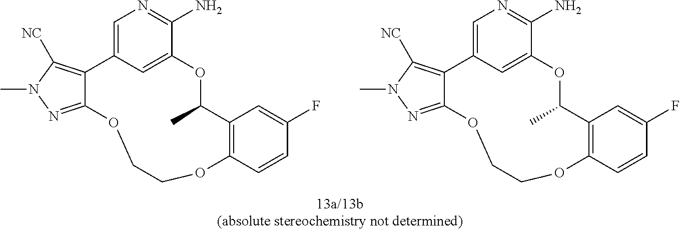

Step 1: Preparation of Compound 13-2.

TEA (21.95 mL, 158.33 mmol) was added to a solution of compound a (10.99 g, 70.37 mmol) in H₂O (50 mL) and MeOH (100 mL) at room temperature. The mixture was stirred for 0.5 h at room temperature, Compound 13-1 (10 g, 70.37 mmol) was added and the mixture was stirred for 18 h at 70° C. The solution was kept at room temperature for 2 h, and the solid was collected by filtration and dried to give the desired compound 13-2 (4.7 g, 47%) as a crude product which was used directly in next step. LCMS m/z 157 [M+1]⁺.

Step 2: Preparation of Compound 13-3.

A solution of compound 13-2 (120 g, 768.55 mmol) in MeOH/NH₃ (5M, 2 L) in a sealed tube was stirred at 50° C. for 24 h. Then the reaction mixture was concentrated to give compound 13-3 (88 g, 90%) as a crude product. LCMS m/z 142 [M+1]⁺. ¹H NMR (400 MHz, DMSO-d₆) δ ppm 7.76 (s, 1H), 7.37 (s, 1H), 6.05 (s, 1H), 3.83 (s, 3H).

Step 3: Preparation of Compound 13-4.

TFAA (416.7 g, 1.98 mol) was added drop wise to a solution of compound 13-3 (80 g, 566.86 mmol) and TEA (143.4 g, 1.42 mol) in THF (1200 mL). The solution was stirred at 25° C. for 1 h, extracted between EtOAc and water, dried over Na₂SO₄ and concentrated to give compound 13-4 (56 g, 93%) as a crude product. LCMS m/z 123[M+1]⁺. ¹H NMR (400 MHz, DMSO-d₆) δ ppm 10.48 (s, 1H), 6.28 (s, 1H), 3.78 (s, 3H).

Step 4: Preparation of Compound 13-5.

To a mixture of compound 13-4 (40.00 g, 324.91 mmol, 1.00 Eq) in CHCl₃ (1500 mL) was added Br₂ (51.92 g, 324.91 mmol, 1.00 Eq) in CHCl₃ (1500 mL) at 15° C. The mixture was stirred at 15° C. for 30 min. The mixture was concentrated to give the crude product compound 13-5 (50.00 g, 76.18% yield) as white solid. LCMS m/z 202/204 [M+1]⁺. ¹H NMR (400 MHz, DMSO-d₆) δ ppm 11.26 (s, 1H), 3.83 (s, 3H).

Step 5: Preparation of Compound 13-6.

To a mixture of compound 13-5 (10.00 g, 49.50 mmol, 1.00 Eq) and 2-bromoethanol (12.37 g, 99.01 mmol, 2.00 Eq) in CH₃CN (100 mL), was added KI (1.76 g, 9.9 mmol, 0.2 Eq) and K₂CO₃ (7.49 g, 49.5 mmol, 1.00 Eq) in one portion at 15° C. under N₂. The mixture was heated to 80° C. and stirred for 6 h. The reaction was extracted with EtOAc. The organic phase was dried with anhydrous Na₂SO₄, filtered and concentrated in vacuo. The residue was purified by silica gel chromatography (PE/EA=50/1) to afford compound 13-6 (7.80 g, crude) as a white solid. LCMS m/z 246/248 [M+1]⁺.

Step 6: Preparation of Compound 13-7.

TBS-Cl (551.28 mg, 3.66 mmol, 1.50 Eq) was added to a solution of compound 13-6 (600.00 mg, 2.44 mmol, 1.00 Eq) and imidazole (332.02 mg, 4.88 mmol, 2.00 Eq) in DMF (5 mL) in portions. The mixture was stirred at 25° C. for 1.5 h, poured into water (10 ml), and extracted with PE (petroleum ether, 20 mL). The organic layer was dried and concentrated to give compound 13-7 (650.00 mg, 73.93% yield) as a white solid. LCMS m/z 200/202[M−158]⁺. ¹H NMR (400 MHz, CDCl₃) δ ppm 4.30-4.28 (m, 2H), 3.96-3.93 (m, 2H), 3.88 (s, 3H), 0.89 (s, 9H), 0.89 (s, 6H).

Step 7: Preparation of Compound 13-9.

To solution of compound 13-8 (50.00 g, 324.38 mmol, 1.00 Eq) and imidazole (66.25 g, 973.14 mmol, 3.00 Eq) in DMF (500 mL) was added TBS-Cl (73.34 g, 486.57 mmol, 1.50 Eq) in portions at 0° C. Then the mixture was stirred at 25° C. for 2 h. The mixture was poured into water (500 mL) and then extracted with a mixture of PE:EtOAc (20:1, 500 mL). The combined organic phase was dried with anhydrous Na₂SO₄, filtered and concentrated in vacuo to afford compound 13-9 (79.00 g, crude) as a light yellow oil. LCMS m/z 269[M+1]⁺. ¹H NMR (400 MHz, CDCl₃) δ ppm 7.21-7.19 (m, 1H), 6.96-6.95 (m, 1H), 6.74-6.71 (m, 1H), 2.50 (s, 3H), 0.90 (s, 9H), 0.16 (s, 6H).

Step 8: Preparation of Compound 13-10.

To a solution of chloro-bis[(1R,2S,3R,5R)-2,6,6-trimethylnorpinan-3-yl]borane (1.7 M, 657.49 mL, 1.50 Eq) in THF (200 mL) was added a solution of compound 13-9 (200.00 g, 745.16 mmol, 1.00 Eq) in THF (100 mL) drop wise at −35˜−30° C. under N₂. After the addition, the mixture was warmed to 25° C. slowly and stirred for 3 h. The mixture was concentrated and the residue was purified by column chromatography to give compound 13-10 (201.50 g, crude, a mixture of two enantiomers with a ratio of 91.08:8.92 based on chiral HPLC analysis) as an oil, which was used directly in the next step. LCMS m/z 252[M−18]⁺.

Step 9: Preparation of Compound 13-11.

DIAD (523.72 mg, 2.59 mmol, 1.40 Eq) was added drop wise to a solution of compound 13-10 (500.00 mg, 1.85 mmol, 1.00 Eq), compound b (259.05 mg, 1.85 mmol, 1.00 Eq) and PPh₃ (679.33 mg, 2.59 mmol, 1.40 Eq) in THF (10 mL) at 0° C. Then the mixture was stirred at 25° C. for 8 h. The mixture was concentrated to give a crude product. The crude product was purified by column chromatography to give compound 13-11 (400.00 mg, 1.02 mmol, 55.09% yield). ¹H NMR (400 MHz, CDCl₃) δ ppm 7.91-7.90 (d, J=3.6 Hz, 1H), 7.24-7.23 (m, 1H), 7.17-7.16 (m, 1H), 7.01-6.98 (m, 1H), 6.76-6.75 (m, 1H), 6.69-6.68 (m, 1H), 5.66-5.62 (m, 1H), 1.54-1.53 (d, J=6.4 Hz, 3H), 0.95 (s, 9H), 0.23-0.15 (m, 6H).

Step 10: Preparation of Compound 13-12.

A solution of compound 13-11 (4.57 g, 11.64 mmol, 1.00 Eq) and Fe (3.25 g, 58.20 mmol, 5.00 Eq) in Sat. NH₄Cl (40 mL) and MeOH (40 mL) was heated to 80° C. for 2 h. The reaction mixture was filtered and the filtrate was concentrated to 50 mL. Then the mixture was extracted with EtOAc (100 mL). The organic layer was concentrated to compound 13-12 (1.50 g, 4.14 mmol, 35.55% yield) as brown solid. LCMS m/z 363[M+1]⁺. ¹H NMR (400 MHz, CDCl₃) δ ppm 7.61 (s, 1H), 7.03-7.00 (m, 1H), 6.83-6.77 (m, 2H), 6.68-6.66 (d, J=8 Hz, 1H), 6.46 (s, 1H), 5.61-5.56 (m, 1H), 4.73 (s, 2H), 1.62-1.60 (d, J=6.4 Hz, 3H), 1.05 (s, 9H), 0.31-0.29 (m, 6H).

Step 11: Preparation of Compound 13-13.

A solution of NBS (108.01 mg, 606.87 umol, 1.10 Eq) in CH₃CN (2 mL) was added to a solution of compound 13-12 (200.00 mg, 551.70 umol, 1.00 Eq) in MeCN (5 mL) at 25° C. The mixture was stirred for 1 h and concentrated to give a crude product. The crude product was purified by column chromatography to give compound 13-13 (202.00 mg, 457.62 umol, 83% yield). LCMS m/z 443/441[M+1]⁺. ¹H NMR (400 MHz, CDCl₃) δ ppm 7.65 (s, 1H), 7.00-6.98 (m, 1H), 6.86-6.78 (m, 3H), 5.59-5.57 (m, 1H), 4.79 (s, 2H), 1.62-1.60 (d, J=6.4 Hz, 3H), 1.06 (s, 9H), 0.35-0.31 (m, 6H).

Step 12: Preparation of Compound 13-14.

Compound 13-13 (2.00 g, 4.53 mmol, 1.00 Eq), compound 13-7 (1.80 g, 4.98 mmol, 1.10 Eq), 4,4,5,5-tetramethyl-2-(4,4,5,5-tetramethyl-1,3,2-dioxaborolan-2-yl)-1,3,2-dioxaborolane (3.45 g, 13.59 mmol, 3.00 Eq) in MeOH (1000 mL) was de-gassed, bis(1-adamantyl)-butyl-phosphane (649.81 mg, 1.81 mmol, 0.40 Eq) and Pd(OAc)₂ (203.45 mg, 906.19 umol, 0.20 Eq) was added to the mixture. After the reaction mixture was stirred for 5 min, a solution of NaOH (362.48 mg, 9.06 mmol, 2.00 Eq) in H₂O (20 mL) was added to the mixture. Then the mixture was heated to 80° C. for 1 h under N₂. TLC (PE:EtOAc=3:1) showed the starting material was consumed completely. The reaction mixture was concentrated to dryness and purified by column chromatography (PE:EA=15:1) to give compound 13-14 (1.06 g, 36.45% yield). LCMS m/z 642[M+1]⁺. ¹H NMR (400 MHz, CDCl₃) δ ppm 7.98 (s, 1H), 7.06-7.01 (m, 2H), 6.84-6.76 (m, 3H), 5.63-5.58 (m, 1H), 4.90 (s, 2H), 4.81 (s, 1H), 4.31-4.23 (m, 2H), 3.86 (s, 3H), 1.64-1.62 (d, J=6.4, 3H), 1.00 (s, 9H), 0.84 (s, 9H), 0.26 (s, 6H), 0.00 (s, 6H).

Step 13: Preparation of Compound 13-15.

Et₃N(HF)₃ (374.21 mg, 2.32 mmol, 2.00 Eq) was added to a solution of compound 13-14 (746.00 mg, 1.16 mmol, 1.00 Eq) in THF (10 mL) at 25° C. and stirred for 3 h. Water (10 ml) was added to the mixture, which was then extracted with EtOAc (20 mL). The organic layer was dried and concentrated to give compound 13-15 (662.00 mg, crude) which was directly used in the next step. LCMS m/z 414[M+1]⁺.

Step 14: Preparation of Compounds 9 and 13.

DIAD (1.47 g, 7.25 mmol, 5.00 Eq) was added drop wise to a solution of PPh₃ (1.90 g, 7.25 mmol, 5.00 Eq) in THF (250 ml) at 0° C. under N₂. The mixture was stirred for 15 min, and then a solution of compound 13-15 (600.00 mg, 1.45 mmol, 1.00 Eq) in THF (50 ml) was added to the mixture drop wise at 0° C. After the addition, the mixture was warmed to 25° C. and stirred for 4 hours. The mixture was concentrated to give a crude product. The crude product was purified by column chromatography, then Prep-HPLC, to give a mixture of Compounds 13a and 13b as a solid. Chiral separation was performed by SFC on a Chiralpak AY-H (5 μm particle size, 3.0 cm I.D.×25 cm L), which was eluted with 30% methanol (0.1% NH4OH) in CO₂, to give peak 1 with a retention time of 7.7 minutes and peak 2 with a retention time of 10.5 minutes.

Compound 13b (peak 1): a solid, 10.00 mg, 1.74% yield, 100% ee, 97.39% purity. ¹H NMR (400 MHz, CDCl₃) δ ppm 8.11 (s, 1H), 8.01 (s, 1H), 7.19-7.16 (m, 1H), 6.93-6.90 (m, 1H), 6.79-6.76 (m, 1H), 5.94-5.90 (m, 1H), 4.88 (s, 2H), 4.67-4.62 (m, 2H), 4.61-4.50 (m, 1H), 4.26-4.23 (m, 1H), 3.91 (s, 3H), 1.69-1.68 (d, J=6.4 Hz, 3H). LCMS M/Z 396 [M+1]⁺.

Compound 13a (peak 2): a solid, 31.00 mg, 5.23% yield, 100% ee, 98.18% purity. ¹H NMR (400 MHz, CDCl₃) δ ppm 8.11 (s, 1H), 8.01 (s, 1H), 7.19-7.16 (m, 1H), 6.93-6.89 (m, 1H), 6.79-6.75 (m, 1H), 5.92-5.91 (m, 1H), 4.88 (s, 2H), 4.68-4.50 (m, 3H), 4.28-4.23 (m, 1H), 3.91 (s, 3H), 1.69-1.68 (d, J=6.4 Hz, 3H). LCMS m/z 396[M+1]⁺.

### Example 14

### Synthesis of Compounds 14 and 15

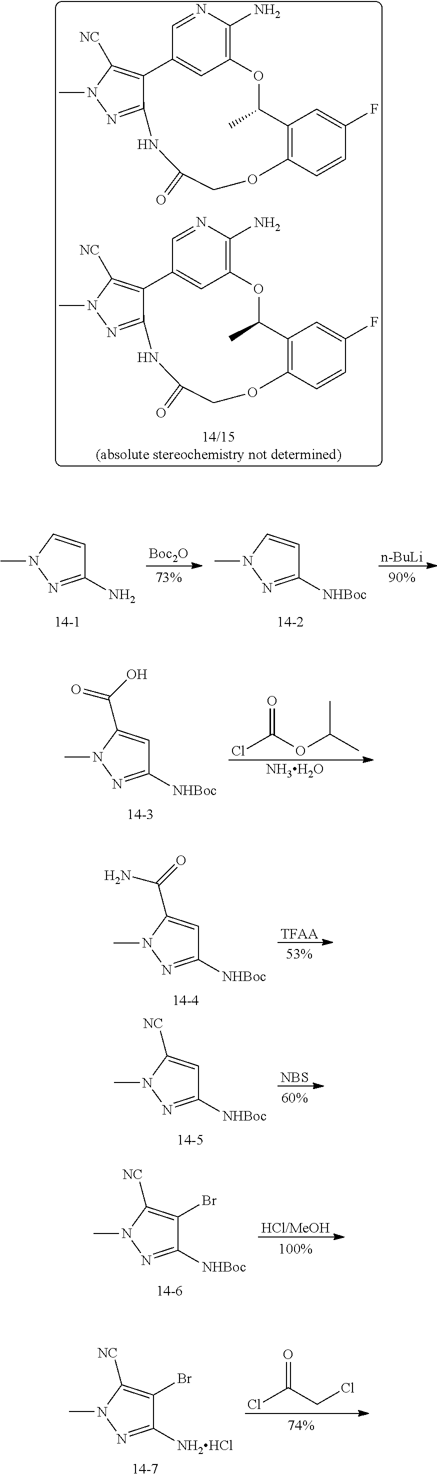

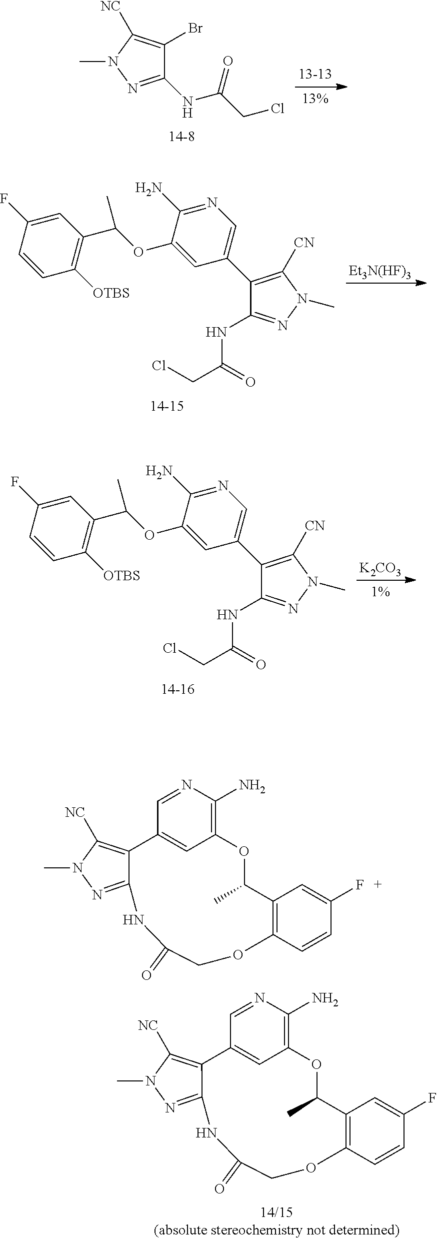

Step 1: Preparation of Compound 14-2.

Boc₂O (134.83 g, 617.79 mmol, 1.00 Eq) was added to a solution of compound 14-1 (60.00 g, 617.79 mmol, 1.00 Eq) and NaOH (27.18 g, 679.57 mmol, 1.10 Eq) in THF/H₂O (1:1) (1200 mL) at 25° C. in portions. The mixture was stirred for 4 h, extracted with EtOAc (800 mL), dried and concentrated to give compound 14-2 (89.00 g, 451.25 mmol, 73.04% yield) as a white solid. LCMS m/z 142[M−55]⁺. ¹H NMR (400 MHz, CDCl₃) δ ppm 8.01 (s, 1H), 7.21-7.20 (d, J=2 Hz, 1H), 6.44 (s, 1H), 3.81 (s, 3H), 1.51 (s, 9H).

Step 2: Preparation of Compound 14-3.

n-BuLi (2.5 M, 356.95 mL, 2.20 Eq) was added drop wise to a solution of compound 14-2 (80.00 g, 405.62 mmol, 1.00 Eq) in THF (800 mL) at −78° C. over 30 minutes. The mixture was stirred for an additional 30 minutes at −78° C., then CO₂ (solid) (178.47 g, 4.06 mol, 10.00 Eq) was added to the mixture. The mixture was warmed to 0° C. slowly and stirred for 30 min. The mixture was poured into water (500 mL), and extracted with EtOAc (200 mL). The aqueous layer was acidified to pH 4 by adding 4N HCl, and the precipitate was filtered and dried to give compound 14-3 (88.00 g, 364.78 mmol, 89.93% yield) as a white solid. LCMS m/z 264 [M+23]⁺. ¹H NMR (400 MHz, CDCl₃) δ ppm 9.72 (s, 1H), 6.73 (s, 1H), 3.94 (s, 3H), 1.44 (s, 9H).

Step 3: Preparation of Compound 14-4.

To a solution of compound 14-3 (42.00 g, 174.10 mmol, 1.00 Eq) and TEA (52.85 g, 522.30 mmol, 3.00 Eq) in DCM (500 mL) was added isopropyl carbonochloridate (32.00 g, 261.15 mmol, 1.50 Eq) drop wise at 0° C. Then the mixture was stirred at 25° C. for 5 h. TLC showed the reaction was complete. NH₃.H₂O (100 mL) was added to the mixture, and the mixture was stirred for another 4 h. The reaction mixture was concentrated to about 100 mL. Then the mixture was extracted with ethyl acetate (150 mL×3). The organic layer was dried and concentrated to give compound 14-4 (41.83 g, crude) which was used directly in next step. LCMS m/z 185[M−55]⁺.

Step 4: Preparation of Compound 14-5.

TFAA (53.76 g, 255.98 mmol, 1.50 Eq) was added to a solution of compound 14-4 (41.00 g, 170.65 mmol, 1.00 Eq) and TEA (34.54 g, 341.30 mmol, 2.00 Eq) in DCM (800 mL) at 25° C., and the mixture was stirred for 3 h. TLC showed the reaction was completed. Water (300 mL) was poured into the mixture, and the mixture was concentrated to about 300 mL. The precipitate was filtered and dried to give compound 14-5 (20.00 g, 89.99 mmol, 52.74% yield) as a light yellow solid. ¹H NMR (400 MHz, CDCl₃) δ ppm 7.67 (s, 1H), 6.98 (s, 1H), 3.94 (s, 3H), 1.51 (s, 9H).

Step 5: Preparation of Compound 14-6.

NBS (25.63 g, 143.98 mmol, 2.00 Eq) was added to a solution of compound 14-5 (16.00 g, 71.99 mmol, 1.00 Eq) in DMF (300 mL) in portions at 25° C. The mixture was stirred for 4 h and then poured into H₂O (600 mL). The precipitate was filtered and dried to give compound 14-6 (13.00 g, 43.17 mmol, 59.97% yield) as a white solid. LCMS m/z 245/247 [M−55]⁺. ¹H (400 MHz, CDCl₃) δ ppm 6.39 (s, 1H), 4.02 (s, 3H). 1.52 (s, 9H).

Step 6: Preparation of Compound 14-7.

A mixture of compound 14-6 (5.00 g, 16.60 mmol, 1.00 Eq) in HCl/MeOH (4N, 120 mL) was stirred at 25° C. for 6 h. LCMS showed the reaction was finished. The reaction mixture was concentrated to give compound 14-7 (3.94 g, 16.59 mmol, 100.00% yield) as a white solid. LCMS m/z 201/203 [M+1]⁺.

Step 7: Preparation of Compound 14-8.

2-chloroacetyl chloride (2.85 g, 25.27 mmol, 2.00 Eq) was added to a solution of compound 14-7 (3.00 g, 12.63 mmol, 1.00 Eq) and TEA (3.83 g, 37.90 mmol, 3.00 Eq) in DCM (50 mL) at 0° C. for 0.5 h. Then the mixture was stirred at 25° C. for 3.5 h. The mixture was extracted between DCM and H₂O. The organic layer was concentrated and purified by column chromatography (P: E=10:1) to give compound 14-8 (2.61 g, 9.41 mmol, 74.49% yield). LCMS m/z 279/277 [M+1]⁺. ¹H NMR (400 MHz, CDCl₃) δ ppm 8.22 (s, 1H), 4.25 (s, 2H), 4.06 (s, 3H).

Step 8: Preparation of Compound 14-15.

A solution of Compound 14-8 (2.51 g, 9.06 mmol, 1.00 Eq), compound 13-13 (prepared as described in Example 13) (4.00 g, 9.06 mmol, 1.00 Eq), 4,4,5,5-tetramethyl-2-(4,4,5,5-tetramethyl-1,3,2-dioxaborolan-2-yl)-1,3,2-dioxaborolane (6.90 g, 27.19 mmol, 3.00 Eq) in MeOH (1.5 L) was de-gassed, and bis(1-adamantyl)-butyl-phosphane (649.81 mg, 1.81 mmol, 0.20 Eq) and diacetoxypalladium (203.45 mg, 906.19 umol, 0.10 Eq) were added to the mixture. After stirring for 5 minutes, a solution of NaOH (724.95 mg, 18.12 mmol, 2.00 Eq) in H₂O (20 mL) was added to the mixture. Then the mixture was heated to 80° C. for 8 h under N₂. TLC (PE:EtOAc=1:1) showed the starting material was consumed completely. The reaction mixture was concentrated to dryness and purified by column chromatography (PE:EA=2:1) to give compound 14-15 (1.30 g, 2.33 mmol, 12.86% yield). LCMS m/z 559 [M+1]⁺. ¹H NMR (400 MHz, CDCl₃) δ ppm 8.17 (s, 1H), 7.70-7.67 (m, 1H), 7.05-7.01 (m, 2H), 6.86-6.79 (m, 3H), 5.62-5.58 (m, 1H), 5.04 (s, 2H), 4.19 (s, 2H), 4.02 (s, 3H), 1.64-1.62 (d, J=6.4 Hz, 3H), 0.99 (s, 9H), 0.28-0.23 (m, 6H).

Step 9: Preparation of Compound 14-16.

Et₃N(HF)₃ (6 M, 344.00 uL, 1.00 Eq) was added to a solution of compound 14-15 (1.15 g, 2.06 mmol, 1.00 Eq) in THF (20 mL) at 25° C. Then the mixture was stirred for 0.5 h. TLC showed the reaction was finished. Water (10 mL) was added to the mixture, and the mixture was extracted with EA (20 mL). The organic layer was dried and concentrated to give compound 14-16 (916.00 mg, crude), which was used directly in next step. LCMS m/z 445 [M+1]⁺.

Step 10: Preparation of Compounds 14 and 15.

A solution of compound 14-16 (898.60 mg, 2.02 mmol, 1.00 Eq) and K₂CO₃ (697.96 mg, 5.05 mmol, 2.50 Eq) in acetone (40 mL) was heated to 80° C. for 8 h. LCMS showed the reaction was finished, filtered, and the filtrate was concentrated and purified by column chromatography (PE: EA=1:4) and followed by Prep-HPLC to give a mixture of Compounds 14 and 15. The chiral separation was performed by SFC on a Chiralpak AD-H (5 μm particle size, 3.0 cm I.D.×25 cm L), which was eluted with 30% isopropyl alcohol (0.1% NH₄OH) in CO₂, to give peak 1 with a retention time of 13.74 minutes and peak 2 with a retention time of 16.81 minutes.

Compound 15 (peak 1): a solid, 10.00 mg, 24.49 umol, 1.21% yield, 96% ee, 95.21% purity, LCMS m/z 409[M+1]⁺. ¹H NMR (400 MHz, CDCl₃) δ ppm 8.01 (s, 1H), 7.79 (s, 1H), 7.25-7.00 (m, 1H), 7.13-7.12 (d, J=1.6 Hz, 1H), 7.03-6.94 (m, 2H), 5.84-5.82 (m, 1H), 4.98-4.95 (d, J=14 Hz, 3H), 4.83-4.80 (d, J=14 Hz, 1H), 4.05 (s, 3H), 1.72-1.70 (d, J=6.4 Hz, 3H).

Compound 14 (peak 2): a solid, 11.00 mg, 26.94 umol, 1.33% yield, 99% ee, 92.28% purity, LCMS m/z 423 [M+1]⁺. ¹H NMR (400 MHz, CDCl₃) δ ppm 7.94 (s, 1H), 7.71 (s, 1H), 7.17-7.14 (m, 1H), 7.05 (s, 1H), 6.95-6.89 (m, 2H), 5.76-5.74 (m, 1H), 4.90-4.87 (d, J=14 Hz, 3H), 4.75-4.72 (d, J=14 Hz, 1H), 3.97 (s, 3H), 1.64-1.62 (d, J=6.8 Hz, 3H).

### Example 15

### Synthesis of Compounds 16 and 17

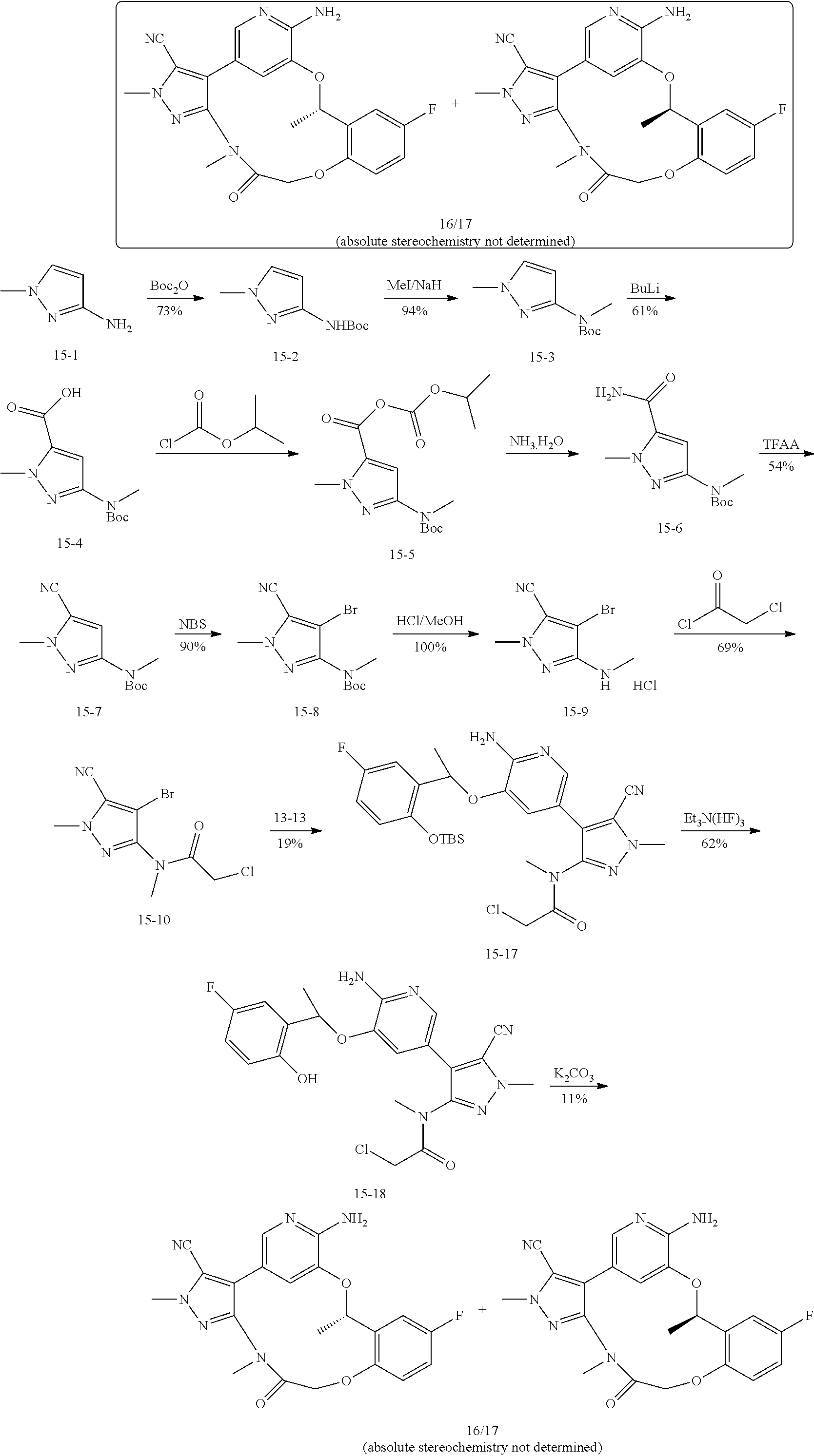

Step 1: Preparation of Compound 15-2.

Boc₂O (134.83 g, 617.79 mmol, 1.00 Eq) was added to a solution of compound 15-1 (60.00 g, 617.79 mmol, 1.00 Eq) and NaOH (27.18 g, 679.57 mmol, 1.10 Eq) in THF/H₂O (1:1) (1200 mL) at 25° C. in portions. The mixture was stirred for 4 h, and extracted with EtOAc (800 ml). The organic phase was dried and concentrated to give compound 15-2 (89.00 g, 451.25 mmol, 73.04% yield) as a white solid. LCMS m/z 142 [M−55]⁺. ¹H NMR (400 MHz, CDCl₃) δ ppm 8.01 (s, 1H), 7.21-7.20 (d, J=2 Hz, 1H), 6.44 (s, 1H), 3.81 (s, 3H), 1.51 (s, 9H).

Step 2: Preparation of Compound 15-3.

NaH (16.22 g, 405.46 mmol, 1.00 Eq) was added to a solution of compound 15-2 (80.00 g, 405.62 mmol, 1.00 Eq) in DMF (1500 mL) at 0° C. in portions. After the mixture was stirred for 30 min, CH₃I (69.20 g, 487.53 mmol, 1.20 Eq) was added. Then the mixture was warmed to 25° C. and stirred for 1 h. The reaction was quenched with Sat. NH₄Cl (200 ml) and then mixed with water (1000 ml), the mixture was extracted with PE:EA (20:1, 1000 mL×2), and the organic layer was separated, dried and concentrated to give compound 15-3 (80.36 g, 380.38 mmol, 93.78% yield) as an oil. LCMS m/z 156 [M−55]⁺. ¹H NMR (400 MHz, CDCl₃) δ ppm 7.20-7.19 (d, J=2.4 Hz, 1H), 6.37 (s, 1H), 3.80 (s, 3H), 3.33 (s, 3H), 1.52 (s, 9H).

Step 3: Preparation of Compound 15-4.

n-BuLi (2.5 M, 63.81 mL, 1.00 Eq) was added drop wise to a solution of compound 15-3 (33.70 g, 159.52 mmol, 1.00 Eq) in THF (400 mL) at −78° C. The mixture was stirred for 30 minutes at −78° C., and then CO₂ (solid) (70.19 g, 1.60 mol, 10.00 Eq) was added to the mixture. The mixture was warmed to 0° C. slowly and stirred for 30 minutes. The mixture was poured into water (500 ml), and extracted with EtOAc (200 mL). The aqueous layer was acidified to pH 4 by adding 2N HCl, and the precipitate was filtered and dried to give compound 15-4 (25.00 g, 97.94 mmol, 61.39% yield) as a white solid. LCMS m/z 200 [M−55]⁺. ¹H NMR (400 MHz, CDCl₃) δ ppm 13.34 (s, 1H), 6.82 (s, 1H), 3.99 (s, 3H), 3.22 (s, 3H), 1.46 (s, 9H).

Step 4: Preparation of Compound 15-5.

To a solution of compound 15-4 (50.00 g, 195.87 mmol, 1.00 Eq) and TEA (59.46 g, 587.61 mmol, 3.00 Eq) in DCM (500 mL) was added isopropyl chloroformate (28.80 g, 235.05 mmol, 1.20 Eq) dropwise at 0° C. Then the mixture was stirred at 25° C. for 1 h. The mixture was concentrated to give compound 15-5 (66.86 g, crude) which was used in next step without further purification.

Step 5: Preparation of Compound 15-6.

To a solution of compound 15-5 (1.00 g, 2.93 mmol, 1.00 Eq) in THF (10 mL) was added NH₃.H₂O (1.82 g, 51.92 mmol, 17.72 Eq) at 25° C. The mixture was stirred for 30 minutes. The mixture was extracted with EtOAc, and the organic extracts were dried over Na₂SO₄, filtered, and concentrated to give compound 15-6 (481.00 mg, crude) which was used in the next step without further purification. LCMS m/z 199 [M−55]⁺. ¹H NMR (400 MHz, CDCl₃) δ ppm 7.92 (s, 1H), 7.42 (s, 1H), 3.94 (s, 3H), 3.20 (s, 3H), 1.46 (s, 9H).

Step 6: Preparation of Compound 15-7.

TFAA (102.83 g, 489.60 mmol, 2.50 Eq) was added to a solution of compound 15-6 (49.80 g, 195.84 mmol, 1.00 Eq) and TEA (79.27 g, 783.36 mmol, 4.00 Eq) in DCM (500 mL) drop wise at 0° C. After addition, the mixture was warmed to 25° C. and stirred for 2 h. Poured water (500 ml) to the mixture and extracted with DCM (1000 mL). The organic layer was dried and concentrated to give a crude product. The crude product was purified by column chromatography (PE:EA=20:1) to give compound 15-7 (25.00 g, 105.81 mmol, 54% yield). LCMS m/z 181 [M−55]⁺. ¹H NMR (400 MHz, CDCl₃) δ ppm 6.96 (s, 1H), 3.94 (s, 3H), 3.32 (s, 3H), 1.52 (s, 9H).

Step 7: Preparation of Compound 15-8.

NBS (6.12 g, 34.38 mmol, 1.20 Eq) was added to a solution of compound 15-7 (6.77 g, 28.65 mmol, 1.00 Eq) in DMF (80 mL) in portions at 25° C. The mixture was stirred for 5 h. The mixture was poured into water (100 ml), and the precipitate was filtered and dried to give compound 15-8 (8.10 g, 25.70 mmol, 89.70% yield) as a white solid. LCMS m/z 259/261 [M−55]⁺. ¹H NMR (400 MHz, CDCl₃) δ ppm 4.01 (s, 3H), 3.18 (s, 3H), 1.44 (s, 9H).

Step 8: Preparation of Compound 15-9.

A solution of compound 15-8 (12.00 g, 38.07 mmol, 1.00 Eq) in HCl/MeOH (4N, 200 mL) was stirred at 25° C. for 5 h. The mixture was concentrated to give compound 15-9 (9.57 g, 38.05 mmol, 100% yield) as a white solid. LCMS m/z 215/217 [M+1]⁺. ¹H NMR (400 MHz, CDCl₃) δ ppm 4.02 (s, 3H), 3.04 (s, 3H), 2.70 (s, 1H).

Step 9: Preparation of Compound 15-10.

Chloromethyl chloroformate (2.69 g, 23.86 mmol, 2.00 Eq) was added to a solution of compound 15-9 (3.00 g, 11.93 mmol, 1.00 Eq) and TEA (3.62 g, 35.78 mmol, 3.00 Eq) in DCM (40 mL) at 0° C. for 0.5 h. Then the mixture was stirred at 25° C. for 1 h. The mixture was extracted between DCM and H₂O. The organic layer was concentrated and purified by column chromatography (PE:EA=20:1) to give compound 15-10 (2.40 g, 11.29 mmol, 69.01% yield). LCMS m/z 293/291 [M+1]⁺. ¹H NMR (400 MHz, CDCl₃) δ ppm 4.08 (s, 3H), 3.98 (s, 2H), 3.26 (s, 3H).

Step 10: Preparation of Compound 15-17.

A solution of Compound 15-10 (2.00 g, 9.42 mmol, 1.25 Eq), compound 13-13 (prepared as described in Example 13) (3.33 g, 7.54 mmol, 1.00 Eq), and 4,4,5,5-tetramethyl-2-(4,4,5,5-tetramethyl-1,3,2-dioxaborolan-2-yl)-1,3,2-dioxaborolane (5.74 g, 22.62 mmol, 3.00 Eq) in MeOH (1 L) was de-gassed, and bis(1-adamantyl)-butyl-phosphine (540.68 mg, 1.51 mmol, 0.20 Eq) and diacetoxypalladium (169.28 mg, 754.00 umol, 0.10 Eq) were added to the mixture. After the reaction mixture was stirred for 5 min, a solution of NaOH (603.20 mg, 15.08 mmol, 2.00 Eq) in H₂O (20 mL) was added to the mixture. Then the mixture was heated to 80° C. for 8 h under N₂. TLC (PE:EtOAc=1:1) showed the starting material was consumed completely. The reaction mixture was concentrated to dryness and purified by column chromatography (PE:EA=5:1) to give compound 15-17 (800.00 mg, 1.40 mmol, 18.57% yield). LCMS m/z 573[M+1]⁺. ¹H NMR (400 MHz, CDCl₃) δ ppm 7.72 (s, 1H), 7.44-7.43 (m, 1H), 6.99-6.96 (m, 2H), 6.81-6.78 (m, 2H), 5.59-5.54 (m, 1H), 4.02 (s, 3H), 3.93-3.85 (m, 2H), 2.95 (s, 3H), 1.65-1.63 (d, J=5.6, 3H), 0.99 (s, 9H), 0.25 (s, 6H).

Step 11: Preparation of Compound 15-18.

Et₃N(HF)₃ (6 M, 232.64 uL, 1.00 Eq) was added to a solution of compound 15-17 (800.00 mg, 1.40 mmol, 1.00 Eq) in THF (40 mL) at 25° C., and the solution was stirred for 1 h. The mixture was concentrated to give a crude product. The crude product was purified by column chromatography (PE:EA=3:1) to give compound 15-18 (400.00 mg, 871.71 umol, 62.27% yield). LCMS m/z 459 [M+1]⁺.

Step 12: Preparation of Compounds 16 and 17.

A solution of compound 15-18 (360.00 mg, 784.54 umol, 1.00 Eq) and K₂CO₃ (216.86 mg, 1.57 mmol, 2.00 Eq) in Acetone (50 mL) was heated to 55° C. and stirred for 3 h. The mixture was filtered and concentrated to give a crude product, which was purified by Prep-HPLC to give a mixture of compounds 16 and 17. Chiral separation was performed by SFC on a Chiralpak OD-H (5 μm particle size, 3.0 cm I.D.×25 cm L), which was eluted with 35% methanol (0.1% NH4OH) in CO₂, to give peak 1 with a retention time of 6.25 minutes and peak 2 with a retention time of 8.85 minutes.

Compound 17 (peak 1): a solid, 23.00 mg, 7% yield, 97% ee, 90.14% purity, LCMS m/z 423 [M+1]⁺, ¹H NMR (400 MHz, DMSO-d₆) δ ppm 7.92 (s, 1H), 7.46-7.38 (m, 2H), 7.14-7.05 (m, 2H), 6.25 (S, 2H), 5.92-5.90 (m, 1H), 5.31-5.28 (d, J=12.8 Hz, 1H), 4.93-4.90 (d, J=12.8 Hz, 1H), 3.99 (s, 3H), 3.57 (s, 3H), 1.61-1.60 (d, J=5.6 Hz, 3H).

Example 16 (peak 2): a solid, 36.00 mg, 11% yield, 100% ee, 93.19% purity, LCMS m/z 423 [M+1]⁺, ¹H NMR (400 MHz, DMSO-d₆) δ ppm 7.89 (s, 1H), 7.43-7.35 (m, 2H), 7.11-7.02 (m, 2H), 6.22 (s, 2H), 5.89-5.88 (m, 1H), 5.29-5.26 (d, J=12.8 Hz, 1H), 4.90-4.87 (d, J=12.8 Hz, 1H), 3.96 (s, 3H), 3.55 (s, 3H), 1.58-1.57 (d, J=5.6 Hz, 3H).

### Kinase Inhibition Assays

Kinase inhibition by the compounds of formula (I) is measured using commercially available assay kits and services that are well-known to a person having ordinary skill in the art. These kits and services are used to measure the inhibition of a variety of kinases, including without limitation ALK, ABL, AXL, Aur B & C, BLK, HPK, IRAM, RON, ROS1, SLK, STK10, TIE2, TRK, c-Met, Lck, Lyn, Src, Fyn, Syk, Zap-70, Itk, Tec, Btk, EGFR, ErbB2, Kdr, Flt-1, Flt-3, Tek, c-Met, InsR, and Atk. Commercial suppliers of these assay kits and services include Promega Corporation and Reaction Biology Corporation, EMD Millipore, and CEREP. In addition to the commercially available assay kits and services, the kinase inhibition activity of the compounds of formula (I) is measured by way of the assays described below.

### Example 16

### Wild-Type ALK and L1196M Mutant ALK Enzyme Assays

Wild-type ALK and L1196M mutant ALK enzyme inhibition are measured using a microfluidic mobility shift assay. The reactions are conducted in 50 μL of DMSO in 96-well plates. The reaction mixtures contain preactivated human recombinant wild-type (1.3 nM) or L1196M (0.5 nM) ALK kinase domain (amino acids 1093-1411), 1.5 μM phosphoacceptor peptide, 5′FAMKKSRGDYMTMQIG-CONH2 (CPC Scientific, Sunnyvale, Calif.), test compound (11-dose 3-fold serial dilutions, 2% DMSO final) or DMSO only, 1 mM DTT, 0.002% Tween-20 and 5 mM MgCl₂ in 25 mM Hepes, pH 7.1, and are initiated by addition of ATP (60 μM final concentration, ˜Kₘ level) following a 20-min preincubation. The reactions are incubated for 1 h at room temperature, stopped by the addition of 0.1 M EDTA, pH 8, and the extent of reactions (˜15-20% conversion with no inhibitor) is determined after electrophoretic separation of the fluorescently labeled peptide substrate and phosphorylated product on an LabChip EZ Reader II (Caliper Life Sciences, Hopkinton, Mass.). The K values are calculated by fitting the % conversion to the equation for competitive inhibition using non-linear regression method (GraphPad Prism, GraphPad Software, San Diego, Calif.) and experimentally measured ATP Kₘ for wildtype and for L1196M enzyme. ALK enzymes are produced by baculoviral expression and are preactivated by auto-phosphorylation of 16 μM non-activated enzyme in the presence of 2 mM ATP, 10 mM MgCl₂ and 4 mM DTT in 20 mM Hepes, pH 7.5, at room temperature for 1 h. The full phosphorylation (−4 phosphates per protein molecule) of ALK kinase domain can be verified by Q-TOF mass-spectrometry.

### Example 17

### Wild Type ALK and L1196M Mutant ALK Enzyme Assays

Wild type ALK and L1196M mutant ALK enzyme inhibition by the compounds of formula (I) was measured using an HTRF assay.

Materials.

ALK wild type and ALK L1196M were acquired from Carna Biosciences (Japan). A standard HTRF kit (containing Eu-labeled TK1 antibody, XL665, biotin conjugated TK1 peptide, 5× enzymatic buffer and 1×HTRF detection buffer) were purchased from Cis-Bio International. Plates were read on an Envision multi-label plate reader (Perkin Elmer).

Methods.

In both assays, the compounds were tested in 11 doses in duplicate with 3-fold dilution as the final concentrations were from 10 μM to 0.17 nM. Compound 13a was tested with lower top concentrations of 1 μM due to high potency.

The enzyme reaction mixture of ALK wild type standard HTRF assay contained 0.5 nM ALK wild type, 1 μM biotin-TK1 peptide, 30 μM ATP and 50 nM SEB in 1× enzymatic reaction buffer containing 50 mM (pH 7.0) HEPES, 5 mM MgCl₂, 0.02% NaN₃, 0.01% BSA, 0.1 mM Orthovanadate and 1 mM DTT at a final volume of 10 μl. The enzyme reaction was carried out at room temperature in white Proxiplate 384-Plus plate (PerkinElmer) for 90 minutes.

The enzyme reaction mixture of ALK L1196M standard HTRF assay contained 0.15 nM ALK L1196M, 1 μM biotin-TK1 peptide, 30 μM ATP and 50 nM SEB in 1× enzymatic reaction buffer containing 50 mM (pH7.0) HEPES, 5 mM MgCl₂, 0.02% NaN₃, 0.01% BSA, 0.1 mM Orthovanadate and 1 mM DTT at a final volume of 10 μl. The enzyme reaction was carried out at room temperature in white Proxiplate 384-Plus plate (PerkinElmer) for 60 minutes.

The detection reagents (10 ml) were added at final concentrations of 2 nM antibody and 62.5 nM XL665. The plates were incubated at room temperature for 60 minutes and then read in the Envision plate reader.

Data Analysis.

The readouts were transformed into inhibition rate % by the equation of (Ratio-Min)/(Max-Min)*100%. Hence the IC50 data of test compounds were generated by using four parameters curve fitting (Model 205 in XLFIT5, iDBS).

Results.

The IC50 data obtained with wild type ALK and L1196M mutant ALK enzyme assays disclosed above are shown in Table 1, where compounds that have no data indicate that those compounds were not tested against the target enzyme listed in Table 1.

TABLE 1
ALK Wild Type and ALK L1196M Enzyme Inhibition Data
for Compounds of Formula (I)

| | IC50 (nM) | IC50 (nM) |
|---|---|---|
| Compound | ALK_wt | ALK_L1196M |
| 13a | 1.4 | 54 |
| 13b | 2269 | 4472 |
| 14 | 55.9 | 1081 |
| 15 | 2371 | |
| 16 | 34.8 | 529 |
| 17 | 7434 | |

### Example 18

### Cellular Phospho-ALK (Tyr1604) ELISA Assay for EML4-ALK

Cell Lines.

NIH-3T3 MEF cell lines are stably transfected with human EML4-ALK wt and EML4-ALK L1196M cDNA to express the proteins in useful amounts. The cells are maintained at 37° C. in a 5% CO₂ incubator in DMEM (Invitrogen, Carlsbad, Calif.) medium supplemented with 1% L-glutamine, 1% penicillin and streptomycin, 1 ug/ml puromycin and 10% NCS in T-75 flasks.

Assay.

Cells are washed with PBS and re-suspended in DMEM medium supplemented with 0.5% NCS and 1% pen/strep and seeded into 96-well plates at density of 20,000 cells/well/100 μL and incubated in the incubator at 37° C. and 5% CO₂. After 20 hours of incubation, 100 uL of assay media (DMEM) containing a specified concentration of the test compound or DMSO (control) is added into plates and incubated for 1 hour in the incubator. Media is then removed and lysis buffer, containing phosphatase inhibitors and PMSF, is added to the wells and shaken at 4° C. for 30 minutes to generate protein lysates.

Subsequently, a PathScan phospho-ALK (Tyr1604) chemiluminescent sandwich ELISA kit (Cell Signal Technology Inc., cat #7020) is used to assess the phosphorylation of ALK as follows: A phospho-ALK (Tyr1604) rabbit antibody is coated onto the 96-well microplates. 50 μL of cell lysates are added to the antibody coated plate and incubated at room temperature for 2 hours. Following extensive washing with 0.1% Tween 20 in PBS to remove unbound materials, ALK mouse mAb is added to detect captured phospho-ALK (Tyr1604) and phospho-ALK fusion proteins. Anti-mouse IgG, HRP-linked antibody is then used to recognize the bound detection antibody. Finally, the chemiluminescent reagent is added and incubated for 10 minutes for signal development. The assay plates are read in the Envision plate reader in the luminescent mode. IC₅₀ values are calculated by a concentration-response curve fitting using a four-parameter analytic method.

### Example 19

### GAS6-Axl Cellular Signaling Assays with Phosphor-AXL and Phosphor-AKT Readout

Pancreatic cancer cells (PSN-1 cell line) are seeded in 1 ml of the appropriate growth media with 10% FBS into 6-well plates (8×10⁵ cells/well) and incubated overnight at 37° C. and 5% CO₂. The following day, serum-containing growth media is replaced by serum-free media and incubated for 4 hr, and then test compounds are added to the cells at desired concentrations and incubated for an additional 2 hr. To stimulate Axl signaling, Gas6 is added to each well to a concentration of 3 μg/ml and incubated for 10 min. The cells are lysed immediately and the lysates are used in a multiplex ELISA kit (Meso Scale Discovery, Gaithersburg, Md.) to quantitate the total AKT and phospho-AKT (Ser473) according to the manufacturer's protocol. The same lysates are analyzed in a phospho-Axl ELISA (R&D Systems) using the protocol provided with the kit from the manufacturer. IC₅₀ values are determined using GraphPad Prism 5 software. The data are entered as an X-Y plot into the software as percent inhibition for each concentration of the drug. The concentration values of the drug are log transformed and the nonlinear regression is carried out using the “sigmoidal dose-response (variable slope)” option within the GraphPad software to model the data and calculate IC₅₀ values. The IC₅₀ values reported are the concentration of drug at which 50% inhibition is reached.

### Example 20

### Other Kinase Inhibition Assays

Assays to determine the inhibition of other kinases by the compounds of formula (I) are performed according to procedures known to a person having ordinary skill in the art. These assays include, but are not limited to, assays directed to the inhibition of the following kinases:
Wild-type c-Met Kinase. Inhibition of wild-type c-Met kinase is determined as described in International Publication No. WO 2011/069761, the entire contents of which are incorporated by reference.
LCK and BLK Kinases. Inhibition of LCK and BLK kinases is determined as described in U.S. Pat. No. 7,125,875, the entire contents of which are incorporated by reference.
Various Other Kinases. Inhibition of various other kinases, including but not limited to Lck, Lyn, Src, Fyn, Syk, Zap-70, ltk, Tec, Btk, EGFR, ErbB2, Kdr, Flt-1, Flt-3, Tek, c-Met, InsR, and Atk is determined as described in U.S. Pat. No. 6,881,737, the entire contents of which are incorporated by reference.

### Animal Models and Tumor Cell Assays

### Example 21

### ALK Xenograft Model (Karpas 299 Tumor Cells)

RNU nude rats are inoculated subcutaneously in the flank with a suspension of 5×10⁶ Karpas 299 tumor cells, and the tumors are allowed to grow to an average size of at least 300 mm³. At this time the animals are randomized into 3 groups, and dosed with vehicle control, and two different doses of the test agent, daily for 14 days via oral gavage in a suitable vehicle such as 0.5% MC/0.5% Tween 80. Tumor sizes are measured every three days by calipers, and volume is determined by an appropriate formulation, and results are reported out as size of treated tumors divided by size of control tumors.

### Example 22

### ALK Xenograft Model (H2228 Tumor Cells)

RNU nude rats are inoculated subcutaneously in the flank with a suspension of 5×10⁶ H2228 tumor cells, and the tumors are allowed to grow to an average size of at least 300 mm³. At this time the animals are randomized into 3 groups, and dosed with vehicle control, and two different doses of the test agent, daily for 14 days via oral gavage in a suitable vehicle such as 0.5% MC/0.5% Tween 80. Tumor sizes are measured every three days by calipers, and volume is determined by an appropriate formulation, and results are reported out as size of treated tumors divided by size of control tumors.

### Example 23

### c-Met Cellular Assays

As described in Cancer Research 70, 1524 (2010), the entire contents of which are incorporated by reference, proliferation and viability of tumor cells are measured using the ViaLight PLUS kit (Cambrex). Analysis of phosphorylation status of c-Met, in cells: Tumor cells are treated for 2 h with drug or vehicle in RPMI 1640 supplemented with 10% fetal bovine serum and 10 mmol/L HEPES. When called for, the cells are stimulated with HGF during the last 10 min of the 2 h incubation. The cells are lysed with a denaturing or nondenaturing buffer containing phosphatase and protease inhibitors and subjected to Western blot or immunoprecipitation-Western blot analysis.

### Example 24

### c-Met Xenograft Models

As described in Cancer Research 70, 1524 (2010), the entire contents of which are incorporated by reference, GTL-16 cells are inoculated subcutaneously into the flank of female nude CD-1 nu/nu mice. When mean tumor size reaches a predetermined range, the mice are randomized and given vehicle or test article by perioral gavage once or twice daily. Tumor volumes are determined using calipers. The percentage increase in the volume of a xenograft tumor on day n versus day 0 (the day when dosing of the test compound began) is calculated as (tumor volume on day n−tumor volume on day 0/tumor volume on day 0)×100. The mean percentage of tumor growth inhibition in each drug-treated group relative to the vehicle-treated group is calculated as (1−mean percent increase of tumor volume in the drug-treated group/mean percent increase of the tumor volume in the vehicle-treated group)×100.

### Example 25

### Measurement of c-Met (Y1349) Phosphorylation in Xenograft Tumors

As described in Cancer Research 70, 1524 (2010), the entire contents of which are incorporated by reference, mice bearing GTL-16 tumors are euthanized at appropriate intervals after perioral administration of drug. The tumors are excised, snap-frozen, and dispersed using a Qiagen Tissue-Lyser in a nondenaturing lysis buffer containing protease and phosphatase inhibitors. The homogenate is lysed at 4° C. for 1 h, clarified by centrifugation, and then analyzed by quantitative Western blotting for phospho-c-Met (Y1349) and total c-Met. The pMet (Y1349) signal of each c-Met band is normalized with its total c-Met signal. To combine or compare data from several gels, the pY1349/total Met ratio for each c-Met band is further normalized to the average pY1349/total c-Met ratio of the vehicle-treated tumor samples on the same gel.

### Example 26

### TrkB Cellular Assays

SY5Y cells are transfected with TrkB to produce the SY5Y-TrkB subclone, which is grown in RPMI 1640 containing 10% fetal bovine serum and 0.3 mg/mL G418 and maintained in 150 cm³ Costar culture flasks in a humidified atmosphere of 95% air and 5% CO₂. These cells are grown in 10 cm³ dishes to 70% to 80% confluency in standard culture medium and harvested for protein extraction. TrkB expression is analyzed by Western blot using an anti-phospho-Trk antibody (phospho-TrkA, Tyr⁴⁹⁰ antibody; Cell Signaling Technologies) or an anti-pan-Trk antibody (Santa Cruz Biotechnology). Cells are exposed to BDNF for 10 min in the absence or presence of increasing concentrations of the test article to determine the concentration that achieved 50% inhibition of receptor phosphorylation (IC₅₀).

### Example 27

### TrkB Xenograft Models

Four to eight week old nu/nu mice are injected subcutaneously in the flank with 1×10⁷ SY5Y-TrkB cells in 0.3 mL Matrigel (BD Biosciences). Tumor sizes are measured twice weekly in three dimensions, and the volume was calculated as follows: (d1×d2×d3)×π/6. Body weights are obtained twice weekly, and the dose of compound is adjusted accordingly. Treatment with test compounds is started ˜10 days after tumor inoculation when the average SY5Y-TrkB tumor size was 200 mm³.

### Example 28

### RON Xenograft Model

HT-29 cells, (2×10⁶) in Matrigel are injected subcutaneously in the flank of six to eight week old CD1 nu/nu mice. When tumors have grown to an average size of 200 mm3, animals are randomized, and treated by oral gavage with either vehicle or a suspension of the test article either qd or bid at appropriate doses. Tumor sizes are measured twice weekly in three dimensions, using calipers, and results are reported as the percentage increase in the volume of a xenograft tumor on day n versus day 0 (the day when dosing of the test compound began) is calculated as (tumor volume on day n−tumor volume on day 0/tumor volume on day 0)×100. The mean percentage of tumor growth inhibition in each drug-treated group relative to the vehicle-treated group is calculated as (1−mean percent increase of tumor volume in the drug-treated group/mean percent increase of the tumor volume in the vehicle-treated group)×100.

### Example 29

### T351I Resistant Bcr-Abl Xenograft Model

The p210 Bcr-Abl oncogene with the Abl T351I mutant is transfected into Ba/F3 cells, and the desired cells are selected for by growth in normal media in the absence of IL-3. Female SCID mice (6 to 8 wk old) are tail vein injected with Ba/F3-p210T315I cells (10⁶ cells in 100 μl of serum-free medium). At 3 days postinjection, the mice are either intravenously infused or orally gavaged with vehicle or test article for 7 d. At 20 d postinjection, leukocytes in the peripheral blood of the mice are separated and analyzed by flow cytometry (FACSCalibur) for Bcr-Abl containing cells.

What is claimed is:

1. A compound of the formula (I) 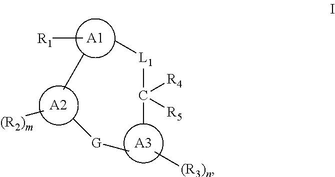
or a pharmaceutically acceptable salt thereof, wherein:
A1 is 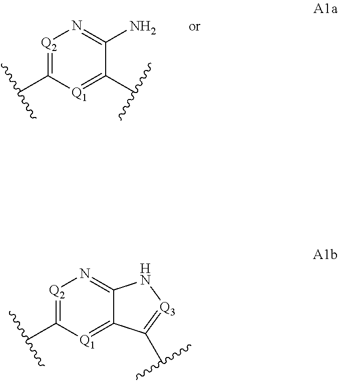
Q₁ is N or CH;
Q₂ is N or CH;
Q₃ is N or C-J-R₁;
A2 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 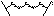
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, —O(CR₉R₁₀)ₒ—, —S(O)ₓ(CR₉R₁₀)ₒ—, or —NR₈(CR₉R₁₀)ₒ— or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 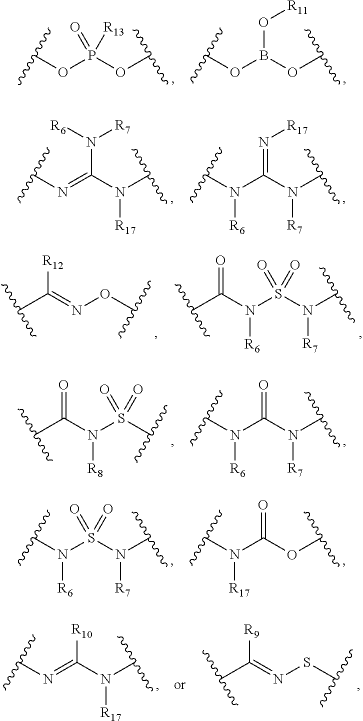
or a divalent ring system selected from the group consisting of: 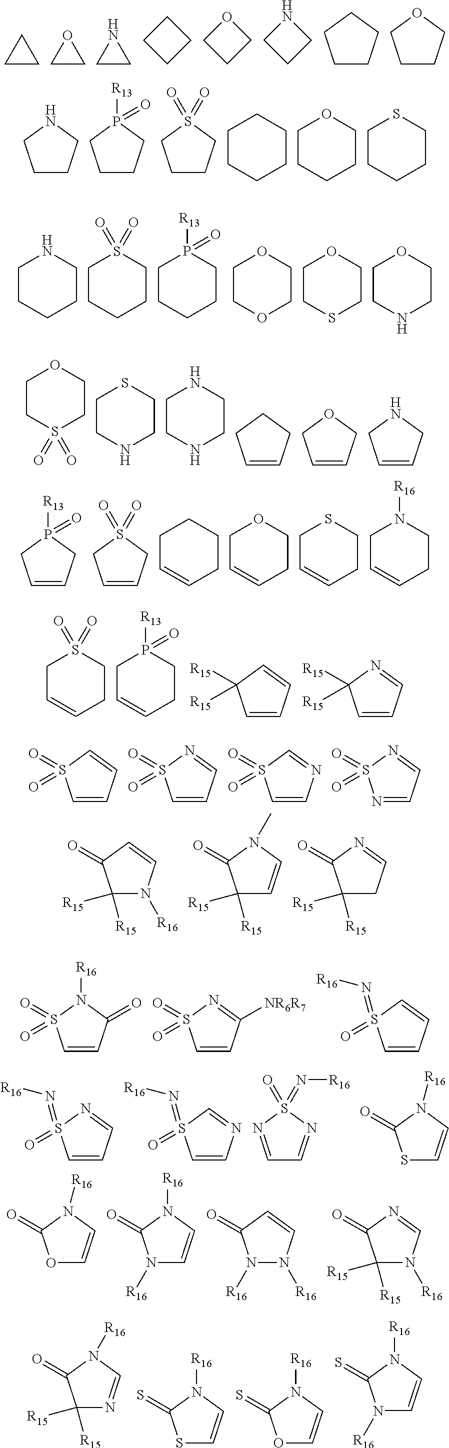 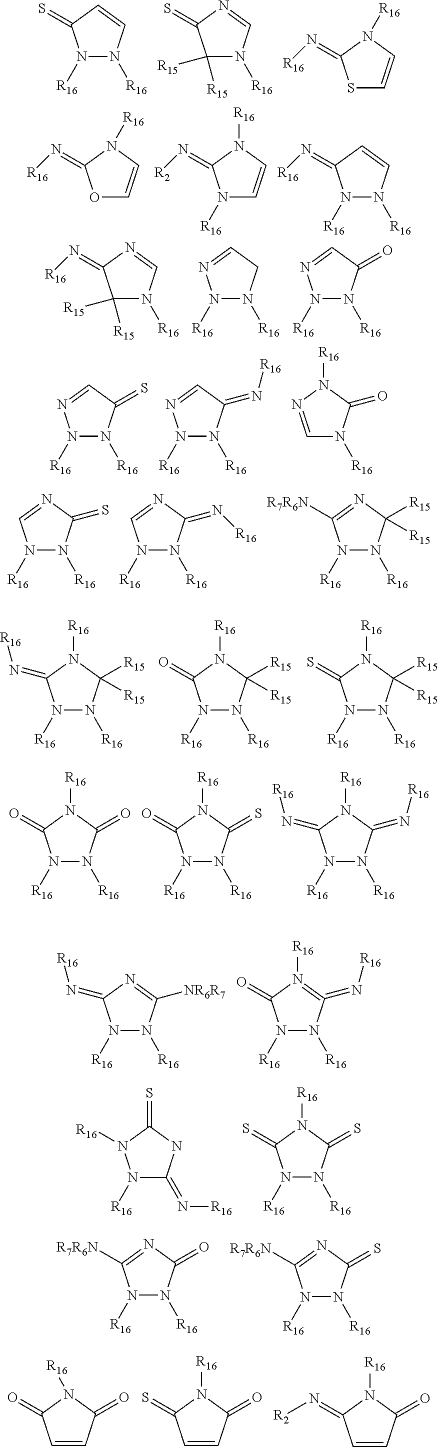 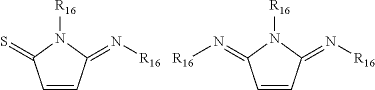 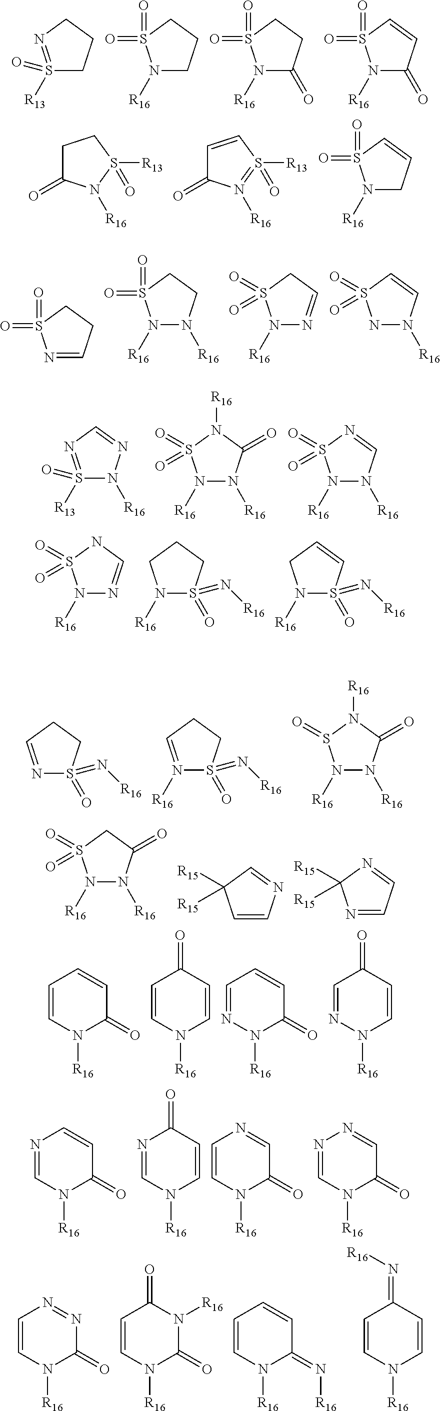 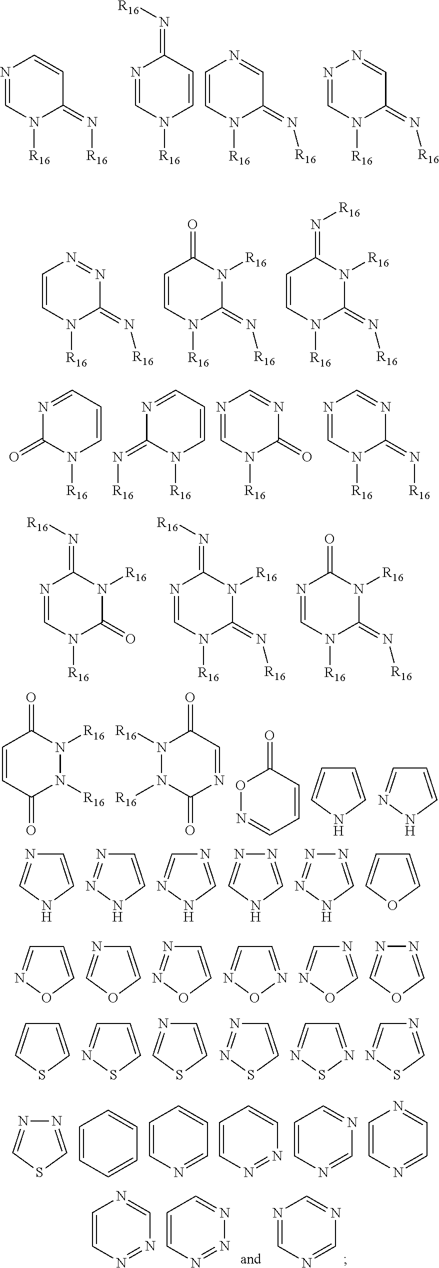
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that when A1 is A1a, and only one out of X and Y is present, and G contains one heteroatom selected from N and O within the direct chain that links A2 and A3, and G does not contain a carbon-carbon double bond in the direct chain that links A2 and A3, and R₃ is halogen or C₁₋₆ alkyl, and none of R₂, R₉, R₁₀, R₁₅ and R₁₆ contain phosphorus, a sulfur-nitrogen double bond, sulfur bonded to two nitrogen atoms, nitrogen bonded to one oxygen atom, a carbonate, a carbamate or a urea; then neither X nor Y is selected from the group consisting of: O, NR₈, CONR₈, NR₈CO₃ 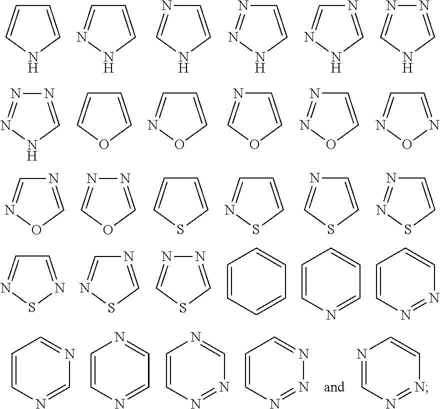
and any R₁₅ or R₁₆ substituent bound to a divalent ring system at a position vicinal to, or vinylogously linked to, C═O, C═N or C═S is not H if tautomerization at such position could lead to aromatization of the divalent ring system.

2. A compound of the formula (I) 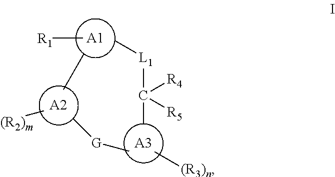
or a pharmaceutically acceptable salt thereof, wherein:
A1 is 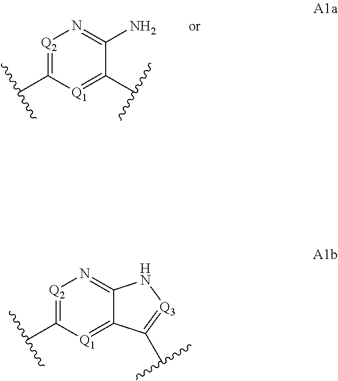
Q₁ is N or CH;
Q₂ is N or CH;
Q₃ is N or C-J-R₁;
A2 is a C₈-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₈-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 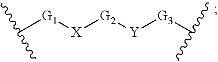
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, or —S(O)ₓ(CR₉R₁₀)ₒ—, or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 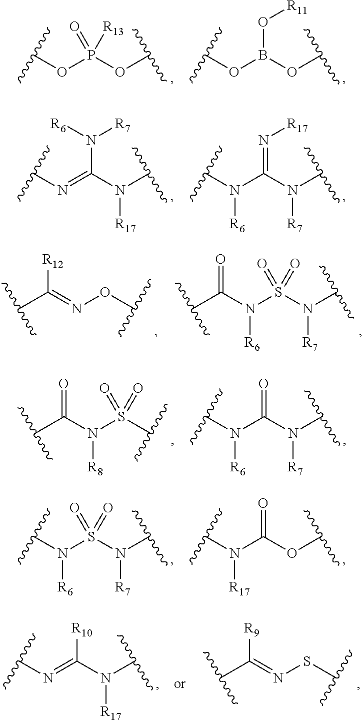
or a divalent ring system selected from the group consisting of: 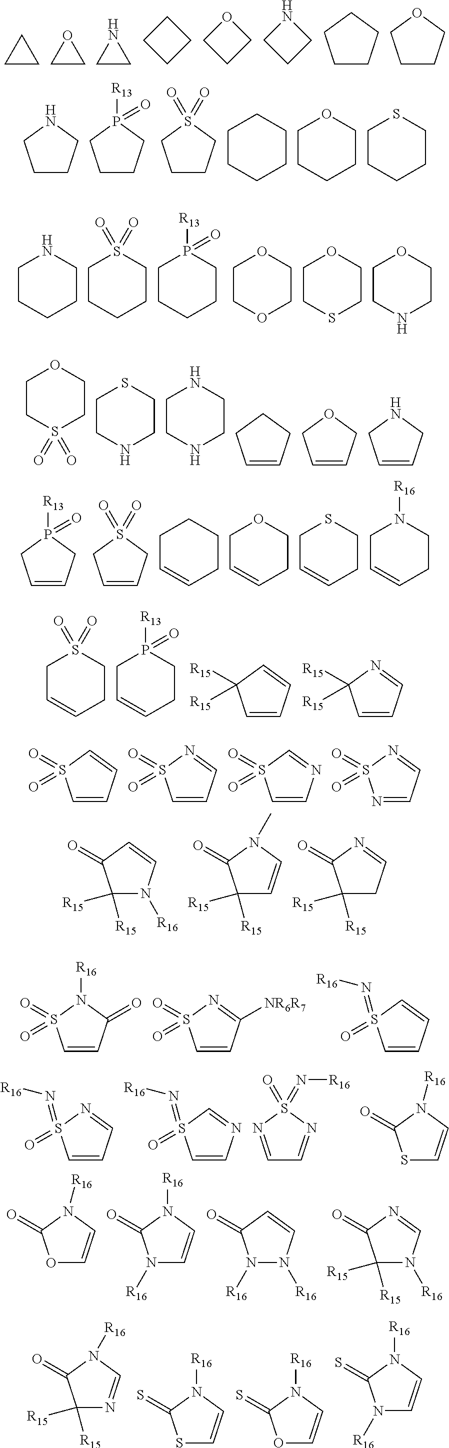 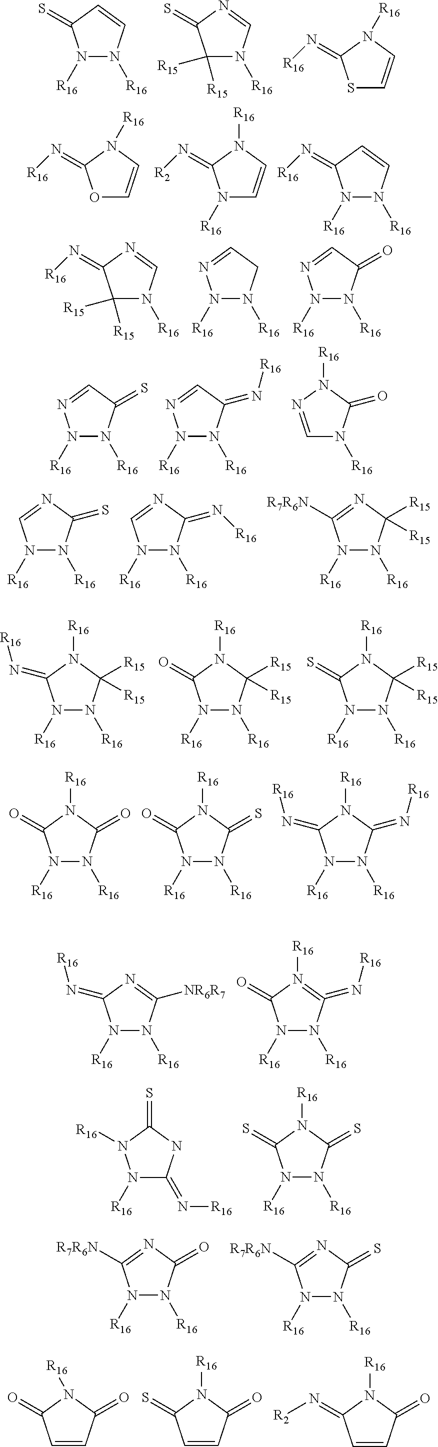 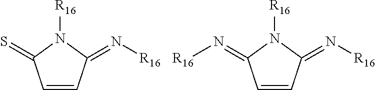 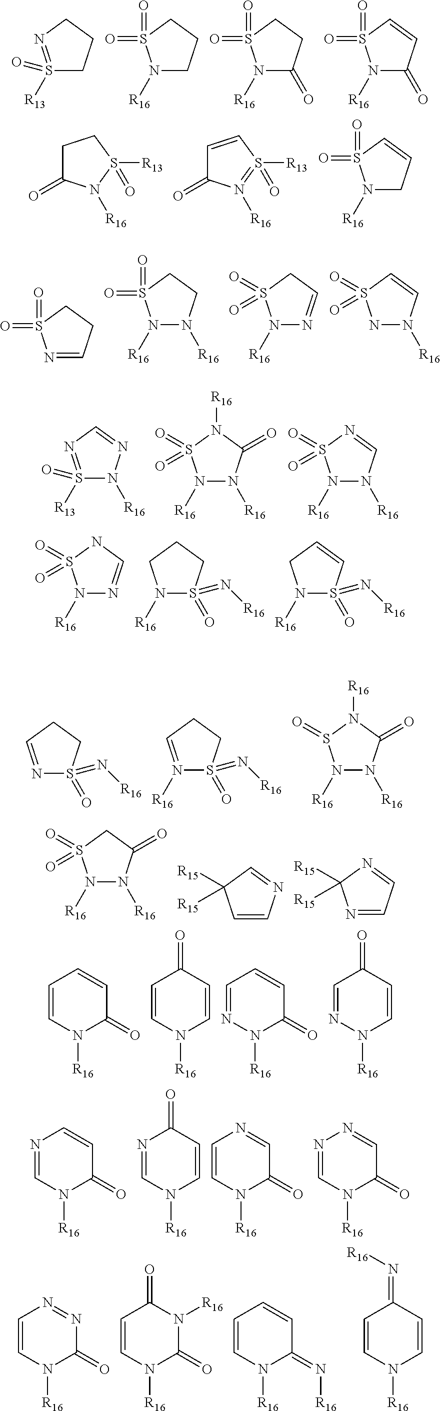 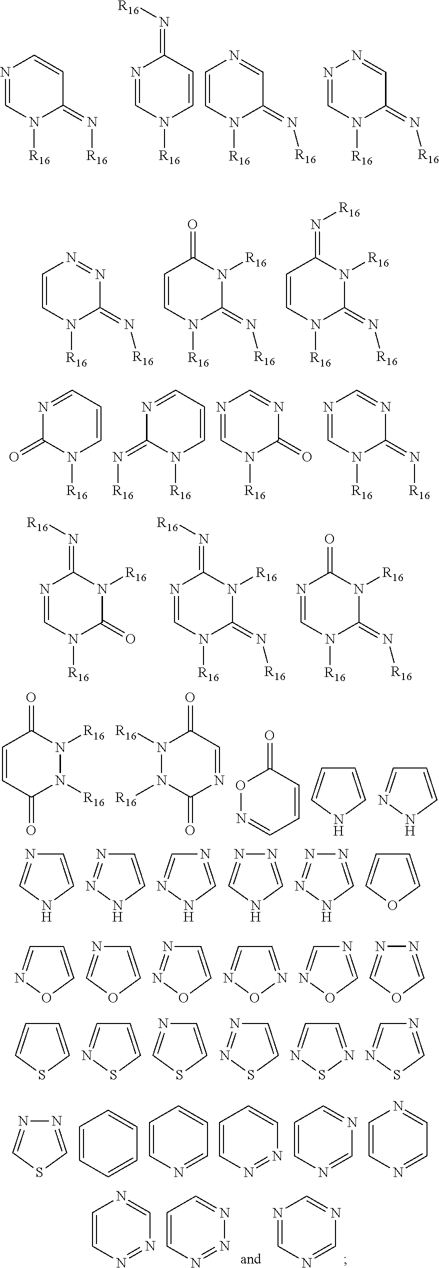
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that, when A1 is A1a, then neither X nor Y is a bond.

3. A compound of the formula (I) 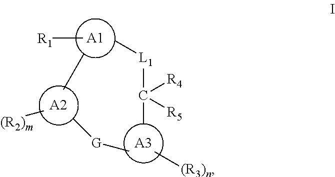
or a pharmaceutically acceptable salt thereof, wherein:
A1 is 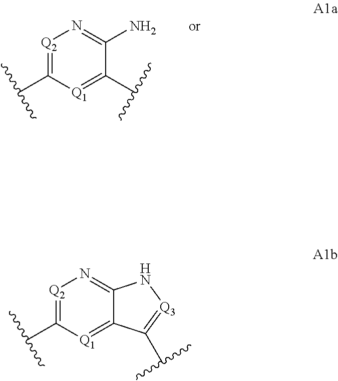
Q₁ is N or CH;
Q₂ is N or CH;
Q₃ is N or C-J-R₁;
A2 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 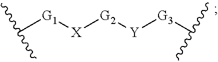
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, or —S(O)ₓ(CR₉R₁₀)ₒ—, or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 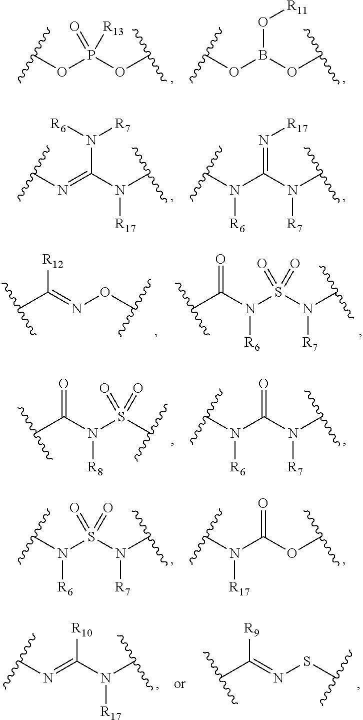
or a divalent ring system selected from the group consisting of: 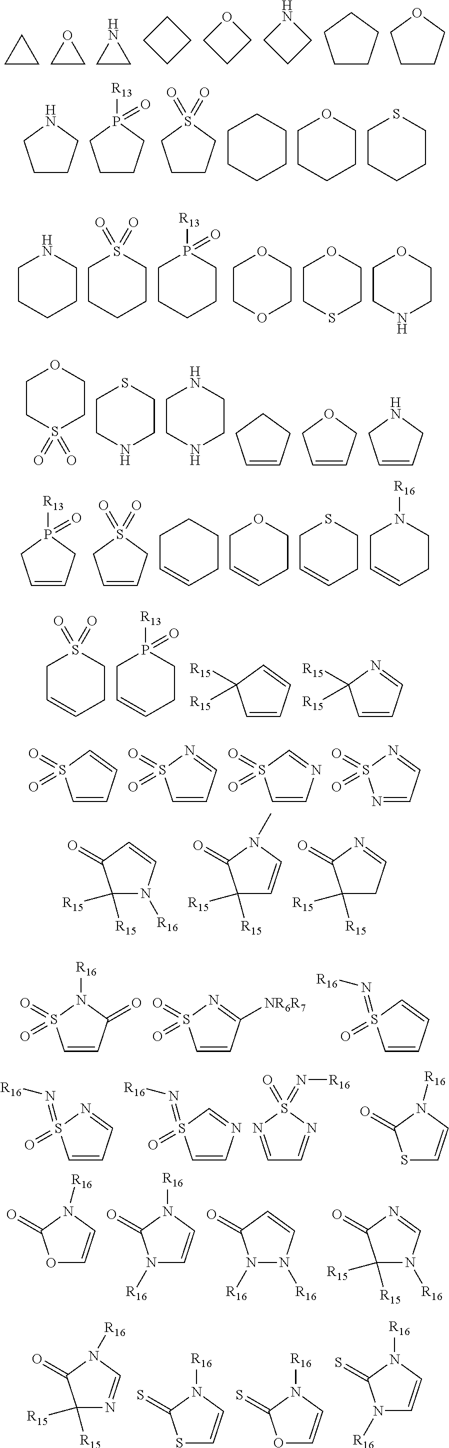 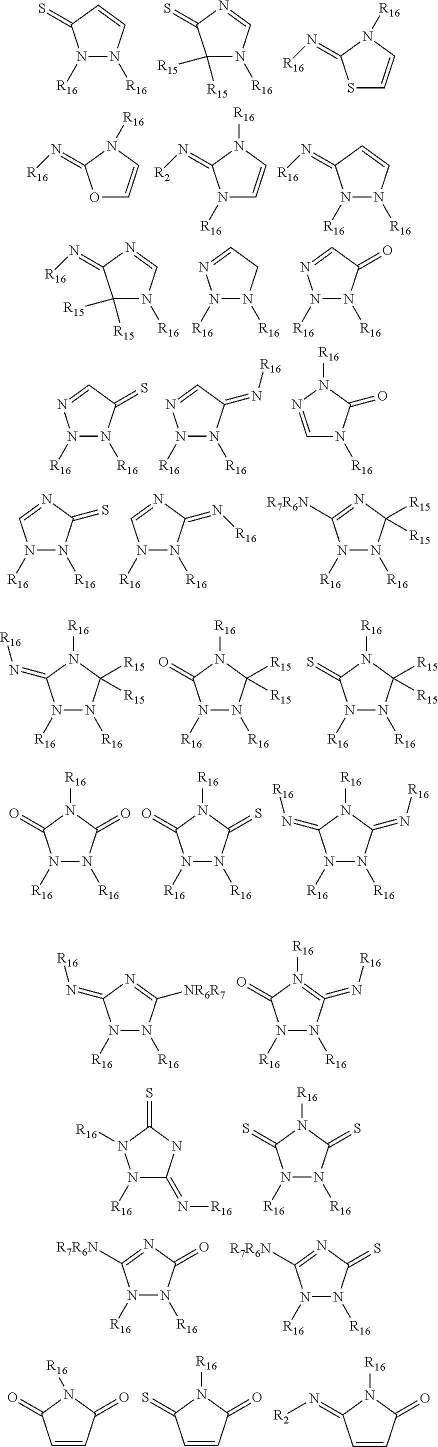 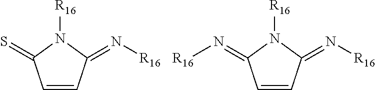 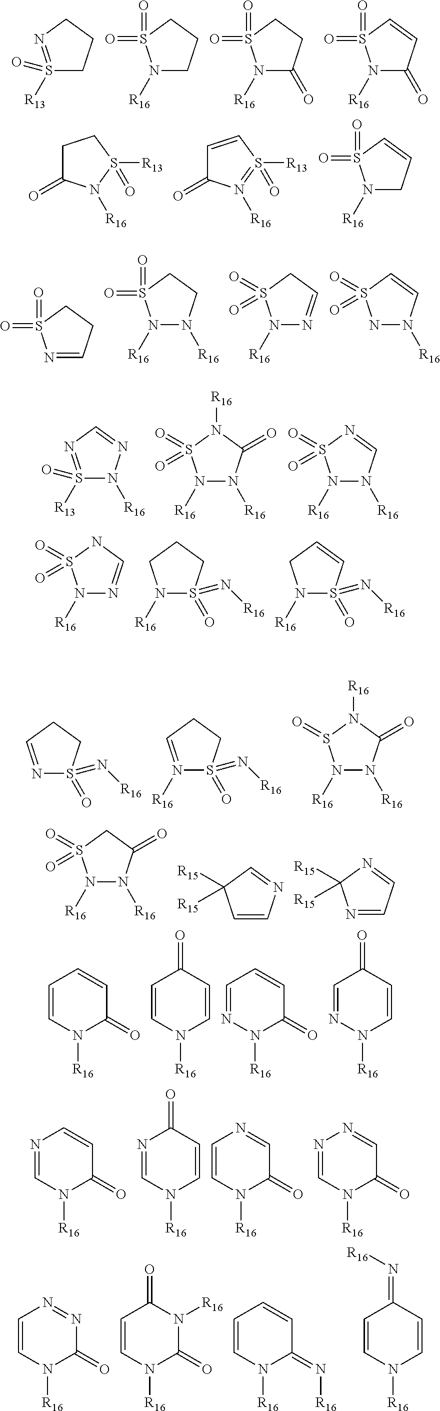 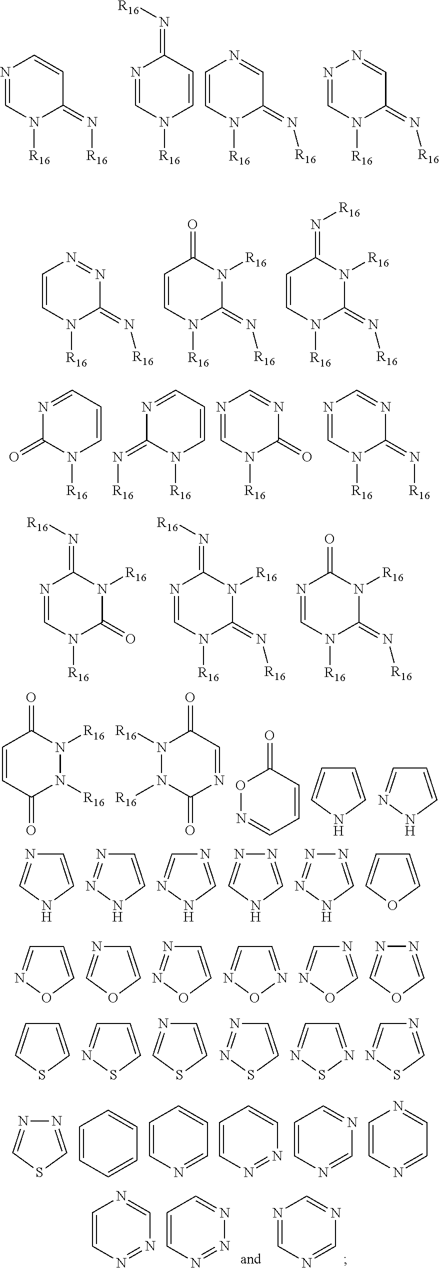
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that, when A1 is A1a and at least one of X and Y is a bond, then neither X nor Y is selected from the group consisting of: —O—, —NR₈—, —C(O)NR₈—, —NR₈C(O)—, arylene, and heteroarylene.

4. A compound of the formula (I) 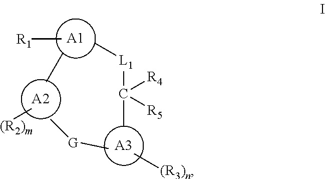
or a pharmaceutically acceptable salt thereof, wherein:
A1 is 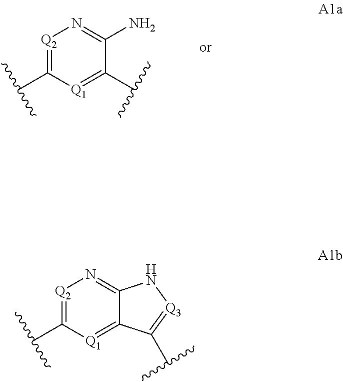
Q₁ is N or CH;
Q₂ is N or CH;
Q₃ is N or C-J-R₁;
A2 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 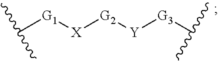
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, or —S(O)ₓ(CR₉R₁₀)ₒ—, or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 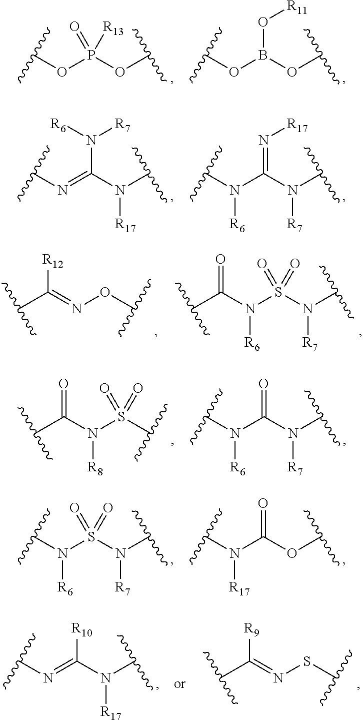
or a divalent ring system selected from the group consisting of: 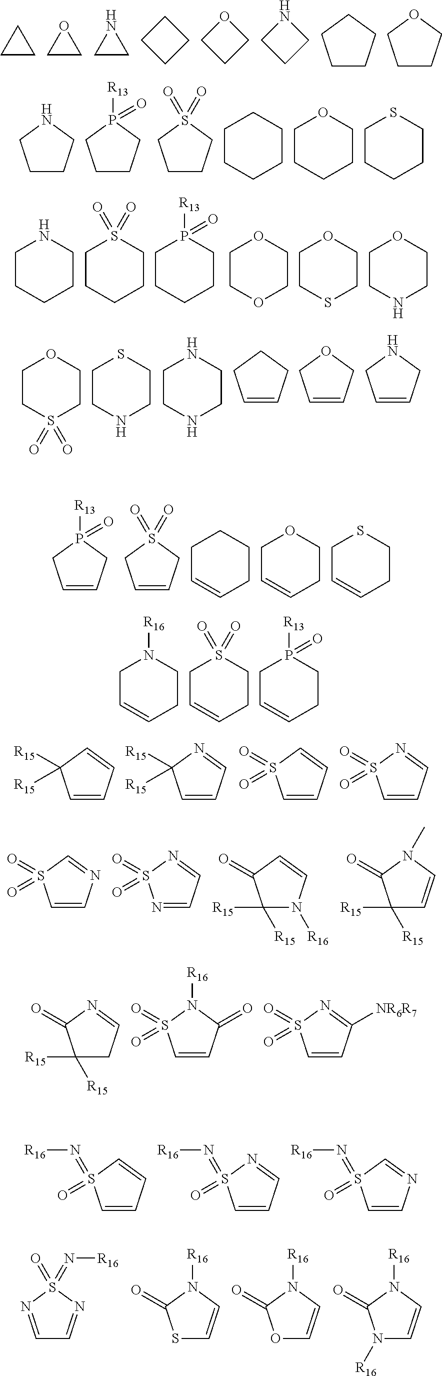 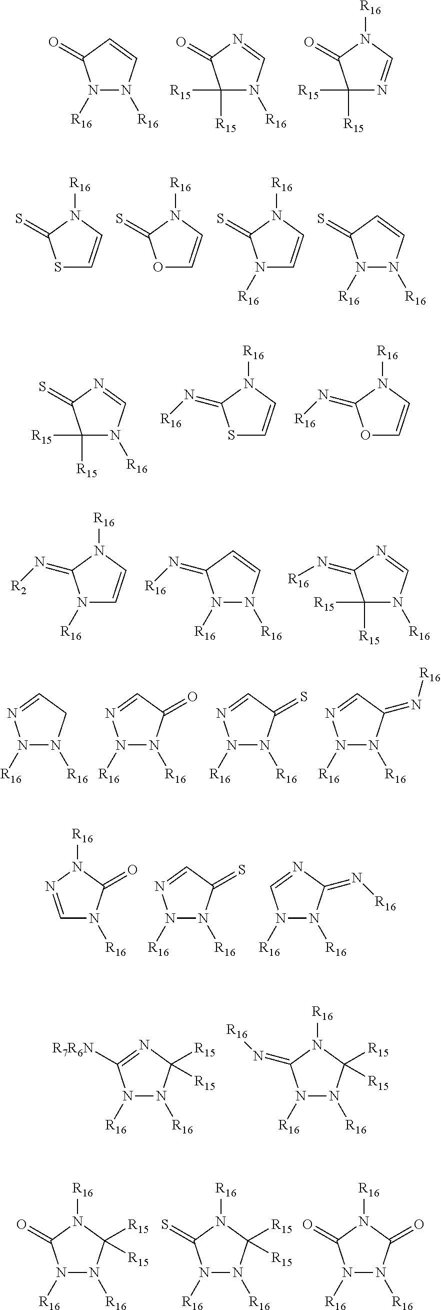 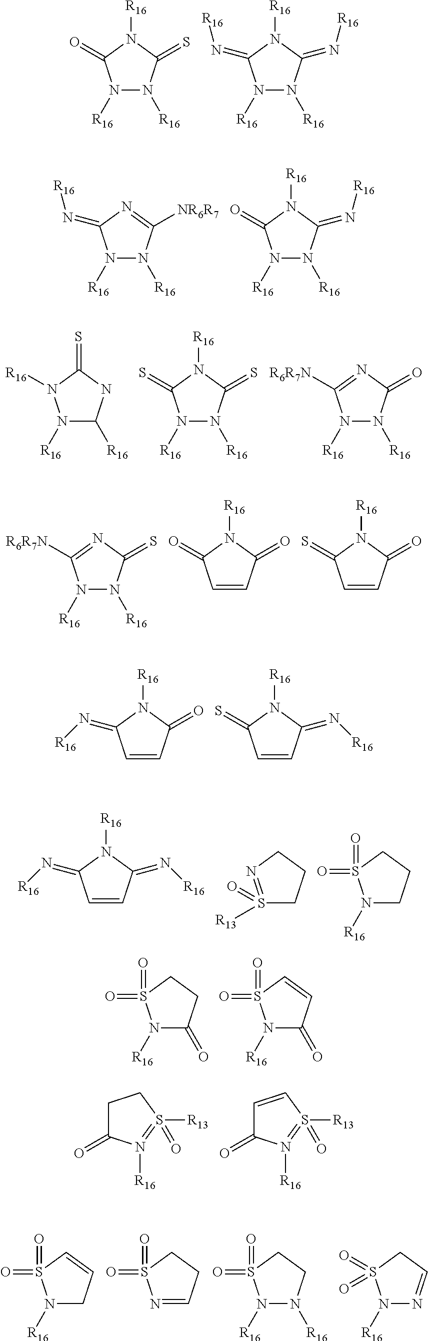 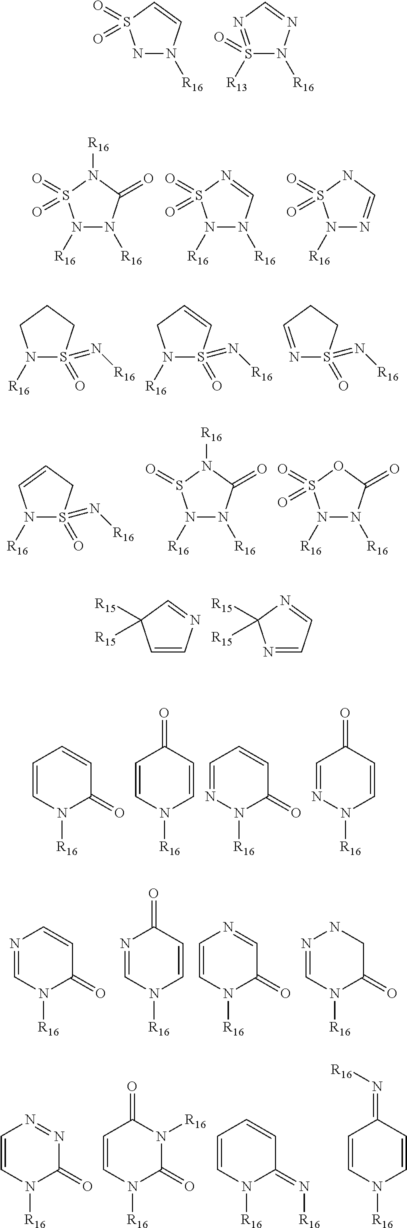 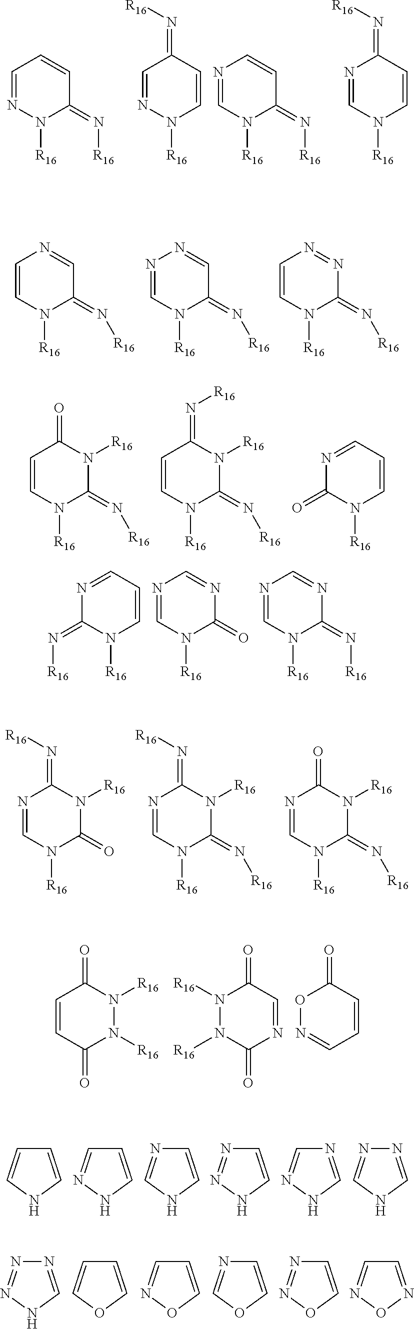 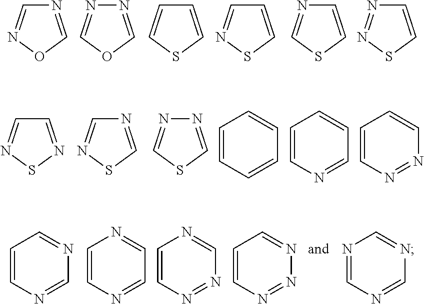
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that, when A1 is A1a, then X and Y are each independently selected from the group consisting of —O—, —NR₈—, —C(O)NR₈—, and —NR₈C(O)—.

5. A compound of the formula (I) 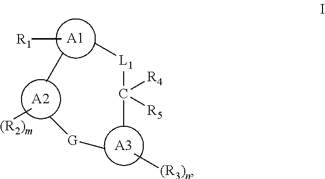
or a pharmaceutically acceptable salt thereof, wherein:
A1 is 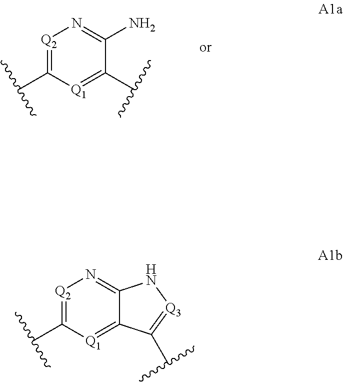
Q₁ is N or CH;
Q₂ is N or CH;
Q₃ is N or C-J-R₁;
A2 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 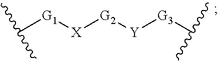
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, or —S(O)ₓ(CR₉R₁₀)ₒ—, or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 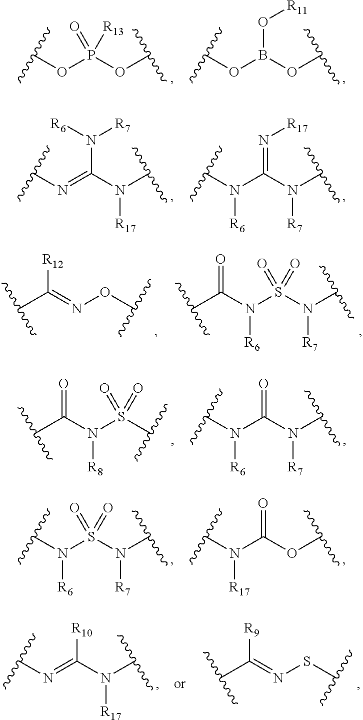
or a divalent ring system selected from the group consisting of: 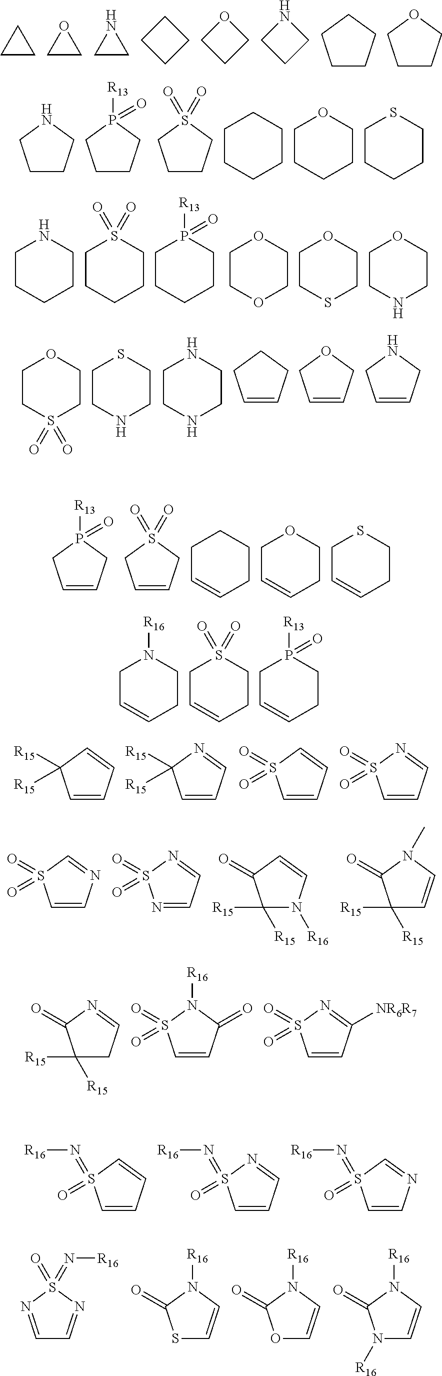 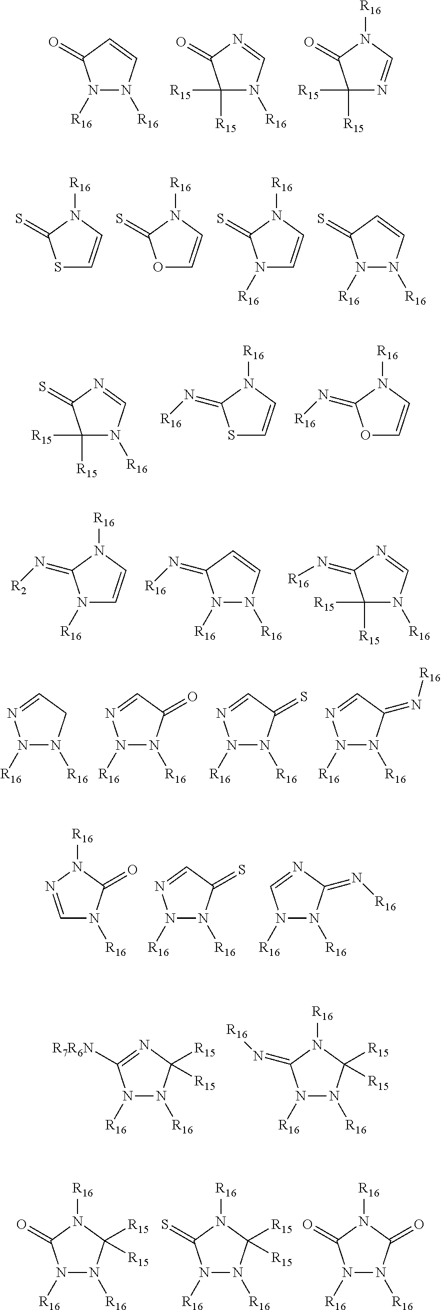 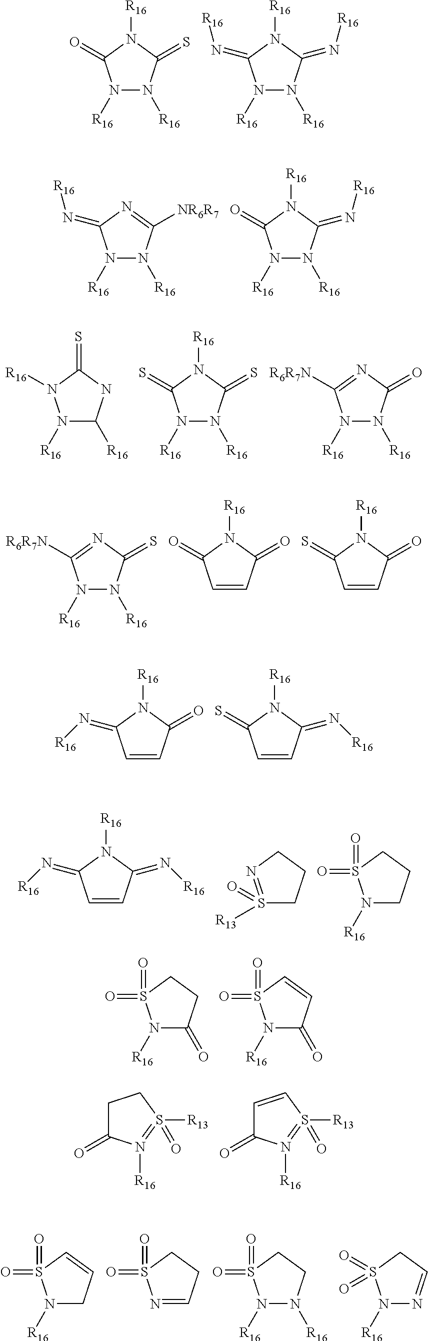 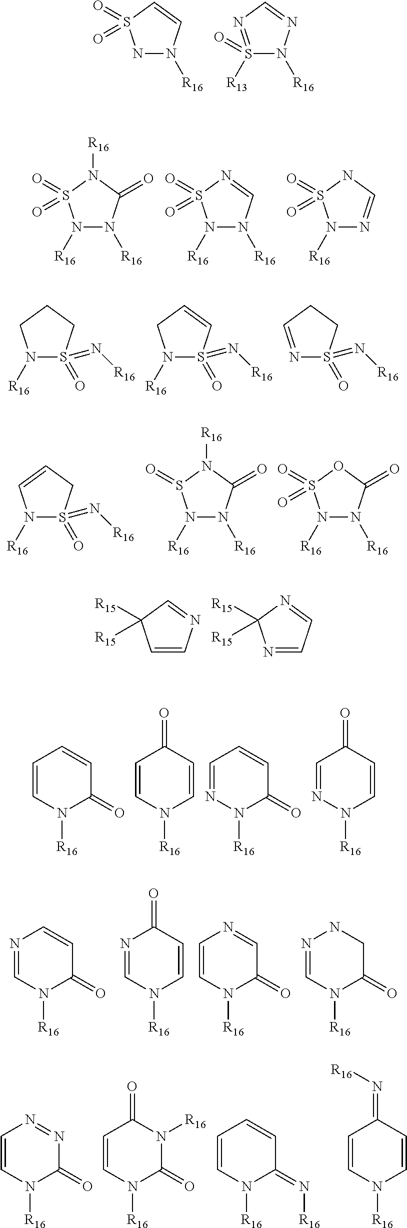 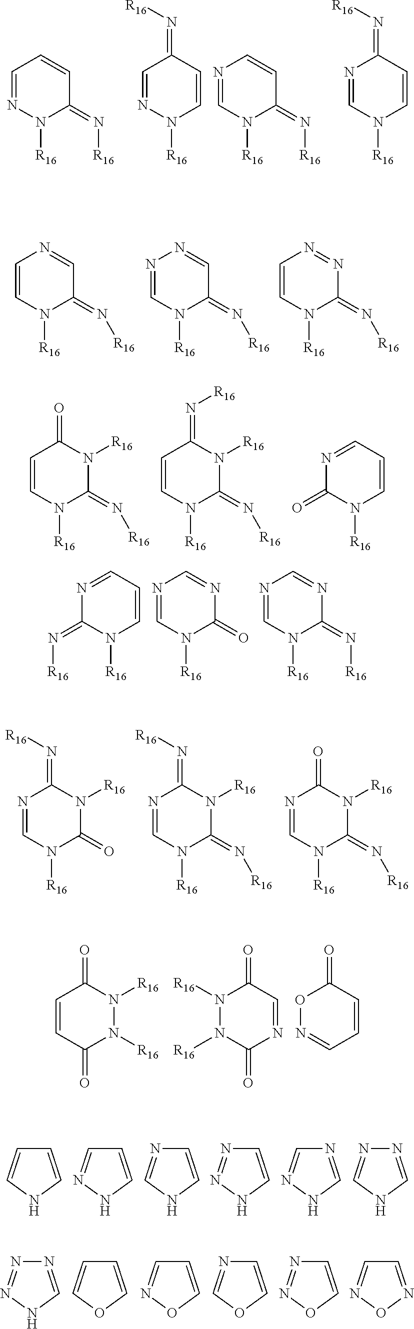 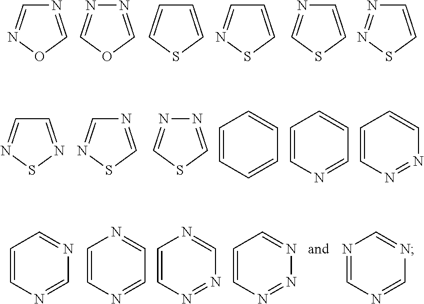
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that, when A1 is A1a, then Y is a cycloalkylene.

6. A compound of the formula (I) 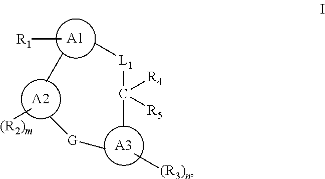
or a pharmaceutically acceptable salt thereof, wherein:
A1 is 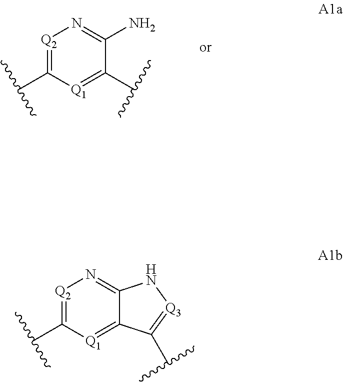
Q₁ is N or CH;
Q₂ is N or CH;
Q₃ is N or C-J-R₁;
A2 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 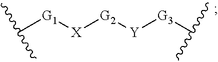
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, or —S(O)ₓ(CR₉R₁₀)ₒ—, or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 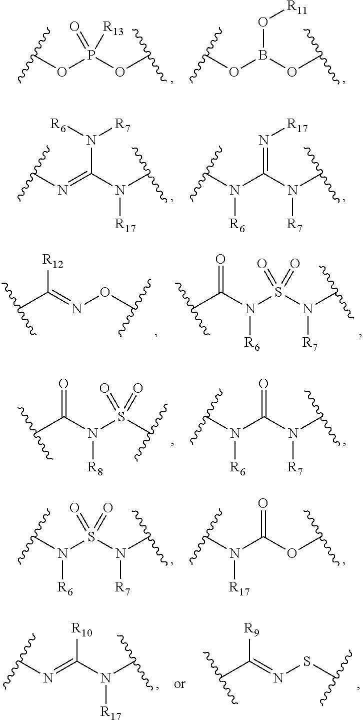
or a divalent ring system selected from the group consisting of: 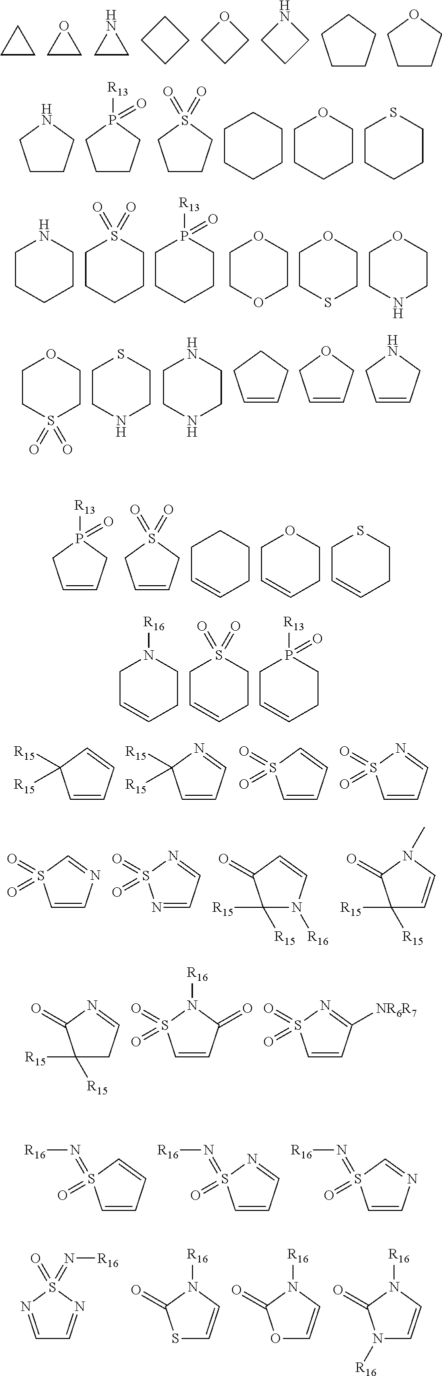 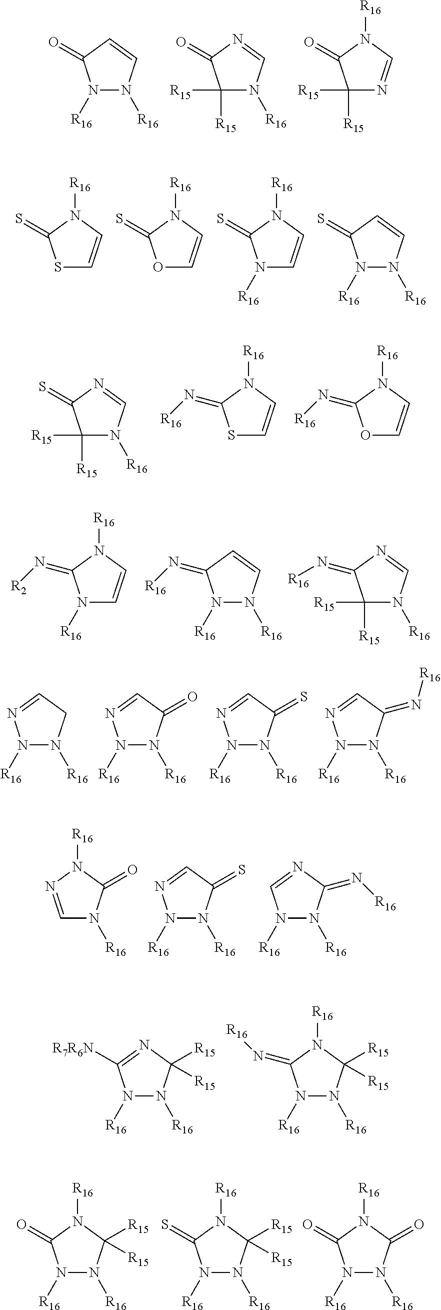 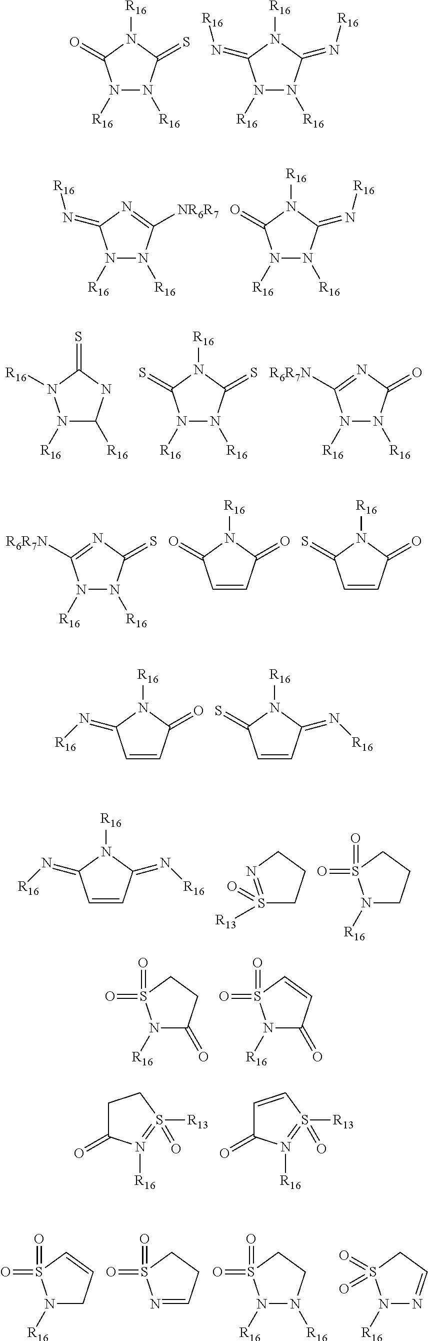 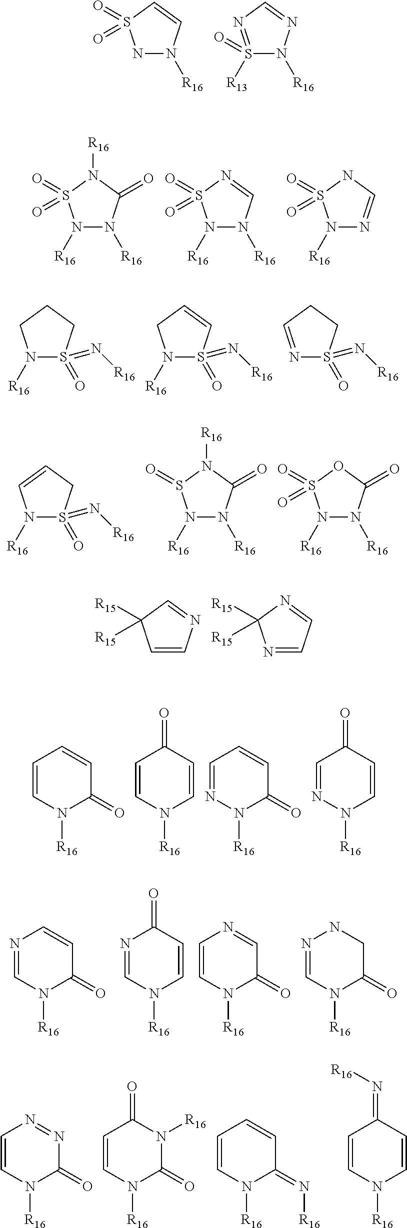 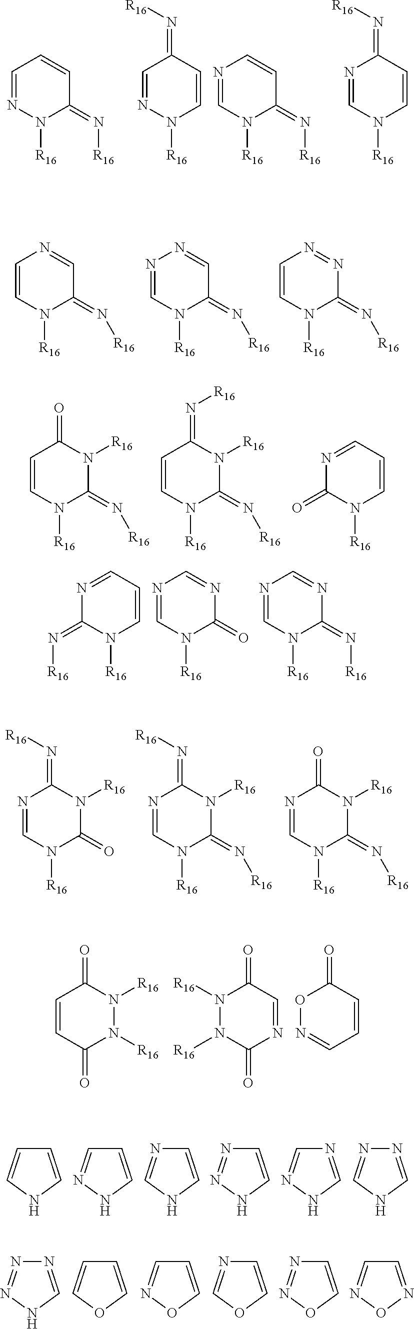 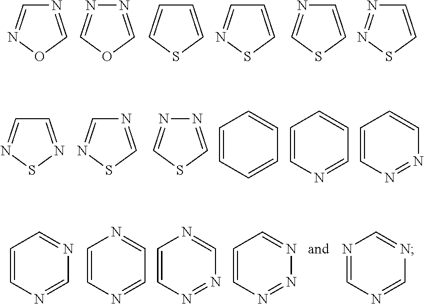
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that, when A1 is A1a, then Y is a heterocyclylene.

7. A compound of the formula (I) 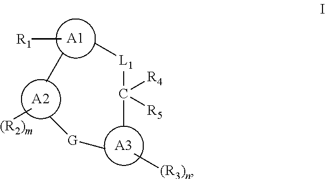
or a pharmaceutically acceptable salt thereof, wherein:
A1 is 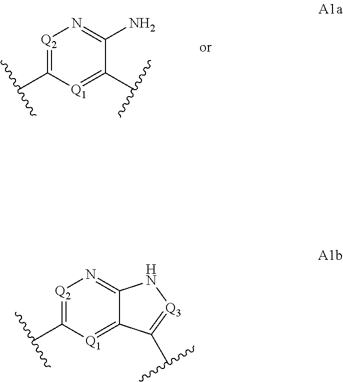
Q₁ is N or CH;
Q₂ is N or CH;
Q₃ is N or C-J-R₁;
A2 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 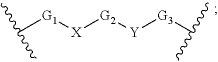
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, or —S(O)ₓ(CR₉R₁₀)ₒ—, or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 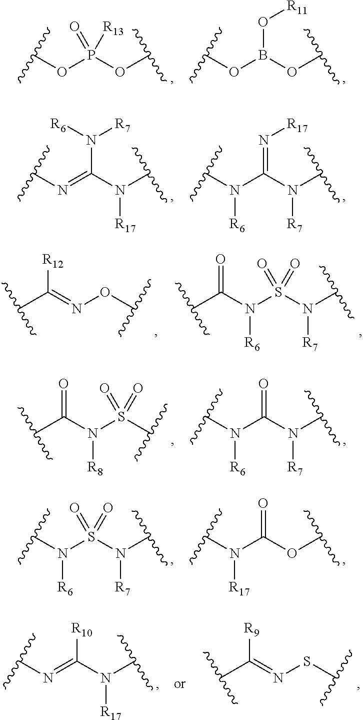
or a divalent ring system selected from the group consisting of: 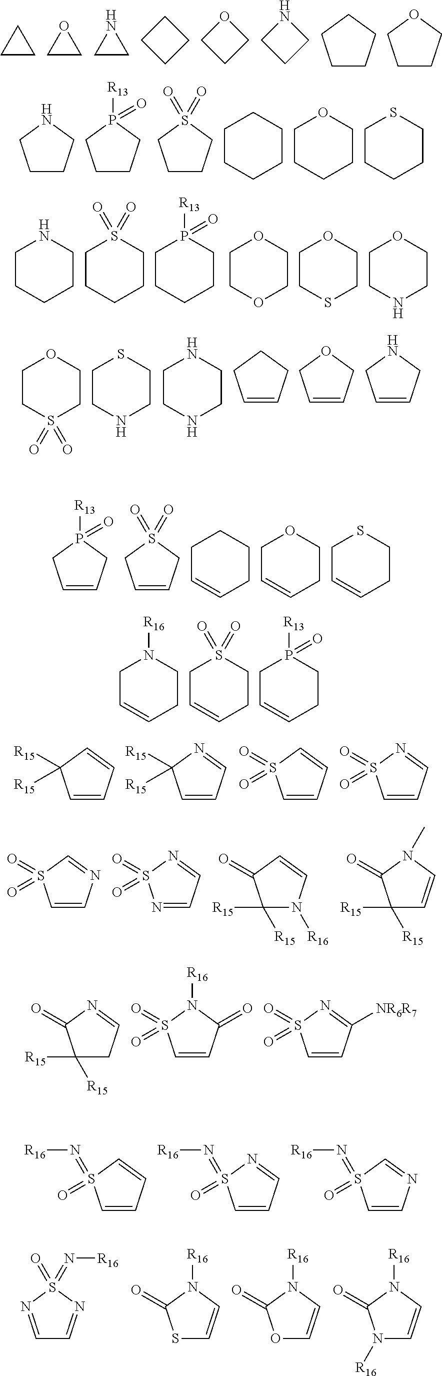 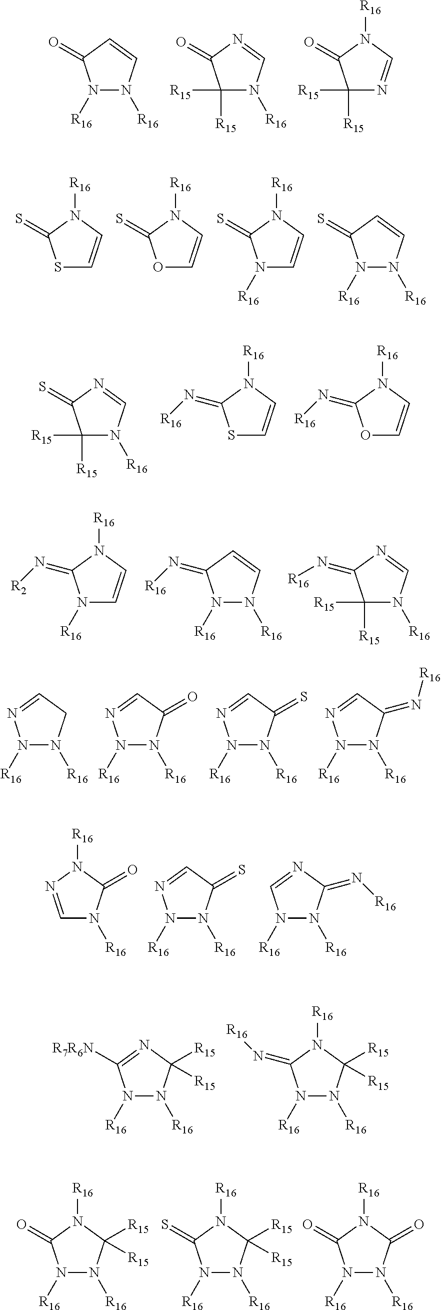 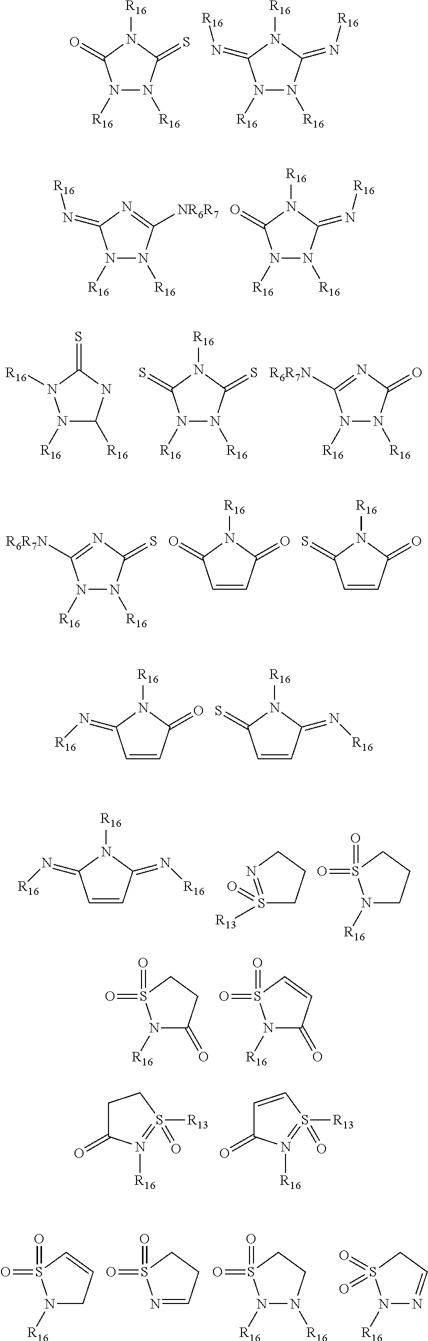 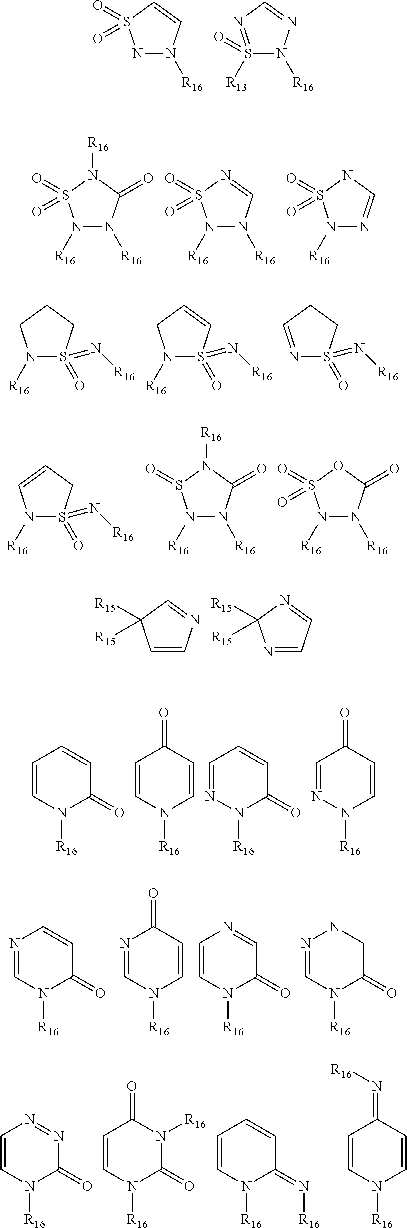 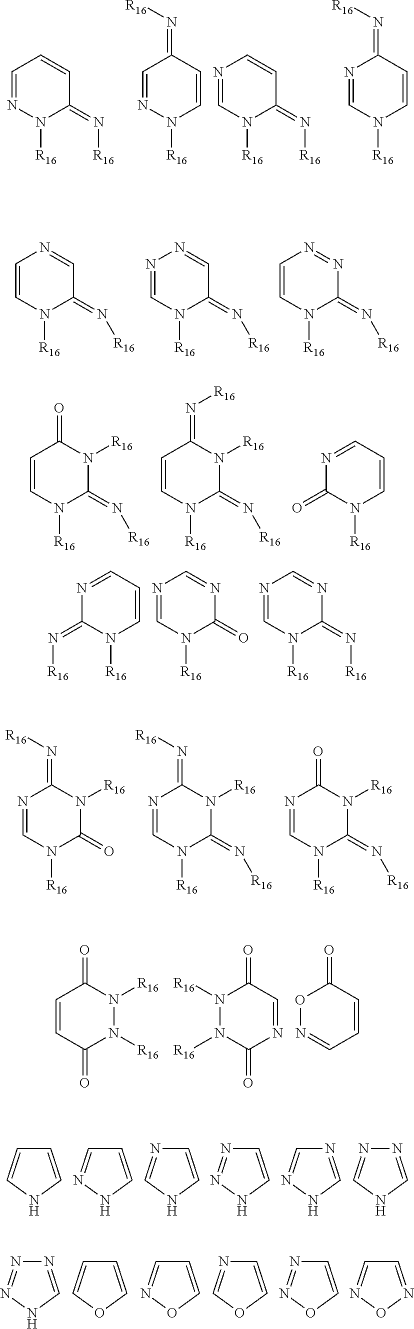 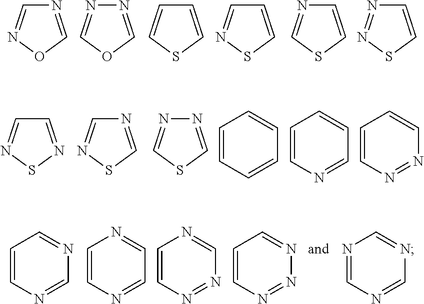
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that, when A1 is A1a, then G contain at least two heteroatoms selected from O, N, S, and P in the direct chain that links A2 and A3.

8. A compound of the formula (I) 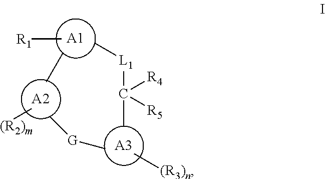
or a pharmaceutically acceptable salt thereof, wherein:
A1 is 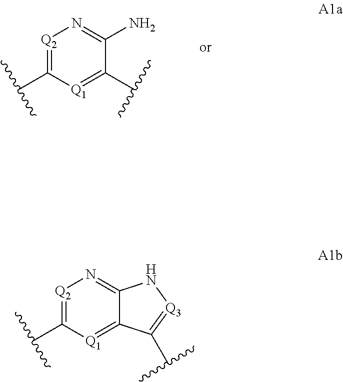
Q₁ is N or CH;
Q₂ is N or CH;
Q₃ is N or C-J-R₁;
A2 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₆-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 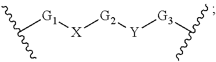
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, or —S(O)ₓ(CR₉R₁₀)ₒ—, or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 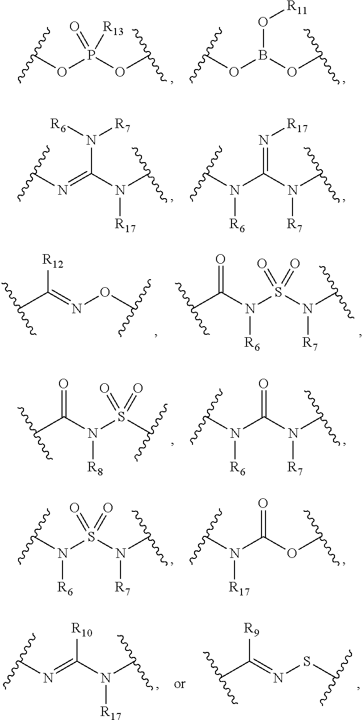
or a divalent ring system selected from the group consisting of: 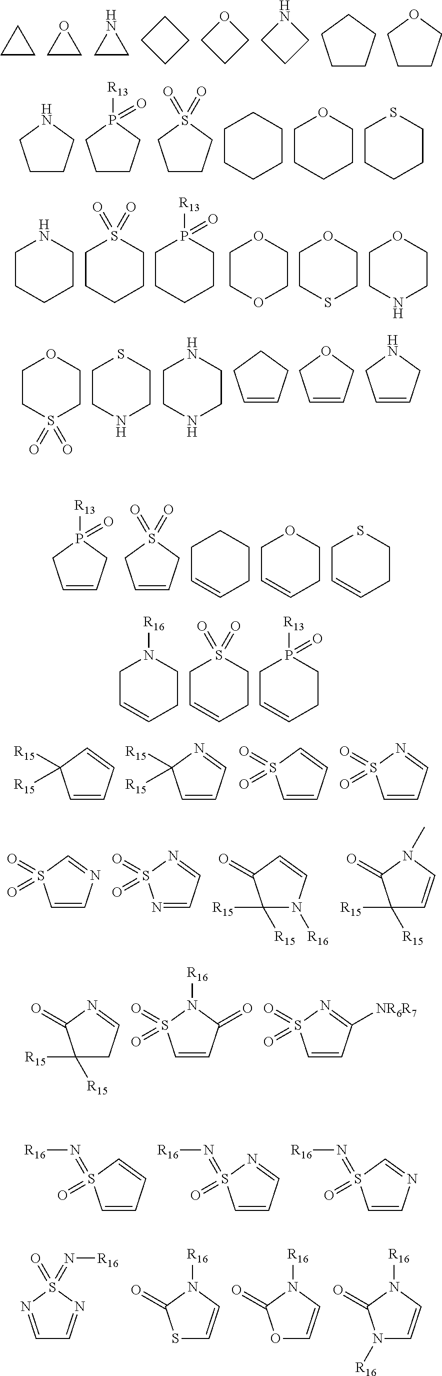 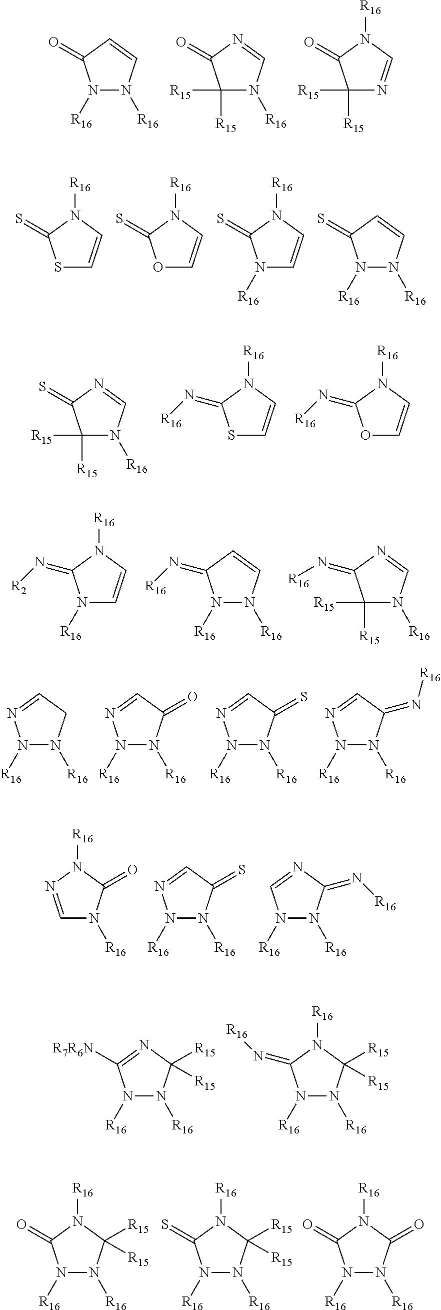 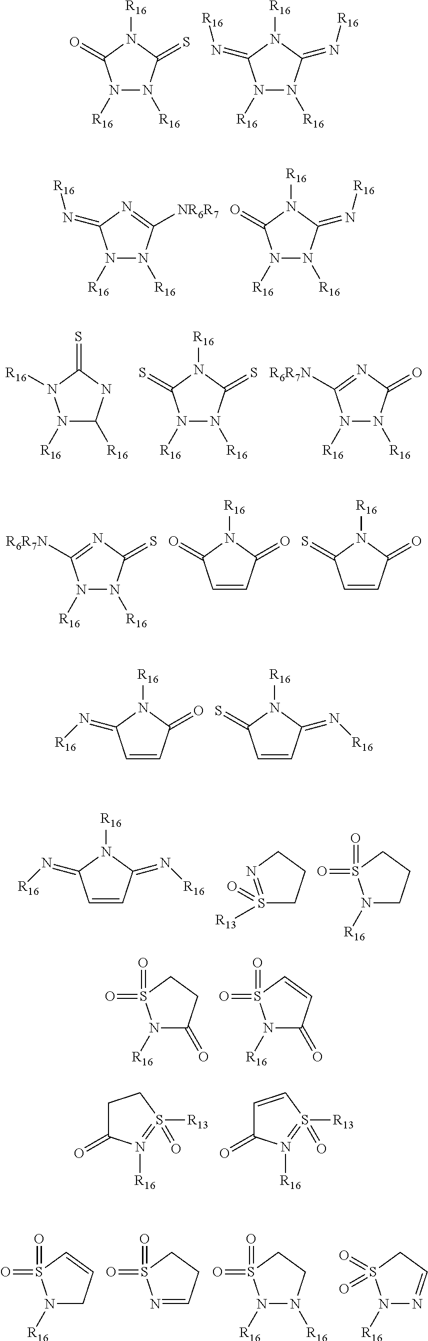 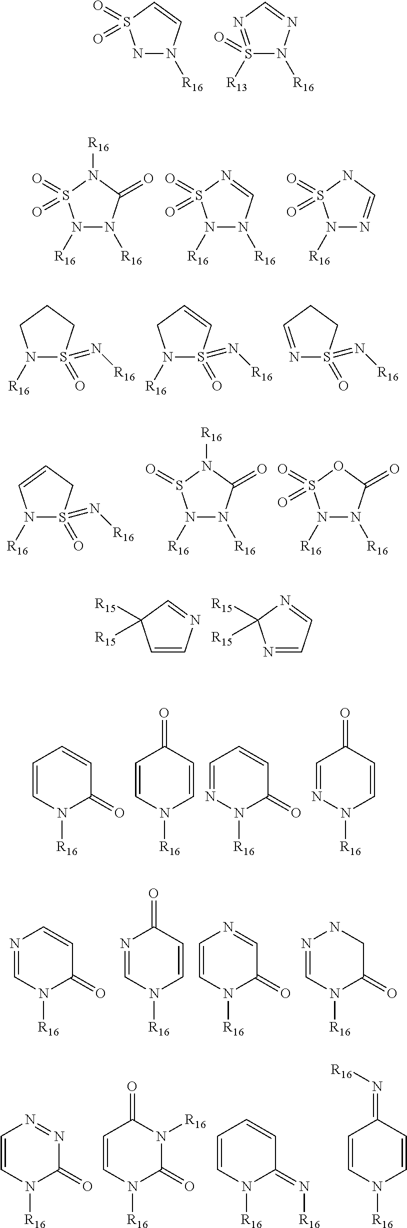 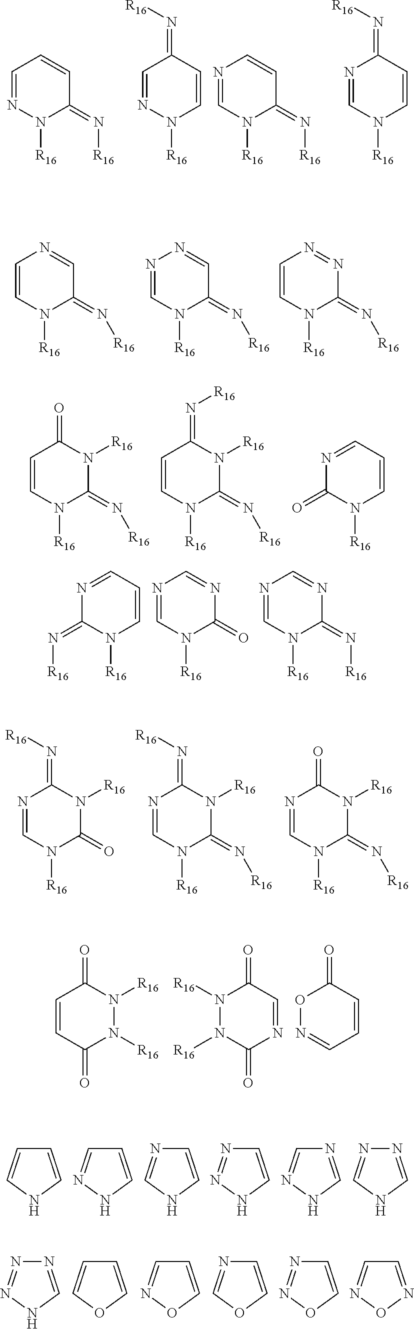 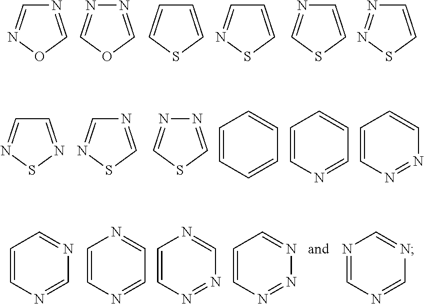
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that, when A1 is A1a, then Y is —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—,

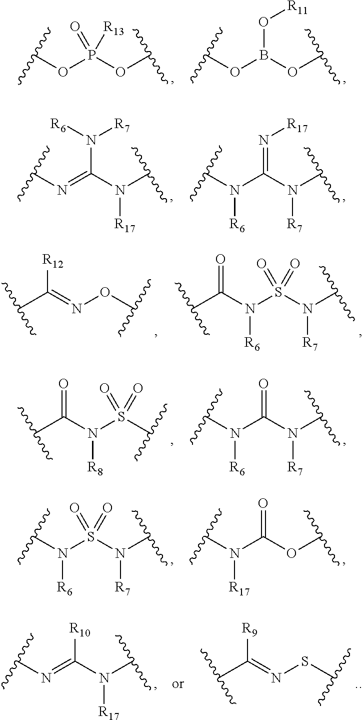

9. A compound of the formula (I) 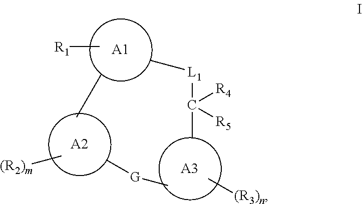
or a pharmaceutically acceptable salt thereof, wherein:
A1 is 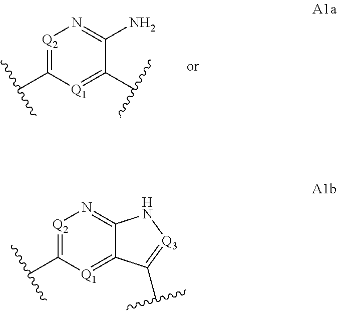
Q₁ is N or CH;
Q₂ is N or CH;
Q₃ is N or C-J-R₁;
A2 is a C₈-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₈-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 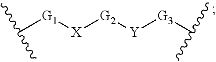
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, or —S(O)ₓ(CR₉R₁₀)ₒ—, or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 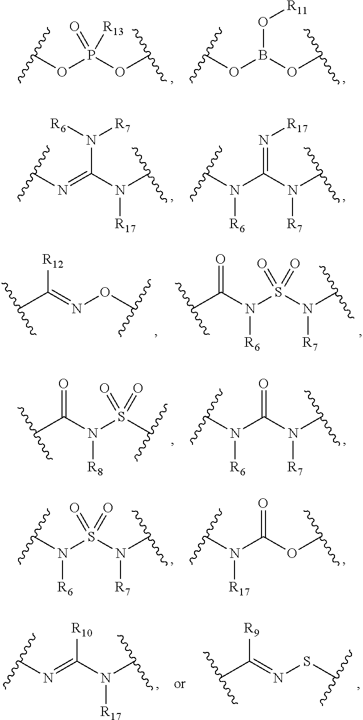
or a divalent ring system selected from the group consisting of: 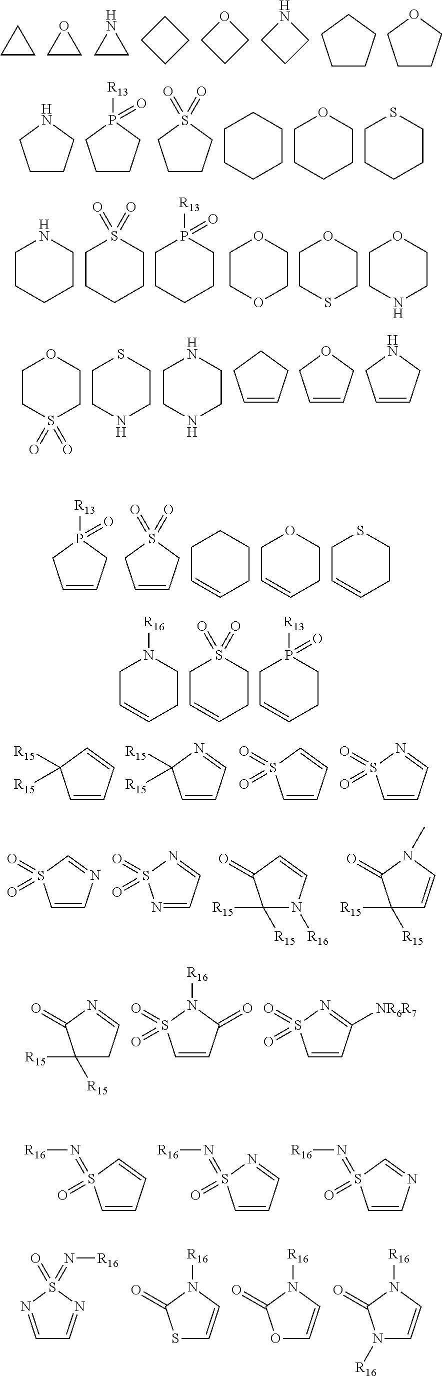 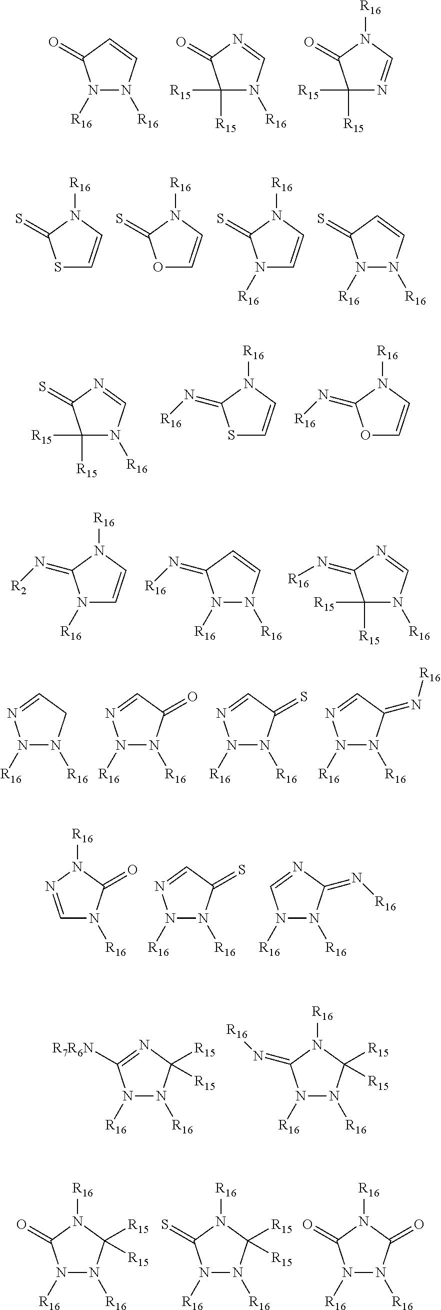 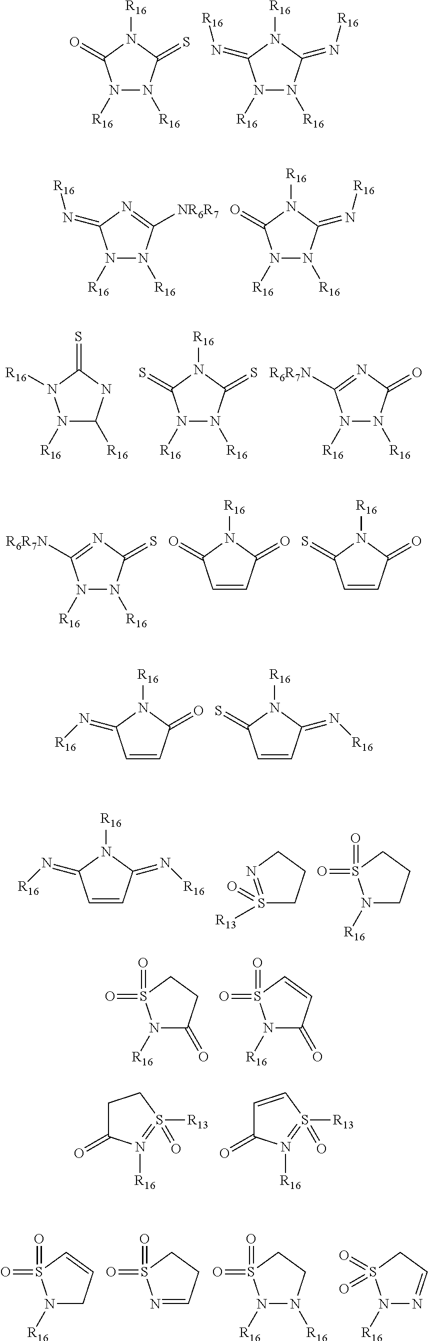 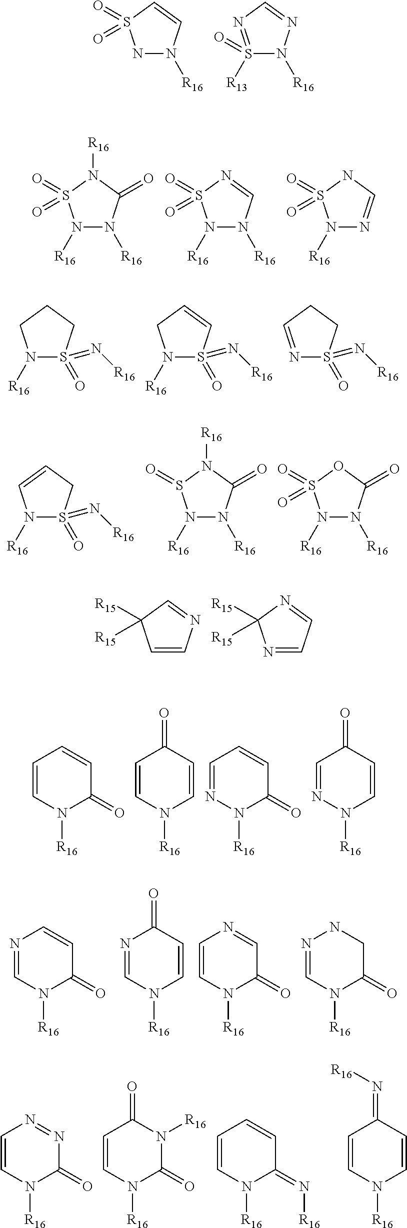 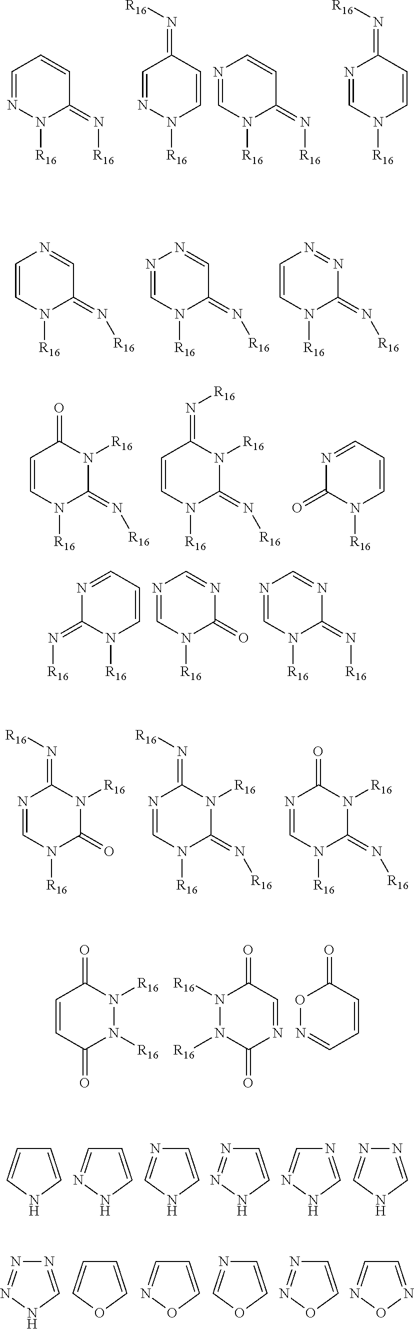 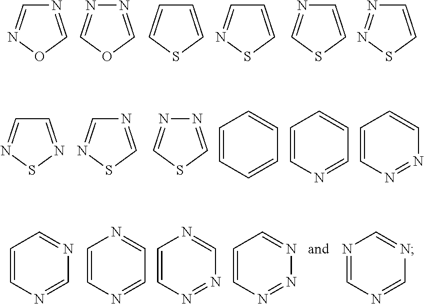
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that, when A1 is A1a, then Y is a divalent ring system selected from the group consisting of: 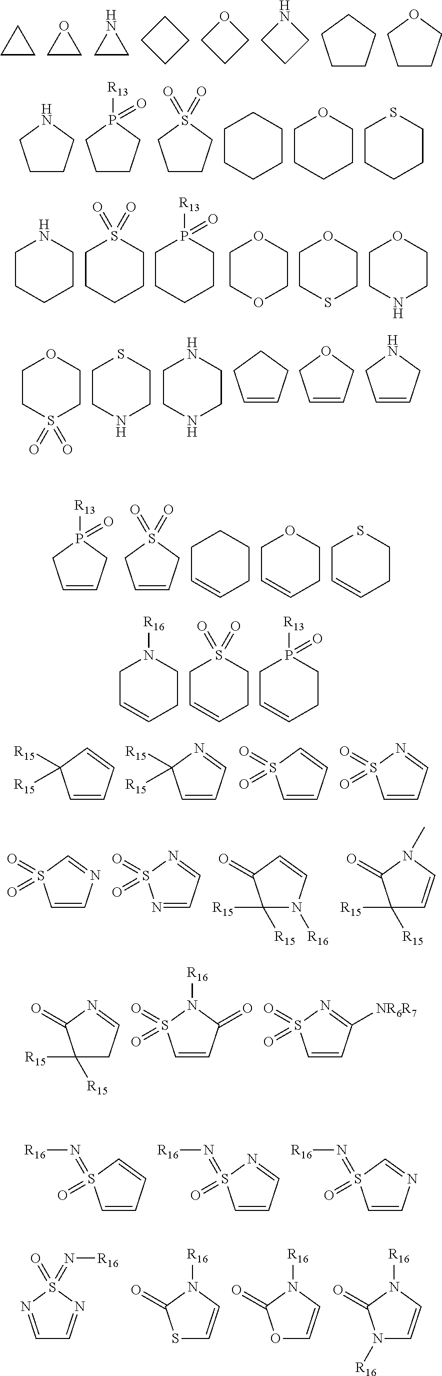 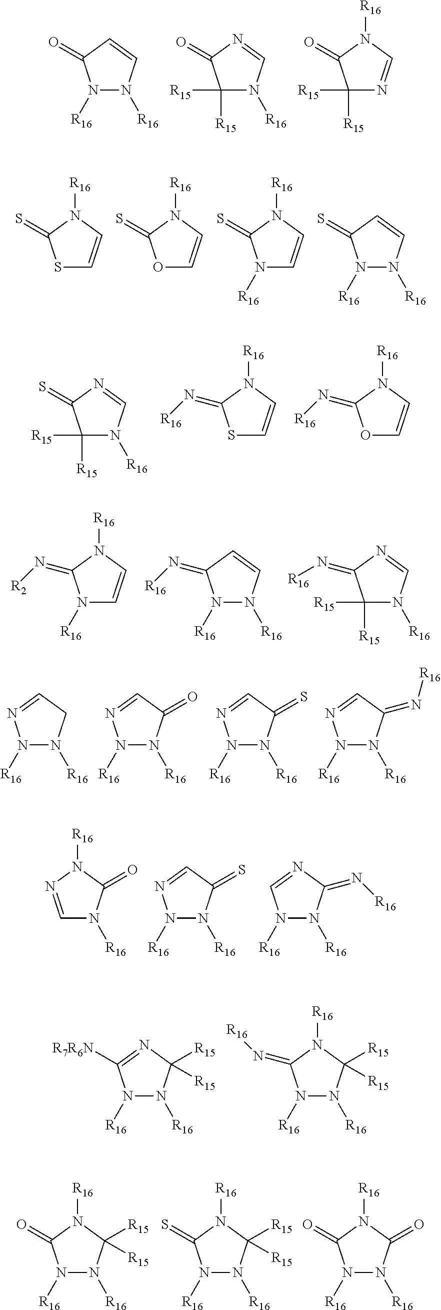 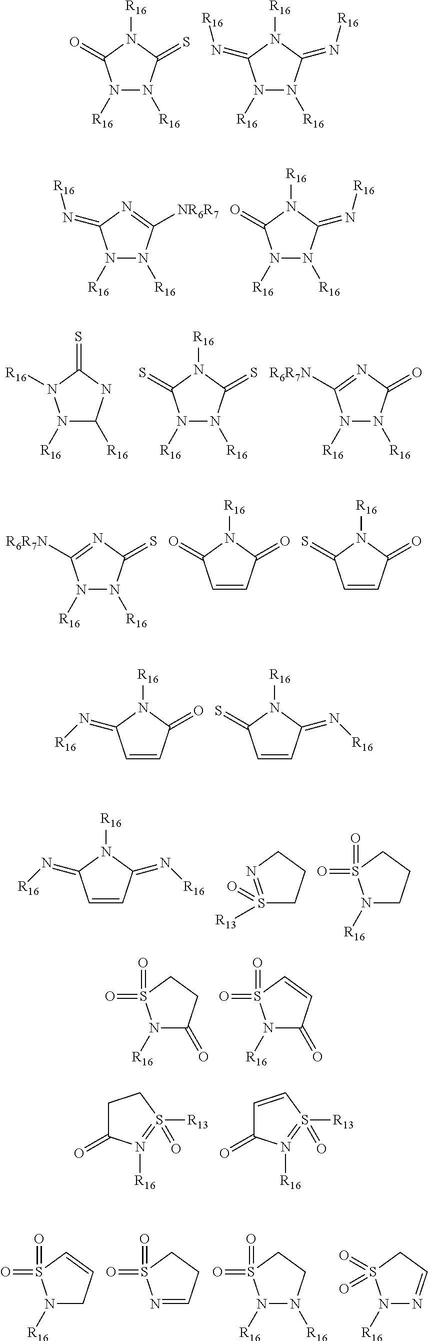 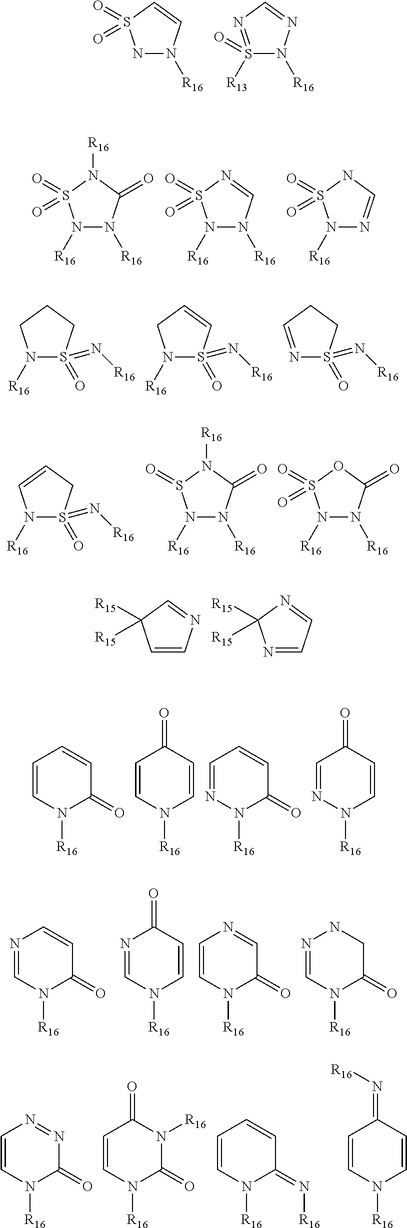 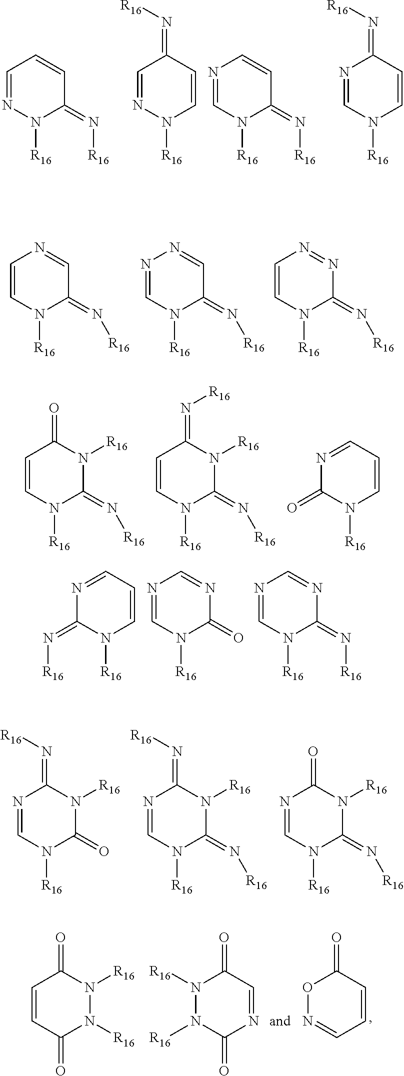
wherein R₁₅ and R₁₆ are not H.

10. A compound of the formula (I) 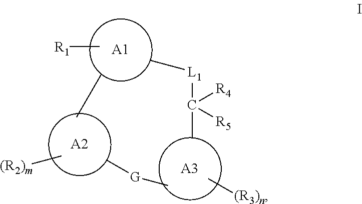
or a pharmaceutically acceptable salt thereof, wherein:
A1 is 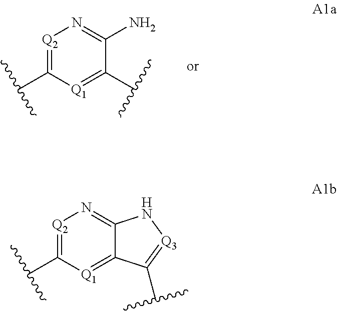
Q₁ is N or CH;
Q₂ is N or CH;
Q₃ is N or C-J-R₁;
A2 is a C₈-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₈-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 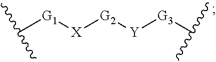
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, or —S(O)ₓ(CR₉R₁₀)ₒ—, or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 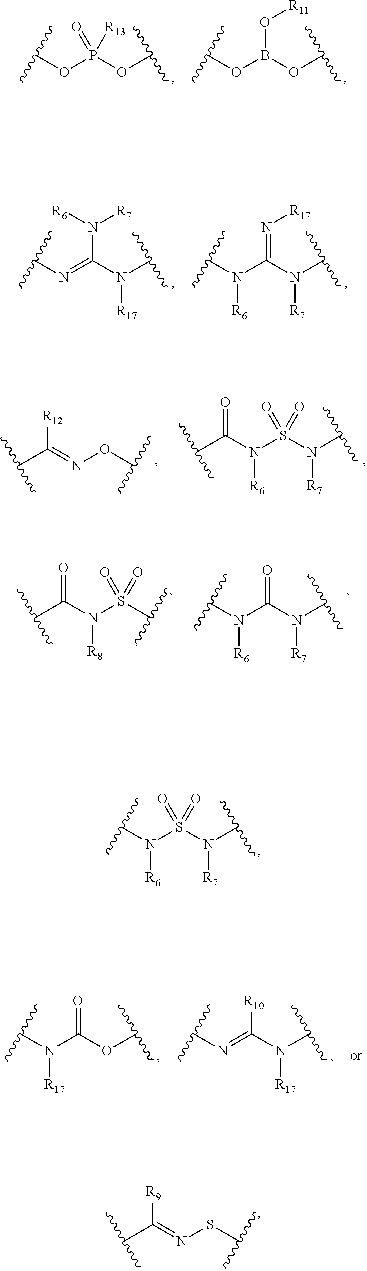
or a divalent ring system selected from the group consisting of: 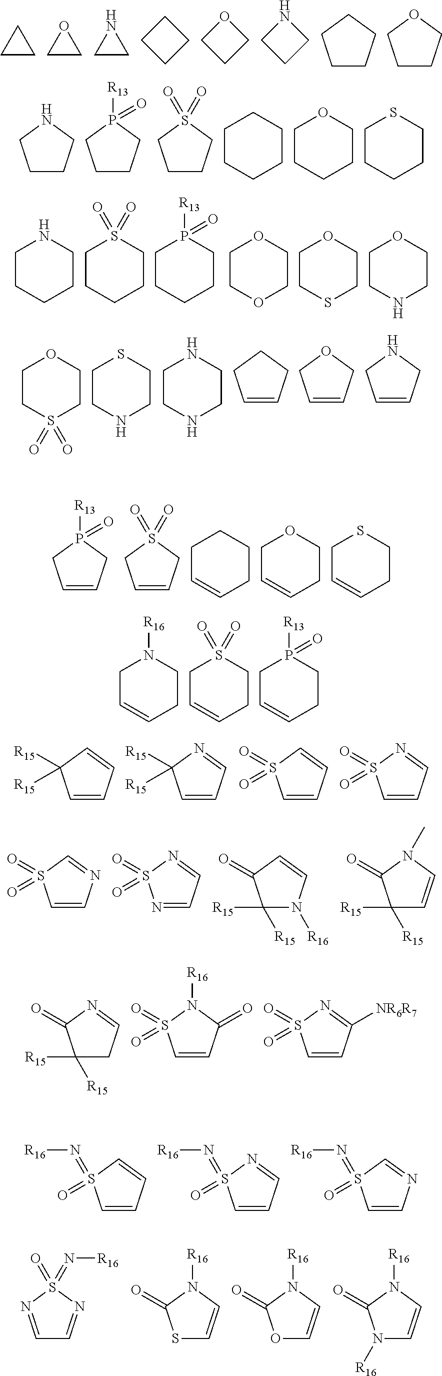 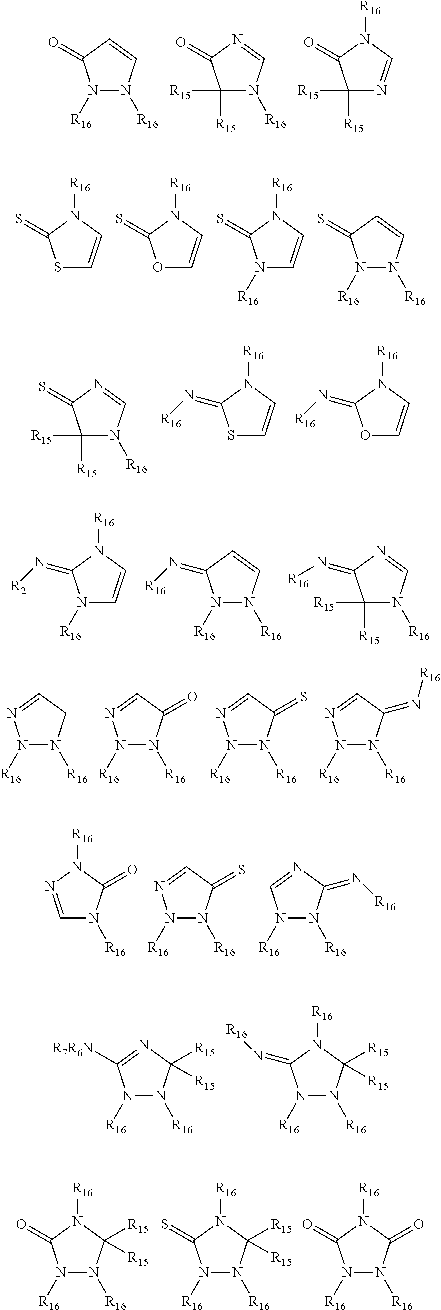 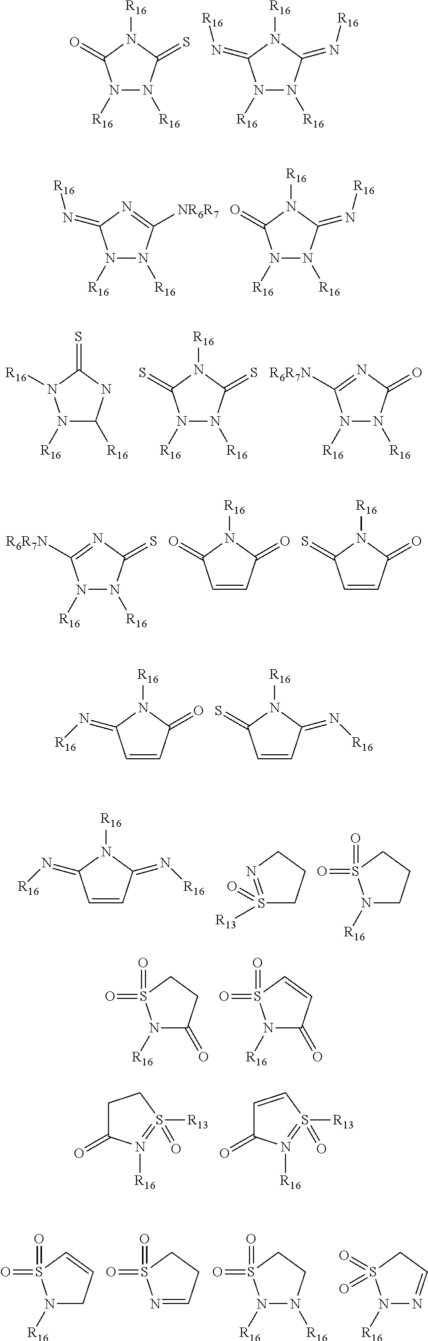 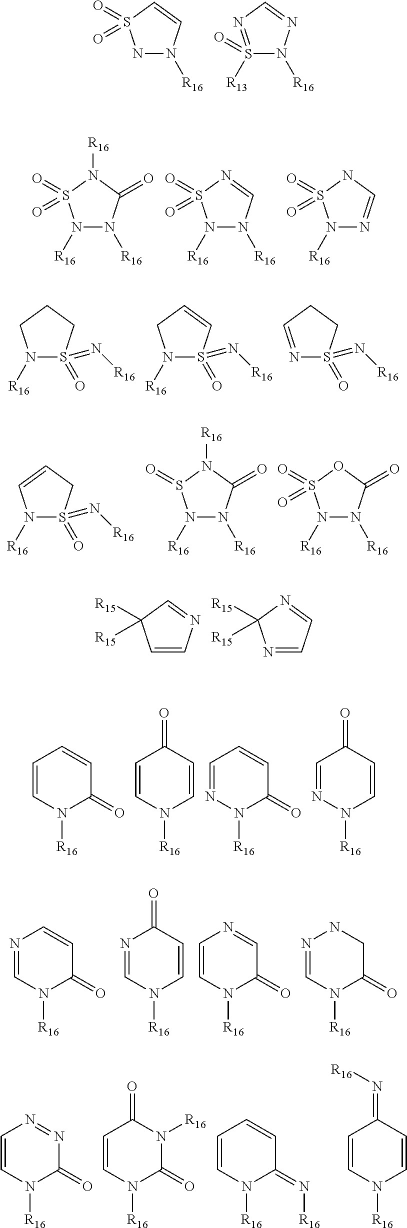 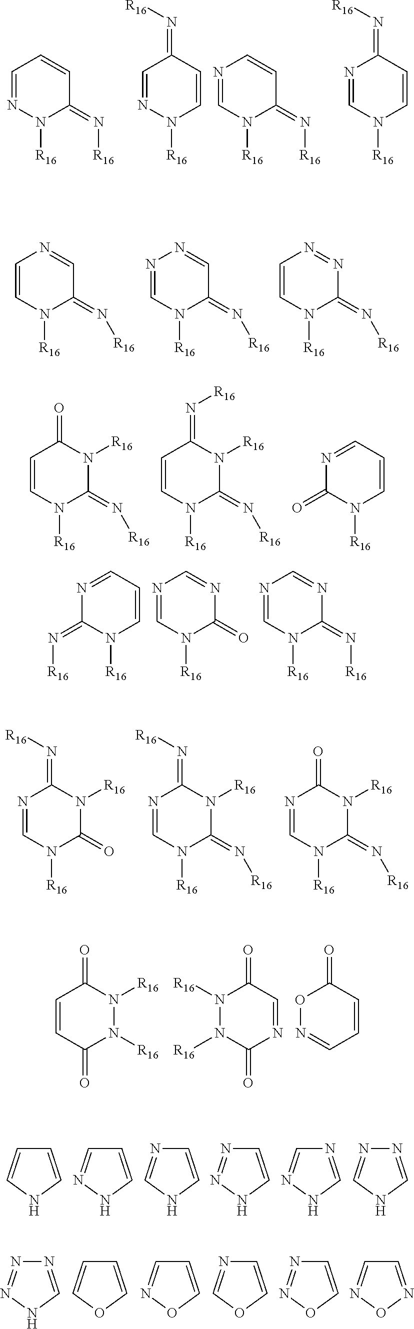 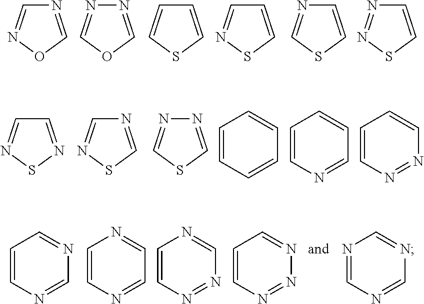
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁; P(O)(NR₆R₇)R₁₃; P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that, when A1 is A1a, then Y is a divalent ring system selected from the group consisting of: 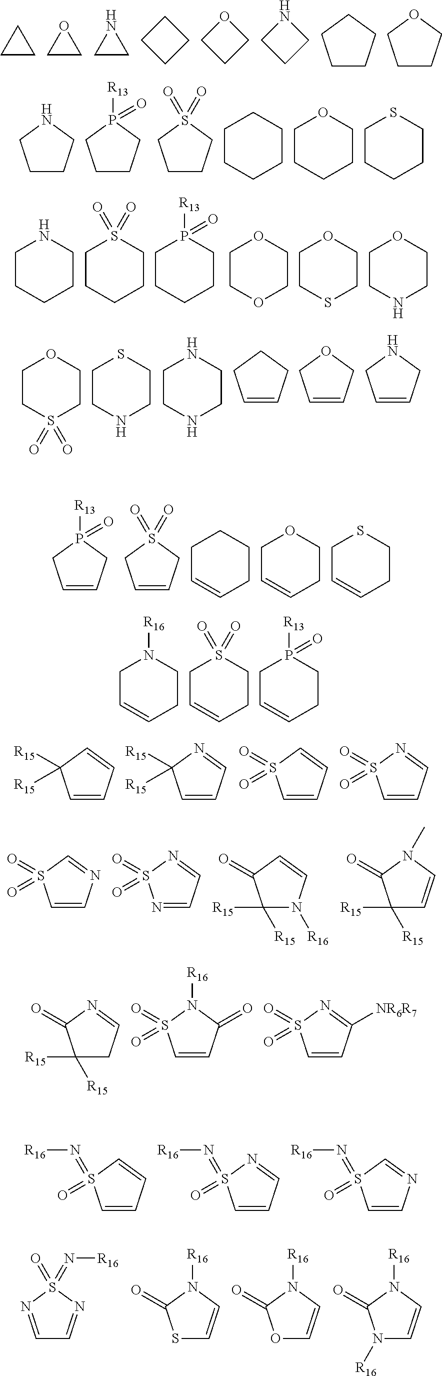 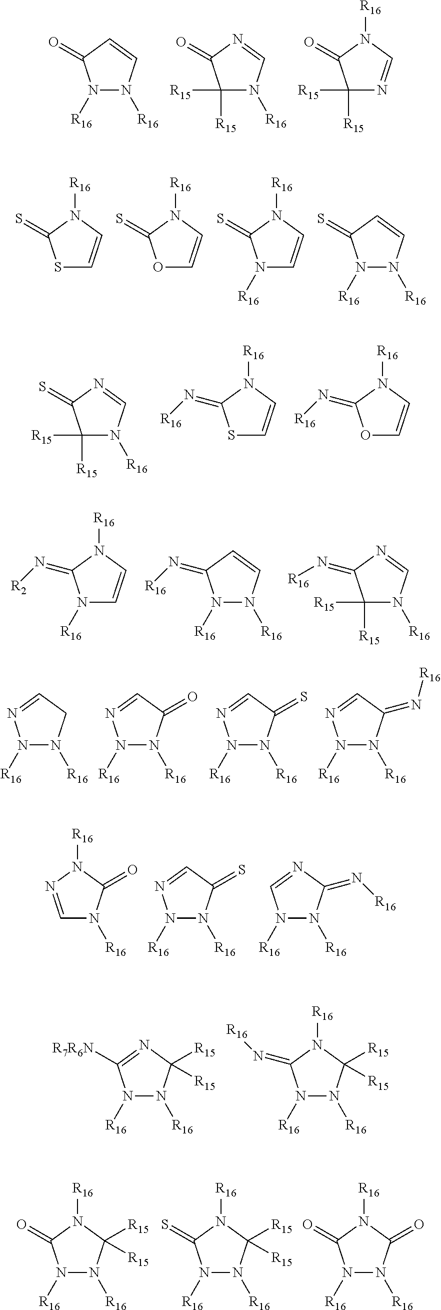 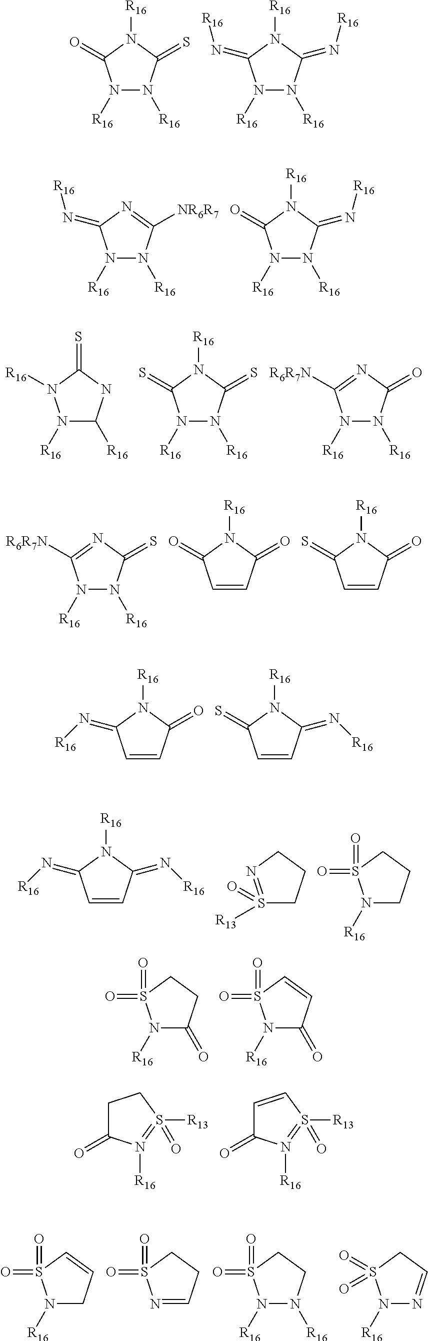 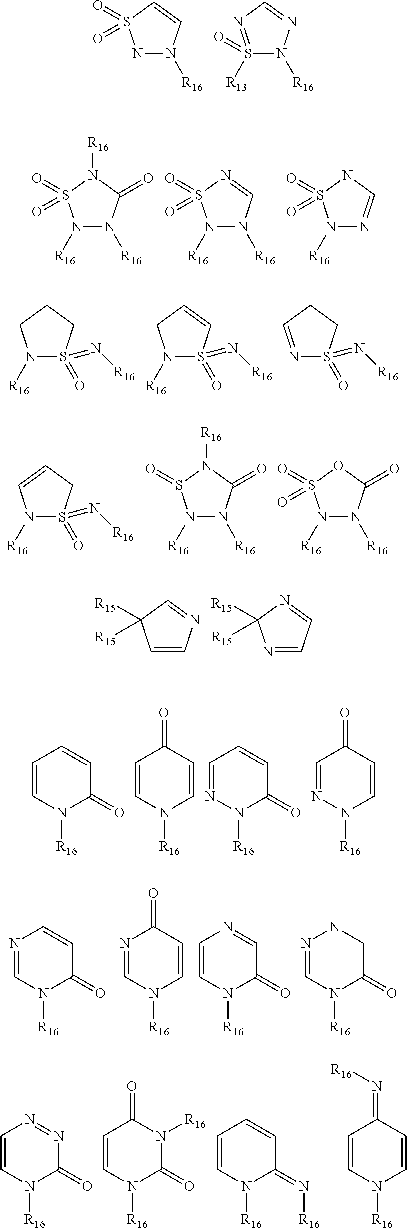 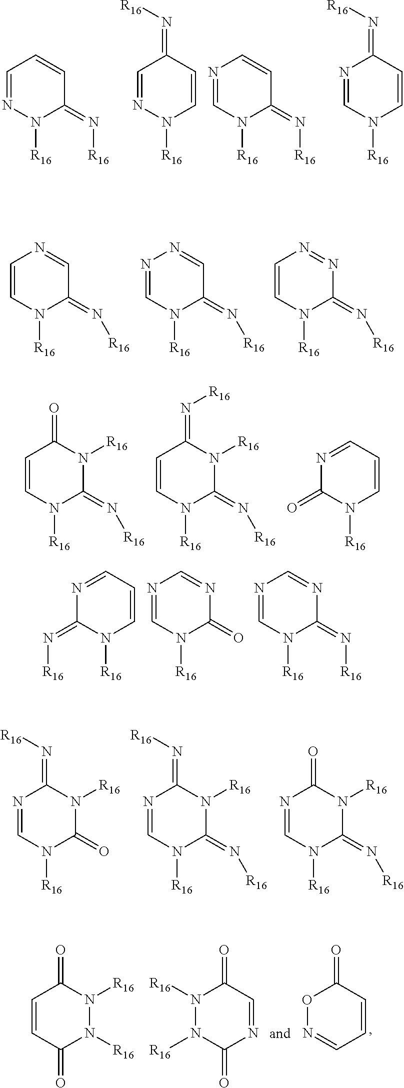
wherein any R₁₅ or R₁₆ substituent bound to a divalent ring system at a position vicinal to, or vinylogously linked to, C═O, C═N or C═S is not H if tautomerization at such position could lead to aromatization of the divalent ring system.

11. A compound of the formula (I) 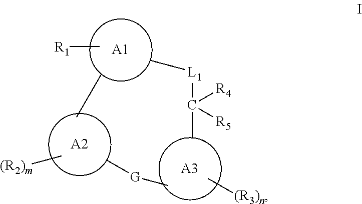
or a pharmaceutically acceptable salt thereof, wherein:
A1 is 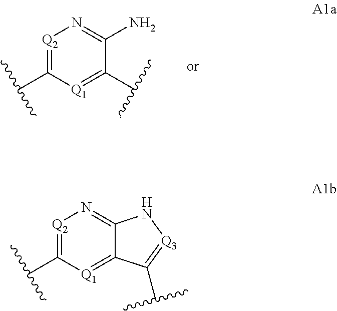
Q₁ is N or CH;
Q₂ is N or CH;
Q₃ is N or C-J-R₁;
A2 is a C₈-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
A3 is a C₈-C₁₂ arylene or a 5- to 12-membered heteroarylene containing up to four heteroatoms selected from S, O, and N, wherein the adjoining groups are attached to the arylene or heteroarylene in a 1,2- or 1,3-relationship;
J is a bond, —CR₉R₁₀—, —O—, —N(R₈)—, —ON(R₈)—, —SOₓ—, —S(O)(NR₈)—, —C(═O)—, —(CR₉R₁₀)₂—, —C(R₁₁)═C(R₁₁)—, —C≡C—, —C(═O)CR₉R₁₀—, —CR₉R₁₀C(═O)—, —OC(R₁₁)₂—, —C(R₁₁)₂O—, —NR₈C(R₁₁)₂—, —ONR₈C(R₁₁)₂—, —C(R₁₁)₂N(R₈)—, —C(═O)N(R₈)—, —N(R₈)C(═O)—, —OC(═O)—, —C(═O)O—, —SO₂N(R₈)—, —N(R₈)SO₂—, —ON(R₈)SO₂—, —S(O)(═NR₈)N(R₈)—, —N(R₈)S(O)(═NR₈)—, —S(O)(R₁₁)═N—, or —N═S(O)(R₁₁)—;
L1 is —O—, —S(O)x-, —C(═O)—, —CF₂—, —C(R₄)₂—, —N(R₄)—, or S(O)(═NR₄)— or is a bond;
G is 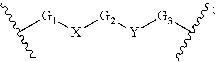
G₁, G₂, and G₃ are each independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, —O(CR₉R₁₀)ₒ—, —S(O)ₓ(CR₉R₁₀)ₒ—, or —NR₈(CR₉R₁₀)ₒ— or a bond;
X and Y are each independently a bond or —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 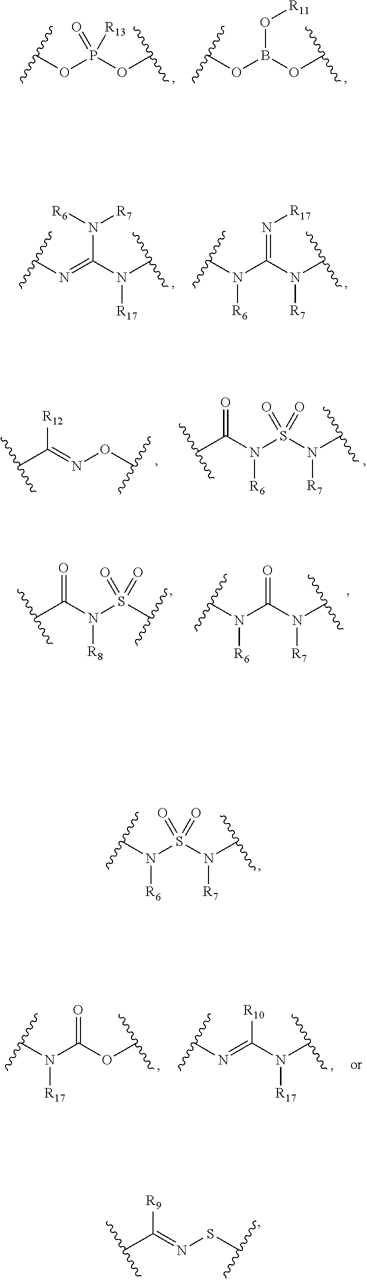
or a C₃—O₆ cylcoalkylene, a 3-6 membered heterocyclylene containing up to four heteroatoms selected from SOₓ, O, N, and NR₈, a C₆ arylene, or a 5-6 membered heteroarylene containing up to four heteroatoms selected from SOₓ, O, N, and NR₈; wherein the adjoining groups are attached to the cylcoalkylene, heterocyclylene, arylene, or heteroarylene in a 1,2- or 1,3-relationship; wherein the cylcoalkylene, heterocyclylene, arylene, or heteroarylene is optionally substituted with 1-3 R₁₅ groups; wherein G contains between 2 and 8 atoms in the direct chain that links A2 and A3;
R₁ is hydrogen, halogen, hydroxyl, NR₆R₇, cyano, C₁-C₆ alkyl, C₂-C₆ alkenyl, C₂-C₆ alkynyl, C₃-C₁₂ cycloalkyl, C₄-C₆ cycloalkenyl, C₆-C₁₂ aryl or 5-12 membered heteroaryl;
each R₂ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, halogen, hydroxy, —NHOH, —NR₈OR₁₁, hydrazino, cyano, nitro, azido, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, ONR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and NR₈(C₁-C₆ alkylene)NR₆R₇;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl);
each R₄ and R₅ is independently selected from hydrogen, CH₂F, CHF₂, CF₃, C₁-C₆ alkyl, C₂-C₆ alkenyl, C₂-C₆ alkynyl, and C₃-C₆ cycloalkyl;
each R₆ and R₇ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl or 5-12 membered heteroaryl; or R₆ and R₇ and the atom to which they are attached form a 4-12 membered monocyclic or bicyclic ring system in which up to two carbon atoms are replaced with N, NR₈, O, S(O)ₓ, and S(O)(NR₈);
each R₈ is independently H, C₁-C₆ alkyl, C₃-C₆ alkenyl, C₃-C₆ alkynyl, C₃-C₆ cycloalkyl, C₄-C₇ cycloalkenyl C₁-C₆ acyl, 4-12 membered heterocyclyl, C₆-C₁₂ aryl and a 5-12 membered heteroaryl;
each R₉ and R₁₀ is independently selected from hydrogen, halogen, C₁-C₆ alkyl, C₂-C₆ alkenyl, C₂-C₆ alkynyl, C₃-C₆ cycloalkyl, C₆-C₁₂ aryl, 4-12 membered heterocyclyl, 5-12 membered heteroaryl, OR₁₁, NR₆R₇, ONR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, ON(R₈)COR₁₂, CO₂R₁₁, CONR₆R₇, NR₈CONR₆R₇, ONR₈CONR₆R₇, NR₈CO₂R₁₁, ONR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, ONR₈SO₂R₁₃, ONR₈SO₂NR₆R₇, NR₈S(═O)(═NR₈)R₁₃, —N═S(═O)(R₁₃)R₁₃, —N═S(═O)(NR₆R₇)R₁₃, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁₁)₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, and NR₈P(O)(NR₆R₇)₂, or R₉ and R₁₀ on the same carbon atom, or two R₉ on contiguous carbon atoms, taken together form 4-12 membered monocyclic or bicyclic ring system in which up to two carbon atoms are replaced with N, NR₈, O, S(O)ₓ, and S(O)(NR₈);
each R₁₁ is independently selected from hydrogen, C₁-C₆ alkyl, C₂-C₆ alkenyl, C₂-C₆ alkynyl, C₃-C₆ cycloalkyl, C₈-C₁₂ aryl, 4-12 membered heterocyclyl, and 5-12 membered heteroaryl, or two R₁₁ and the direct chain linking the two R₁₁ groups form a 5-8 membered heterocyclyl;
each R₁₂ is independently selected from hydrogen, C₁-C₆ alkyl, C₂-C₆ alkenyl, C₂-C₆ alkynyl, C₃-C₆ cycloalkyl, C₈-C₁₂ aryl, 4-12 membered heterocyclyl, and 5-12 membered heteroaryl;
each R₁₃ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl;
each R₁₄ is independently selected from C₁-C₆ alkyl, C₂-C₆ alkenyl, C₂-C₆ alkynyl, C₃-C₆ cycloalkyl, C₆-C₁₂ aryl, 4-12 membered heterocyclyl, and 5-12 membered heteroaryl, or two R₁₄ and the atom to which they are attached form a 5-8 membered monocyclic or bicyclic ring system in which up to one carbon atom is replaced with NR₈, O, S(O)ₓ, or S(O)(NR₈);
each R₁₅ is independently selected from the group consisting of C₁-C₆ alkyl, C₂-C₆ alkenyl, C₂-C₆ alkynyl, C₃-C₆ cycloalkyl, C₃-C₇ cycloalkenyl, CF₃, OCF₃, hydrogen, halogen, hydroxy, —NHOH, hydrazino, cyano, nitro, azido, NR₆R₇, O(C₁-C₆ alkyl), O(C₃-C₆ alkenyl), O(C₃-C₆ alkynyl), O(C₃-C₆ cycloalkyl), O(C₃-C₇ cycloalkenyl), COR₁₂, OCOR₁₂, N(R₈)COR₁₂, CO₂R₁₁, CONR₆R₇, NR₈CONR₆R₇, NR₈CO₂R₁₁, OCO₂R₁₂, OCONR₆R₇, S(O)ₓR₁₃, S(R₁₃)(═O)═NR₈, S(═O)(═NR₈)NR₆R₇, SO₂NR₆R₇, NR₈SO₂R₁₃, NR₈SO₂NR₆R₇, NR₈S(═O)(═NR₈)R₁₃, —N═S(═O)(R₁₃)R₁₃, —N═S(═O)(NR₆R₇)R₁₃, ONR₆R₇, ON(R₈)COR₁₂, ONR₈CONR₆R₇, ONR₈CO₂R₁₁, ONR₈SO₂R₁₃, ONR₈SO₂NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, P(O)(NR₆R₇)₂, OP(O)(R₁₄)₂, OP(O)(OR₁₁)R₁₃, OP(O)(OR₁O₂, OP(O)(NR₆R₇)OR₁₁, OP(O)(NR₆R₇)R₁₃, OP(O)(NR₆R₇)₂, NR₈P(O)(R₁₄)₂, NR₈P(O)(OR₁₁)R₁₃, NR₈P(O)(OR₁₁)₂, NR₈P(O)(NR₆R₇)OR₁₁, NR₈P(O)(NR₆R₇)R₁₃, NR₈P(O)(NR₆R₇)₂, 4-12 membered heterocyclyl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, —NR₈(C₁-C₆ alkylene)OR₁₁, and —NR₈(C₁-C₆ alkylene)NR₆R₇, or is a bond to A2, A3, X, Y, G₁, G₂ or G₃;
each R₁₆ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, hydroxy, O(C₁-C₆ alkyl), COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, —O(C₁-C₆ alkylene)OR₁₁, —O(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, and P(O)(NR₆R₇)₂, or is a bond to A2, A3, X, Y, G₁, G₂ or G₃;
each R₁₇ is independently selected from hydrogen, C₁-C₆ alkyl, C₂-C₆ alkenyl, C₂-C₆ alkynyl, C₃-C₆ cycloalkyl, C₃-C₇ cycloalkenyl, CF₃, COR₁₂, CO₂R₁₁, CONR₆R₇, S(O)ₓR₁₃, S(R₁₃)(═O)═NR₈, SO₂NR₆R₇, C₆-C₁₂ aryl, 4-12 membered heterocyclyl, 5-12 membered heteroaryl, —(C₁-C₆ alkylene)OR₁₁, —(C₁-C₆ alkylene)NR₆R₇, P(O)(R₁₄)₂, P(O)(OR₁₁)R₁₃, P(O)(OR₁₁)₂, P(O)(NR₆R₇)OR₁₁, P(O)(NR₆R₇)R₁₃, or P(O)(NR₆R₇)₂, or two R₁₇ groups together with the nitrogen atom to which they are attached form a 4-7 membered heterocyclyl;
m is 0-3;
n is 0-2;
o is 0-3;
p is 0-2; and
x is 0-2;
with the proviso that when A1 is A1a, and only one out of X and Y is present, and G contains one heteroatom selected from N and O within the direct chain that links A2 and A3, and G does not contain a carbon-carbon double bond in the direct chain that links A2 and A3, and R₃ is halogen or C₁₋₆ alkyl, and none of R₂, R₉, R₁₀, R₁₅ and R₁₆ contain phosphorus, a sulfur-nitrogen double bond, sulfur bonded to two nitrogen atoms, nitrogen bonded to one oxygen atom, a carbonate, a carbamate or a urea; then neither X nor Y is selected from the group consisting of: O, NR₈, CONR₈, NR₈CO, arylene and heteroarylene.

12. The compound of claim 1-11 or a pharmaceutically acceptable salt thereof, wherein:
A1 is 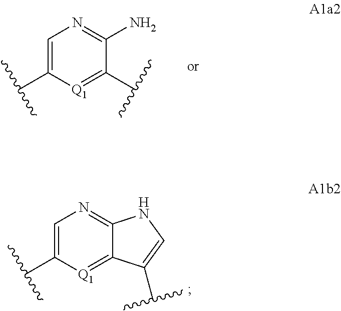
L1 is O—, —CH₂—, or —NH— or is a bond.

13. The compound of claim 12 of the formula (II) 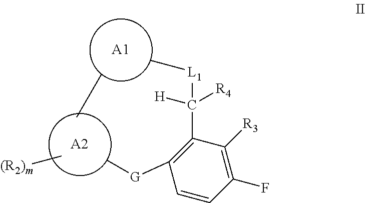
or a pharmaceutically acceptable salt thereof, wherein:
R₃ is hydrogen, halogen, CH₃, CH₂F, CF₃, or cyano; and
R₄ is —CH₃, —CH₂F, CHF₂, or CF₃.

14. The compound of claim 1-11 of the formula (III) 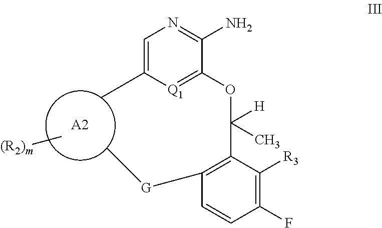
or a pharmaceutically acceptable salt thereof, wherein:
A2 is a benzene ring or a 5- to 6-membered heteroarylene containing up to three heteroatoms selected from S, O, and N, wherein the adjoining groups are attached to the benzene ring or heteroarylene in a 1,2- or 1,3-relationship;
each R₃ is independently selected from hydrogen, halogen, C₁-C₆ alkyl, C₂-C₆ alkenyl, C₂-C₆ alkynyl, cyano, nitro, azido, —CHO, CH₂F, CHF₂, CF₃, O(C₁-C₆ alkyl), and —S(O)ₓ(C₁-C₆ alkyl).

15. The compound of claim 1-11 of the formula (IV) 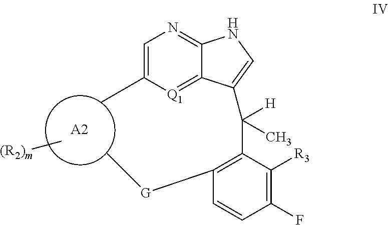
or a pharmaceutically acceptable salt thereof, wherein:
A2 is a benzene ring or a 5- to 6-membered heteroarylene containing up to three heteroatoms selected from S, O, and N, wherein the adjoining groups are attached to the benzene ring or heteroarylene in a 1,2- or 1,3-relationship;
each R₃ is independently selected from H, F, Cl, CH₃, CH₂F, CF₃, and cyano.

16. The compound of any one of claims 1-15, or a pharmaceutically acceptable salt thereof, wherein:
G is —X-G₂-Y—;
G₂ is —(CR₉R₁₀)ₒ;
X and Y are each independently selected from —O—, —NR₈—, —B(OR₁₁)—, —S(O)ₓ—, —S(O)(═NR₈)—, —C(O)NR₈—, —C(R₁₂)═N—, —C(NR₁₇)═N—, —C(OR₁₁)═N—, —C(═NR₆)N(R₇)—, and —C(═NR₁₇)N(R₁₇)—; and
o is 1-3.

17. The compound of any one of claims 1-15, or a pharmaceutically acceptable salt thereof, wherein:
G is —X-G₂-Y—;
G₂ is —(CR₉R₁₀)ₒ;
one of X and Y selected from —O—, —NR₈—, —S(O)ₓ—, and —S(O)(═NR₈)—;
one of X and Y is a divalent ring system selected from the group consisting of; 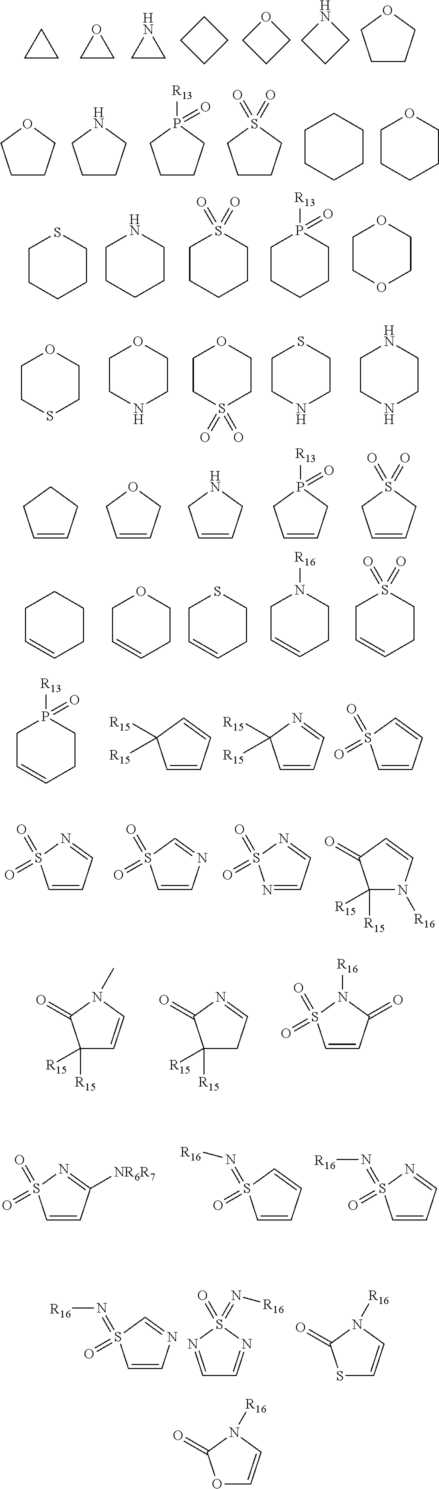 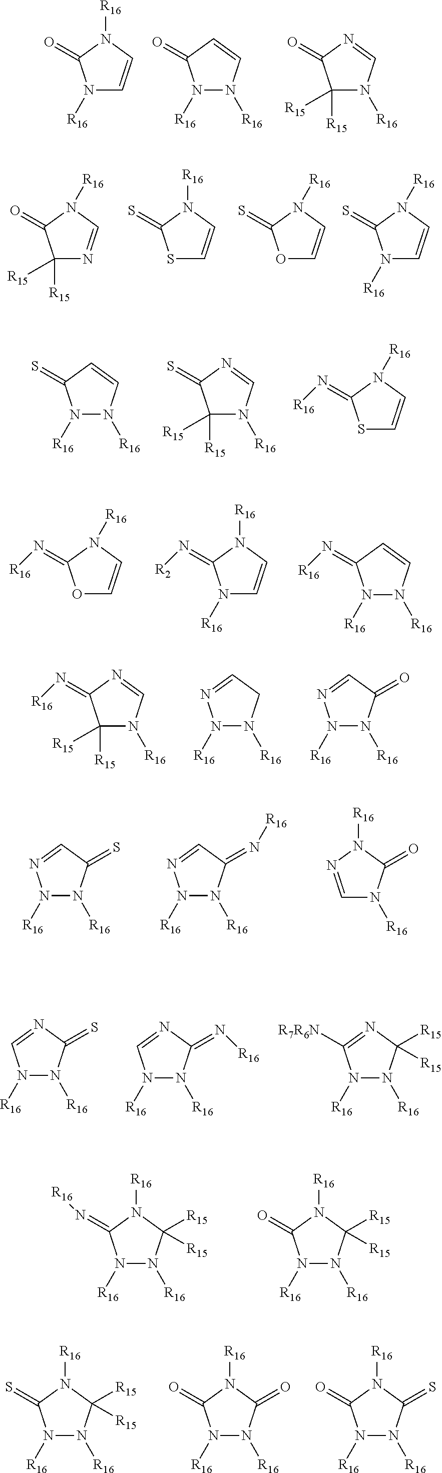 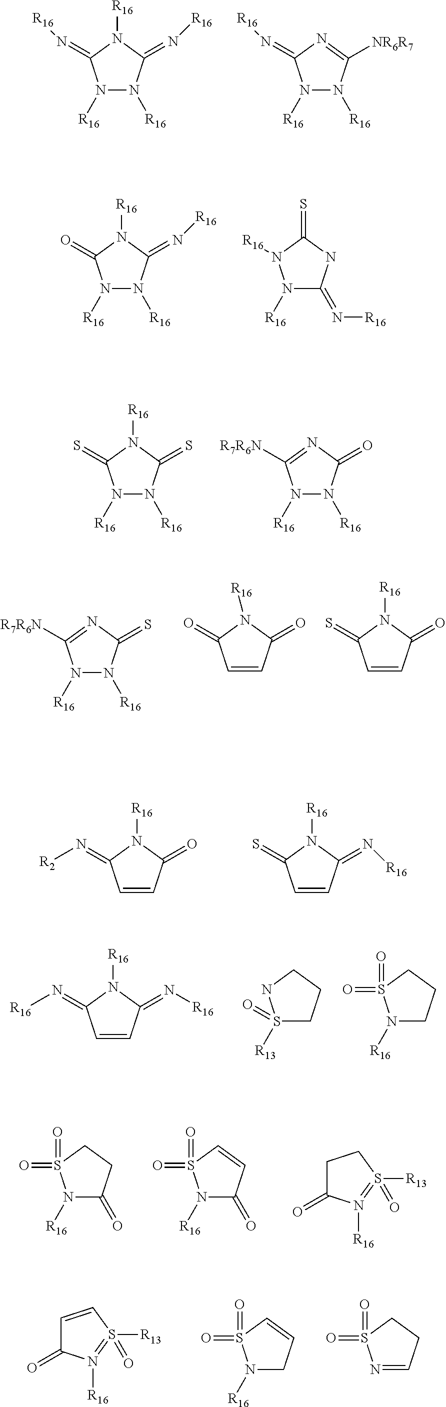 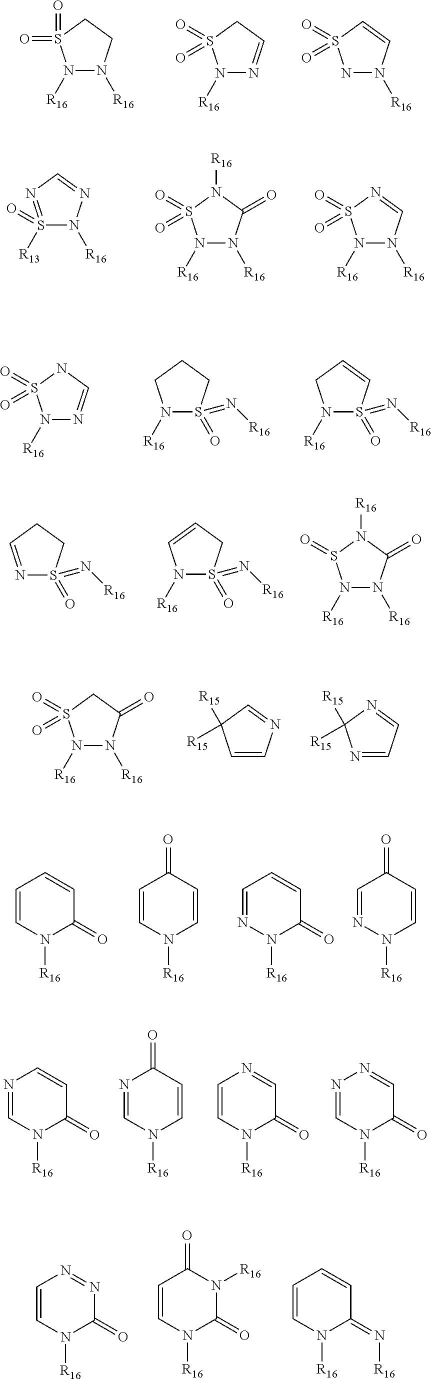 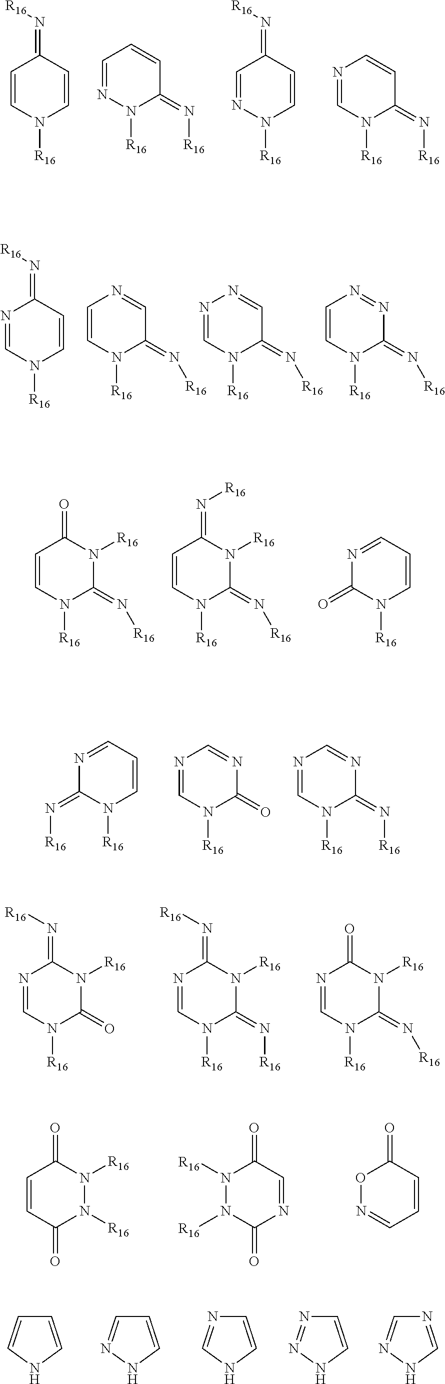 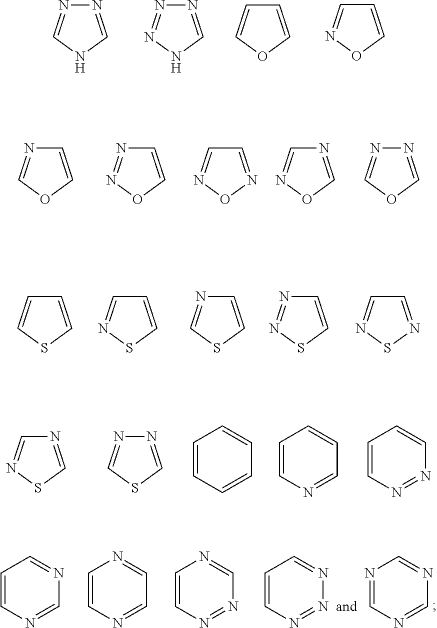
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system;
o is 1-3.

18. The compound of claim 17, or a pharmaceutically acceptable salt thereof, wherein the adjoining groups are attached to the divalent ring system of X or Y in a 1,2-relationship; and wherein the divalent ring system is a heteroarylene selected from the group consisting of pyrrolylene, thienylene, furanylene, imidazylene, pyrazolylene, thiazolylene, oxazolylene, isothiazolylene, isoxazolylene, 1,2,3-triazolylene, 1,2,4-triazolylene, 1,2,3-oxadiazolylene, 1,3,2-oxadiazolylene, 1,2,3-thiadiazolylene, 1,3,2-thiadiazolylene and tetrazolylene, wherein said heteroarylene is optionally substituted with 1-3 R₁₅ or R₁₆ groups.

19. The compound of claim 17, or a pharmaceutically acceptable salt thereof, wherein the adjoining groups are attached to the divalent ring system in a 1,2-relationship; and wherein the divalent ring system is a heteroarylene selected from the group consisting of phenylene, pyridylene, pyrazinylene, pyrimidylene, pyridazinylene, 1,2,4-triazenylene, 1,3,5-triazenylene and 1,2,3-triazenylene, wherein said heteroarylene is optionally substituted with 1-3 R₁₅ or R₁₆ groups.

20. The compound of any one of claims 1-15, or a pharmaceutically acceptable salt thereof, wherein
each of G₁, G₂, and G₃ is independently —(CR₉R₁₀)ₒ—, —CR₉═CR₁₀—, —C(═O)—, —C(═NR₈)—, —C(═NOR₁₁)—, —C(═NNR₆R₇)—, —CF₂—, —S(O)ₓ(CR₉R₁₀)ₒ—, or —CR₉═CR₁₀CR₉R₁₀—, or a bond; wherein at least one of G₁, G₂, and G₃ is not a bond; and
X and Y are each a bond.

21. The compound of any one of claims 1-15, or a pharmaceutically acceptable salt thereof, wherein:
each of G₁, G₂, and G₃ is independently —(CR₉R₁₀)ₒ—, —CR₉═CR₁₀—, —C(═O)—, —C(═NR₈)—, —C(═NOR₁₁)—, —C(═NNR₆R₇)—, —CF₂—, or CR₉═CR₁₀CR₉R₁₀—, or a bond; wherein at least one of G₁, G₂, and G₃ is not a bond
one of X and Y is a bond; and
one of X and Y is —S(O)ₓ—, —S(O)(═NR₈)—, —P(O)(R₁₃)—, or —P(O)(OR₁₁)—.

22. The compound of any one of claims 1-15, or a pharmaceutically acceptable salt thereof, wherein one or two of G₁, G₂, and G₃ is a bond; wherein X and Y are each independently a bond, —S(O)ₓ—, —S(O)(═NR₈)—, —P(R₁₃)—, —P(O)(R₁₃)—, —P(O)(OR₁₁)—, —SO₂NR₈—, —S(O)(R₁₃)═N—, —S(O)(═NR₆)N(R₇)—, —S(O)(N(R₆R₇)═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, C(═NR₆)N(R₇)—, —C(═NR₁₇)N(R₁₇)—, 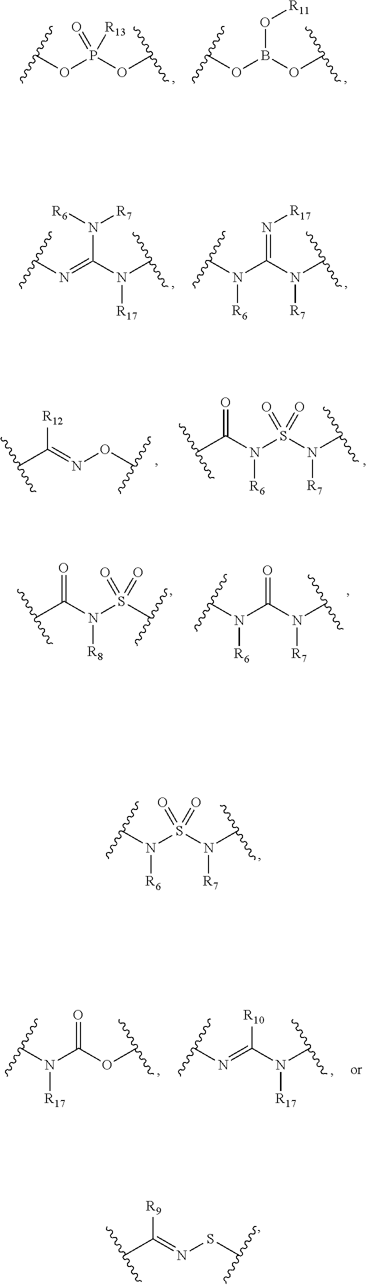
wherein one of X and Y is a bond.

23. The compound of claim 22, or a pharmaceutically acceptable salt thereof, wherein X and Y are each independently a bond or —S(O)ₓ—, —S(O)(═NR₈)—, —P(R₁₃)—, —P(O)(R₁₃)—, —P(O)(OR₁₁)—, —SO₂NR₈—, —S(O)(R₁₃)═N—, —S(O)(═NR₆)N(R₇)—, —S(O)(N(R₆R₇)═N—, —P(O)(R₁₃)O—, or —P(O)(OR₁₁)O—.

24. The compound of any one of claims 1-15, or a pharmaceutically acceptable salt thereof, wherein one of X and Y is a divalent ring system selected from the group consisting of: 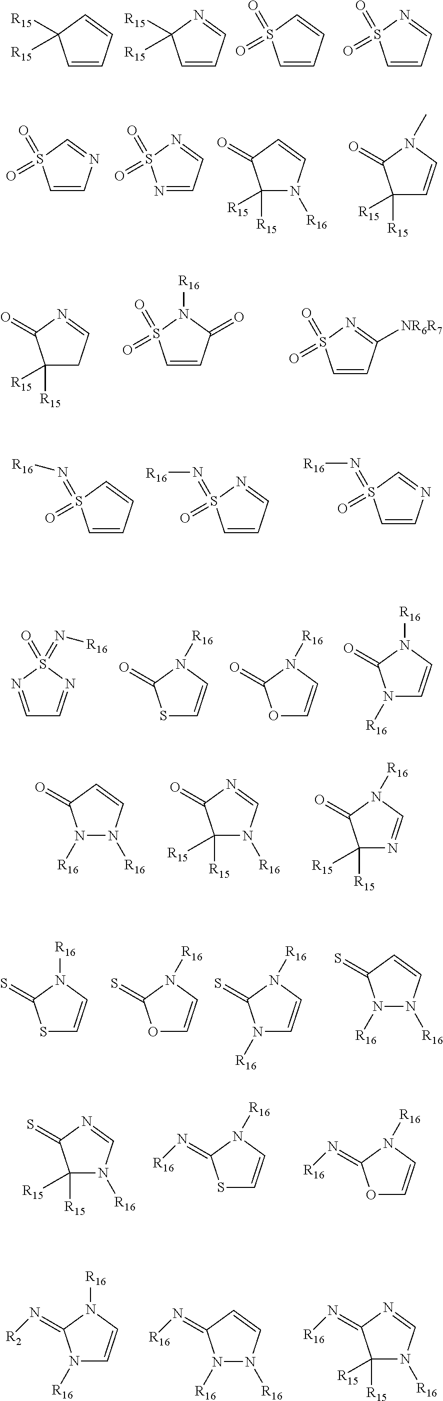 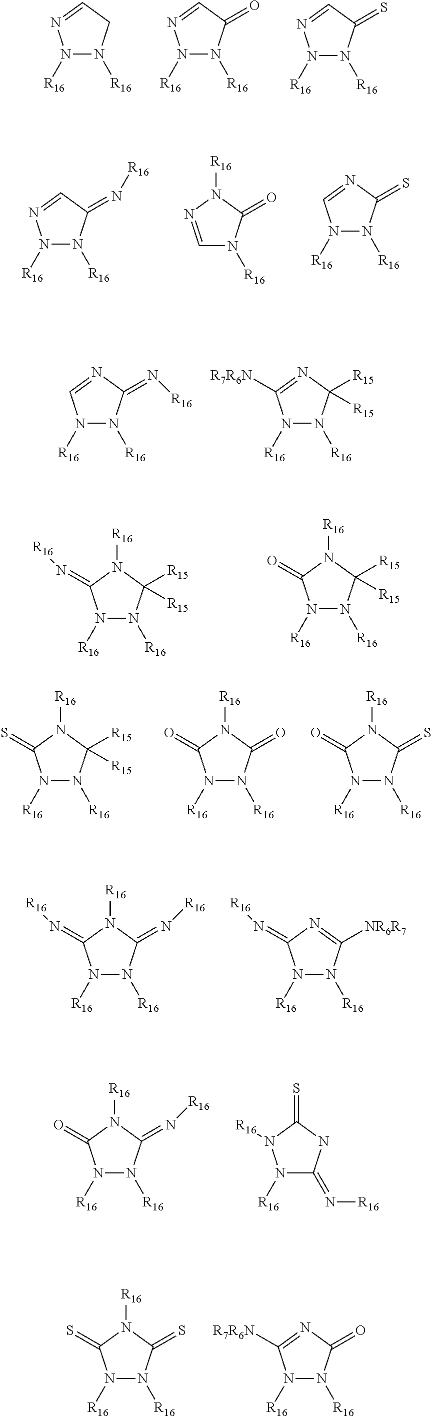 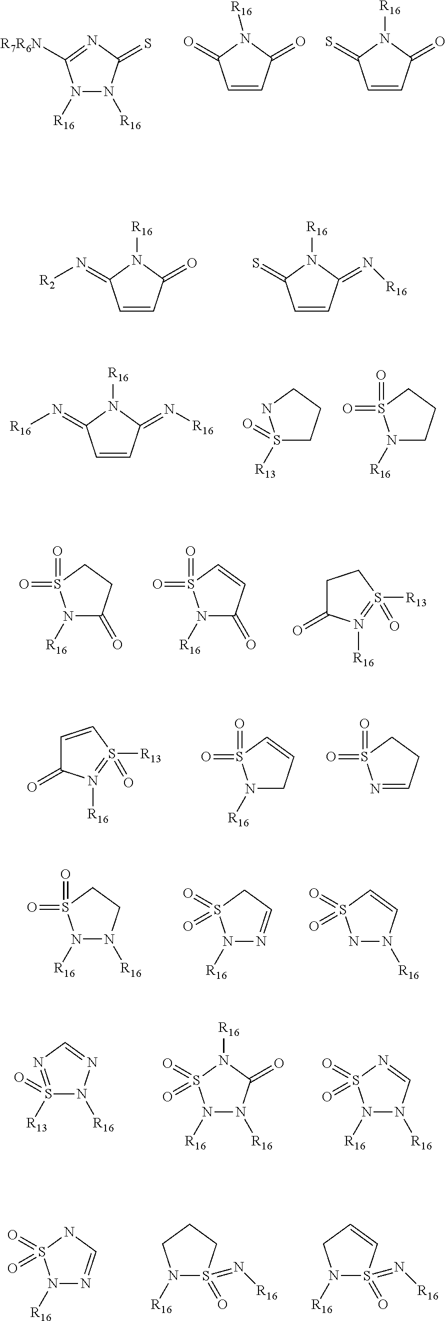 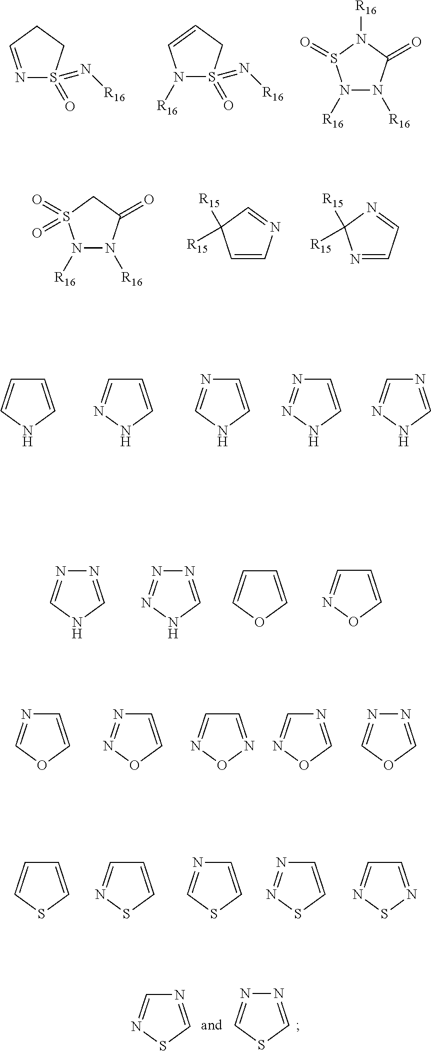
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system.

25. The compound of claim 24, or a pharmaceutically acceptable salt thereof, wherein:
G₁ is CR₉R₁₀, —C(═O)—, —C(═NR₈)—, —C(═NOR₁₁)—, —C(═NNR₆R₇)—, —CF₂—, —O(CR₉R₁₀)ₒ—, —S(O)ₓ(CR₉R₁₀)ₒ—, or —NR₈(CR₉R₁₀)ₒ— or a bond;
G₂ is a bond;
G₃ is CR₉R₁₀, —C(═O)—, —C(═NR₈)—, —C(═NOR₁₁)—, —C(═NNR₆R₇)—, —CF₂—, —O(CR₉R₁₀)ₒ—, —S(O)ₓ(CR₉R₁₀)ₒ—, or —NR₈(CR₉R₁₀)ₒ— or a bond; wherein G₁ and G₃ are not both a bond; and
one of X and Y is selected from —O—, —NR₈—, —S(O)ₓ— and —S(O)(═NR₈)—.

26. The compound of any one of claims 1-15, or a pharmaceutically acceptable salt thereof, wherein one of X and Y is a divalent ring system selected from the group consisting of: 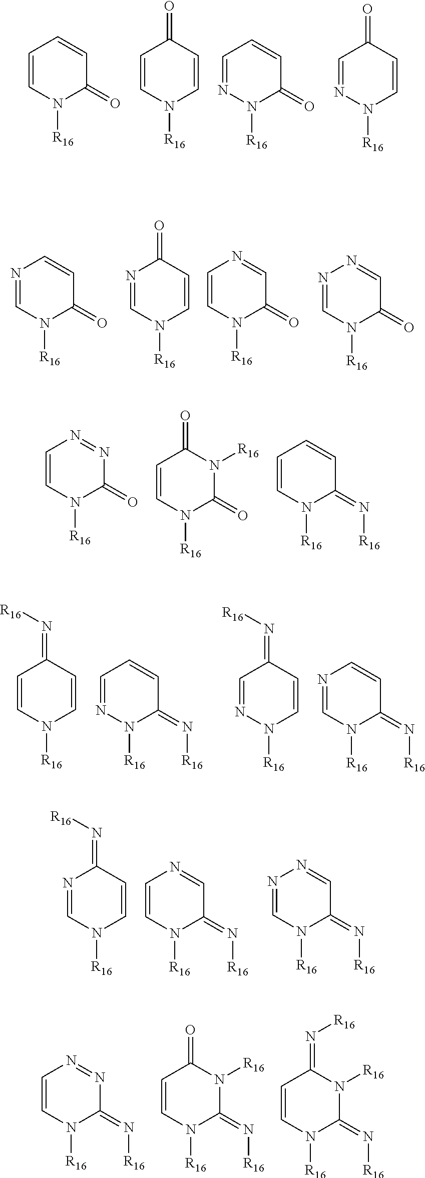 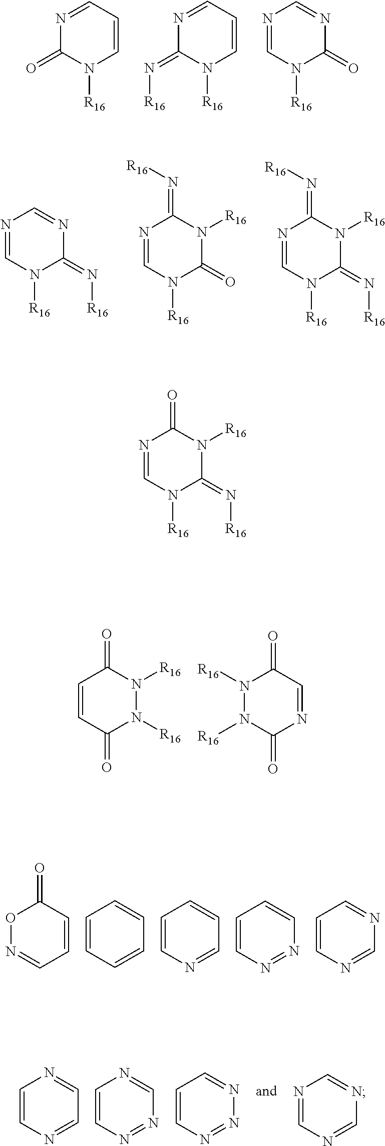
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system.

27. The compound of claim 26, or a pharmaceutically acceptable salt thereof, wherein:
G₁ is CR₉R₁₀, —C(═O)—, —C(═NR₈)—, —C(═NOR₁₁)—, —C(═NNR₆R₇)—, —CF₂—, O(CR₉R₁₀)ₒ—, —S(O)ₓ(CR₉R₁₀)ₒ—, or —NR₈(CR₉R₁₀)ₒ— or a bond;
G₂ is a bond;
G₃ is CR₉R₁₀, —C(═O)—, —C(═NR₈)—, —C(═NOR₁₁)—, —C(═NNR₆R₇)—, —CF₂—, —O(CR₉R₁₀)ₒ—, —S(O)ₓ(CR₉R₁₀)ₒ—, or —NR₈(CR₉R₁₀)ₒ— or a bond; wherein G₁ and G₃ are not both a bond; and
one of X and Y is selected from —O—, —NR₈—, —S(O)ₓ— and —S(O)(═NR₈)—.

28. The compound of any one of claims 1-15, or a pharmaceutically acceptable salt thereof, wherein:
G₂ is —(CR₉R₁₀)ₒ—, —CR₉═CR₁₀—, —C(═O)—, —C(═NR₈)—, —C(═NOR₁₁)—, —C(═NNR₆R₇)—, —CF₂—, —CR₉═CR₁₀CR₉R₁₀—, —O(CR₉R₁₀)ₒ—, —S(O)ₓ(CR₉R₁₀)ₒ—, or —NR₈(CR₉R₁₀)ₒ—; wherein at least one of G₁ and G₃ is a bond;
one of X and Y is a divalent ring system selected from the group consisting of: 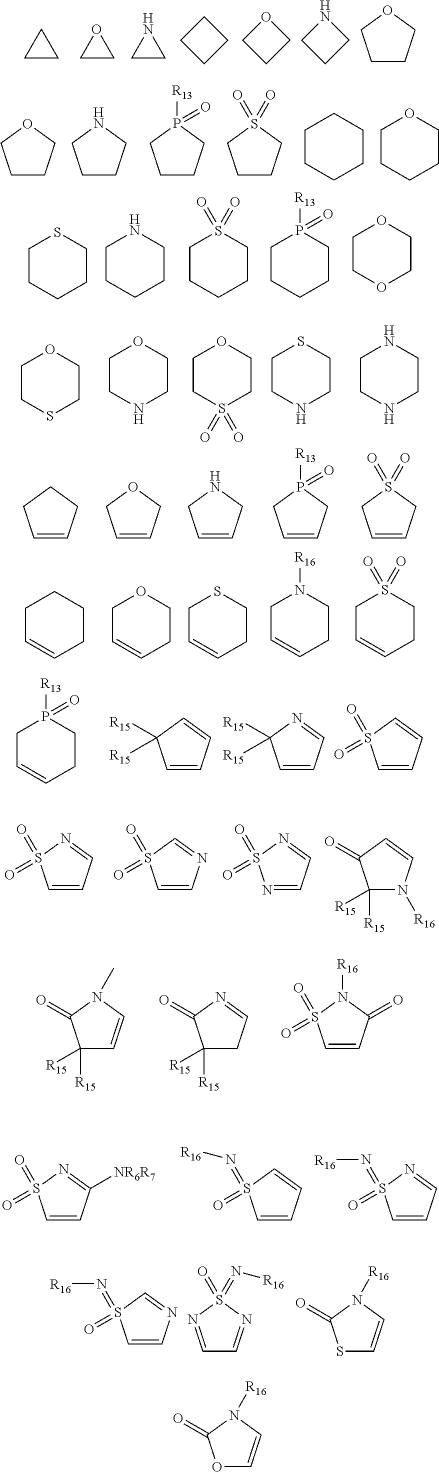 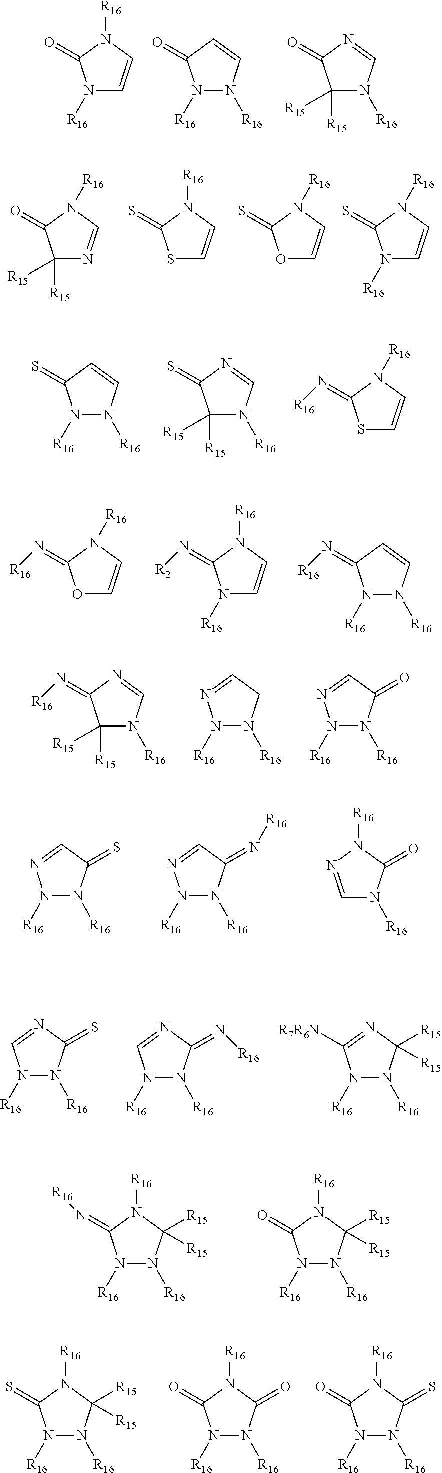 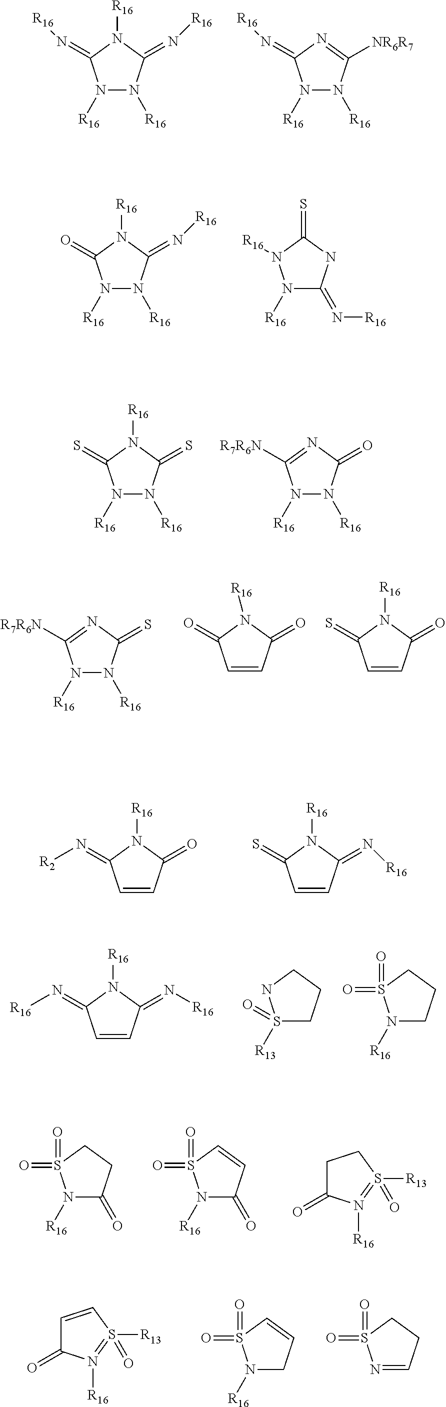 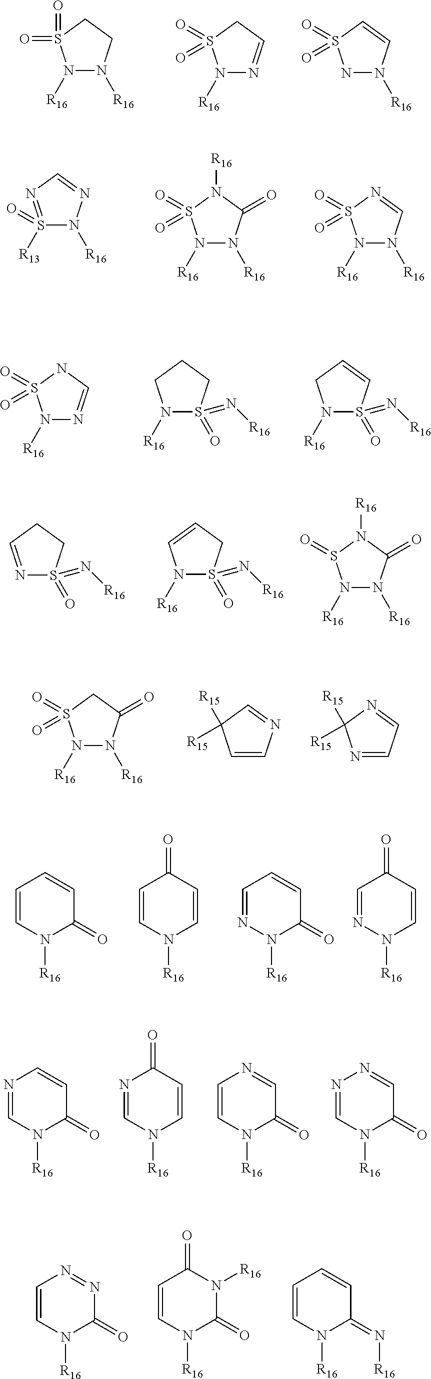 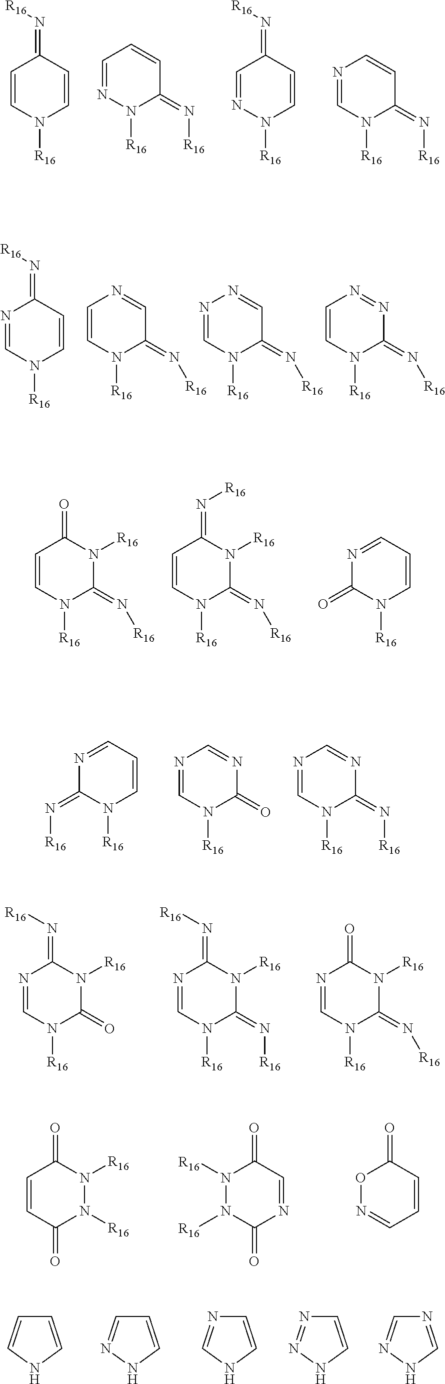 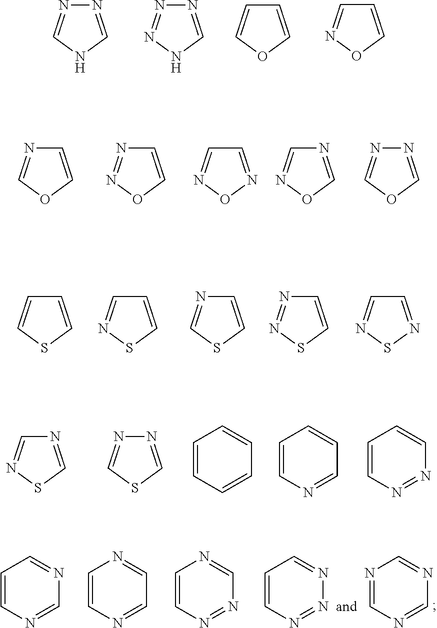
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; and
one of X and Y is —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇)═N—, —P(O)(R₁₃)O—, —P(O)(OR₁₁)O—, —B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—,

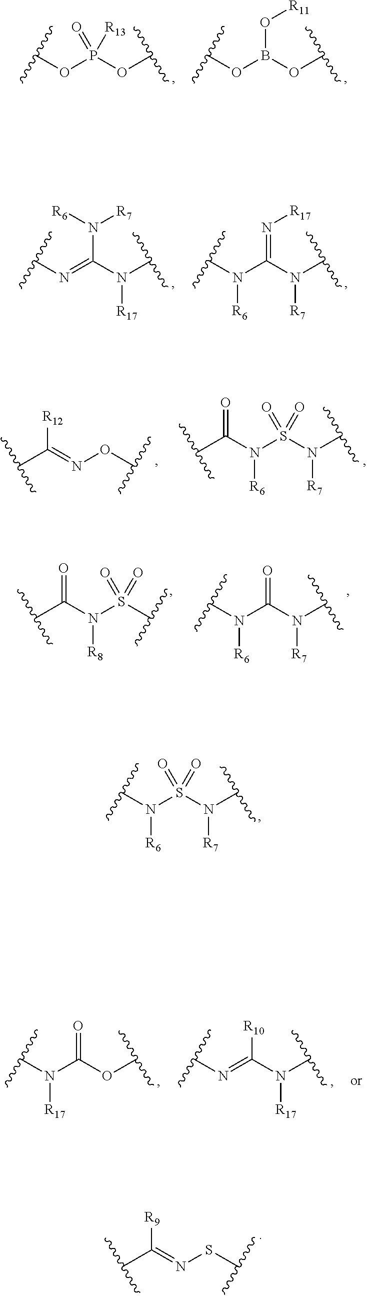

29. The compound of claim 28, or a pharmaceutically acceptable salt thereof, wherein:
one of X and Y is the divalent ring system, wherein the adjoining groups are attached to the divalent ring system in a 1,2-relationship, wherein the divalent ring system is a heteroarylene selected from the group consisting of pyrrolylene, thienylene, furanylene, imidazylene, pyrazolylene, thiazolylene, oxazolylene, isothiazolylene, isoxazolylene, 1,2,3-triazolylene, 1,2,4-triazolylene, 1,2,3-oxadiazolylene, 1,3,2-oxadiazolylene, 1,2,3-thiadiazolylene, 1,3,2-thiadiazolylene and tetrazolylene, wherein said heteroarylene is optionally substituted with 1-3 R₁₅ or R₁₆ groups; and
one of X and Y is —O—, —NR₈—, —B(OR₁₁)—, —S(O)ₓ—, —S(O)(═NR₈)—, —C(O)NR₈—, —C(R₁₂)═N—, —C(NR₁₇)═N—, —C(OR₁₁)═N—, —C(═NR₆)N(R₇)—, or —C(═NR₁₇)N(R₁₇)—.

30. The compound of any one of claims 1-15, or a pharmaceutically acceptable salt thereof, wherein:
at least one of G₁ and G₃ is not a bond;
G2 is a bond;
one of X and Y is a divalent ring system selected from the group consisting of: 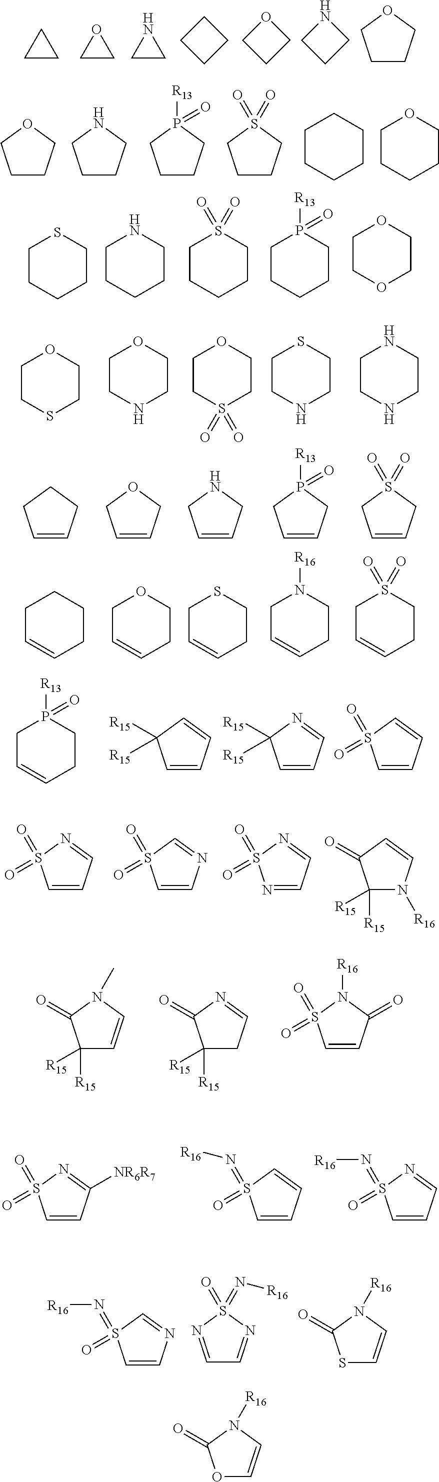 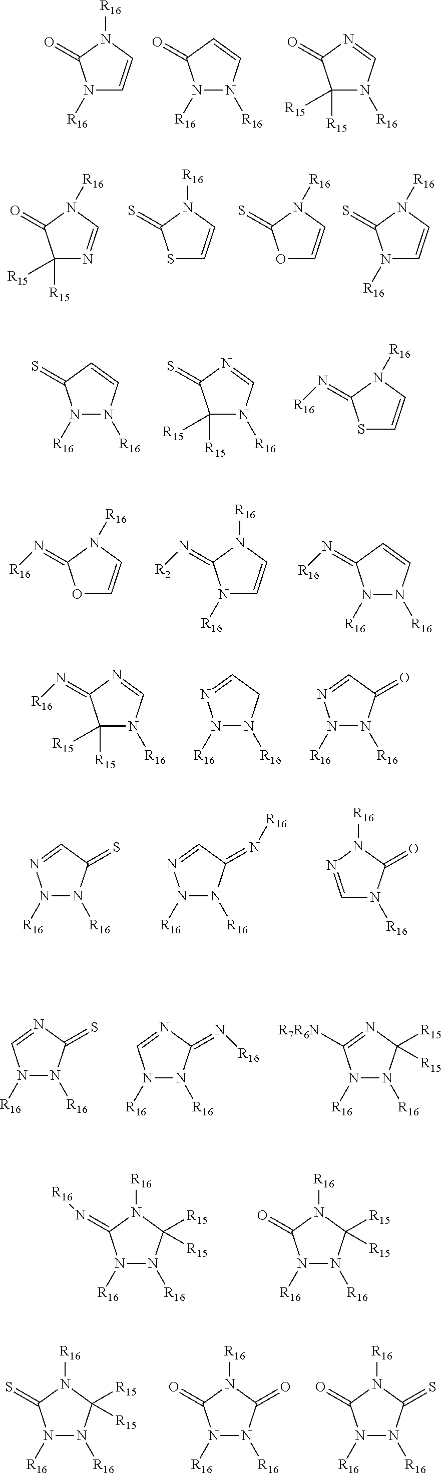 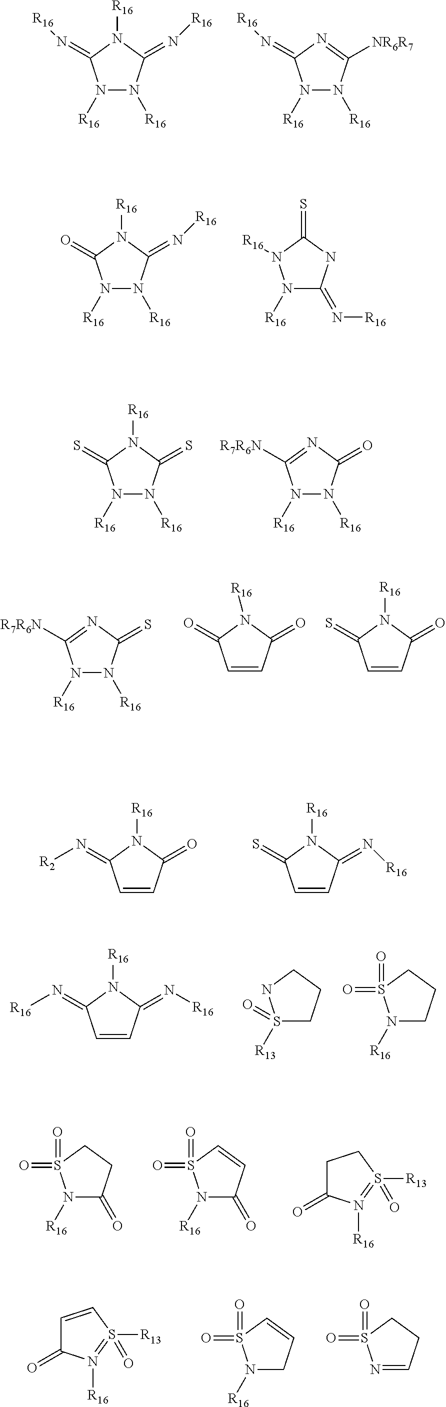 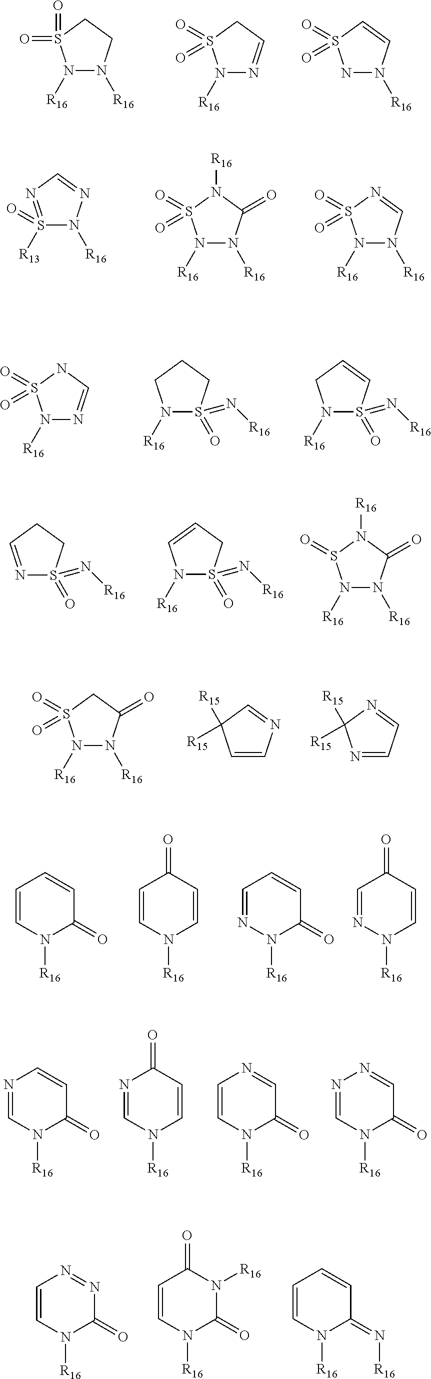 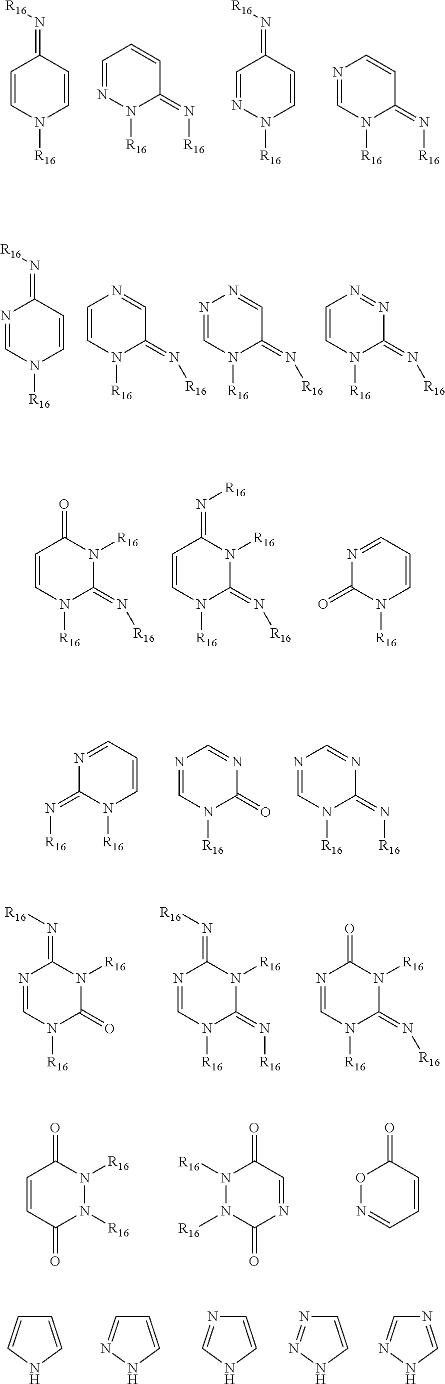 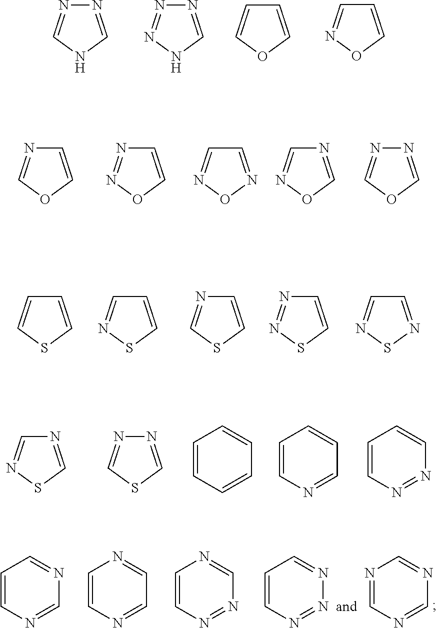
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; and
one of X and Y is —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇)═N—, —P(O)(R₁₃)O—, —P(O)(OR₁₁)O—, —B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—,

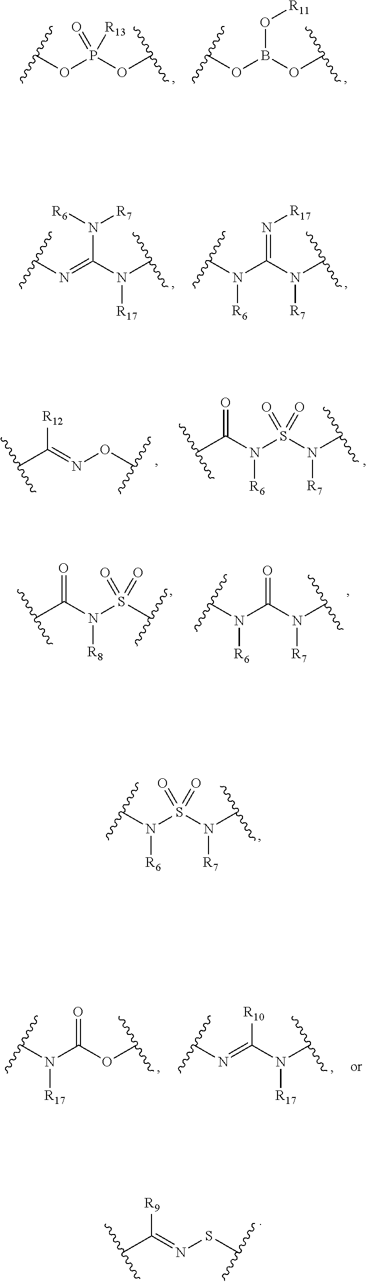

31. The compound of claim 1-15, or a pharmaceutically acceptable salt thereof, wherein:
G is —X—Y—;
one of X and Y is a divalent ring system selected from the group consisting of: 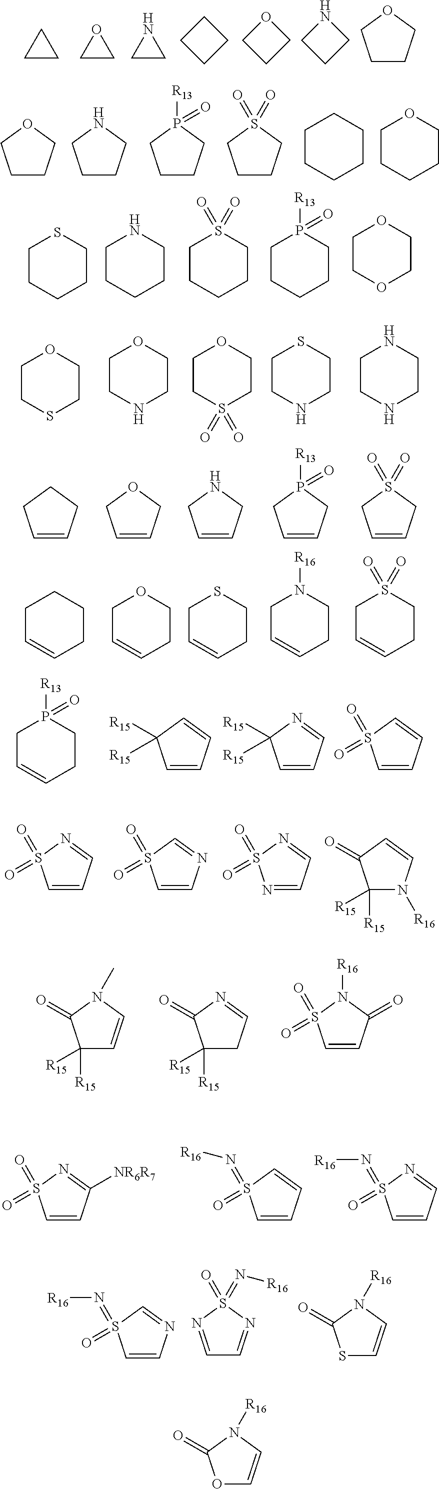 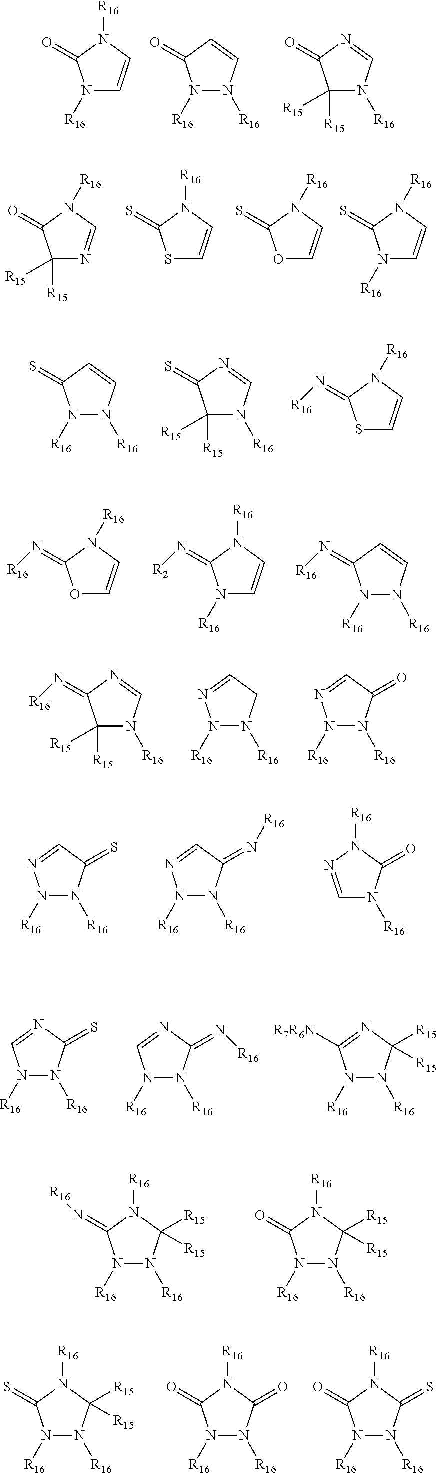 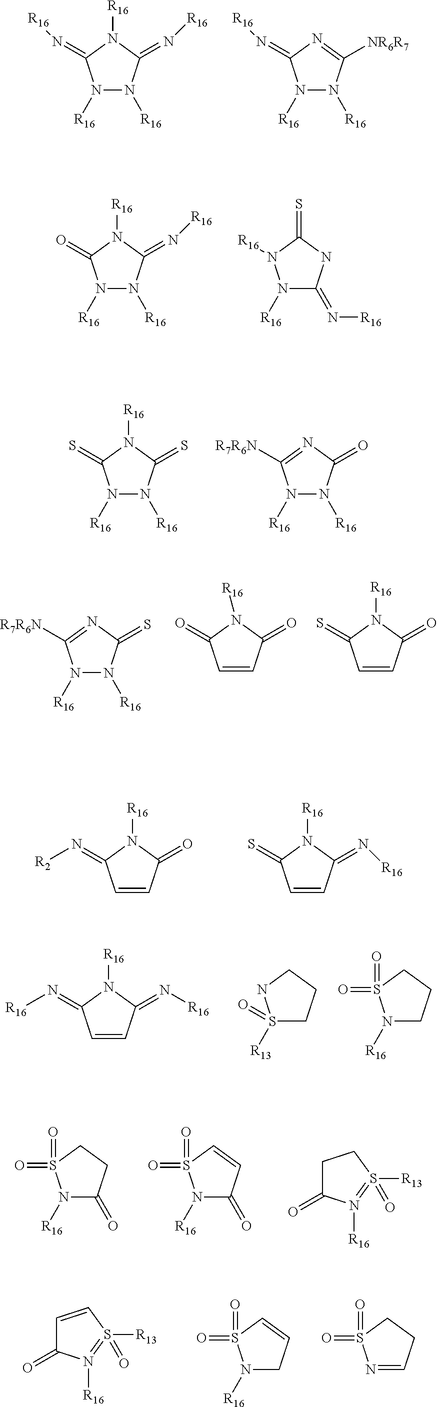 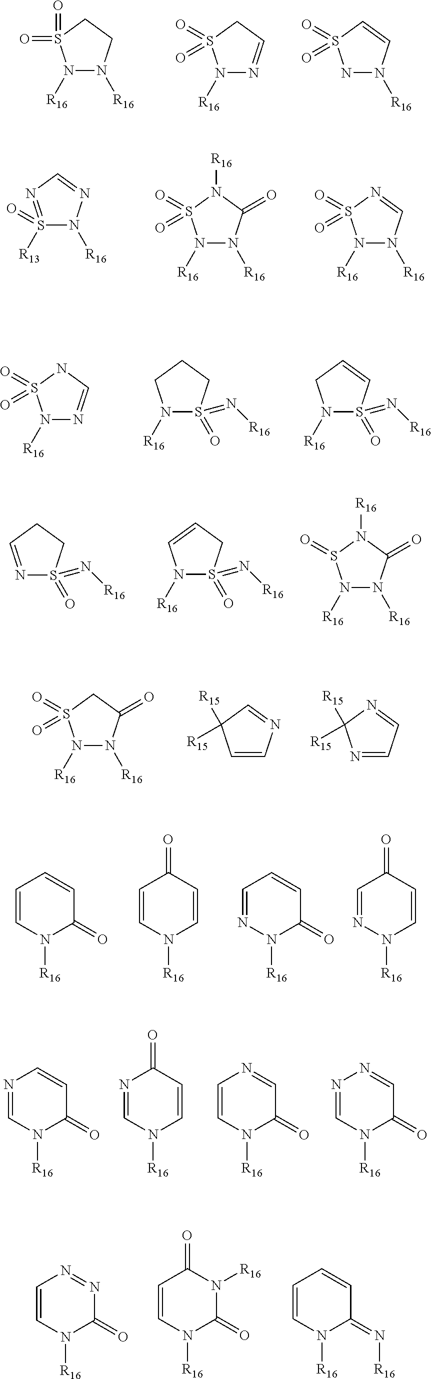 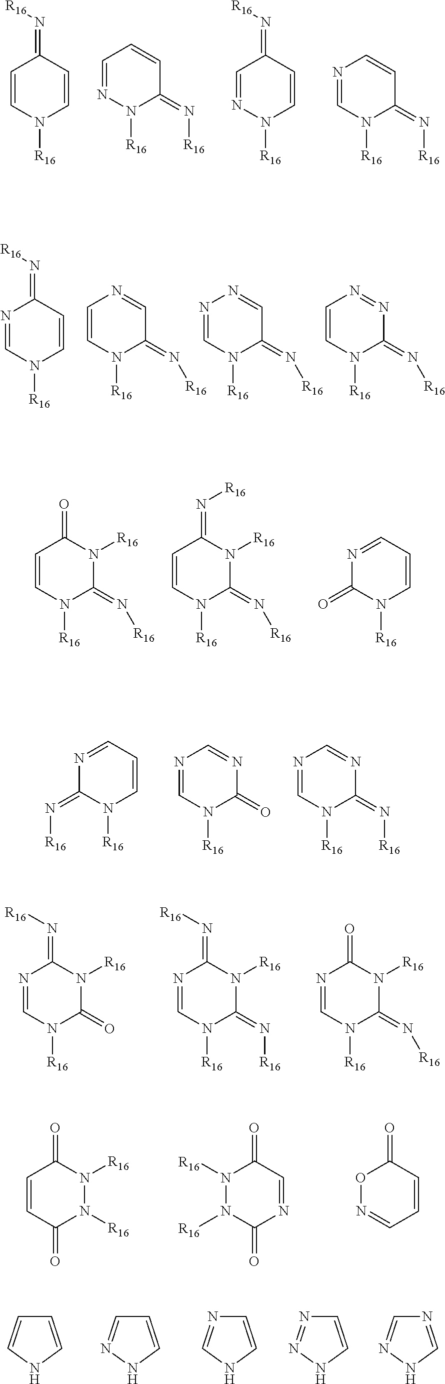 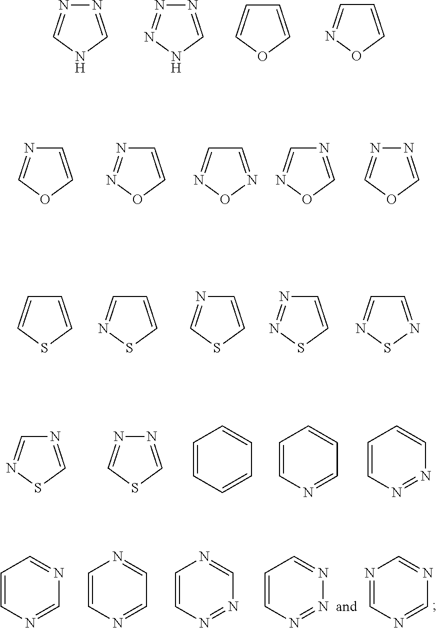
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; and
one of X and Y is —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇)═N—, —P(O)(R₁₃)O—, —P(O)(OR₁₁)O—, —B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—,

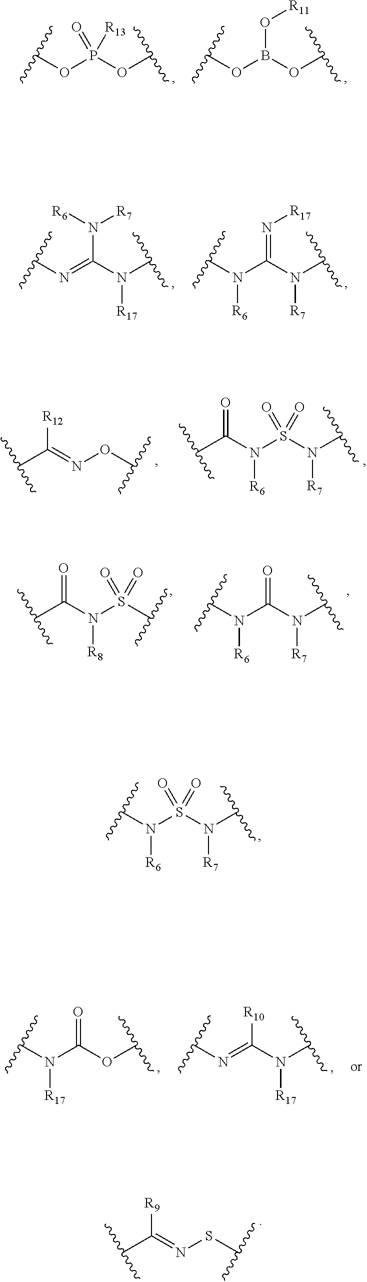

32. The compound of any one of claims 1-14 of the formula (V) 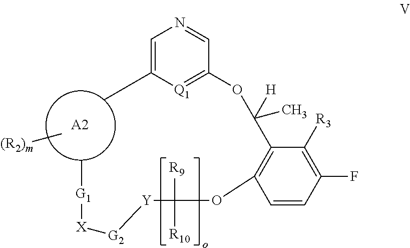
or a pharmaceutically acceptable salt thereof, wherein when neither X nor Y is a bond:
one of X and Y a divalent ring system selected from the group consisting of: 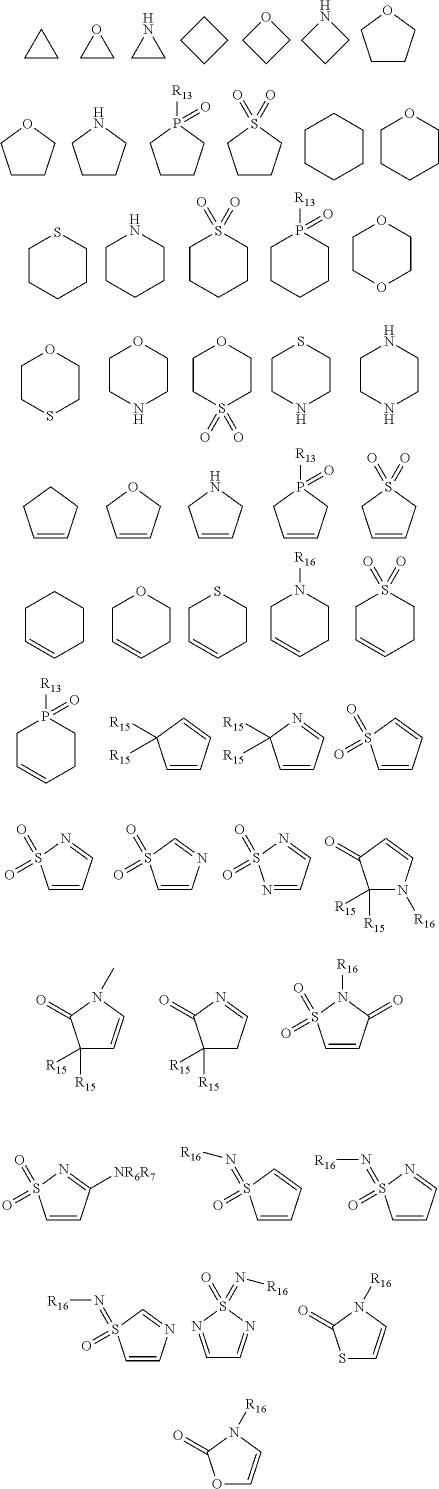 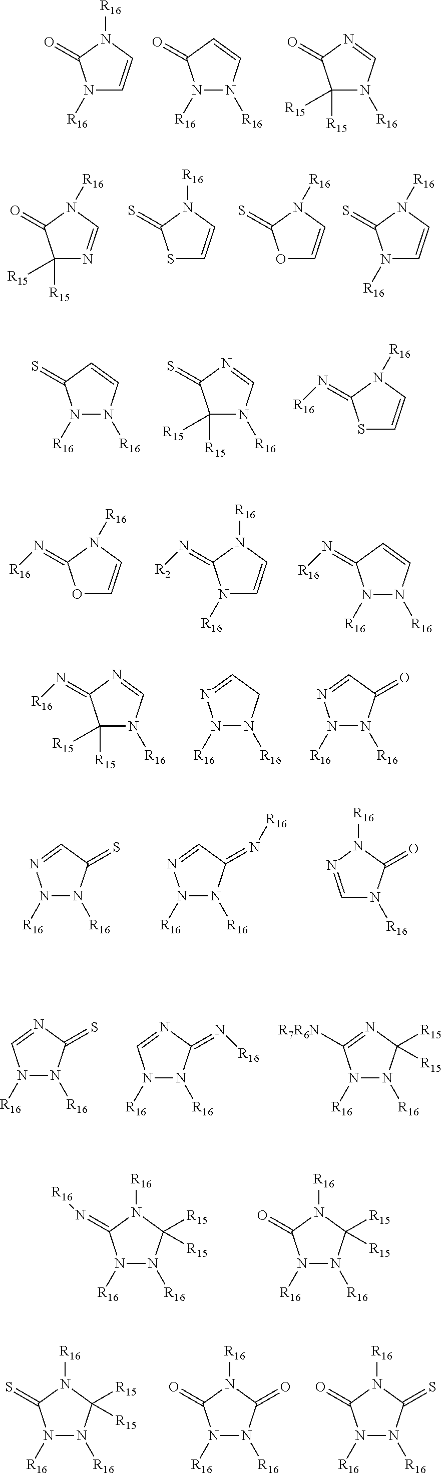 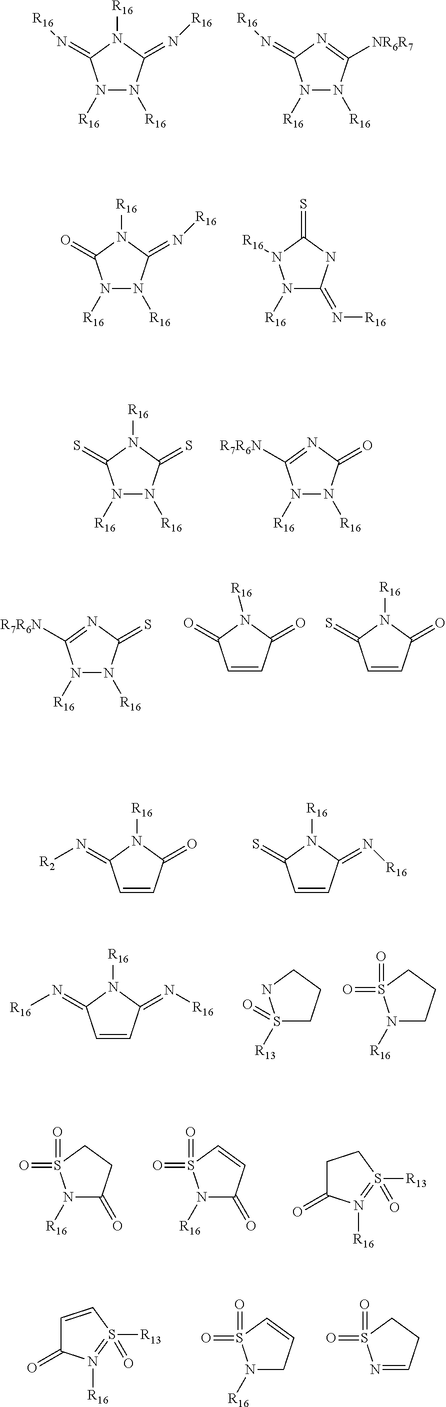 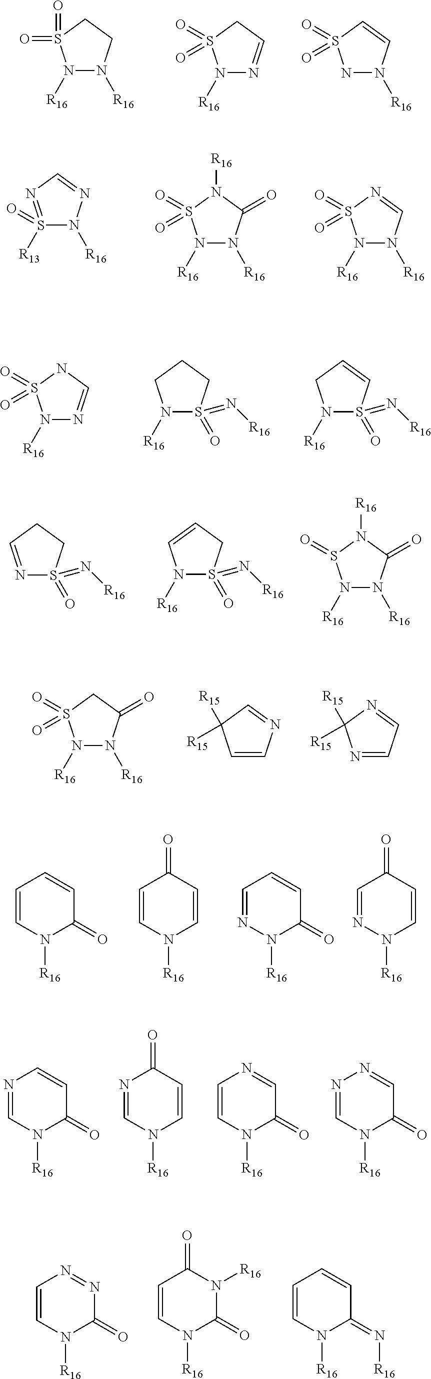 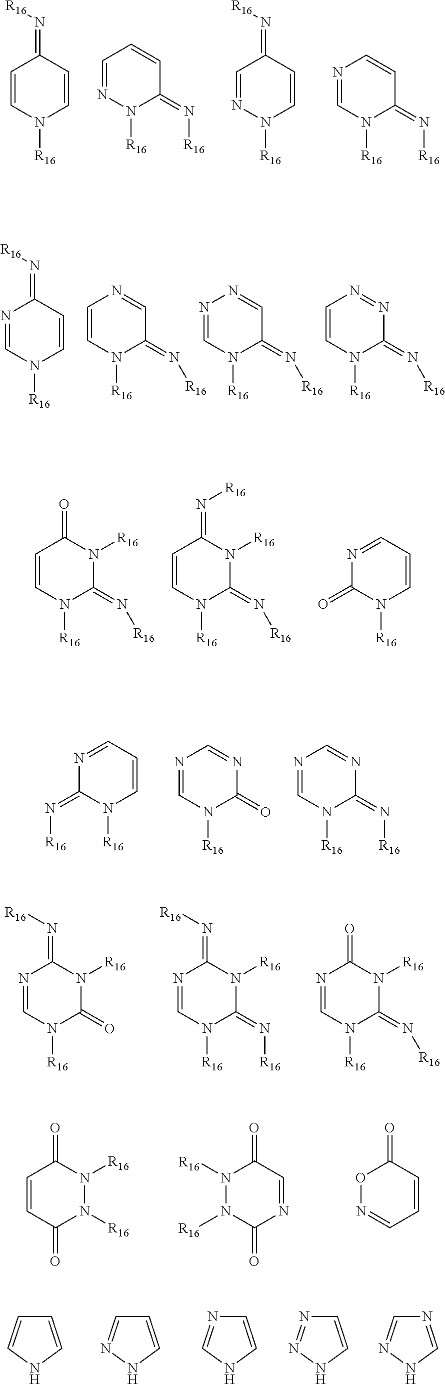 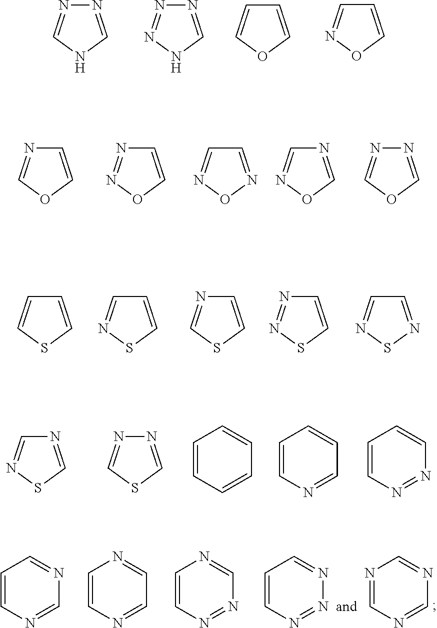
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system; and
one of X and Y is —O—, —NR₈—, —(N(OR₁₁)—, —(N(NR₆R₇)—, —B(OR₁₁)—, —S(O)ₓ—, —S(O)(═NR₈)—, —P(R₁₃)—, —P(O)(R₁₃)—, —P(O)(OR₁₁)—, —C(O)NR₈—, —SO₂NR₈—, —S(O)(R₁₃)═N—, —S(O)(═NR₆)N(R₇)—, —S(O)(N(R₆R₇)═N—, —P(O)(R₁₃)O—, —P(O)(OR₁₁)O—, —B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—

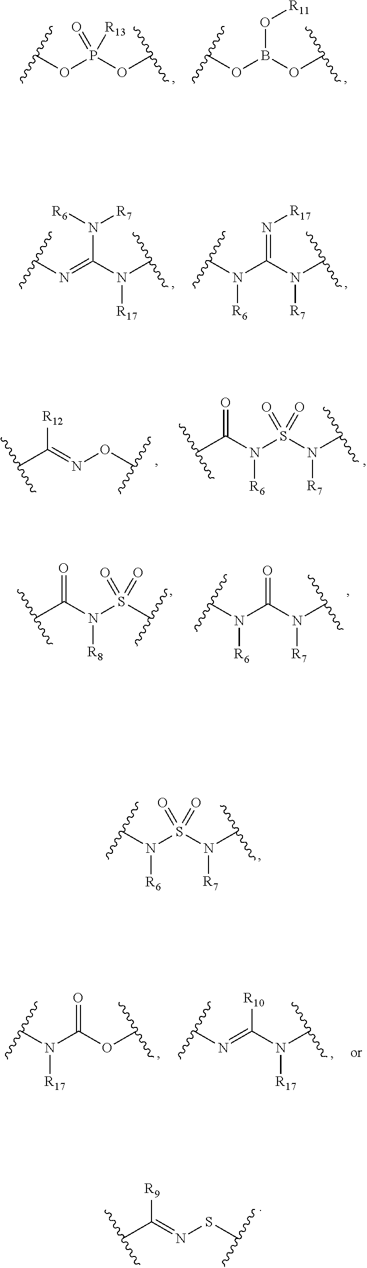

33. The compound of claim 32, or a pharmaceutically acceptable salt thereof, wherein:
G₁ is a bond;
X is O or NR₈;
G₂ is a bond or (CR₉R₁₀)ₒ;
Y is a divalent ring system selected from the group consisting of: 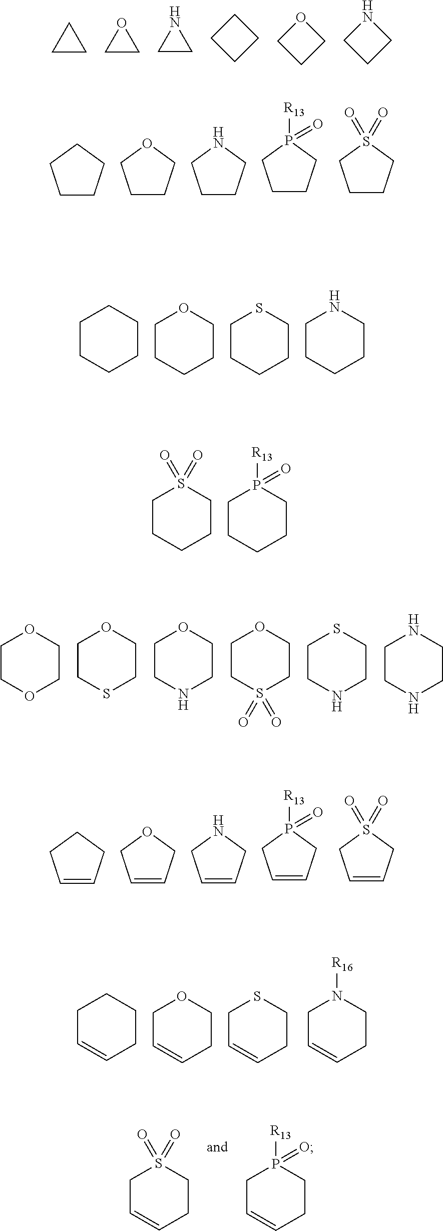
wherein the adjoining groups are attached to the divalent ring system in a 1,2- or 1,3-relationship; wherein the divalent ring system is optionally substituted with 1-3 R₁₅ groups.

34. The compound of any one of claims 1-15, or a pharmaceutically acceptable salt thereof, wherein:
G is —X-G₃- or -G₁-Y—;
G₁ and G₃ are each —(CR₉R₁₀)ₒ—, —C(═O)—, —C(═NR₈)—, —C(═NOR₁₁)—, —C(═NNR₆R₇)—, —CF₂—, —O(CR₉R₁₀)ₒ—, —S(O)ₓ(CR₉R₁₀)ₒ—, or —NR₈(CR₉R₁₀)ₒ—; and
X and Y are each a divalent ring system selected from the group consisting of: 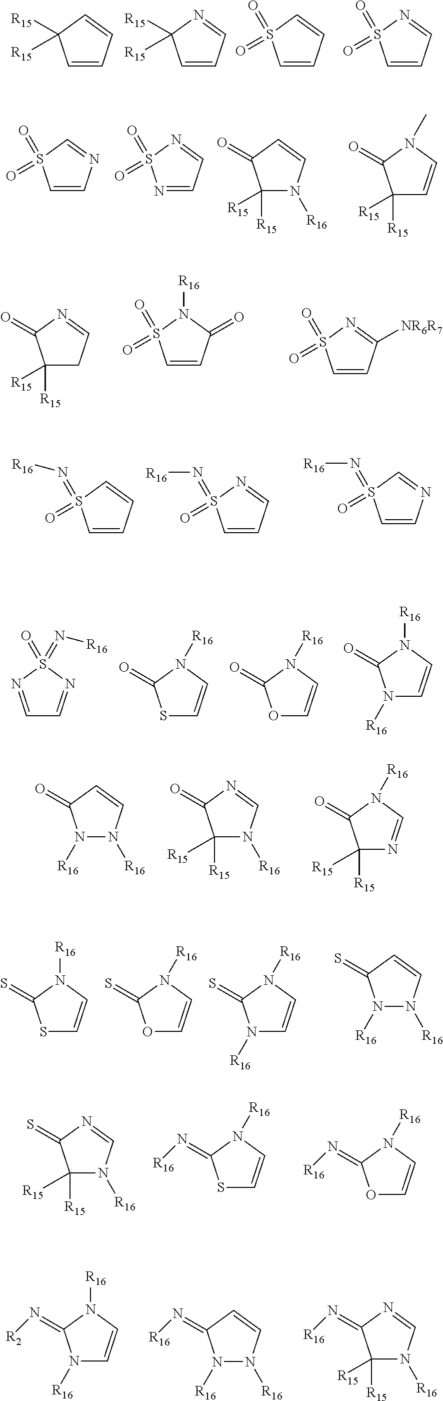 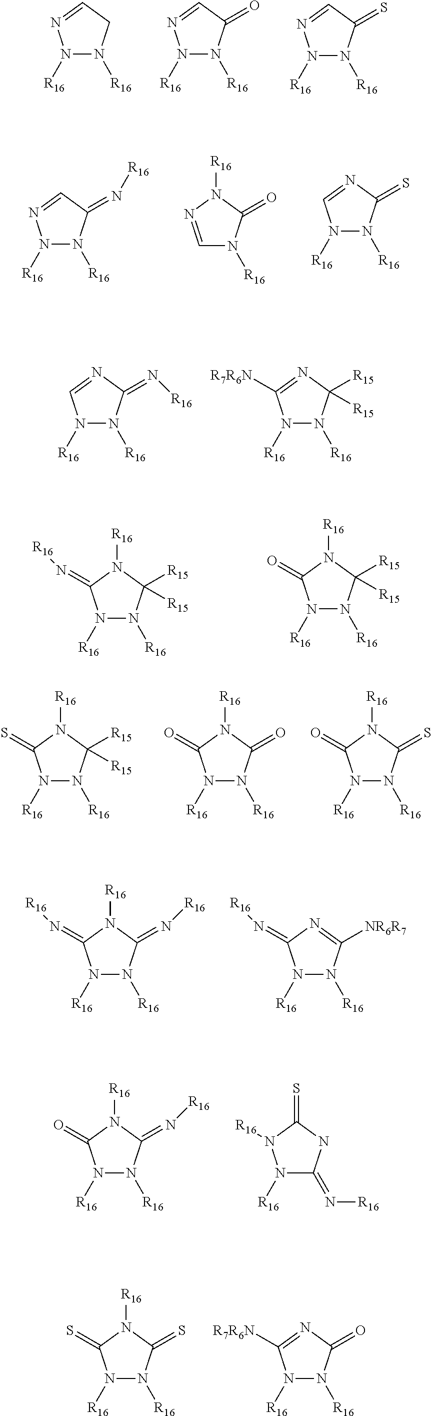 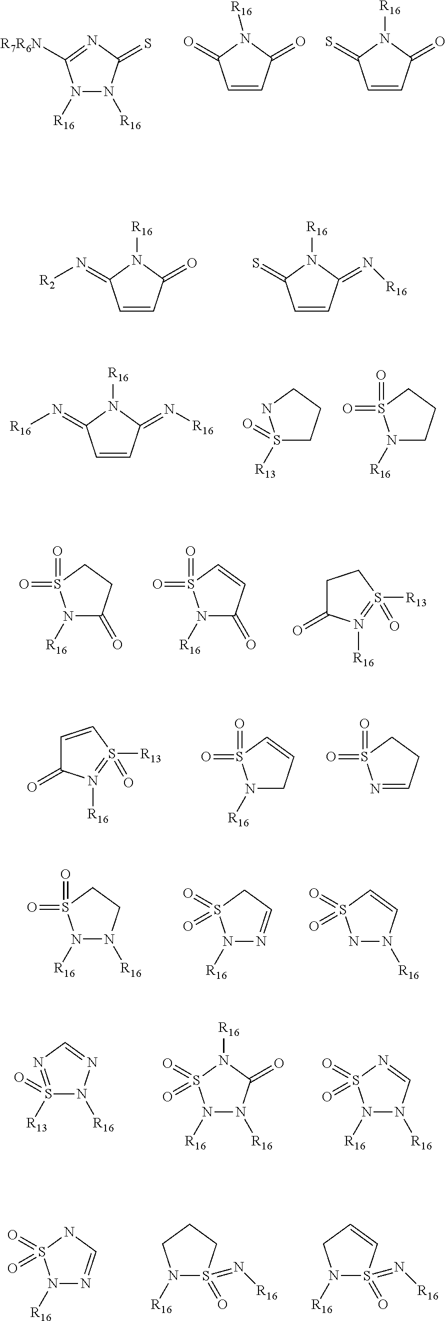 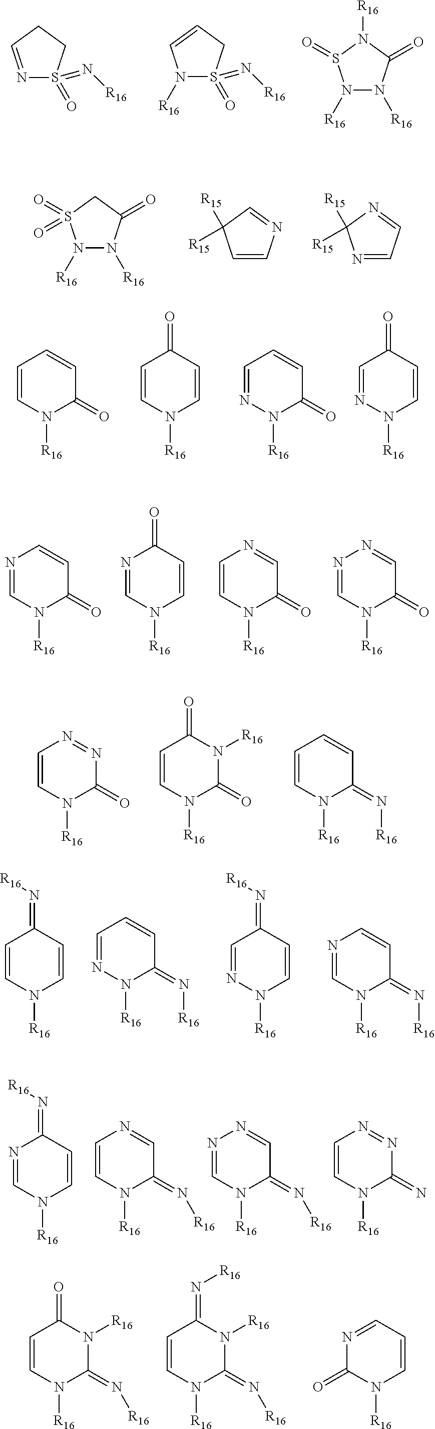 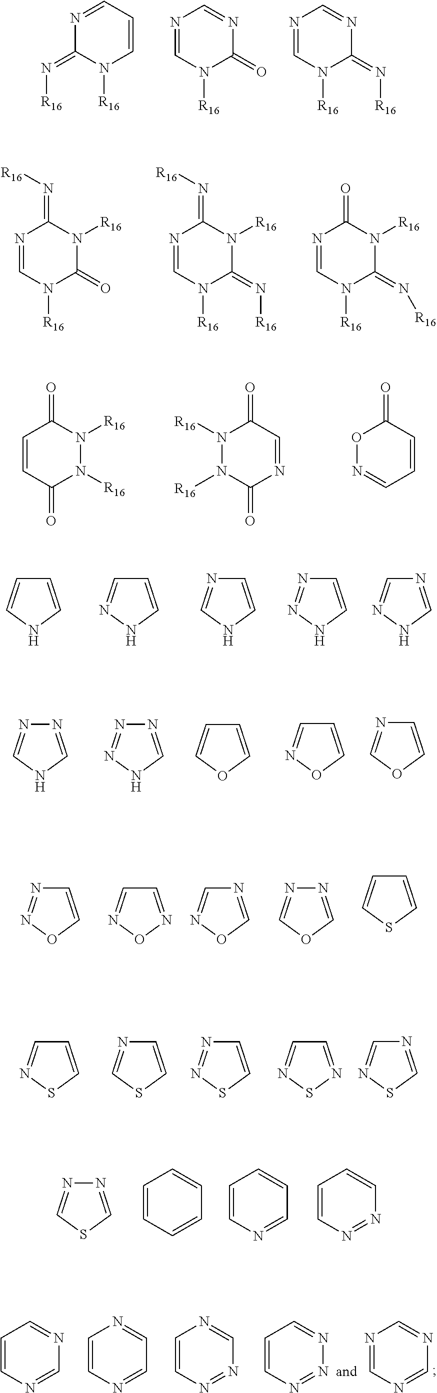
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system.

35. The compound of claim 34, or a pharmaceutically acceptable salt thereof, wherein:
G₁ and G₃ are each —CR₉R₁₀—, —C(═O)—, —C(═NR₈)—, —C(═NOR₁₁)—, —C(═NNR₆R₇)—, —CF₂—, —O—, —S(O)ₓ—, or —NR₈—; and
X and Y are each a divalent ring system selected from the group consisting of: 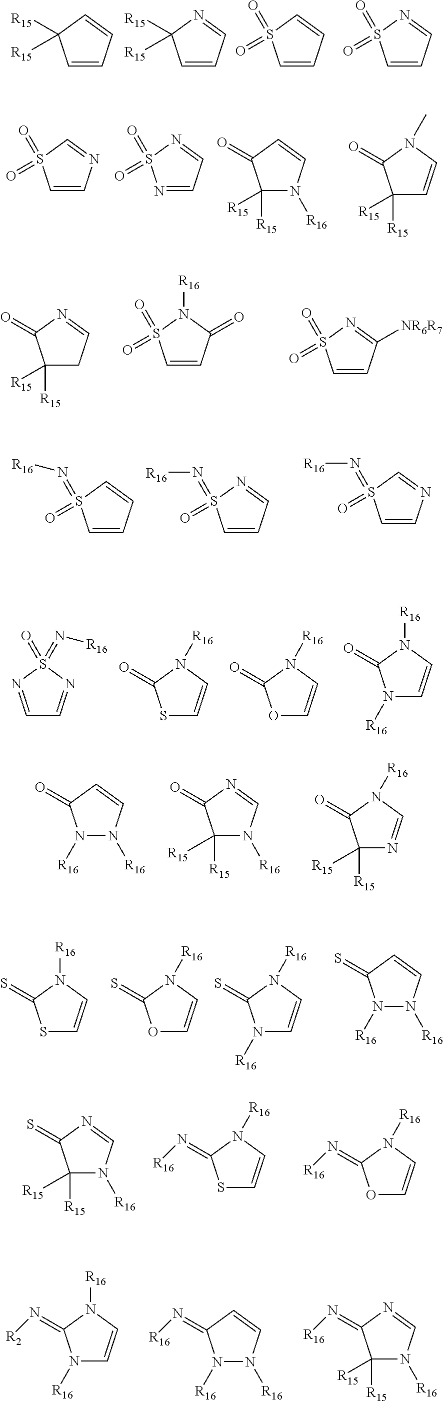 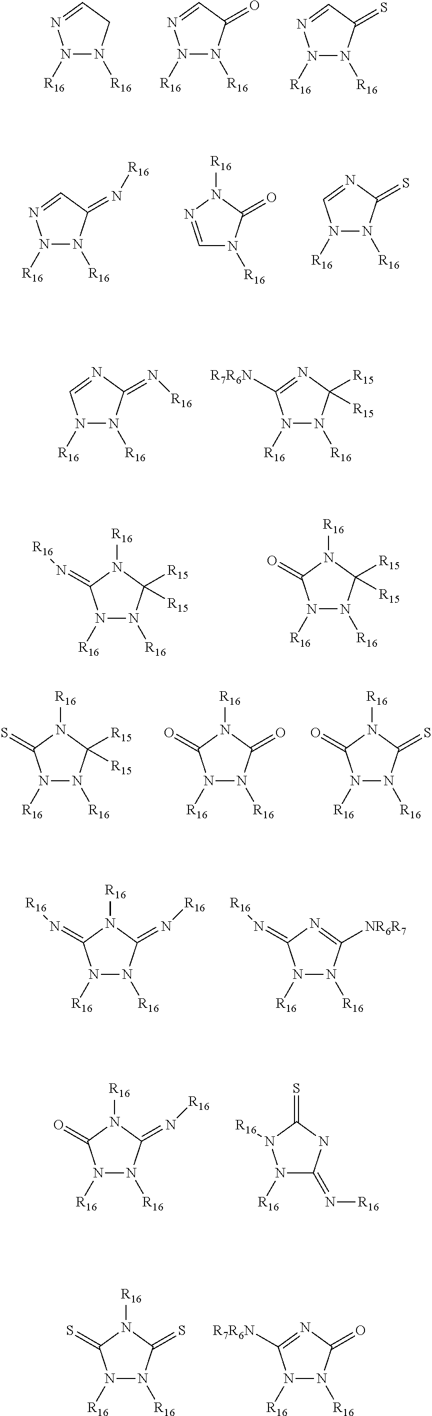 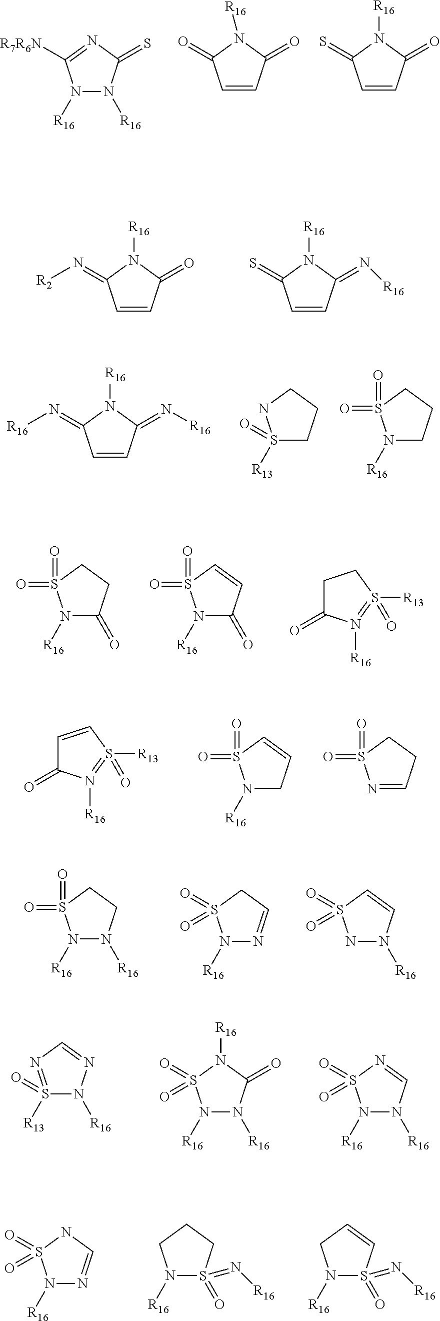 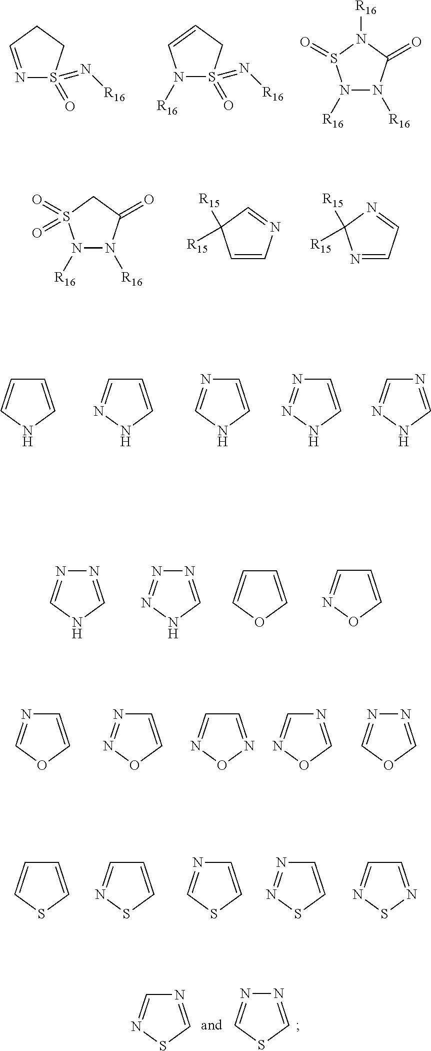
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system.

36. The compound of any one of claims 1-15, or a pharmaceutically acceptable salt thereof, wherein:
G is -G₁-X— or —X-G₃-; and
G contains 2 atoms in the direct chain that links A2 and A3; with the proviso that X is not a divalent ring system selected from the group consisting of: 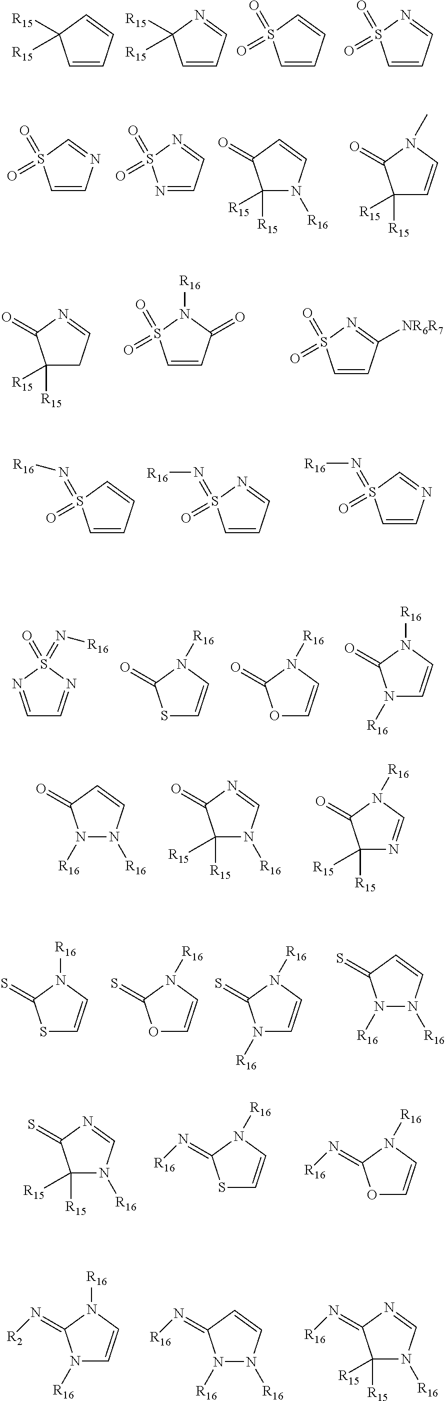 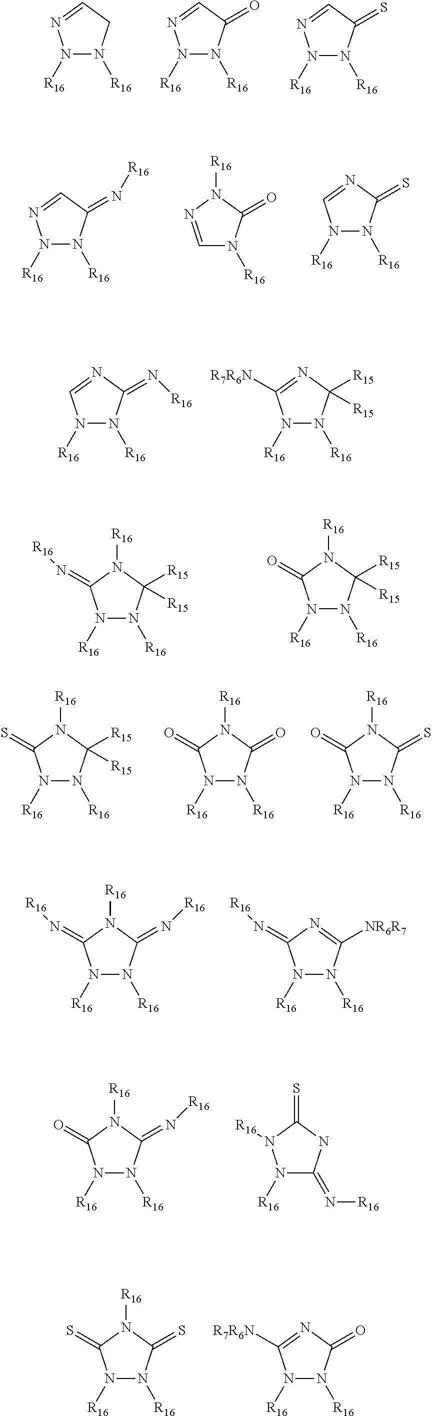 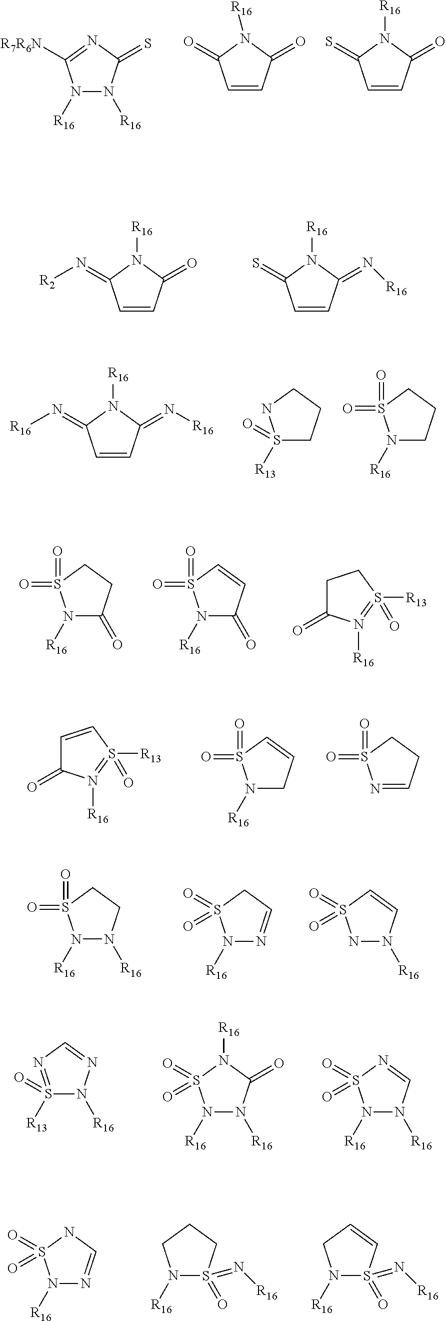 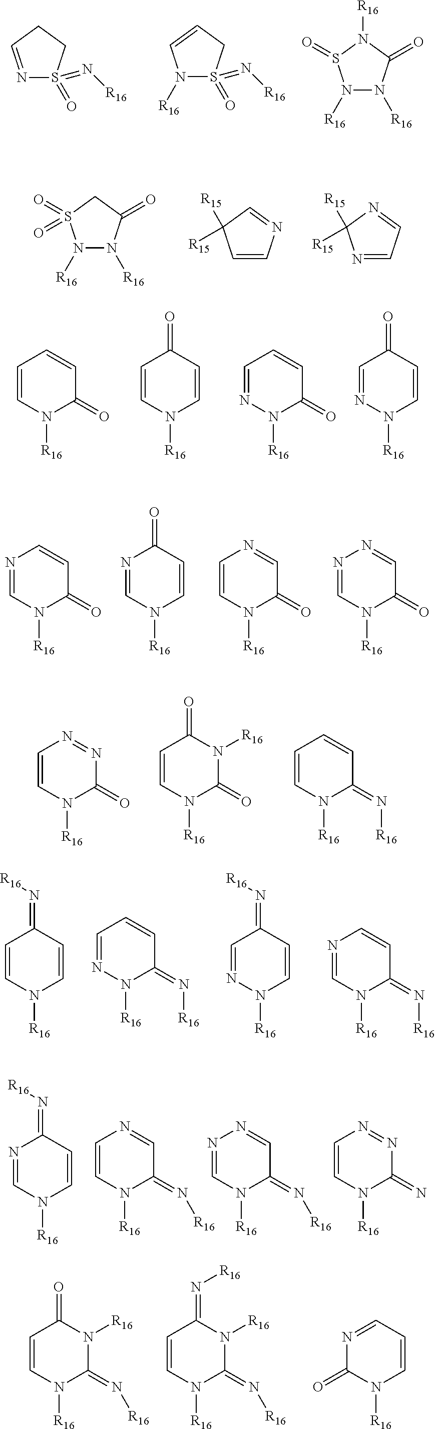 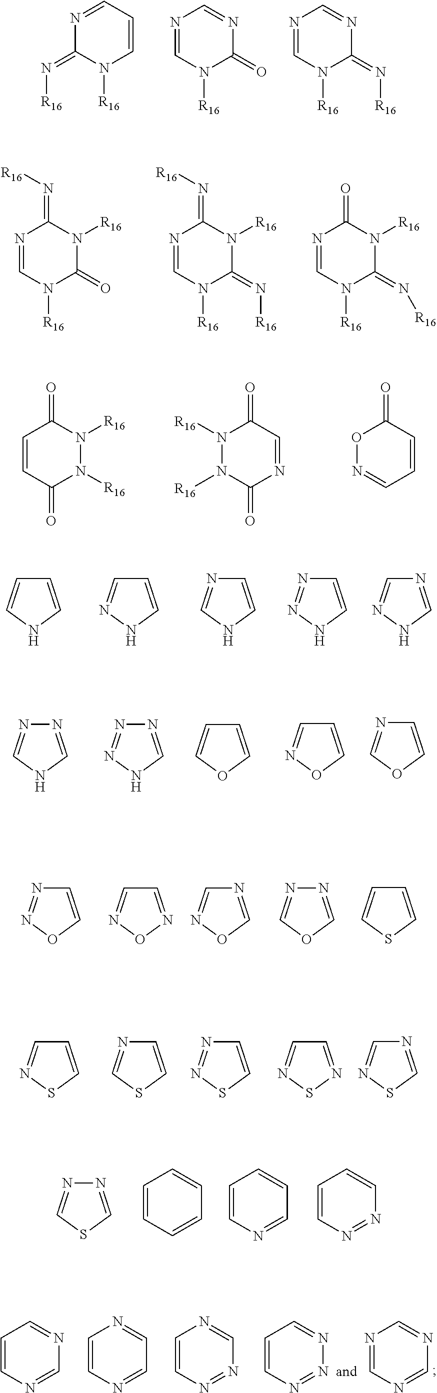
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system.

37. The compound of claim 1-15, or a pharmaceutically acceptable salt thereof, wherein:
G is X; and
X is —SO₂NR₈—, —S(O)(R₁₃)═N—, —S(O)(═NR₆)N(R₇)—, —S(O)(N(R₆R₇))═N—, —P(O)(R₁₃)O—, —P(O)(OR₁₁)O—, B(OR₁₁)O—, —N(R₆)N(R₇)—, —N(R₁₇)N(R₇)—, —N═N—, —N(R₁₇)O—, —C(R₁₂)═N—, —C(NR₁₇)═N—, —C(OR₁₁)═N—, —C(═NR₆)N(R₇)—, —C(═NR₁₇)N(R₁₇)—, 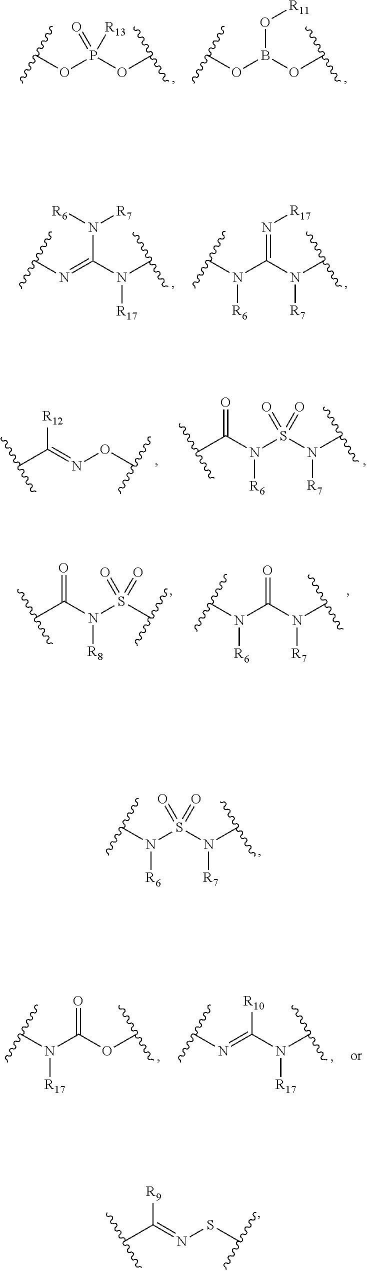
or a divalent ring system selected from the group consisting of: 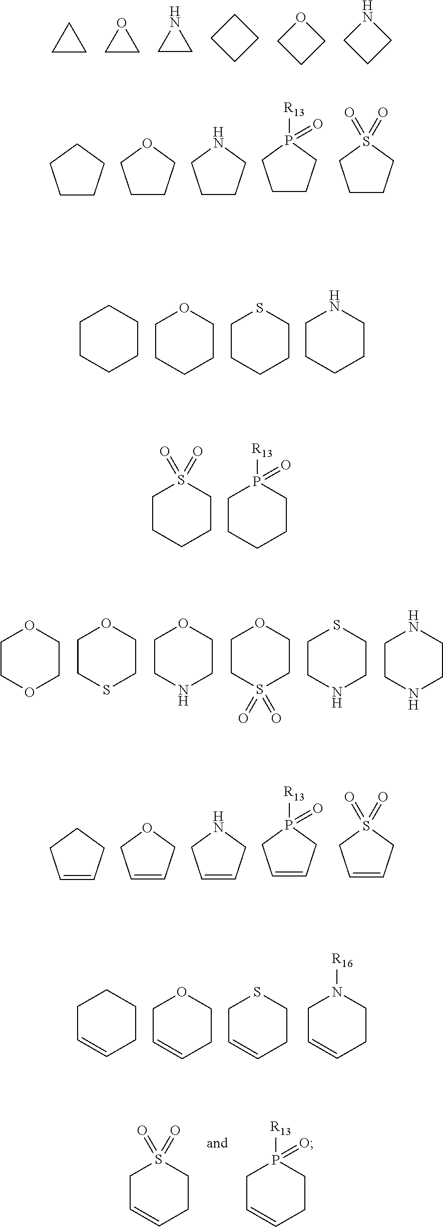
wherein the adjoining groups are attached to the divalent ring system in a 1,2- or 1,3-relationship; wherein the divalent ring system is optionally substituted with 1-3 R₁₅ groups.

38. The compound of any one of claims 1-15, or a pharmaceutically acceptable salt thereof, wherein:
G is -G₁-X—;
G₁ is —CH₂—; and
X is a divalent ring system selected from the group consisting of: 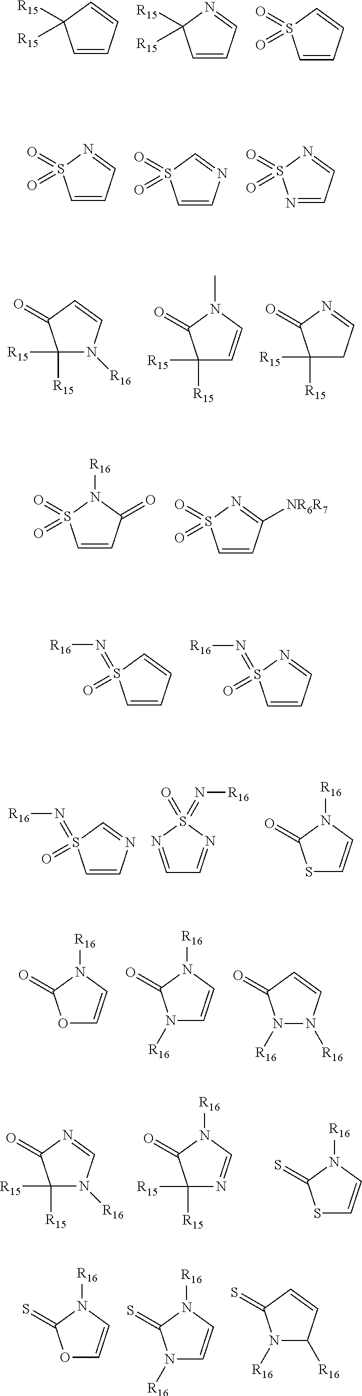 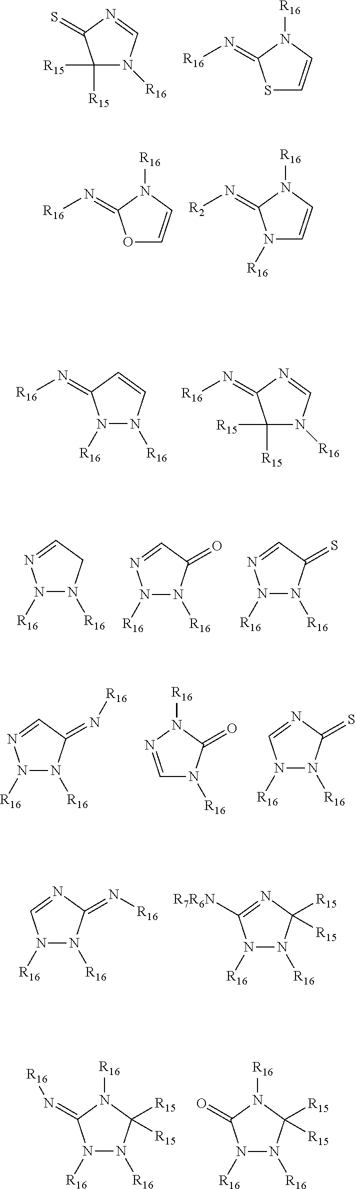 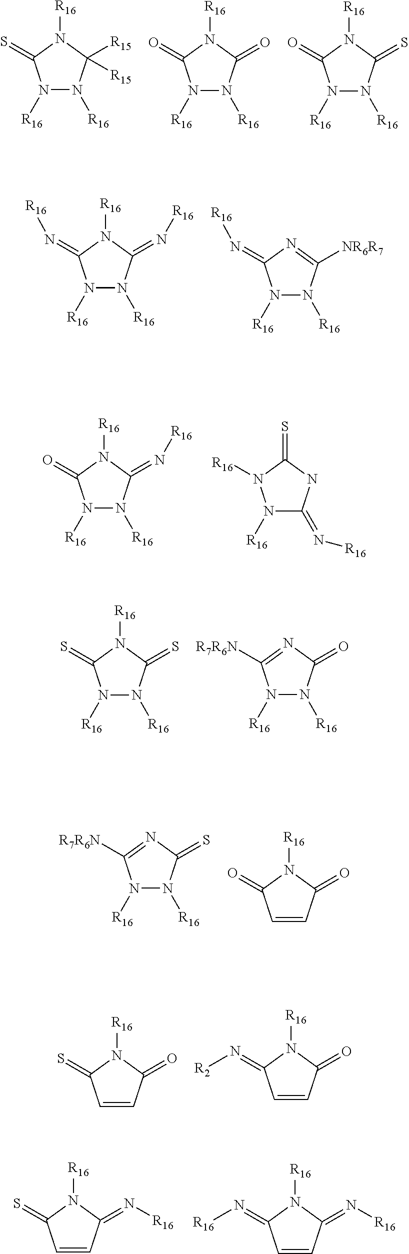 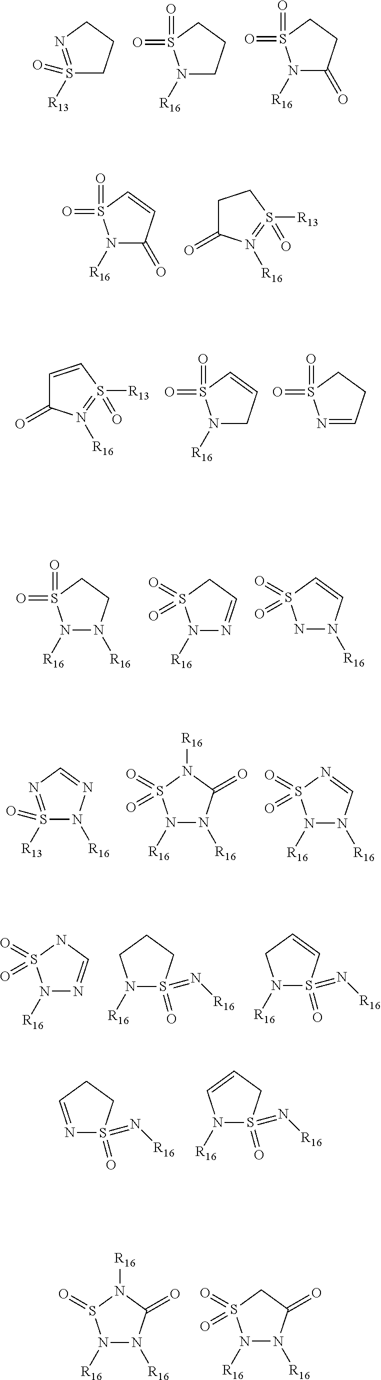 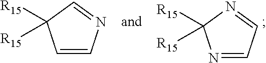
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system.

39. The compound of any one of claims 1-15, or a pharmaceutically acceptable salt thereof, wherein:
G is -G₁-X—;
G₁ is —CH₂—; and
X is a divalent ring system selected from the group consisting of: 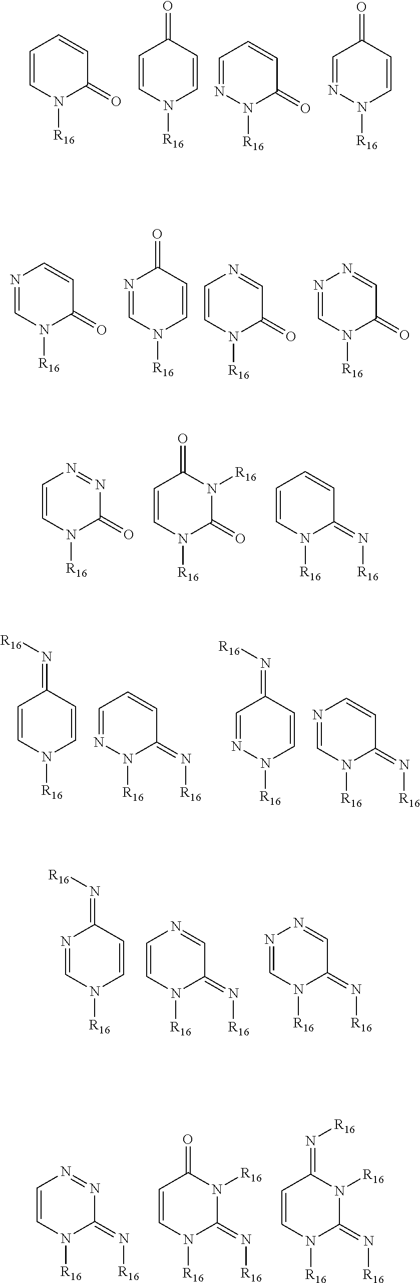 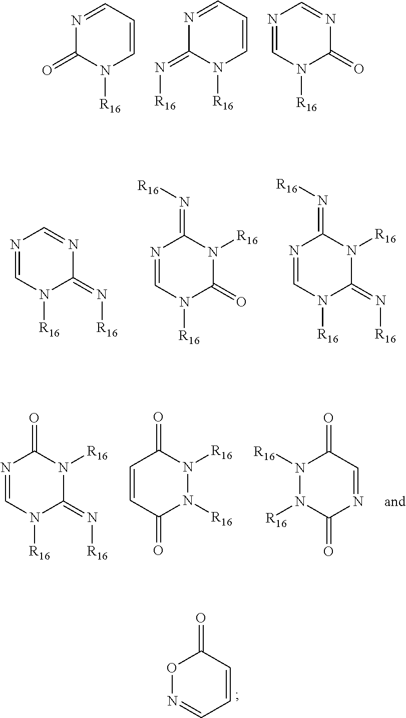
wherein the adjoining groups are attached to the divalent ring system in a 1,2- or 1,3-relationship; wherein the divalent ring system is optionally substituted with 1-3 R₁₅ groups; wherein said 1-3 R₁₅ groups include but are not limited to the R₁₅ groups shown in the structures of the divalent ring system.

40. A compound of the formula (XI) 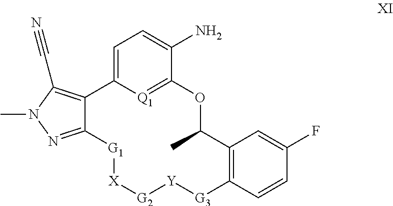
or a pharmaceutically acceptable salt thereof, wherein:
Q₁ is CH or N;
G₁ is —CR₉R₁₀— or a bond;
X is a bond;
G₂ is —(CR₉R₁₀)₂— or —CR₉═CR₁₀—;
Y is a bond;
G₃ is —CR₉R₁₀— or a bond;
each R₉ is independently H or C₁-C₆ alkyl; and
each R₁₀ is independently H or C₁-C₆ alkyl.

41. A compound of the formula (XI) 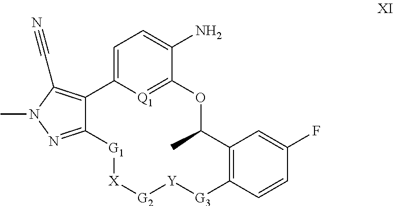
or a pharmaceutically acceptable salt thereof, wherein:
Q₁ is CH or N;
G₁ is —CH₂—, —CH₂CH₂— or a bond;
X is a bond;
G₂ is a bond;
Y is —SO₂—, —S(═O)(═NR₈)—, —SO₂NR₈—, —S(═O)(═NR₆)NR₇—, —N═S(═O)(R₁₃)—, —N═S(═O)(NR₆R₇)—, —N═S(═O)(R₁₃)N(R₇)—, —N(R₈)S(═O₂)N(R₇)—, —N(R₈)C(O)N(R₇)—, —N(R₈)C(O)O—, —N(R₇)C(═NR₆)—, —N═C(N(R₁₇)₂)N(R₈)—, —N═C(N(R₁₇)₂)—, —N(R₈)N(R₇)—, —N(R₈)O—, —N(R₈)S—, —C(O)N(R₈)SO₂—, —C(O)N(R₈)C(O)—, —C(O)N(R₈)N(R₇)—, —C(O)N(R₈)O—, —C(O)N(R₈)S—, —P(═O)(R₁₃)—, —P(═O)(OR₁₁)O—, —OP(═O)(R₁₃)O—, —B(OH)—, or —B(R₁₃)O—;
G₃ is —CH₂—, —CH₂CH₂—, or a bond; and
R₆ is H, C₁-C₆ alkyl, or C₁-C₆ acyl;
R₇ is H or C₁-C₆ alkyl;
each R₈ is independently H, C₁-C₆ alkyl, —CH₂CH₂OH, —CH₂C(CH₃)₂OH;
R₁₁ is H or C₁-C₆ alkyl;
R₁₃ is C₁-C₆ alkyl or —CH₂CH₂OH; and
each R₁₇ is independently C₁-C₆ alkyl, or two R₁₇ groups together with the nitrogen atom to which they are attached form a 5-6 membered heterocyclyl.

42. A compound of the formula (XI) 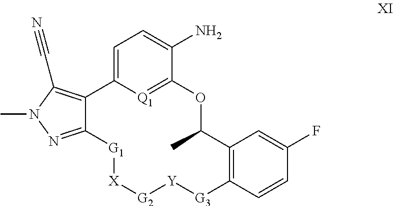
or a pharmaceutically acceptable salt thereof, wherein:
Q₁ is CH or N;
G₁ is a bond;
X is —O— or —NR₈—;
G₂ is —(CR₉R₁₀)₂—, —(CR₉R₁₀)₃—, or —CR₉═CR₁₀—;
Y is —O— or —NR₈—;
G₃ is a bond;
each R₈ is independently H or C₁-C₆ alkyl;
each R₉ is independently H or C₁-C₆ alkyl; and
each R₁₀ is independently H or C₁-C₆ alkyl.

43. A compound of the formula (XII) 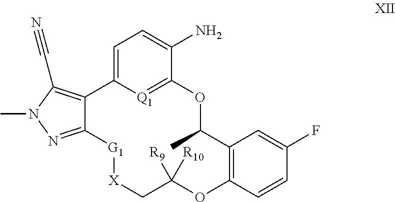
or a pharmaceutically acceptable salt thereof, wherein:
Q₁ is CH or N;
G₁ is CH₂ or a bond;
X is O, NR₈, or a bond;
R₈ is H or C₁-C₆ alkyl; and
R₉ and R₁₀ together with the carbon atom to which they are attached form a 3-5 membered cycloalkyl or heterocyclyl.

44. A compound of the formula (XIII) 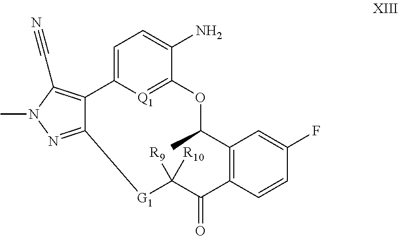
or a pharmaceutically acceptable salt thereof, wherein:
Q₁ is CH or N;
G₁ is —CH₂—, —O—, —NR₈—, —CH₂O—, or —CH₂NR₈—;
each R₈ is independently H or C₁-C₆ alkyl; and
R₉ and R₁₀ are each independently H, C₁-C₆ alkyl, —CH₂OH, or —CH₂N(R₈), or R₉ and R₁₀ together with the carbon atom to which they are attached form a 3-5 membered cycloalkyl or heterocyclyl.

45. A compound of the formula (XIV) 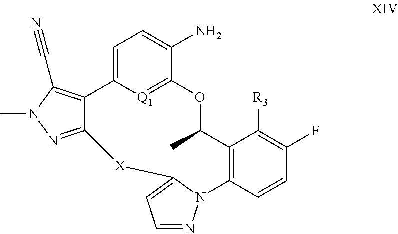
or a pharmaceutically acceptable salt thereof, wherein:
Q₁ is CH or N;
R₃ is H or Cl;
X is O or NR₈;
R₈ is H or C₁-C₆ alkyl.

46. A compound of the formula (XV) 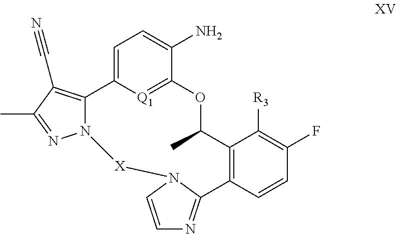
or a pharmaceutically acceptable salt thereof, wherein:
Q₁ is CH or N;
R₃ is H or Cl;
X is O, NR₈, or SO₂;
R₈ is H or C₁-C₆ alkyl.

47. A compound of the formula (XI) 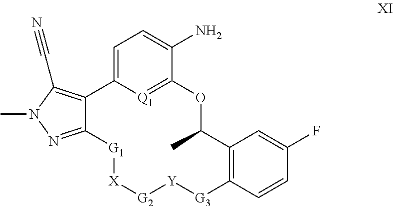
or a pharmaceutically acceptable salt thereof, wherein:
Q₁ is CH or N;
G₁ is —CH₂— or a bond;
X is O, NR₈, or a bond;
G₂ is selected from the group consisting of 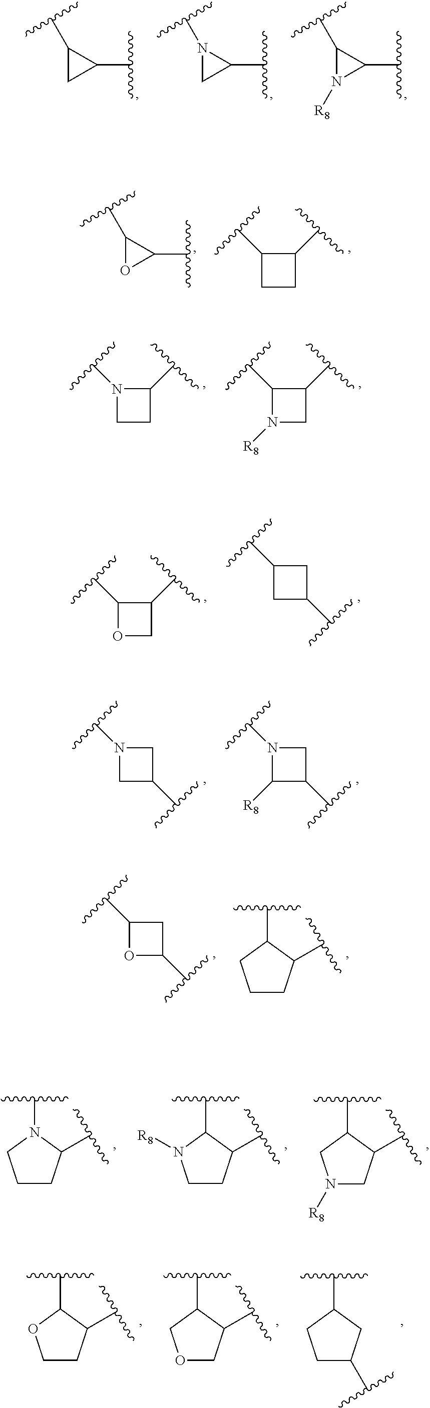 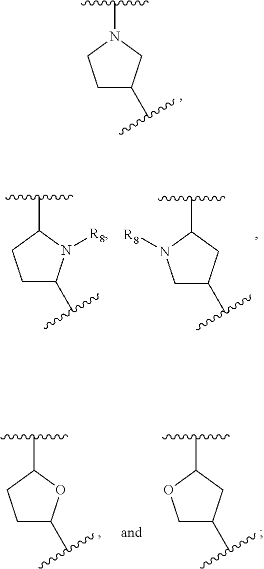
Y is O, NR₈, or a bond;
G₃ is —CH₂— or a bond; and
each R₈ is independently H or C₁-C₆ alkyl.

48. A compound of the formula (XI) 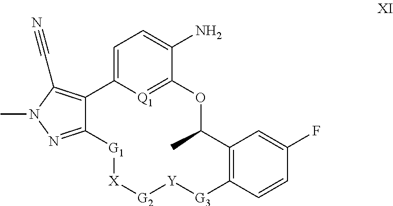
or a pharmaceutically acceptable salt thereof, wherein:
Q₁ is CH or N;
G₁ is —CH₂— or —CH₂CH₂—;
X is a bond;
G₂ is a bond;
Y is selected from the group consisting of 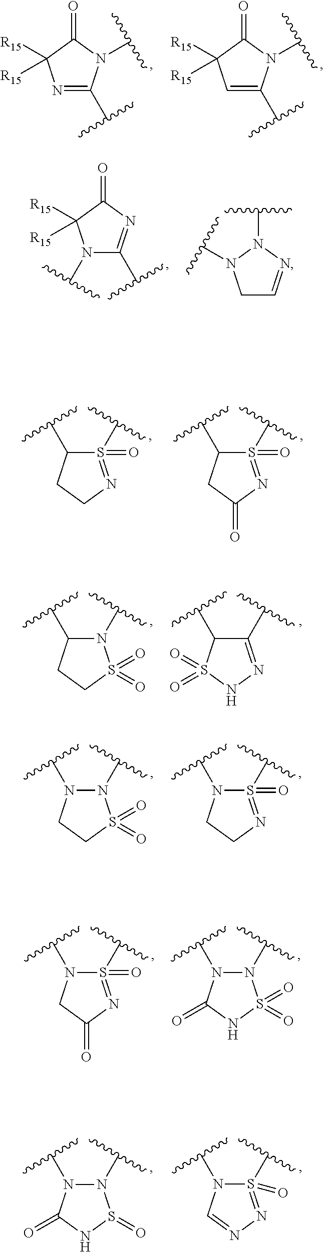 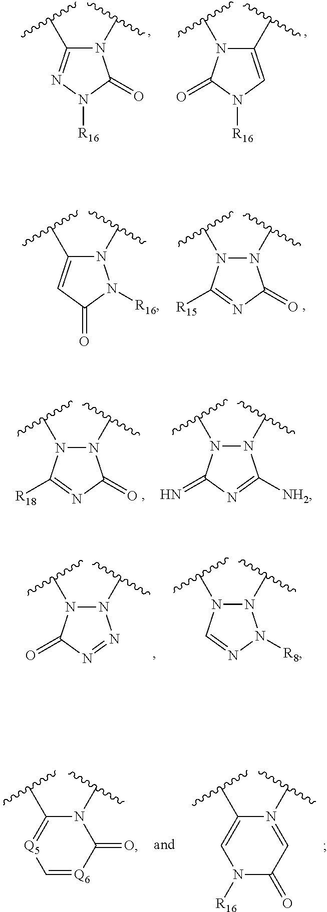
G₃ is a bond; and
Q₅ is CH or N;
Q₆ is CH or N;
each R₈ is independently H or C₁-C₆ alkyl;
each R₁₅ is —CH₃ or —CH₂OH; and
each R₁₆ is C₁-C₄ alkyl; and
each R₁₈ is —OH or —NH₂.

49. A compound of the formula (XVI) 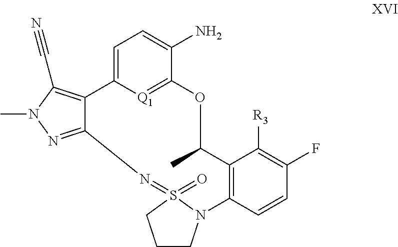
or a pharmaceutically acceptable salt thereof, wherein:
Q₁ is CH or N; and
R₃ is H or Cl.

50. A compound of the formula (XVII) 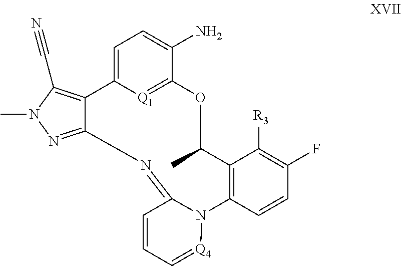
or a pharmaceutically acceptable salt thereof, wherein:
Q₁ is CH or N;
Q₄ is CH, or N; and
R₃ is H or Cl.

51. A compound of the formula (XVIII) 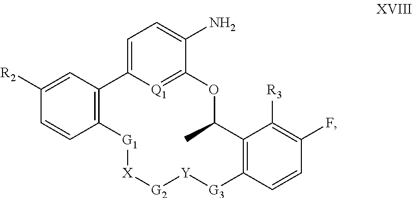
or a pharmaceutically acceptable salt thereof, wherein:
Q₁ is CH or N;
R₂ is —S(═O)(═NR₈)R₁₃ or —N═S(═O)(R₁₃)₂;
R₃ is H or Cl;
G₁ is —CH₂— or a bond;
X is —O—, —NR₈—, —C(O)NR₈—, or a bond;
G₂ is —CH₂CH₂— or a bond;
Y is —O—, —NR₈—, —C(O)NR₈—, a 5-6 membered hetereoarylene, or a bond;
G₃ is —CH₂— or a bond; wherein at least two but no more than three of G₁, G₂, G₃, X, and Y is a bond; wherein G₂ is —CH₂CH₂— when neither X nor Y is a bond;
R₈ is H or C₁-C₆ alkyl; and
R₁₃ is C₁-C₆ alkyl.

52. A compound of the formula (XIX) 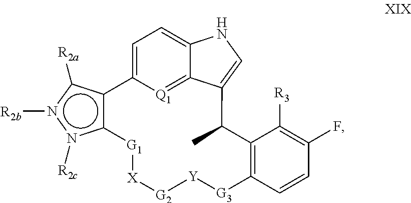
or a pharmaceutically acceptable salt thereof, wherein:
Q₁ is CH or N;
R₂ₐ is H, C₁-C₆ alkyl, or CN;
R_{2b} is H or C₁-C₆ alkyl, or is absent;
R_{2c} is H, C₁-C₆ alkyl, or —CH₂CH₂OH, or is absent; wherein one of R_{2b} and R_{2c} is absent;
R₃ is H or Cl;
G₁ is —CH₂— or a bond;
X is —O—, —NR₈—, —C(O)NR₈—, or a bond;
G₂ is —CH₂CH₂— or a bond;
Y is —O—, —NR₈—, —C(O)NR₈—, a 5-6 membered hereoarylene, or a bond;
G₃ is —CH₂— or a bond; wherein at least two but no more than three of G₁, G₂, G₃, X, and Y is a bond; wherein G₂ is —CH₂CH₂— when neither X nor Y is a bond; and
R₈ is H or C₁-C₆ alkyl.

53. A compound of the formula (XX) 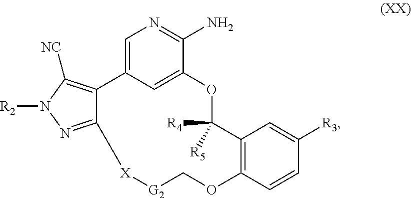
or a pharmaceutically acceptable salt thereof, wherein:
G₂ is —CH₂— or —C(O)—;
X is —O— or —NR₈—;
R₂ is C₁-C₄ alkyl;
R₃ is F or Cl;
R₄ and R₅ are each independently H, CH₃, CH₂CH₃, CH₂F, CHF₂, or CF₃; and
R₈ is H or C₁-C₄ alkyl.

54. The compound or salt of claim 53, wherein R₄ is CH₃; and R₅ is H.

55. The compound or salt of claim 53, wherein R₄ is H; and R₅ is CH₃.

56. A compound selected from the group consisting of: 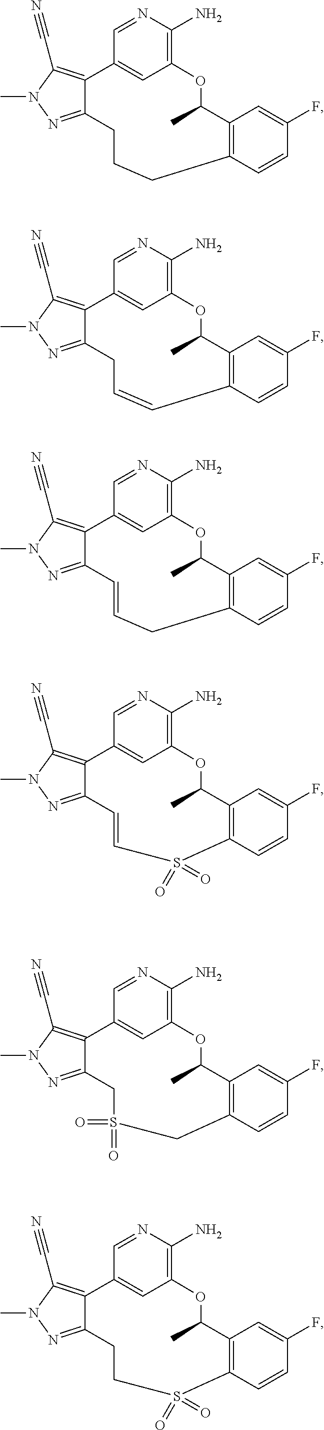 [SMILES: C[C@H]1Oc2cc(cnc2N)-c2c(nn(C)c2C#N)CCCc2ccc(F)cc21 ; C[C@H]1Oc2cc(cnc2N)-c2c(nn(C)c2C#N)C/C=C\c2ccc(F)cc21 ; C[C@H]1Oc2cc(cnc2N)-c2c(nn(C)c2C#N)/C=C/Cc2ccc(F)cc21 ; C[C@H]1Oc2cc(cnc2N)-c2c(nn(C)c2C#N)/C=C/S(=O)(=O)c2ccc(F)cc21 ; C[C@H]1Oc2cc(cnc2N)-c2c(nn(C)c2C#N)CS(=O)(=O)Cc2ccc(F)cc21 ; C[C@H]1Oc2cc(cnc2N)-c2c(nn(C)c2C#N)CCS(=O)(=O)c2ccc(F)cc21] 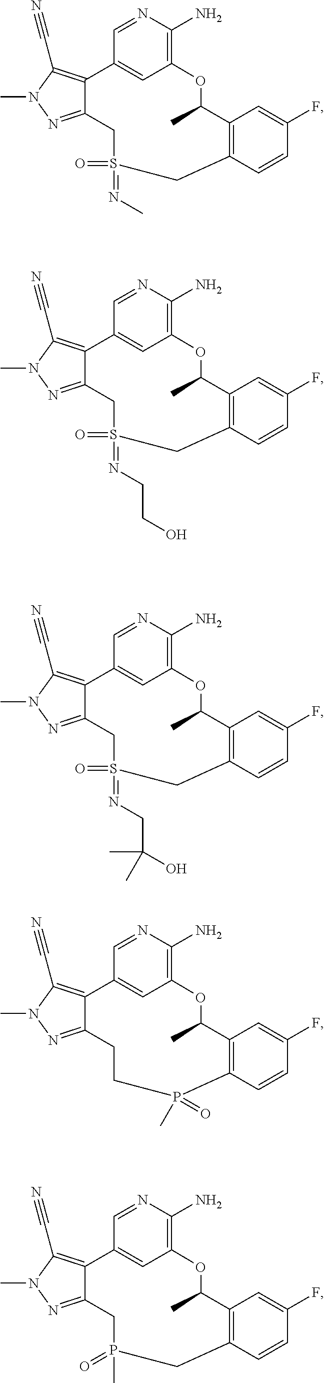 [SMILES: CN=S1(=O)Cc2ccc(F)cc2[C@@H](C)Oc2cc(cnc2N)-c2c(nn(C)c2C#N)C1 ; C[C@H]1Oc2cc(cnc2N)-c2c(nn(C)c2C#N)CS(=O)(=NCCO)Cc2ccc(F)cc21 ; C[C@H]1Oc2cc(cnc2N)-c2c(nn(C)c2C#N)CS(=O)(=NCC(C)(C)O)Cc2ccc(F)cc21 ; C[C@H]1Oc2cc(cnc2N)-c2c(nn(C)c2C#N)CCP(C)(=O)c2ccc(F)cc21 ; C[C@H]1Oc2cc(cnc2N)-c2c(nn(C)c2C#N)CP(C)(=O)Cc2ccc(F)cc21] 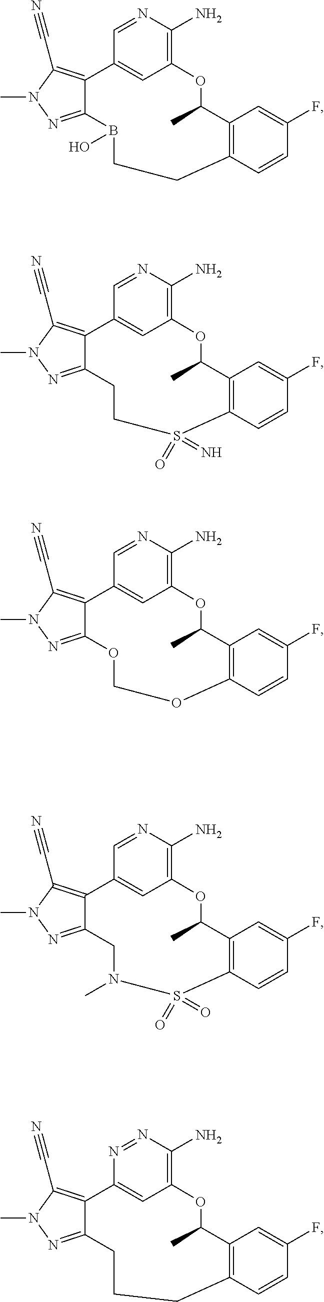 [SMILES: C[C@H]1Oc2cc(cnc2N)-c2c(nn(C)c2C#N)B(O)CCc2ccc(F)cc21 ; C[C@H]1Oc2cc(cnc2N)-c2c(nn(C)c2C#N)CCS(=N)(=O)c2ccc(F)cc21 ; C[C@H]1Oc2cc(cnc2N)-c2c(nn(C)c2C#N)OCOc2ccc(F)cc21 ; C[C@H]1Oc2cc(cnc2N)-c2c(nn(C)c2C#N)CN(C)S(=O)(=O)c2ccc(F)cc21 ; C[C@H]1Oc2cc(nnc2N)-c2c(nn(C)c2C#N)CCCc2ccc(F)cc21] 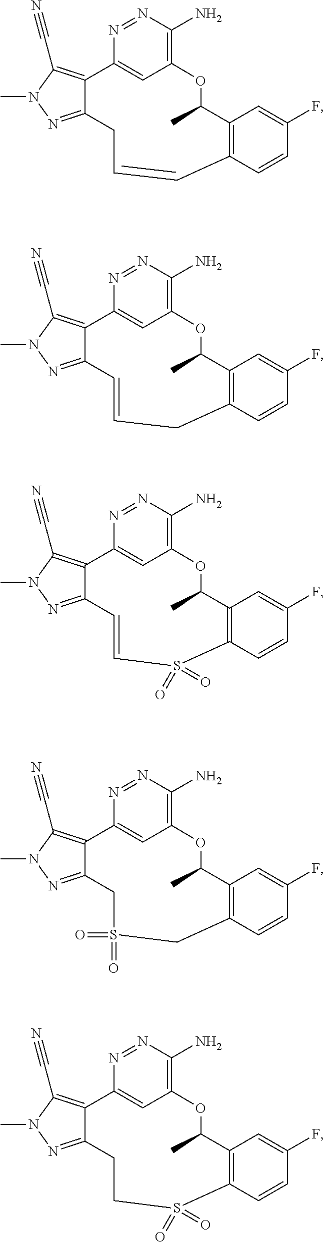 [SMILES: C[C@H]1Oc2cc(nnc2N)-c2c(nn(C)c2C#N)C/C=C\c2ccc(F)cc21 ; C[C@H]1Oc2cc(nnc2N)-c2c(nn(C)c2C#N)/C=C/Cc2ccc(F)cc21 ; C[C@H]1Oc2cc(nnc2N)-c2c(nn(C)c2C#N)/C=C/S(=O)(=O)c2ccc(F)cc21 ; C[C@H]1Oc2cc(nnc2N)-c2c(nn(C)c2C#N)CS(=O)(=O)Cc2ccc(F)cc21 ; C[C@H]1Oc2cc(nnc2N)-c2c(nn(C)c2C#N)CCS(=O)(=O)c2ccc(F)cc21] 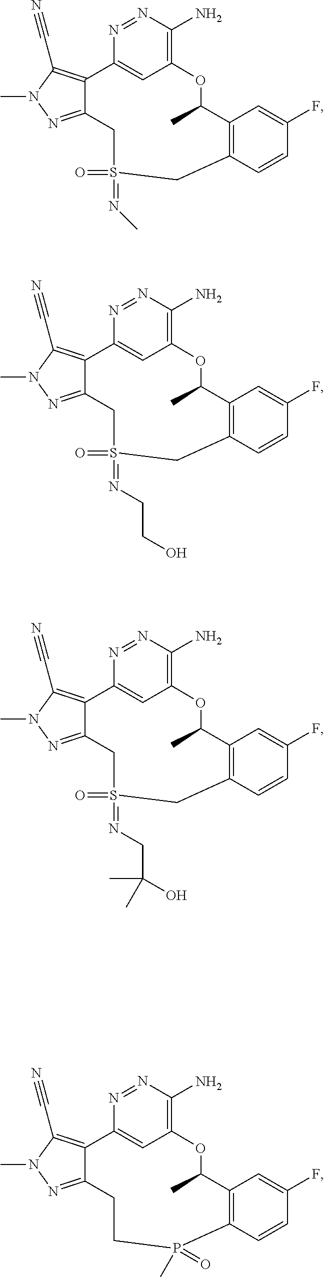 [SMILES: CN=S1(=O)Cc2ccc(F)cc2[C@@H](C)Oc2cc(nnc2N)-c2c(nn(C)c2C#N)C1 ; C[C@H]1Oc2cc(nnc2N)-c2c(nn(C)c2C#N)CS(=O)(=NCCO)Cc2ccc(F)cc21 ; C[C@H]1Oc2cc(nnc2N)-c2c(nn(C)c2C#N)CS(=O)(=NCC(C)(C)O)Cc2ccc(F)cc21 ; C[C@H]1Oc2cc(nnc2N)-c2c(nn(C)c2C#N)CCP(C)(=O)c2ccc(F)cc21] 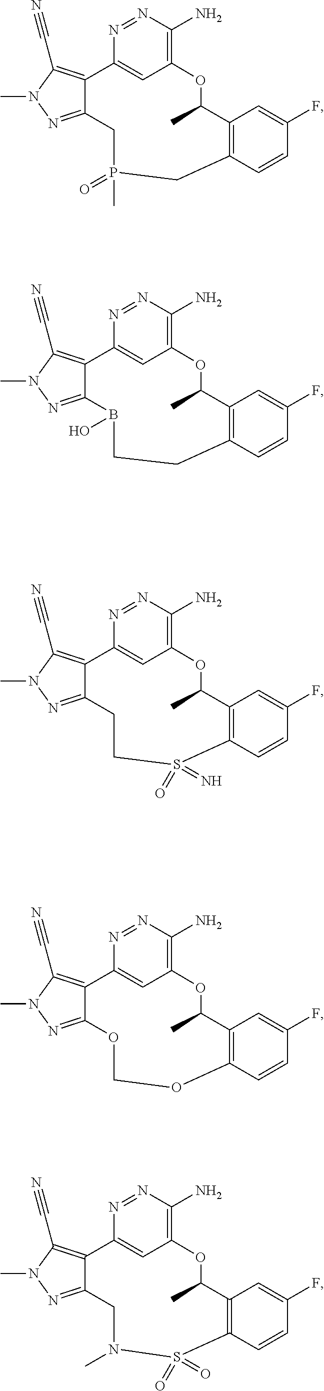 [SMILES: C[C@H]1Oc2cc(nnc2N)-c2c(nn(C)c2C#N)CP(C)(=O)Cc2ccc(F)cc21 ; C[C@H]1Oc2cc(nnc2N)-c2c(nn(C)c2C#N)B(O)CCc2ccc(F)cc21 ; C[C@H]1Oc2cc(nnc2N)-c2c(nn(C)c2C#N)CCS(=N)(=O)c2ccc(F)cc21 ; C[C@H]1Oc2cc(nnc2N)-c2c(nn(C)c2C#N)OCOc2ccc(F)cc21 ; C[C@H]1Oc2cc(nnc2N)-c2c(nn(C)c2C#N)CN(C)S(=O)(=O)c2ccc(F)cc21] 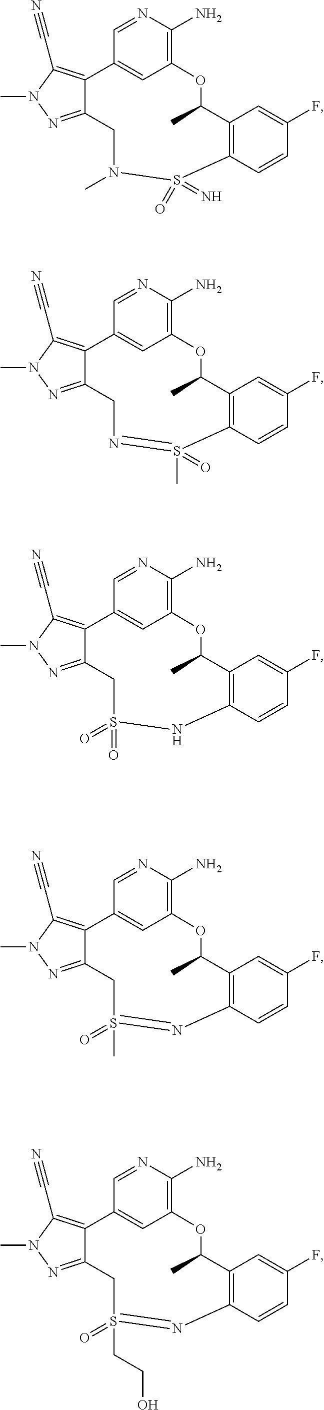 [SMILES: C[C@H]1Oc2cc(cnc2N)-c2c(nn(C)c2C#N)CN(C)S(=N)(=O)c2ccc(F)cc21 ; C[C@H]1Oc2cc(cnc2N)-c2c(nn(C)c2C#N)CN=S(C)(=O)c2ccc(F)cc21 ; C[C@H]1Oc2cc(cnc2N)-c2c(nn(C)c2C#N)CS(=O)(=O)Nc2ccc(F)cc21 ; C[C@H]1Oc2cc(cnc2N)-c2c(nn(C)c2C#N)CS(C)(=O)=Nc2ccc(F)cc21 ; C[C@H]1Oc2cc(cnc2N)-c2c(nn(C)c2C#N)CS(=O)(CCO)=Nc2ccc(F)cc21] 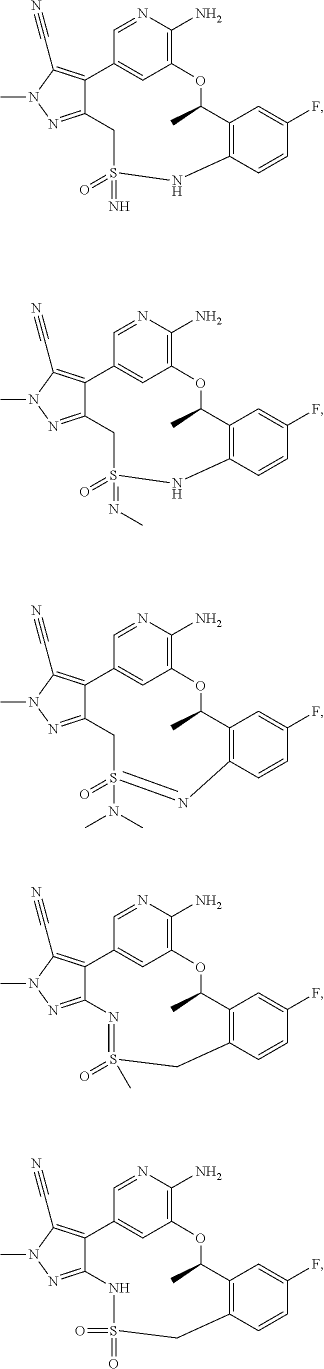 [SMILES: C[C@H]1Oc2cc(cnc2N)-c2c(nn(C)c2C#N)CS(=N)(=O)Nc2ccc(F)cc21 ; CN=S1(=O)Cc2nn(C)c(C#N)c2-c2cnc(N)c(c2)O[C@H](C)c2cc(F)ccc2N1 ; C[C@H]1Oc2cc(cnc2N)-c2c(nn(C)c2C#N)CS(=O)(N(C)C)=Nc2ccc(F)cc21 ; C[C@H]1Oc2cc(cnc2N)-c2c(nn(C)c2C#N)N=S(C)(=O)Cc2ccc(F)cc21 ; C[C@H]1Oc2cc(cnc2N)-c2c(nn(C)c2C#N)NS(=O)(=O)Cc2ccc(F)cc21] 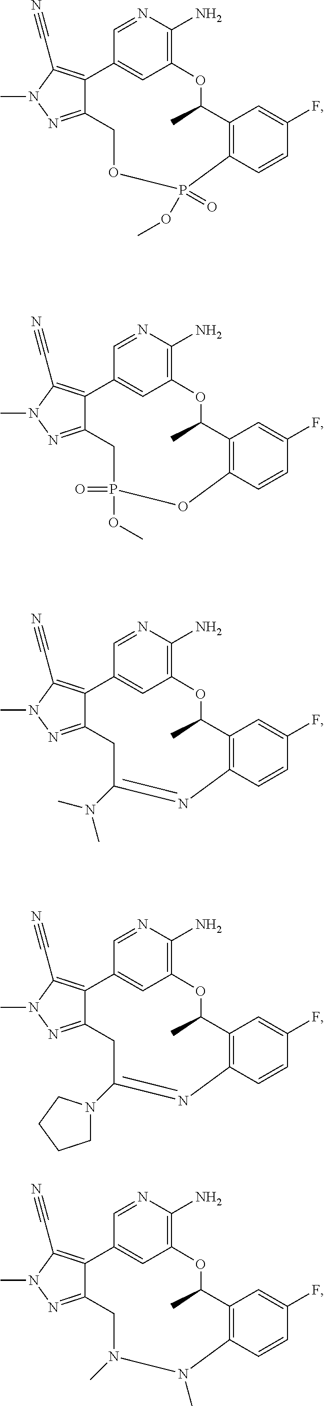 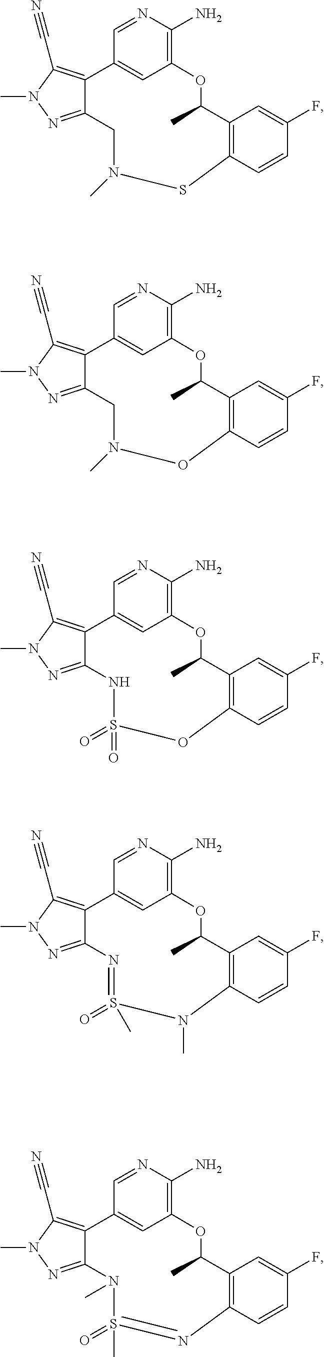 [SMILES: C[C@H]1Oc2cc(cnc2N)-c2c(nn(C)c2C#N)CN(C)Sc2ccc(F)cc21 ; C[C@H]1Oc2cc(cnc2N)-c2c(nn(C)c2C#N)CN(C)Oc2ccc(F)cc21 ; C[C@H]1Oc2cc(cnc2N)-c2c(nn(C)c2C#N)NS(=O)(=O)Oc2ccc(F)cc21 ; C[C@H]1Oc2cc(cnc2N)-c2c(nn(C)c2C#N)N=S(C)(=O)N(C)c2ccc(F)cc21 ; C[C@H]1Oc2cc(cnc2N)-c2c(nn(C)c2C#N)N(C)S(C)(=O)=Nc2ccc(F)cc21] 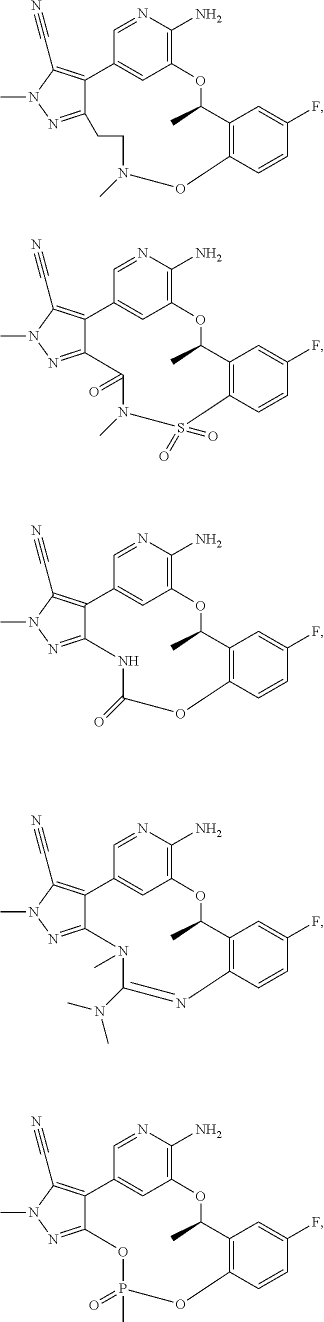 [SMILES: C[C@H]1Oc2cc(cnc2N)-c2c(nn(C)c2C#N)CCN(C)Oc2ccc(F)cc21 ; C[C@H]1Oc2cc(cnc2N)-c2c(nn(C)c2C#N)C(=O)N(C)S(=O)(=O)c2ccc(F)cc21 ; C[C@H]1Oc2cc(cnc2N)-c2c(nn(C)c2C#N)NC(=O)Oc2ccc(F)cc21 ; C[C@H]1Oc2cc(cnc2N)-c2c(nn(C)c2C#N)N(C)/C(N(C)C)=N\c2ccc(F)cc21 ; C[C@H]1Oc2cc(cnc2N)-c2c(nn(C)c2C#N)OP(C)(=O)Oc2ccc(F)cc21] 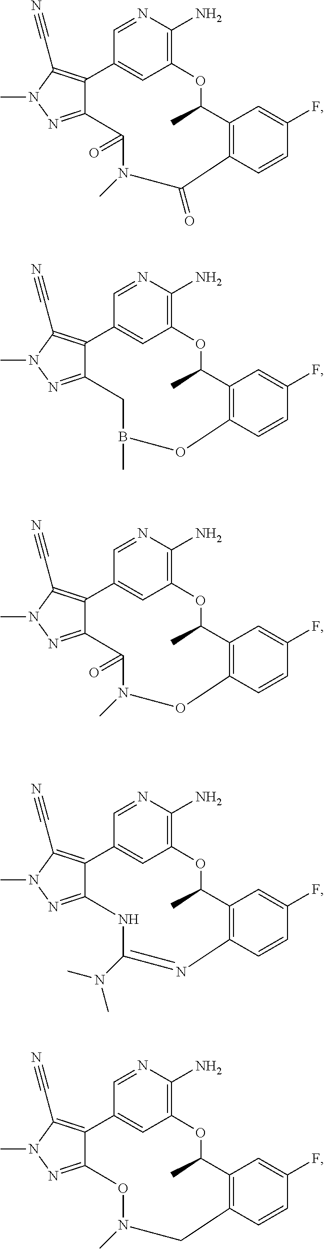 [SMILES: C[C@H]1Oc2cc(cnc2N)-c2c(nn(C)c2C#N)C(=O)N(C)C(=O)c2ccc(F)cc21 ; CB1Cc2nn(C)c(C#N)c2-c2cnc(N)c(c2)O[C@H](C)c2cc(F)ccc2O1 ; C[C@H]1Oc2cc(cnc2N)-c2c(nn(C)c2C#N)C(=O)N(C)Oc2ccc(F)cc21 ; C[C@H]1Oc2cc(cnc2N)-c2c(nn(C)c2C#N)N/C(N(C)C)=N\c2ccc(F)cc21 ; C[C@H]1Oc2cc(cnc2N)-c2c(nn(C)c2C#N)ON(C)Cc2ccc(F)cc21] 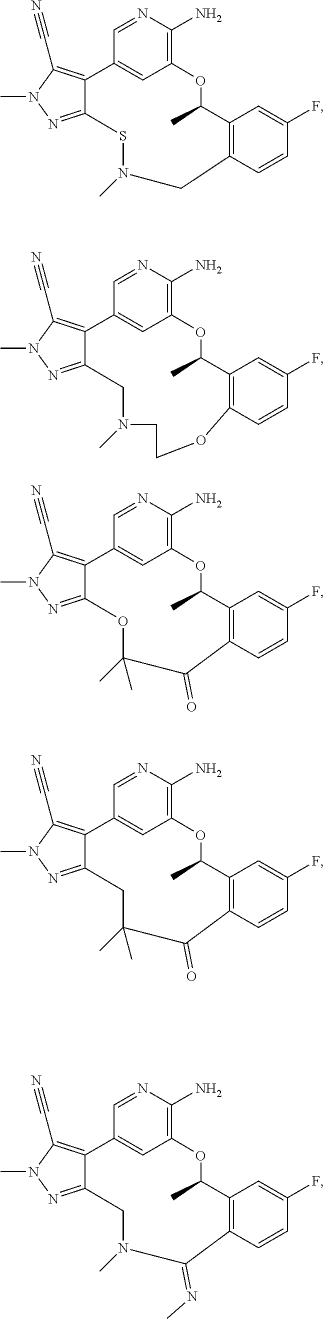 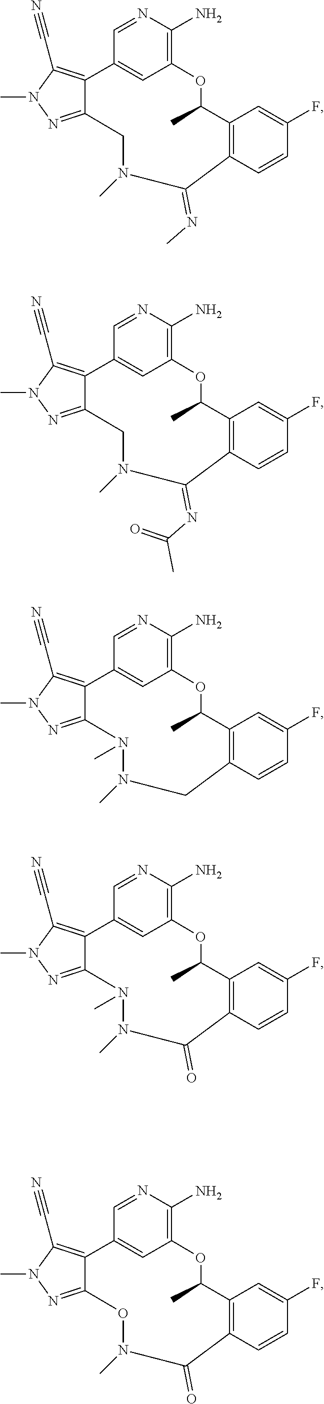 [SMILES: C/N=C1/c2ccc(F)cc2[C@@H](C)Oc2cc(cnc2N)-c2c(nn(C)c2C#N)CN1C ; CC(=O)/N=C1/c2ccc(F)cc2[C@@H](C)Oc2cc(cnc2N)-c2c(nn(C)c2C#N)CN1C ; C[C@H]1Oc2cc(cnc2N)-c2c(nn(C)c2C#N)N(C)N(C)Cc2ccc(F)cc21 ; C[C@H]1Oc2cc(cnc2N)-c2c(nn(C)c2C#N)N(C)N(C)C(=O)c2ccc(F)cc21 ; C[C@H]1Oc2cc(cnc2N)-c2c(nn(C)c2C#N)ON(C)C(=O)c2ccc(F)cc21] 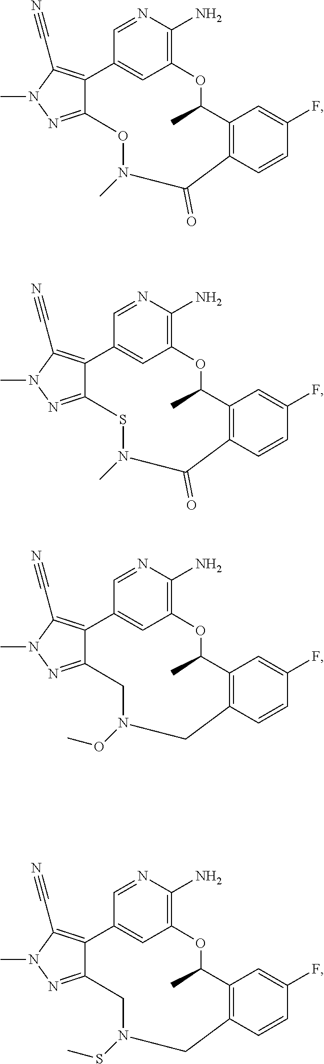 [SMILES: C[C@H]1Oc2cc(cnc2N)-c2c(nn(C)c2C#N)ON(C)C(=O)c2ccc(F)cc21 ; C[C@H]1Oc2cc(cnc2N)-c2c(nn(C)c2C#N)SN(C)C(=O)c2ccc(F)cc21 ; CON1Cc2ccc(F)cc2[C@@H](C)Oc2cc(cnc2N)-c2c(nn(C)c2C#N)C1 ; CSN1Cc2ccc(F)cc2[C@@H](C)Oc2cc(cnc2N)-c2c(nn(C)c2C#N)C1] 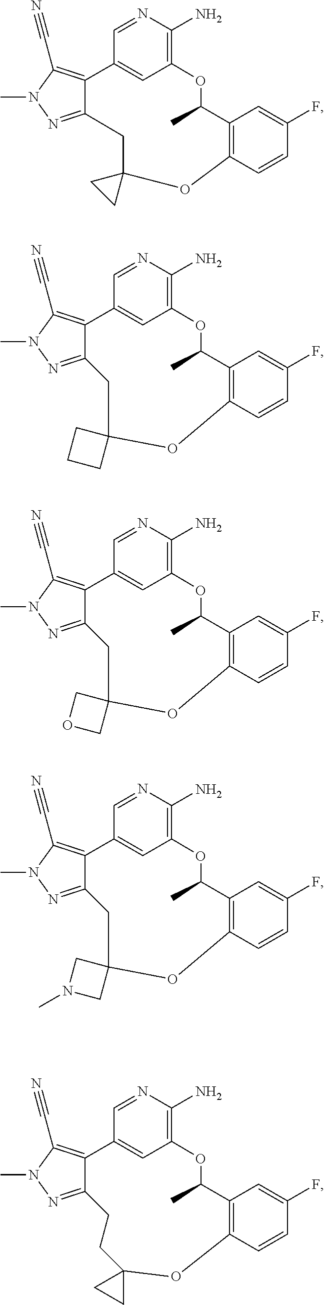 [SMILES: C[C@H]1Oc2cc(cnc2N)-c2c(nn(C)c2C#N)CC2(CC2)Oc2ccc(F)cc21 ; C[C@H]1Oc2cc(cnc2N)-c2c(nn(C)c2C#N)CC2(CCC2)Oc2ccc(F)cc21 ; C[C@H]1Oc2cc(cnc2N)-c2c(nn(C)c2C#N)CC2(COC2)Oc2ccc(F)cc21 ; C[C@H]1Oc2cc(cnc2N)-c2c(nn(C)c2C#N)CC2(CN(C)C2)Oc2ccc(F)cc21 ; C[C@H]1Oc2cc(cnc2N)-c2c(nn(C)c2C#N)CCC2(CC2)Oc2ccc(F)cc21] 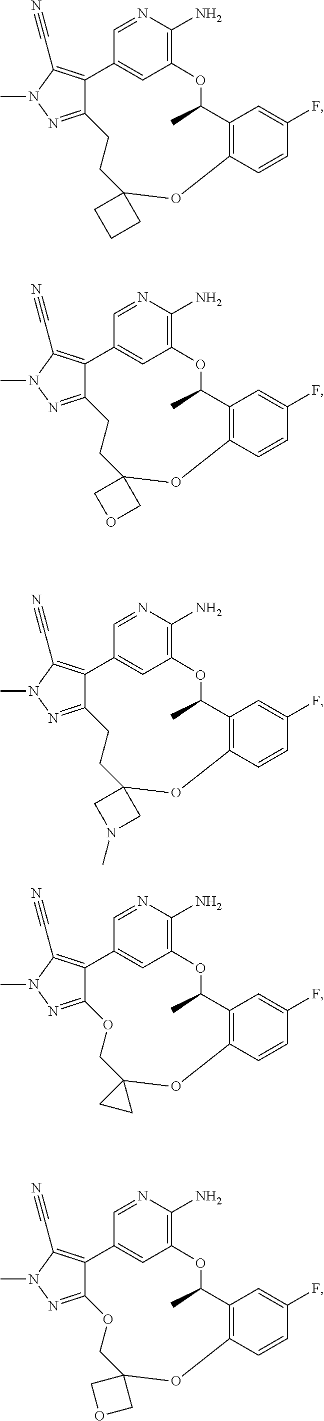 [SMILES: C[C@H]1Oc2cc(cnc2N)-c2c(nn(C)c2C#N)CCC2(CCC2)Oc2ccc(F)cc21 ; C[C@H]1Oc2cc(cnc2N)-c2c(nn(C)c2C#N)CCC2(COC2)Oc2ccc(F)cc21 ; C[C@H]1Oc2cc(cnc2N)-c2c(nn(C)c2C#N)CCC2(CN(C)C2)Oc2ccc(F)cc21 ; C[C@H]1Oc2cc(cnc2N)-c2c(nn(C)c2C#N)OCC2(CC2)Oc2ccc(F)cc21 ; C[C@H]1Oc2cc(cnc2N)-c2c(nn(C)c2C#N)OCC2(COC2)Oc2ccc(F)cc21] 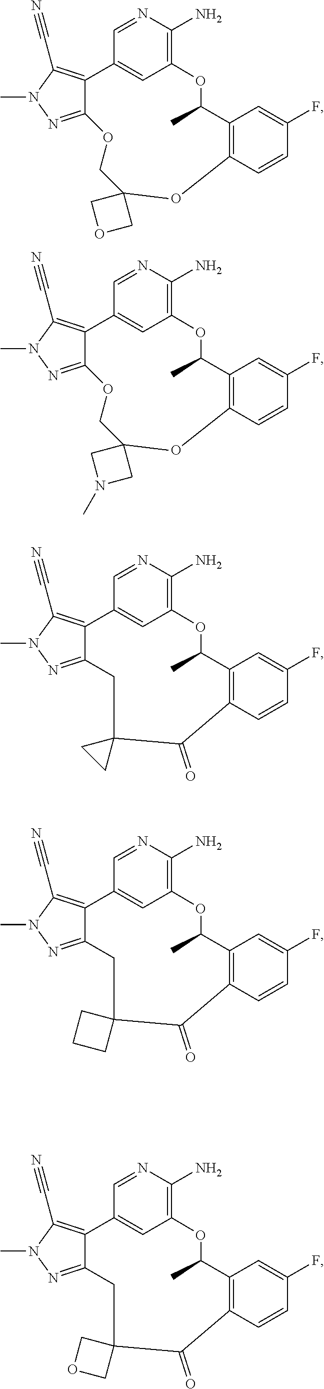 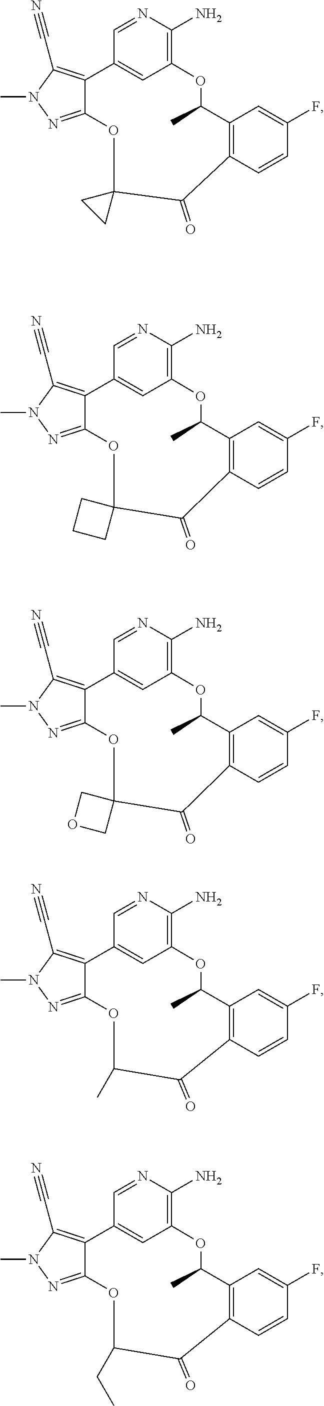 [SMILES: C[C@H]1Oc2cc(cnc2N)-c2c(nn(C)c2C#N)OC2(CC2)C(=O)c2ccc(F)cc21 ; C[C@H]1Oc2cc(cnc2N)-c2c(nn(C)c2C#N)OC2(CCC2)C(=O)c2ccc(F)cc21 ; C[C@H]1Oc2cc(cnc2N)-c2c(nn(C)c2C#N)OC2(COC2)C(=O)c2ccc(F)cc21 ; CC1Oc2nn(C)c(C#N)c2-c2cnc(N)c(c2)O[C@H](C)c2cc(F)ccc2C1=O ; CCC1Oc2nn(C)c(C#N)c2-c2cnc(N)c(c2)O[C@H](C)c2cc(F)ccc2C1=O] 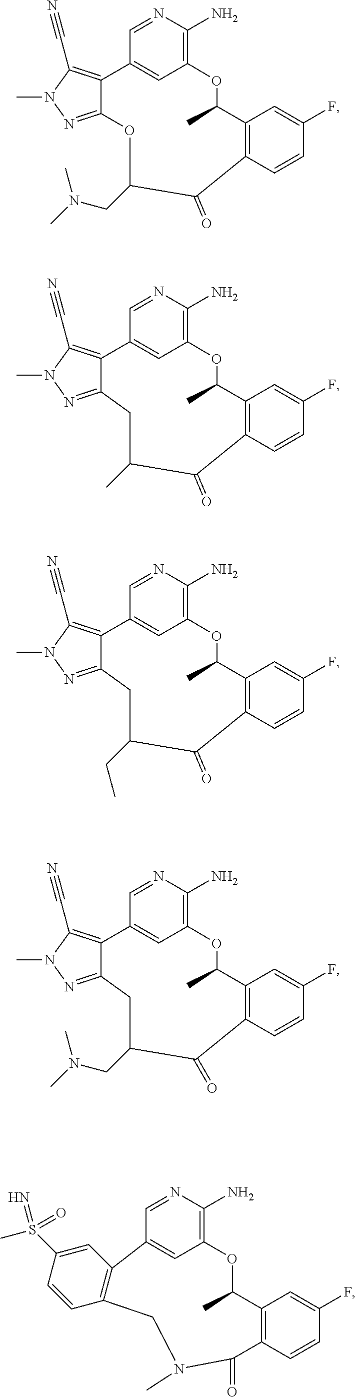 [SMILES: C[C@H]1Oc2cc(cnc2N)-c2c(nn(C)c2C#N)OC(CN(C)C)C(=O)c2ccc(F)cc21 ; CC1Cc2nn(C)c(C#N)c2-c2cnc(N)c(c2)O[C@H](C)c2cc(F)ccc2C1=O ; CCC1Cc2nn(C)c(C#N)c2-c2cnc(N)c(c2)O[C@H](C)c2cc(F)ccc2C1=O ; C[C@H]1Oc2cc(cnc2N)-c2c(nn(C)c2C#N)CC(CN(C)C)C(=O)c2ccc(F)cc21 ; C[C@H]1Oc2cc(cnc2N)-c2cc(S(C)(=N)=O)ccc2CN(C)C(=O)c2ccc(F)cc21] 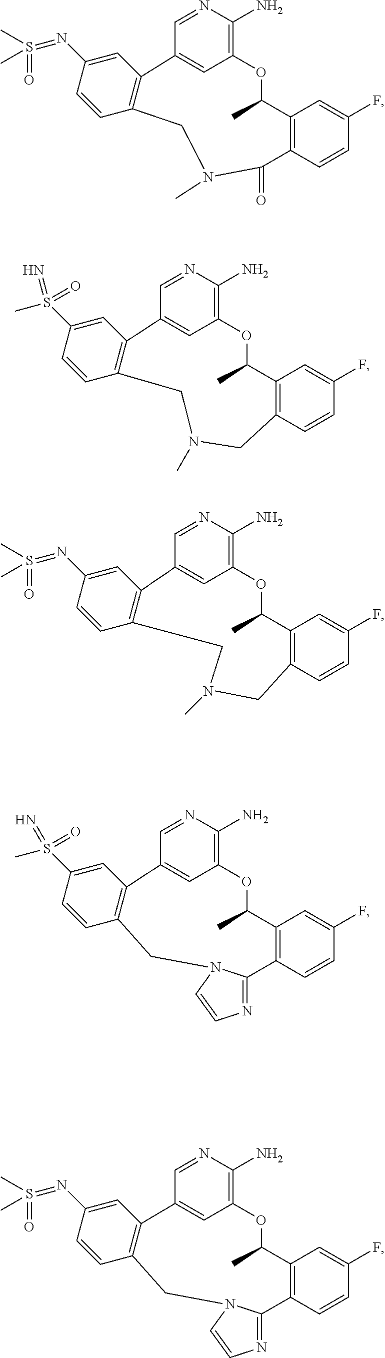 [SMILES: C[C@H]1Oc2cc(cnc2N)-c2cc(N=S(C)(C)=O)ccc2CN(C)C(=O)c2ccc(F)cc21 ; C[C@H]1Oc2cc(cnc2N)-c2cc(S(C)(=N)=O)ccc2CN(C)Cc2ccc(F)cc21 ; C[C@H]1Oc2cc(cnc2N)-c2cc(N=S(C)(C)=O)ccc2CN(C)Cc2ccc(F)cc21 ; C[C@H]1Oc2cc(cnc2N)-c2cc(S(C)(=N)=O)ccc2Cn2ccnc2-c2ccc(F)cc21 ; C[C@H]1Oc2cc(cnc2N)-c2cc(N=S(C)(C)=O)ccc2Cn2ccnc2-c2ccc(F)cc21] 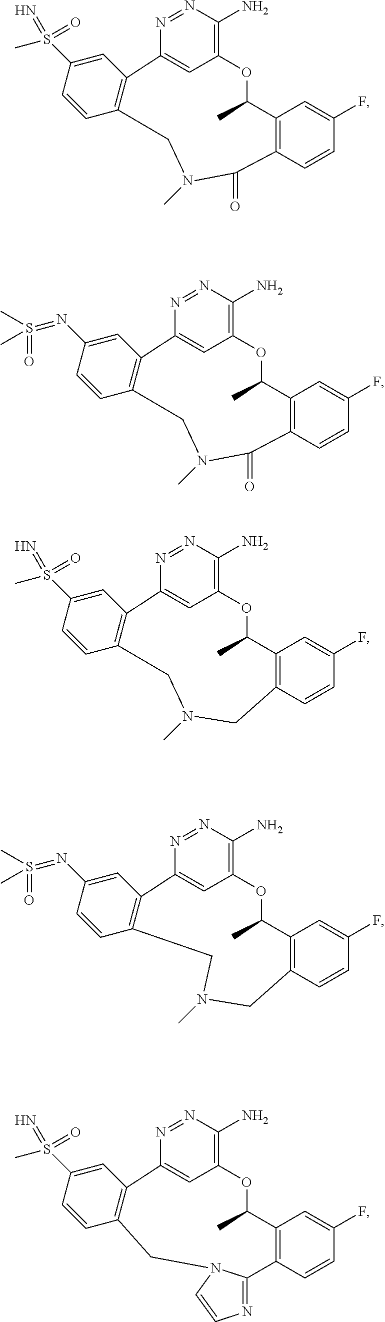 [SMILES: C[C@H]1Oc2cc(nnc2N)-c2cc(S(C)(=N)=O)ccc2CN(C)C(=O)c2ccc(F)cc21 ; C[C@H]1Oc2cc(nnc2N)-c2cc(N=S(C)(C)=O)ccc2CN(C)C(=O)c2ccc(F)cc21 ; C[C@H]1Oc2cc(nnc2N)-c2cc(S(C)(=N)=O)ccc2CN(C)Cc2ccc(F)cc21 ; C[C@H]1Oc2cc(nnc2N)-c2cc(N=S(C)(C)=O)ccc2CN(C)Cc2ccc(F)cc21 ; C[C@H]1Oc2cc(nnc2N)-c2cc(S(C)(=N)=O)ccc2Cn2ccnc2-c2ccc(F)cc21] 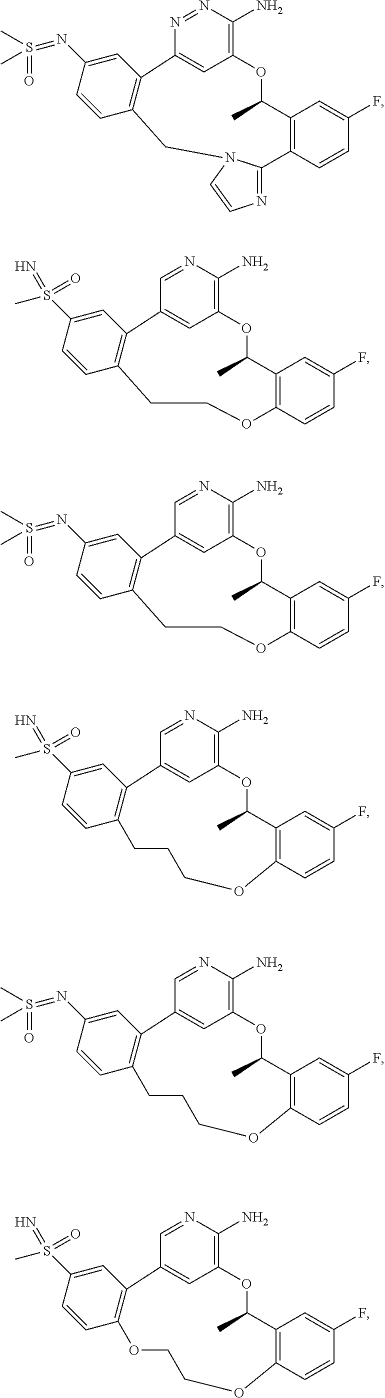 [SMILES: C[C@H]1Oc2cc(nnc2N)-c2cc(N=S(C)(C)=O)ccc2Cn2ccnc2-c2ccc(F)cc21 ; C[C@H]1Oc2cc(cnc2N)-c2cc(S(C)(=N)=O)ccc2CCOc2ccc(F)cc21 ; C[C@H]1Oc2cc(cnc2N)-c2cc(N=S(C)(C)=O)ccc2CCOc2ccc(F)cc21 ; C[C@H]1Oc2cc(cnc2N)-c2cc(S(C)(=N)=O)ccc2CCCOc2ccc(F)cc21 ; C[C@H]1Oc2cc(cnc2N)-c2cc(N=S(C)(C)=O)ccc2CCCOc2ccc(F)cc21 ; C[C@H]1Oc2cc(cnc2N)-c2cc(S(C)(=N)=O)ccc2OCCOc2ccc(F)cc21] 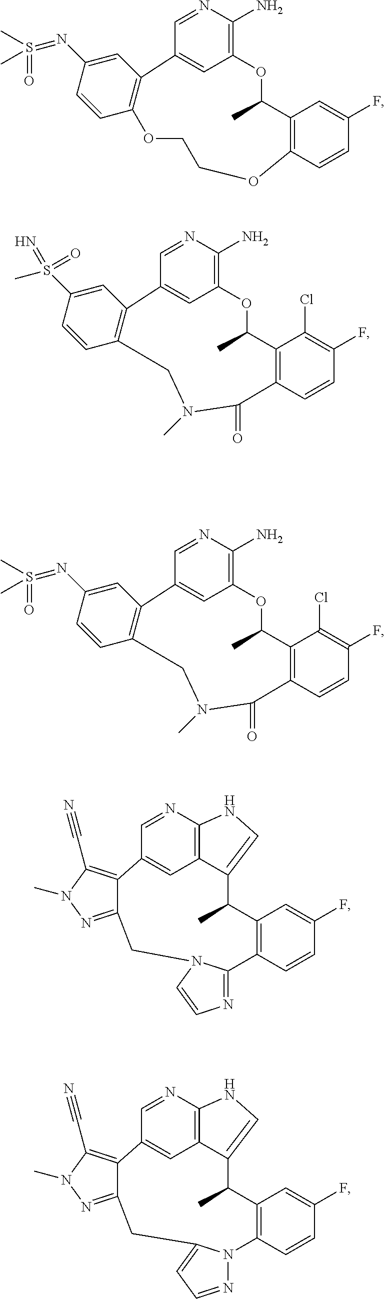 [SMILES: C[C@H]1Oc2cc(cnc2N)-c2cc(N=S(C)(C)=O)ccc2OCCOc2ccc(F)cc21 ; C[C@H]1Oc2cc(cnc2N)-c2cc(S(C)(=N)=O)ccc2CN(C)C(=O)c2ccc(F)c(Cl)c21 ; C[C@H]1Oc2cc(cnc2N)-c2cc(N=S(C)(C)=O)ccc2CN(C)C(=O)c2ccc(F)c(Cl)c21 ; C[C@@H]1c2cc(F)ccc2-c2nccn2Cc2nn(C)c(C#N)c2-c2cnc3[nH]cc1c3c2 ; C[C@@H]1c2cc(F)ccc2-n2nccc2Cc2nn(C)c(C#N)c2-c2cnc3[nH]cc1c3c2] 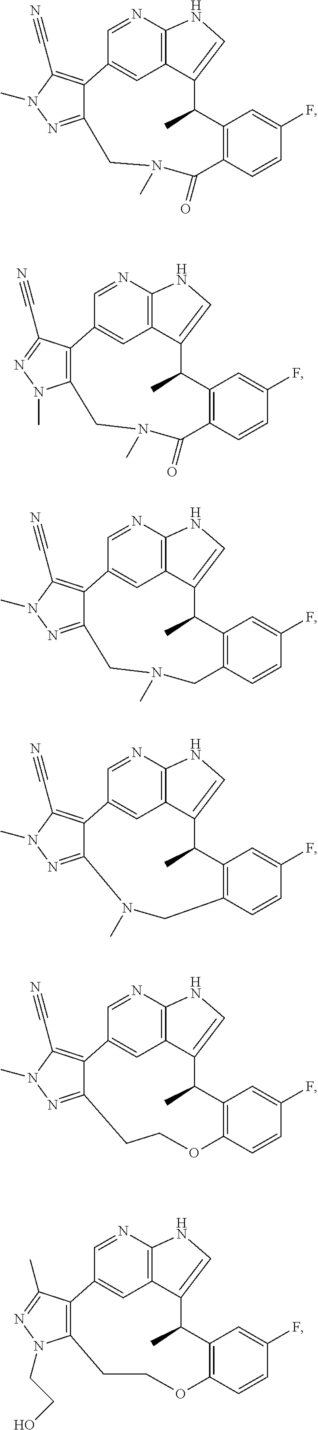 [SMILES: C[C@@H]1c2cc(F)ccc2C(=O)N(C)Cc2nn(C)c(C#N)c2-c2cnc3[nH]cc1c3c2 ; C[C@@H]1c2cc(F)ccc2C(=O)N(C)Cc2c(c(C#N)nn2C)-c2cnc3[nH]cc1c3c2 ; C[C@@H]1c2cc(F)ccc2CN(C)Cc2nn(C)c(C#N)c2-c2cnc3[nH]cc1c3c2 ; C[C@@H]1c2cc(F)ccc2CN(C)c2nn(C)c(C#N)c2-c2cnc3[nH]cc1c3c2 ; C[C@@H]1c2cc(F)ccc2OCCc2nn(C)c(C#N)c2-c2cnc3[nH]cc1c3c2 ; Cc1nn(CCO)c2c1-c1cnc3[nH]cc(c3c1)[C@H](C)c1cc(F)ccc1OCC2] 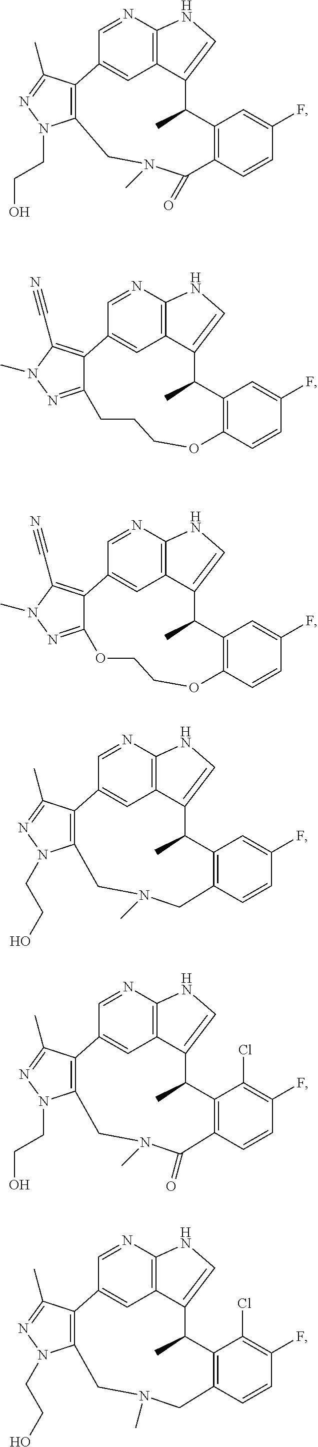 [SMILES: Cc1nn(CCO)c2c1-c1cnc3[nH]cc(c3c1)[C@H](C)c1cc(F)ccc1C(=O)N(C)C2 ; C[C@@H]1c2cc(F)ccc2OCCCc2nn(C)c(C#N)c2-c2cnc3[nH]cc1c3c2 ; C[C@@H]1c2cc(F)ccc2OCCOc2nn(C)c(C#N)c2-c2cnc3[nH]cc1c3c2 ; Cc1nn(CCO)c2c1-c1cnc3[nH]cc(c3c1)[C@H](C)c1cc(F)ccc1CN(C)C2 ; Cc1nn(CCO)c2c1-c1cnc3[nH]cc(c3c1)[C@H](C)c1c(ccc(F)c1Cl)C(=O)N(C)C2 ; Cc1nn(CCO)c2c1-c1cnc3[nH]cc(c3c1)[C@H](C)c1c(ccc(F)c1Cl)CN(C)C2] 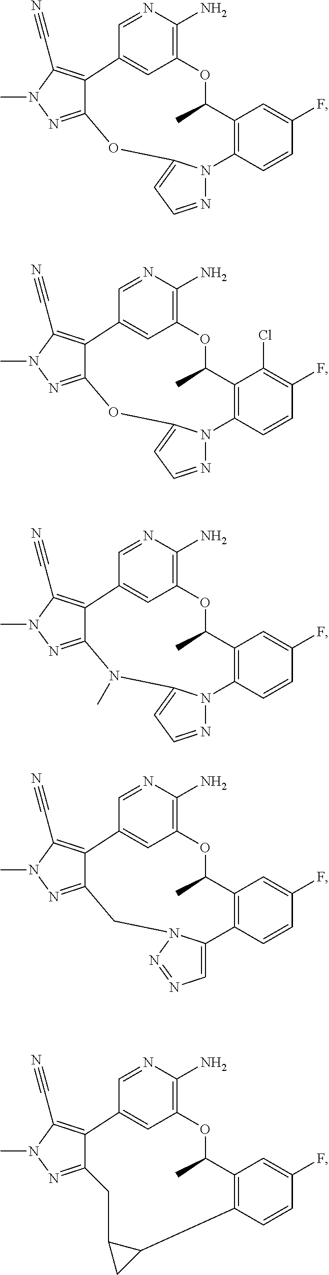 [SMILES: C[C@H]1Oc2cc(cnc2N)-c2c(nn(C)c2C#N)Oc2ccnn2-c2ccc(F)cc21 ; C[C@H]1Oc2cc(cnc2N)-c2c(nn(C)c2C#N)Oc2ccnn2-c2ccc(F)c(Cl)c21 ; C[C@H]1Oc2cc(cnc2N)-c2c(nn(C)c2C#N)N(C)c2ccnn2-c2ccc(F)cc21 ; C[C@H]1Oc2cc(cnc2N)-c2c(nn(C)c2C#N)Cn2nncc2-c2ccc(F)cc21 ; C[C@H]1Oc2cc(cnc2N)-c2c(nn(C)c2C#N)CC2CC2c2ccc(F)cc21] 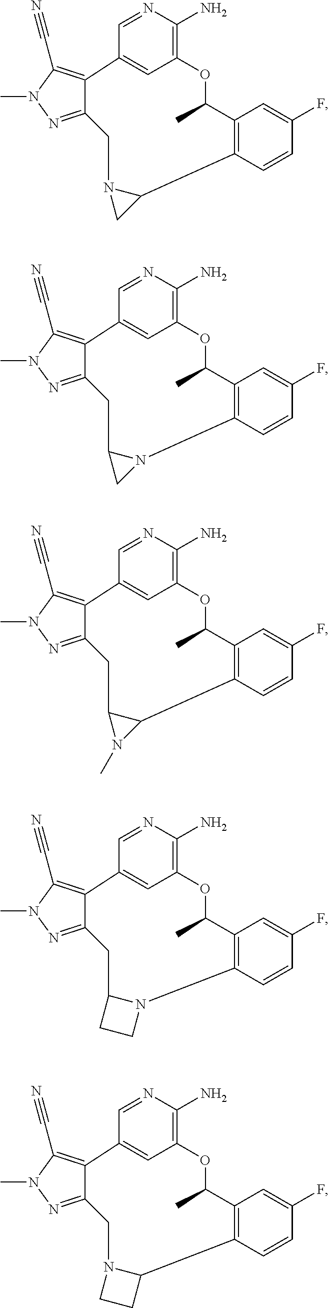 [SMILES: C[C@H]1Oc2cc(cnc2N)-c2c(nn(C)c2C#N)CN2CC2c2ccc(F)cc21 ; C[C@H]1Oc2cc(cnc2N)-c2c(nn(C)c2C#N)CC2CN2c2ccc(F)cc21 ; C[C@H]1Oc2cc(cnc2N)-c2c(nn(C)c2C#N)CC2C(c3ccc(F)cc31)N2C ; C[C@H]1Oc2cc(cnc2N)-c2c(nn(C)c2C#N)CC2CCN2c2ccc(F)cc21 ; C[C@H]1Oc2cc(cnc2N)-c2c(nn(C)c2C#N)CN2CCC2c2ccc(F)cc21] 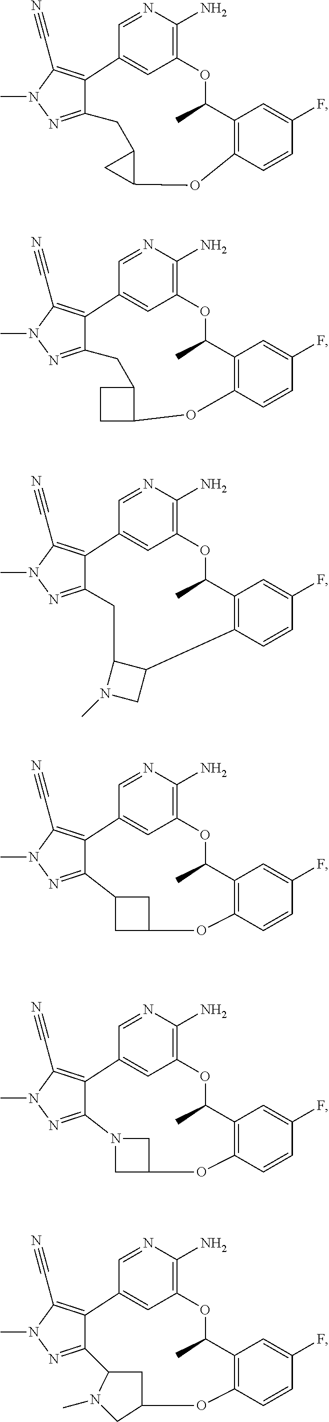 [SMILES: C[C@H]1Oc2cc(cnc2N)-c2c(nn(C)c2C#N)CC2CC2Oc2ccc(F)cc21 ; C[C@H]1Oc2cc(cnc2N)-c2c(nn(C)c2C#N)CC2CCC2Oc2ccc(F)cc21 ; C[C@H]1Oc2cc(cnc2N)-c2c(nn(C)c2C#N)CC2C(CN2C)c2ccc(F)cc21 ; C[C@H]1Oc2cc(cnc2N)-c2c(nn(C)c2C#N)C2CC(C2)Oc2ccc(F)cc21 ; C[C@H]1Oc2cc(cnc2N)-c2c(nn(C)c2C#N)N2CC(C2)Oc2ccc(F)cc21 ; C[C@H]1Oc2cc(cnc2N)-c2c(nn(C)c2C#N)C2CC(CN2C)Oc2ccc(F)cc21] 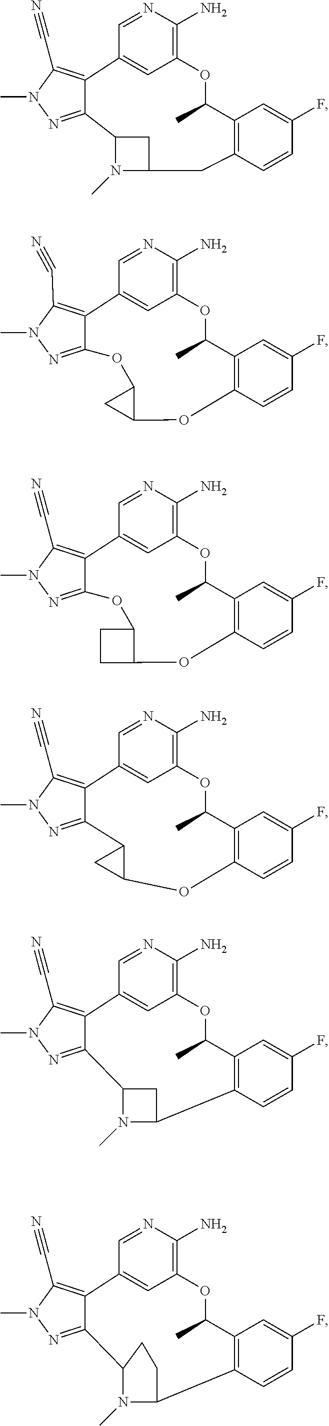 [SMILES: C[C@H]1Oc2cc(cnc2N)-c2c(nn(C)c2C#N)C2CC(Cc3ccc(F)cc31)N2C ; C[C@H]1Oc2cc(cnc2N)-c2c(nn(C)c2C#N)OC2CC2Oc2ccc(F)cc21 ; C[C@H]1Oc2cc(cnc2N)-c2c(nn(C)c2C#N)OC2CCC2Oc2ccc(F)cc21 ; C[C@H]1Oc2cc(cnc2N)-c2c(nn(C)c2C#N)C2CC2Oc2ccc(F)cc21 ; C[C@H]1Oc2cc(cnc2N)-c2c(nn(C)c2C#N)C2CC(c3ccc(F)cc31)N2C ; C[C@H]1Oc2cc(cnc2N)-c2c(nn(C)c2C#N)C2CCC(c3ccc(F)cc31)N2C] 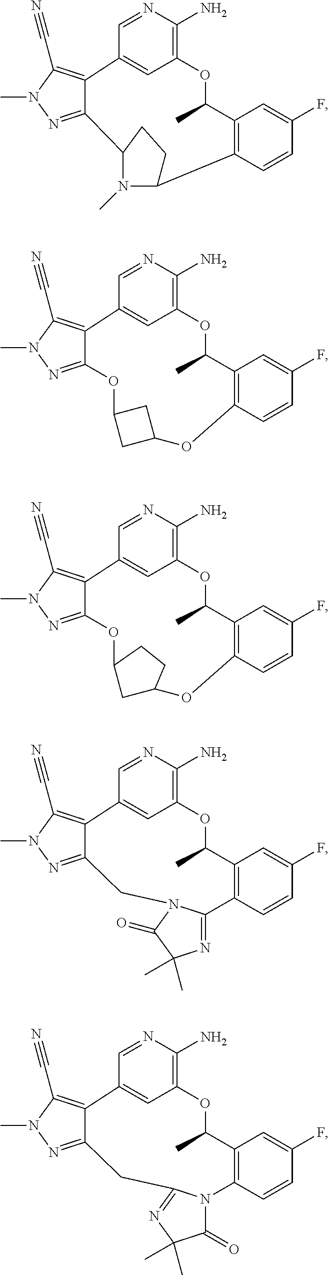 [SMILES: C[C@H]1Oc2cc(cnc2N)-c2c(nn(C)c2C#N)C2CCC(c3ccc(F)cc31)N2C ; C[C@H]1Oc2cc(cnc2N)-c2c(nn(C)c2C#N)OC2CC(C2)Oc2ccc(F)cc21 ; C[C@H]1Oc2cc(cnc2N)-c2c(nn(C)c2C#N)OC2CCC(C2)Oc2ccc(F)cc21 ; C[C@H]1Oc2cc(cnc2N)-c2c(nn(C)c2C#N)CN2C(=O)C(C)(C)N=C2c2ccc(F)cc21 ; C[C@H]1Oc2cc(cnc2N)-c2c(nn(C)c2C#N)CC2=NC(C)(C)C(=O)N2c2ccc(F)cc21] 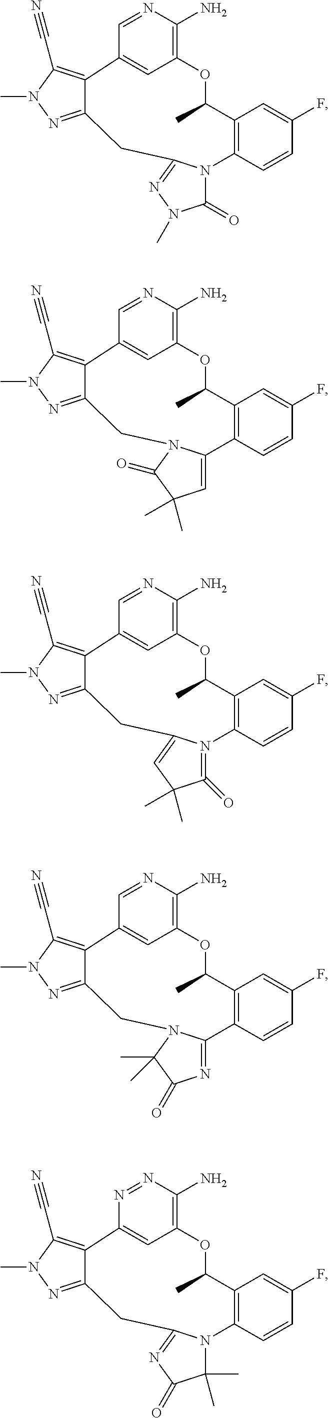 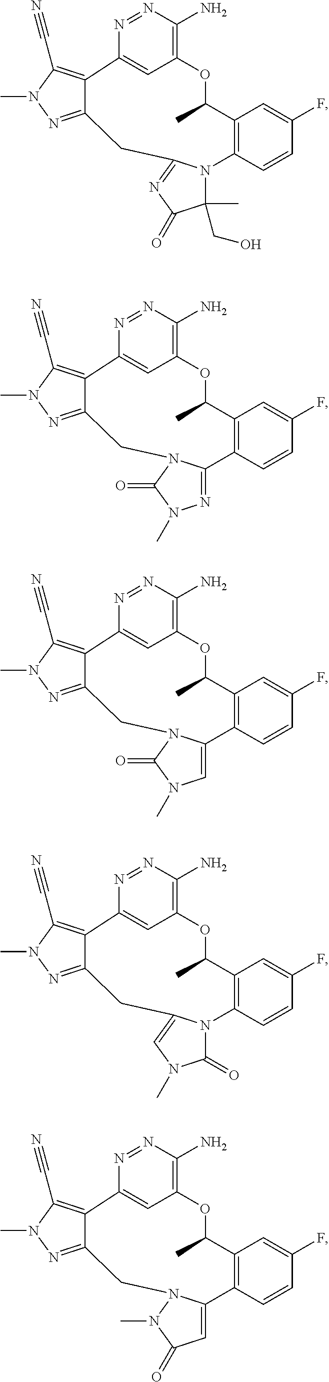 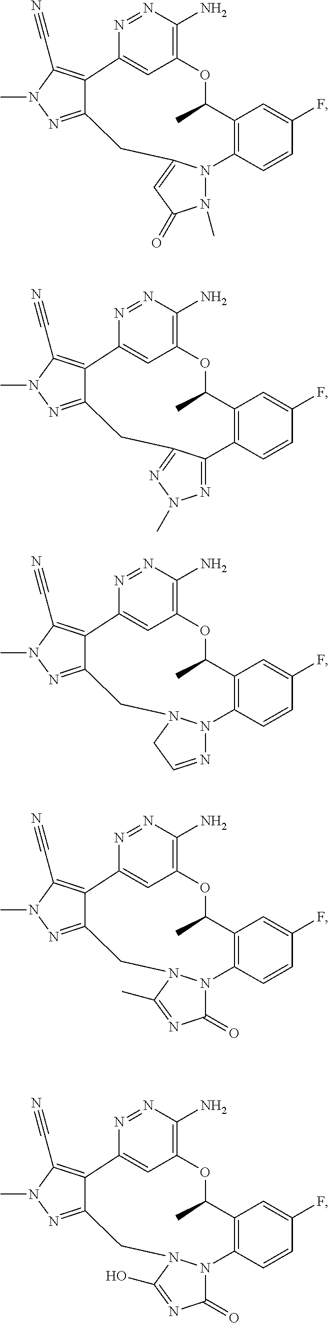 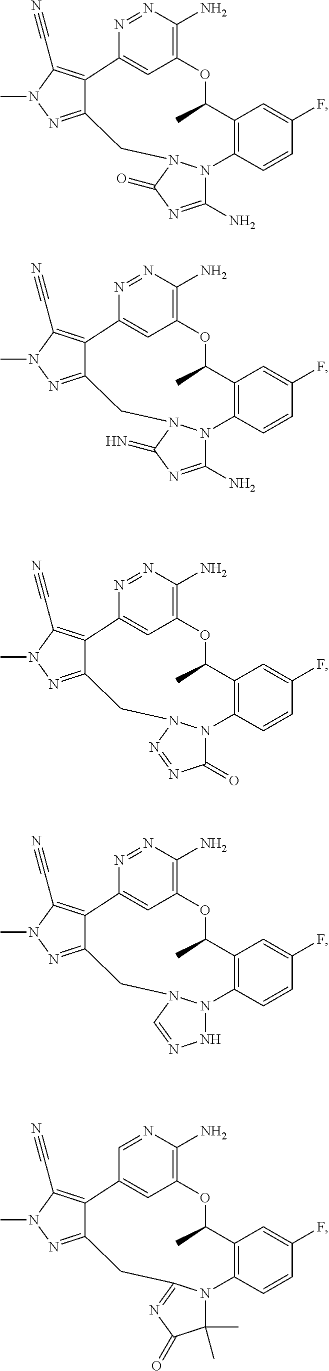 [SMILES: C[C@H]1Oc2cc(nnc2N)-c2c(nn(C)c2C#N)Cn2c(=O)nc(N)n2-c2ccc(F)cc21 ; C[C@H]1Oc2cc(nnc2N)-c2c(nn(C)c2C#N)Cn2c(=N)nc(N)n2-c2ccc(F)cc21 ; C[C@H]1Oc2cc(nnc2N)-c2c(nn(C)c2C#N)Cn2nnc(=O)n2-c2ccc(F)cc21 ; C[C@H]1Oc2cc(nnc2N)-c2c(nn(C)c2C#N)CN2C=NNN2c2ccc(F)cc21 ; C[C@H]1Oc2cc(cnc2N)-c2c(nn(C)c2C#N)CC2=NC(=O)C(C)(C)N2c2ccc(F)cc21] 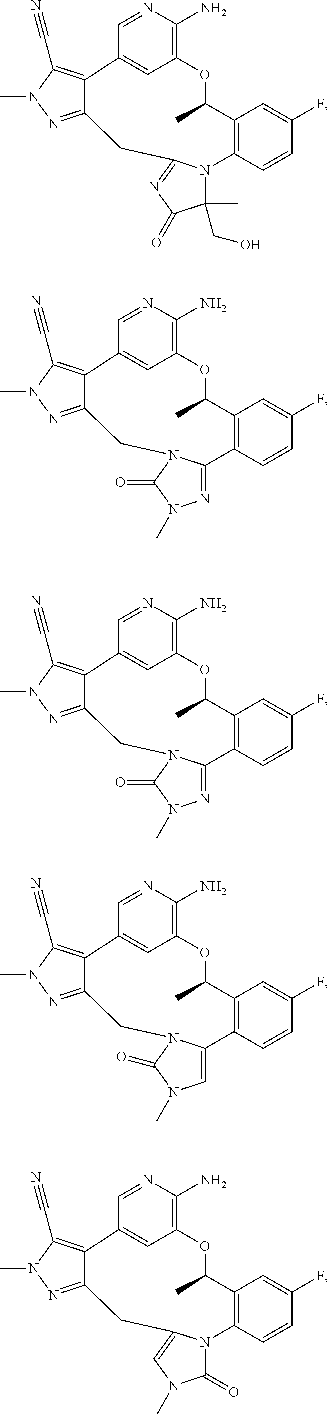 [SMILES: C[C@H]1Oc2cc(cnc2N)-c2c(nn(C)c2C#N)CC2=NC(=O)C(C)(CO)N2c2ccc(F)cc21 ; C[C@H]1Oc2cc(cnc2N)-c2c(nn(C)c2C#N)Cn2c(nn(C)c2=O)-c2ccc(F)cc21 ; C[C@H]1Oc2cc(cnc2N)-c2c(nn(C)c2C#N)Cn2c(nn(C)c2=O)-c2ccc(F)cc21 ; C[C@H]1Oc2cc(cnc2N)-c2c(nn(C)c2C#N)Cn2c(cn(C)c2=O)-c2ccc(F)cc21 ; C[C@H]1Oc2cc(cnc2N)-c2c(nn(C)c2C#N)Cc2cn(C)c(=O)n2-c2ccc(F)cc21] 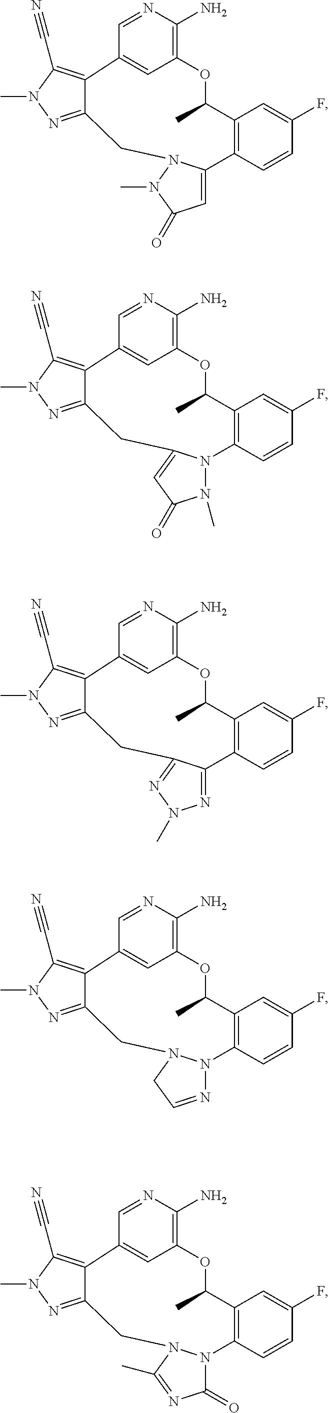 [SMILES: C[C@H]1Oc2cc(cnc2N)-c2c(nn(C)c2C#N)Cn2c(cc(=O)n2C)-c2ccc(F)cc21 ; C[C@H]1Oc2cc(cnc2N)-c2c(nn(C)c2C#N)Cc2cc(=O)n(C)n2-c2ccc(F)cc21 ; C[C@H]1Oc2cc(cnc2N)-c2c(nn(C)c2C#N)Cc2nn(C)nc2-c2ccc(F)cc21 ; C[C@H]1Oc2cc(cnc2N)-c2c(nn(C)c2C#N)CN2CC=NN2c2ccc(F)cc21 ; Cc1nc(=O)n2n1Cc1nn(C)c(C#N)c1-c1cnc(N)c(c1)O[C@H](C)c1cc(F)ccc1-2] 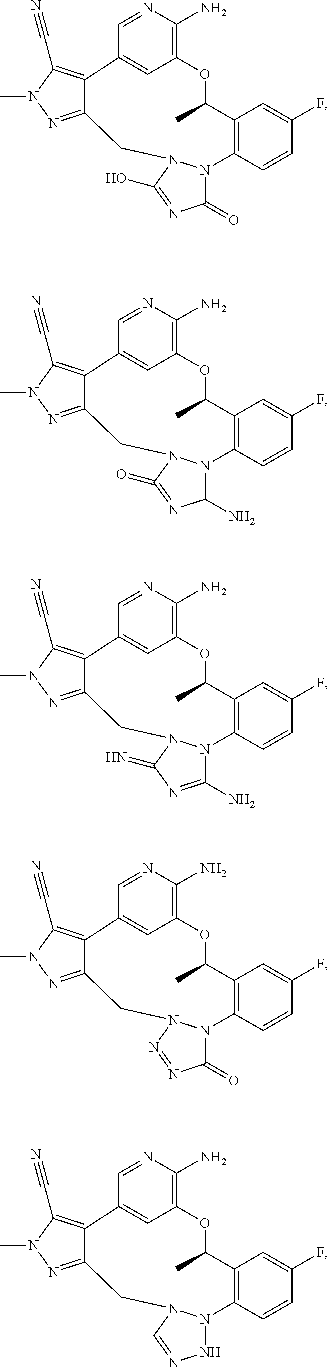 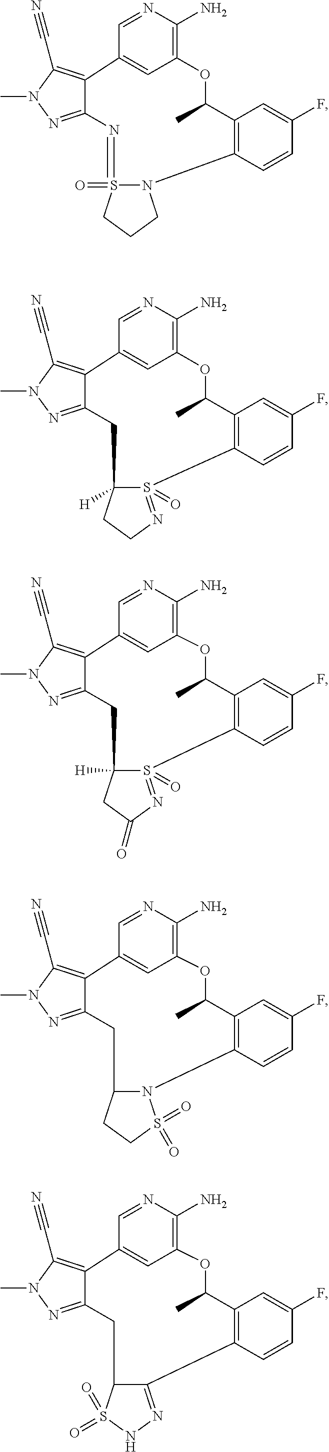 [SMILES: C[C@H]1Oc2cc(cnc2N)-c2c(nn(C)c2C#N)N=S2(=O)CCCN2c2ccc(F)cc21 ; C[C@H]1Oc2cc(cnc2N)-c2c(nn(C)c2C#N)C[C@@H]2CCN=S2(=O)c2ccc(F)cc21 ; C[C@H]1Oc2cc(cnc2N)-c2c(nn(C)c2C#N)C[C@@H]2CC(=O)N=S2(=O)c2ccc(F)cc21 ; C[C@H]1Oc2cc(cnc2N)-c2c(nn(C)c2C#N)CC2CCS(=O)(=O)N2c2ccc(F)cc21 ; C[C@H]1Oc2cc(cnc2N)-c2c(nn(C)c2C#N)CC2C(=NNS2(=O)=O)c2ccc(F)cc21] 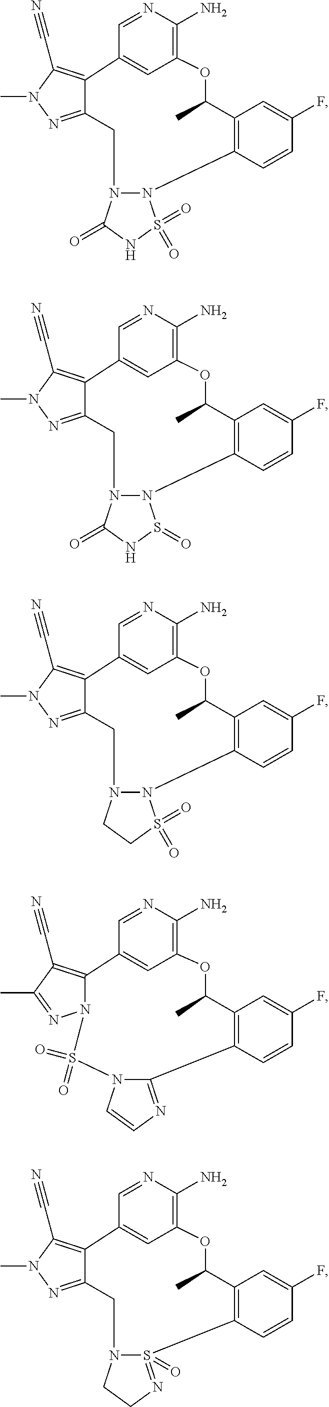 [SMILES: C[C@H]1Oc2cc(cnc2N)-c2c(nn(C)c2C#N)CN2C(=O)NS(=O)(=O)N2c2ccc(F)cc21 ; C[C@H]1Oc2cc(cnc2N)-c2c(nn(C)c2C#N)CN2C(=O)NS(=O)N2c2ccc(F)cc21 ; C[C@H]1Oc2cc(cnc2N)-c2c(nn(C)c2C#N)CN2CCS(=O)(=O)N2c2ccc(F)cc21 ; Cc1nn2c(c1C#N)-c1cnc(N)c(c1)O[C@H](C)c1cc(F)ccc1-c1nccn1S2(=O)=O ; C[C@H]1Oc2cc(cnc2N)-c2c(nn(C)c2C#N)CN2CCN=S2(=O)c2ccc(F)cc21] 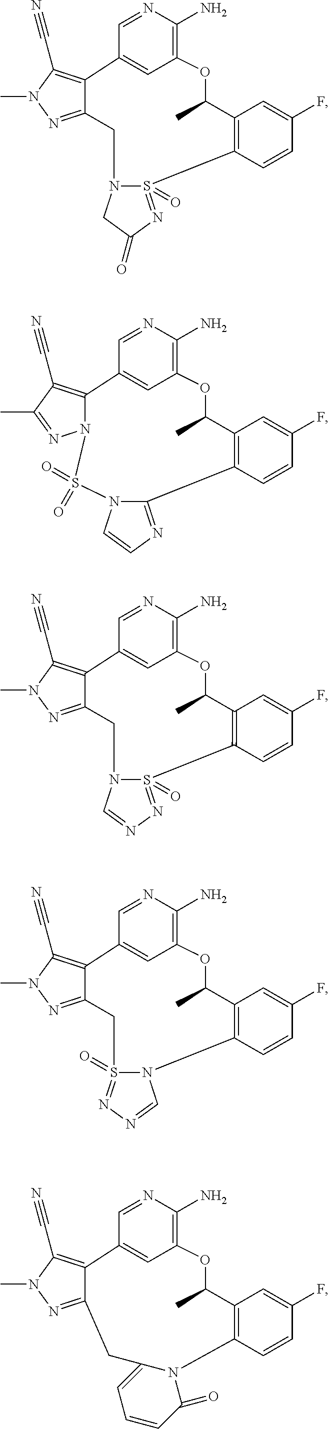 [SMILES: C[C@H]1Oc2cc(cnc2N)-c2c(nn(C)c2C#N)CN2CC(=O)N=S2(=O)c2ccc(F)cc21 ; Cc1nn2c(c1C#N)-c1cnc(N)c(c1)O[C@H](C)c1cc(F)ccc1-c1nccn1S2(=O)=O ; C[C@H]1Oc2cc(cnc2N)-c2c(nn(C)c2C#N)CN2C=NN=S2(=O)c2ccc(F)cc21 ; C[C@H]1Oc2cc(cnc2N)-c2c(nn(C)c2C#N)CS2(=O)=NN=CN2c2ccc(F)cc21 ; C[C@H]1Oc2cc(cnc2N)-c2c(nn(C)c2C#N)Cc2cccc(=O)n2-c2ccc(F)cc21] 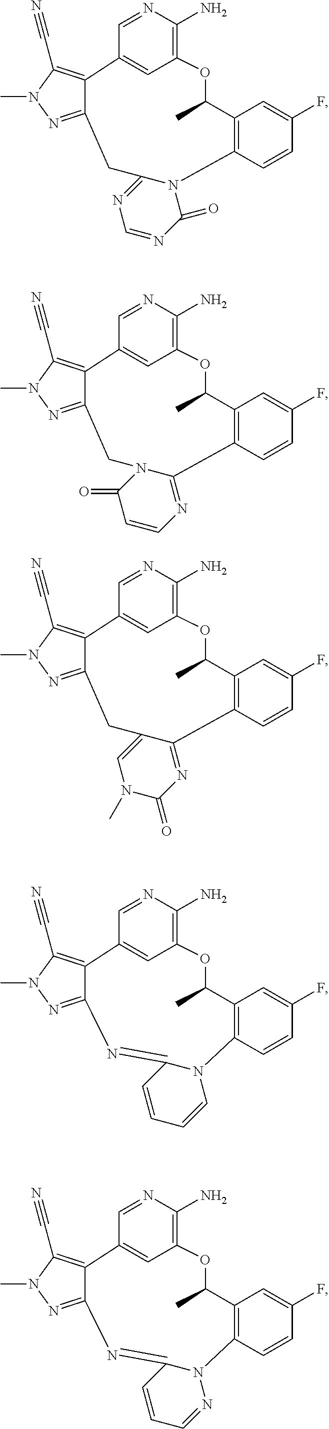 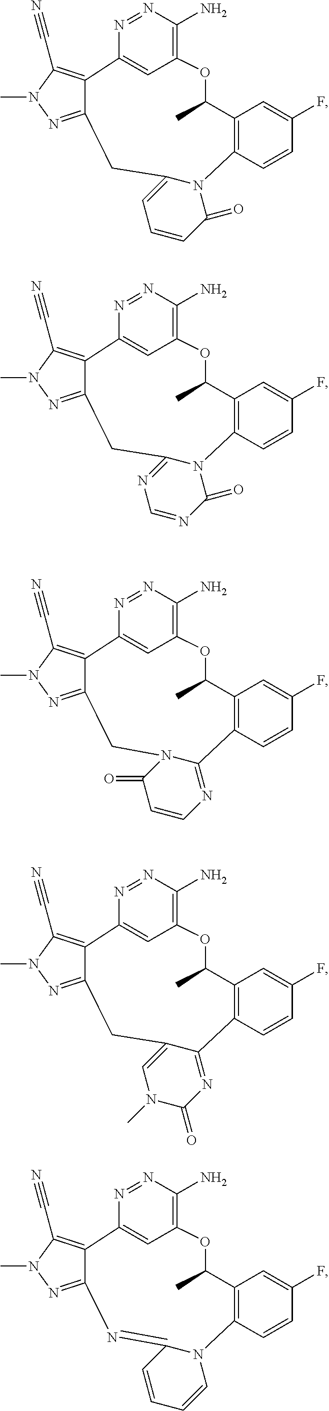 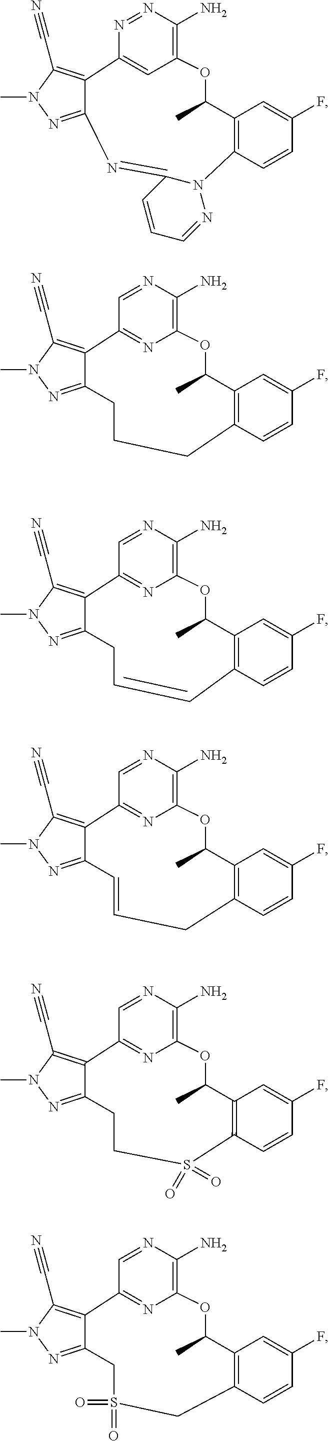 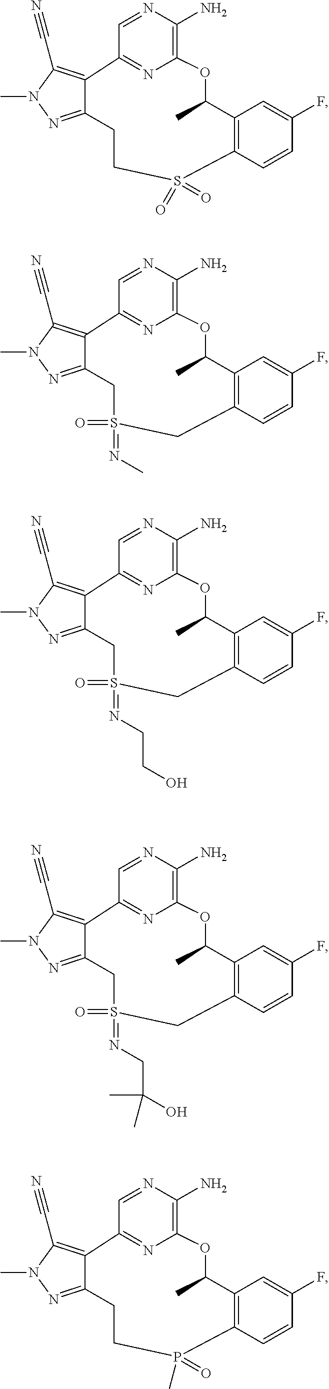 [SMILES: C[C@H]1Oc2nc(cnc2N)-c2c(nn(C)c2C#N)CCS(=O)(=O)c2ccc(F)cc21 ; CN=S1(=O)Cc2ccc(F)cc2[C@@H](C)Oc2nc(cnc2N)-c2c(nn(C)c2C#N)C1 ; C[C@H]1Oc2nc(cnc2N)-c2c(nn(C)c2C#N)CS(=O)(=NCCO)Cc2ccc(F)cc21 ; C[C@H]1Oc2nc(cnc2N)-c2c(nn(C)c2C#N)CS(=O)(=NCC(C)(C)O)Cc2ccc(F)cc21 ; C[C@H]1Oc2nc(cnc2N)-c2c(nn(C)c2C#N)CCP(C)(=O)c2ccc(F)cc21] 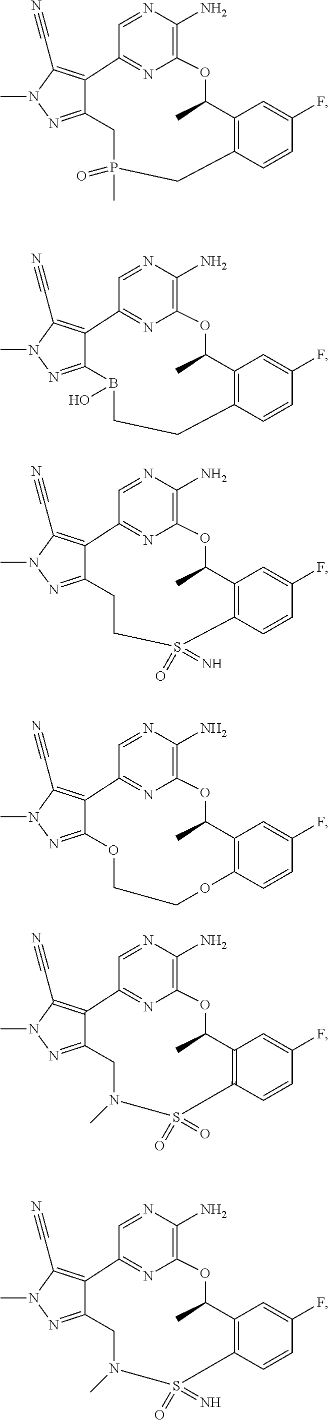 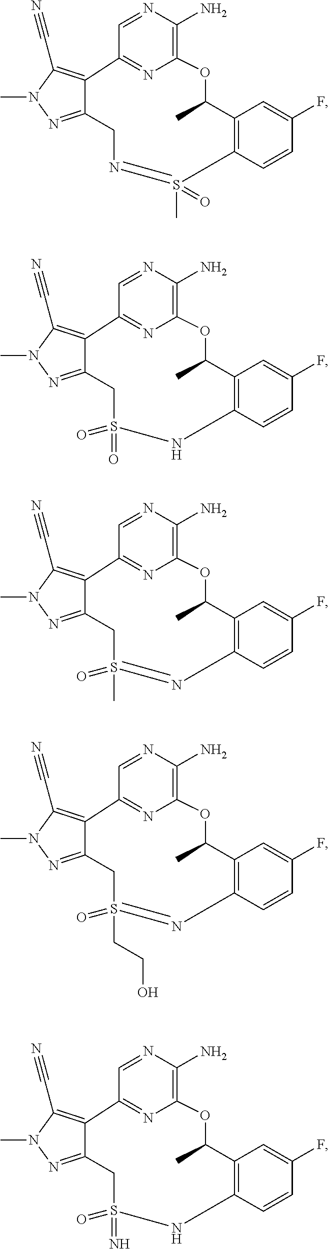 [SMILES: C[C@H]1Oc2nc(cnc2N)-c2c(nn(C)c2C#N)CN=S(C)(=O)c2ccc(F)cc21 ; C[C@H]1Oc2nc(cnc2N)-c2c(nn(C)c2C#N)CS(=O)(=O)Nc2ccc(F)cc21 ; C[C@H]1Oc2nc(cnc2N)-c2c(nn(C)c2C#N)CS(C)(=O)=Nc2ccc(F)cc21 ; C[C@H]1Oc2nc(cnc2N)-c2c(nn(C)c2C#N)CS(=O)(CCO)=Nc2ccc(F)cc21 ; C[C@H]1Oc2nc(cnc2N)-c2c(nn(C)c2C#N)CS(=N)(=O)Nc2ccc(F)cc21] 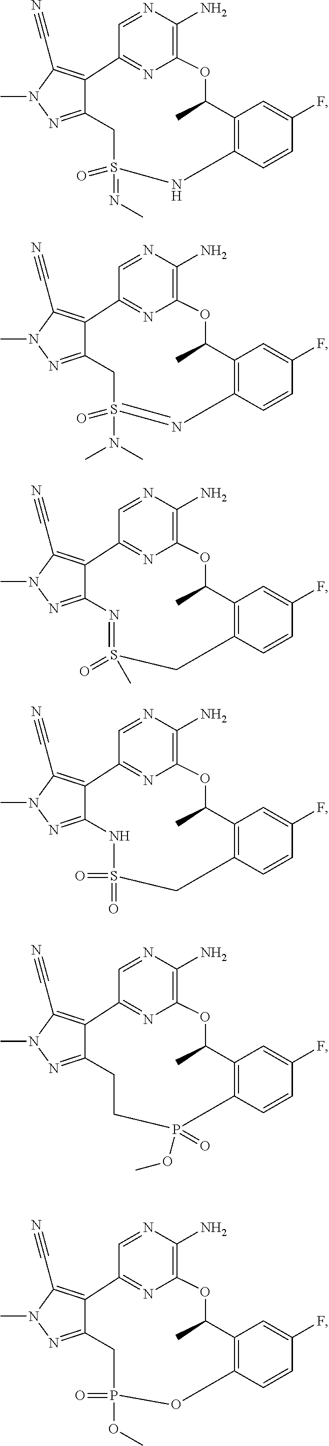 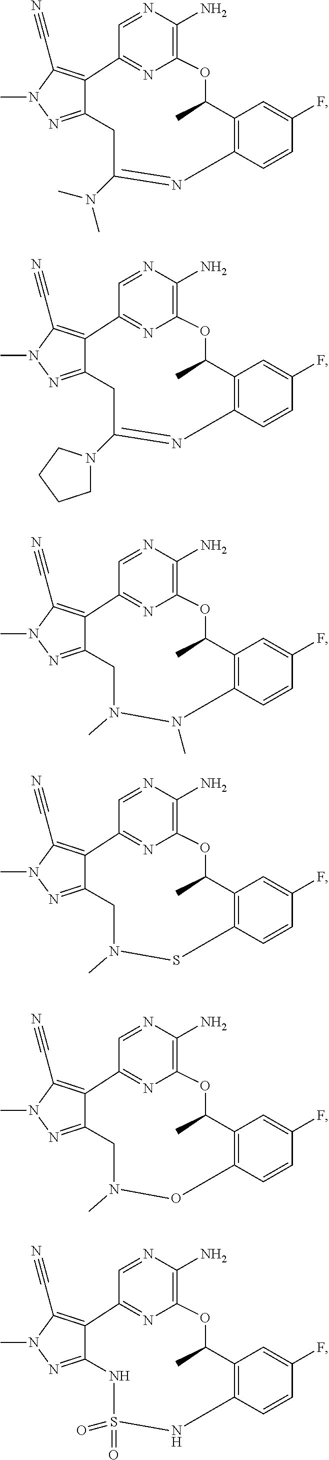 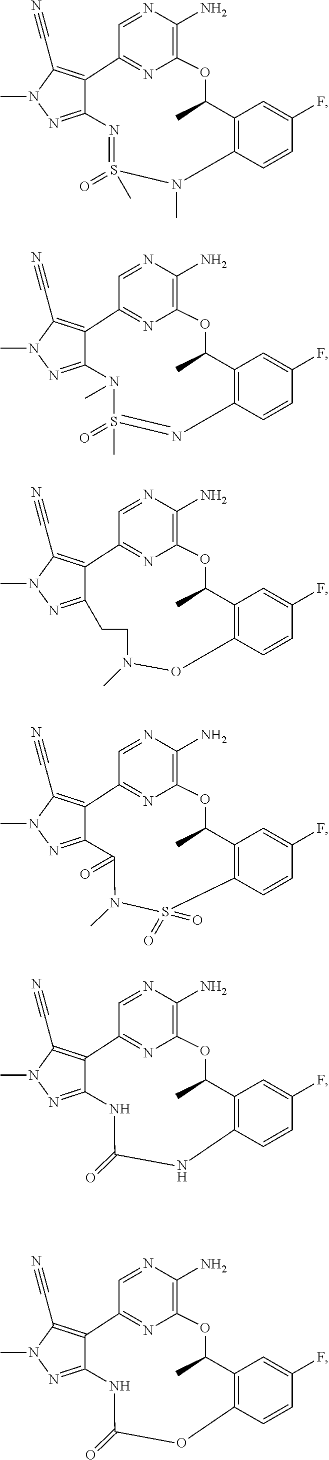 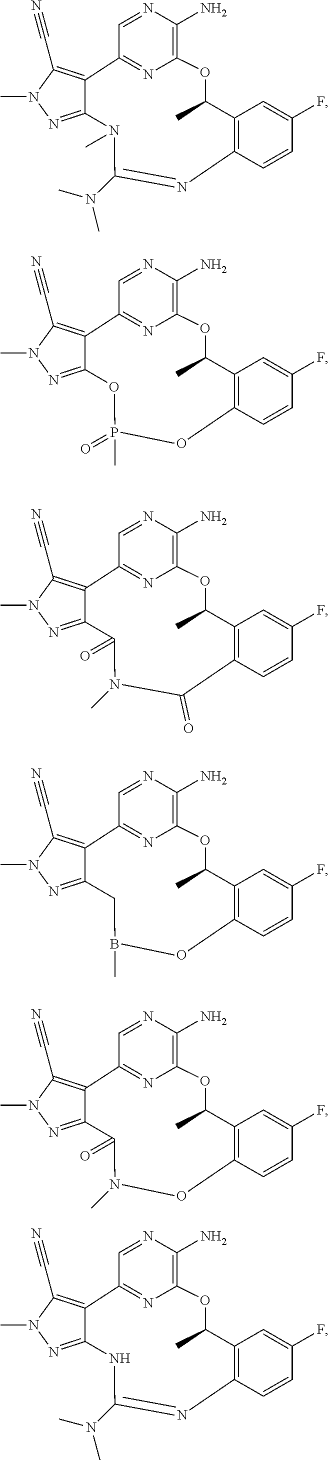 [SMILES: C[C@H]1Oc2nc(cnc2N)-c2c(nn(C)c2C#N)N(C)/C(N(C)C)=N\c2ccc(F)cc21 ; C[C@H]1Oc2nc(cnc2N)-c2c(nn(C)c2C#N)OP(C)(=O)Oc2ccc(F)cc21 ; C[C@H]1Oc2nc(cnc2N)-c2c(nn(C)c2C#N)C(=O)N(C)C(=O)c2ccc(F)cc21 ; CB1Cc2nn(C)c(C#N)c2-c2cnc(N)c(n2)O[C@H](C)c2cc(F)ccc2O1 ; C[C@H]1Oc2nc(cnc2N)-c2c(nn(C)c2C#N)C(=O)N(C)Oc2ccc(F)cc21 ; C[C@H]1Oc2nc(cnc2N)-c2c(nn(C)c2C#N)N/C(N(C)C)=N\c2ccc(F)cc21] 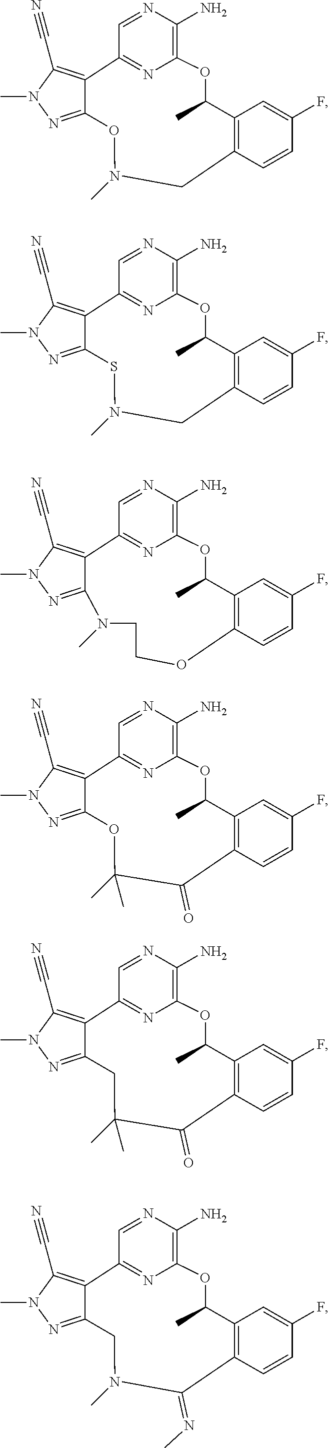 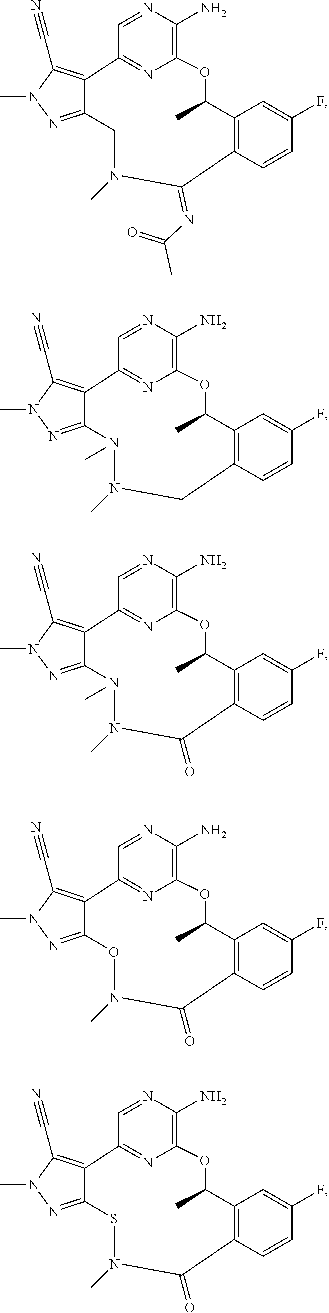 [SMILES: CC(=O)/N=C1/c2ccc(F)cc2[C@@H](C)Oc2nc(cnc2N)-c2c(nn(C)c2C#N)CN1C ; C[C@H]1Oc2nc(cnc2N)-c2c(nn(C)c2C#N)N(C)N(C)Cc2ccc(F)cc21 ; C[C@H]1Oc2nc(cnc2N)-c2c(nn(C)c2C#N)N(C)N(C)C(=O)c2ccc(F)cc21 ; C[C@H]1Oc2nc(cnc2N)-c2c(nn(C)c2C#N)ON(C)C(=O)c2ccc(F)cc21 ; C[C@H]1Oc2nc(cnc2N)-c2c(nn(C)c2C#N)SN(C)C(=O)c2ccc(F)cc21] 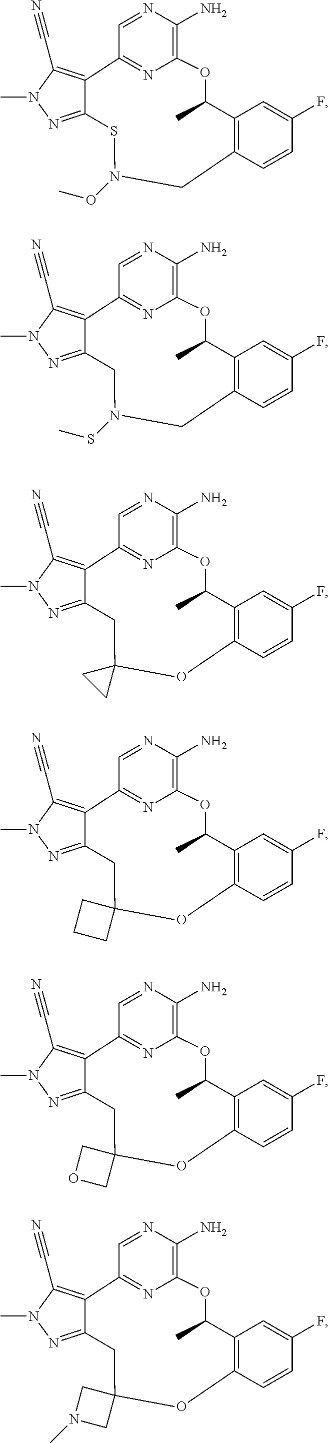 [SMILES: CON1Cc2ccc(F)cc2[C@@H](C)Oc2nc(cnc2N)-c2c(nn(C)c2C#N)S1 ; CSN1Cc2ccc(F)cc2[C@@H](C)Oc2nc(cnc2N)-c2c(nn(C)c2C#N)C1 ; C[C@H]1Oc2nc(cnc2N)-c2c(nn(C)c2C#N)CC2(CC2)Oc2ccc(F)cc21 ; C[C@H]1Oc2nc(cnc2N)-c2c(nn(C)c2C#N)CC2(CCC2)Oc2ccc(F)cc21 ; C[C@H]1Oc2nc(cnc2N)-c2c(nn(C)c2C#N)CC2(COC2)Oc2ccc(F)cc21 ; C[C@H]1Oc2nc(cnc2N)-c2c(nn(C)c2C#N)CC2(CN(C)C2)Oc2ccc(F)cc21] 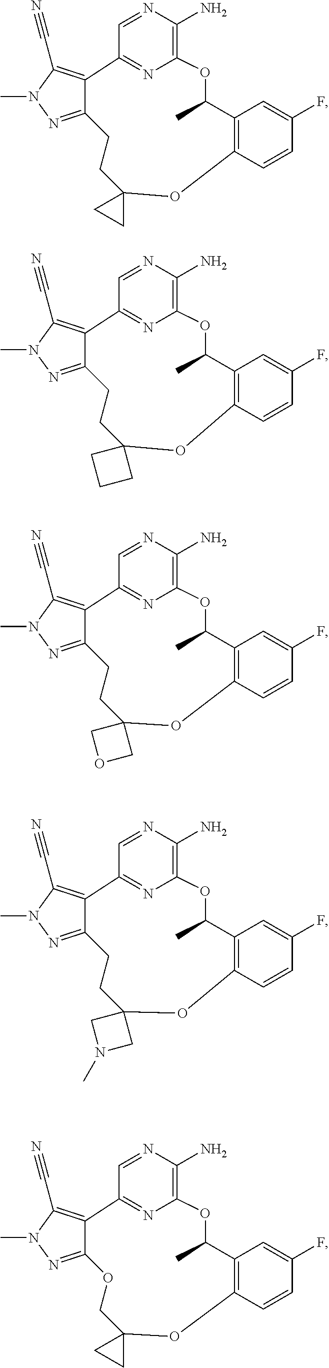 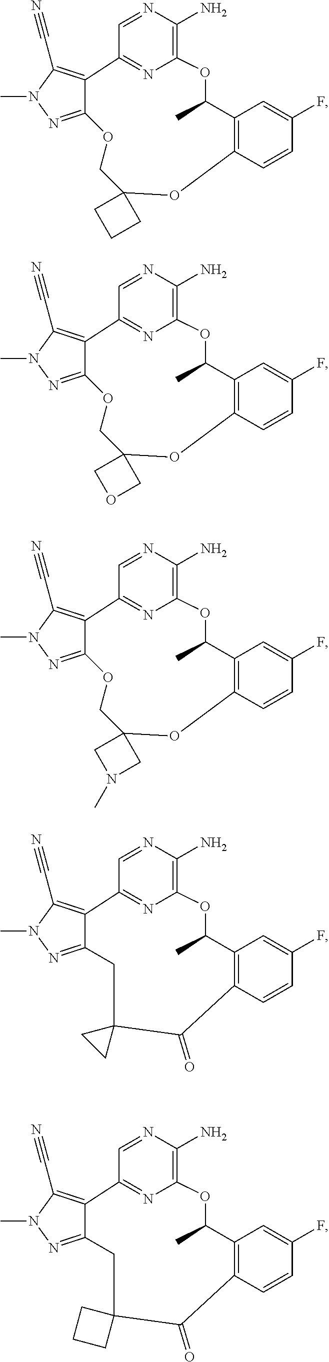 [SMILES: C[C@H]1Oc2nc(cnc2N)-c2c(nn(C)c2C#N)OCC2(CCC2)Oc2ccc(F)cc21 ; C[C@H]1Oc2nc(cnc2N)-c2c(nn(C)c2C#N)OCC2(COC2)Oc2ccc(F)cc21 ; C[C@H]1Oc2nc(cnc2N)-c2c(nn(C)c2C#N)OCC2(CN(C)C2)Oc2ccc(F)cc21 ; C[C@H]1Oc2nc(cnc2N)-c2c(nn(C)c2C#N)CC2(CC2)C(=O)c2ccc(F)cc21 ; C[C@H]1Oc2nc(cnc2N)-c2c(nn(C)c2C#N)CC2(CCC2)C(=O)c2ccc(F)cc21] 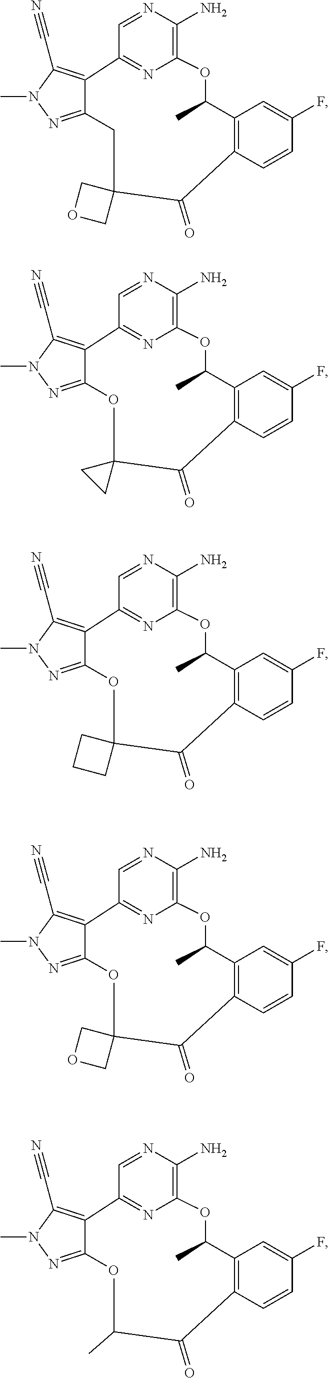 [SMILES: C[C@H]1Oc2nc(cnc2N)-c2c(nn(C)c2C#N)CC2(COC2)C(=O)c2ccc(F)cc21 ; C[C@H]1Oc2nc(cnc2N)-c2c(nn(C)c2C#N)OC2(CC2)C(=O)c2ccc(F)cc21 ; C[C@H]1Oc2nc(cnc2N)-c2c(nn(C)c2C#N)OC2(CCC2)C(=O)c2ccc(F)cc21 ; C[C@H]1Oc2nc(cnc2N)-c2c(nn(C)c2C#N)OC2(COC2)C(=O)c2ccc(F)cc21 ; CC1Oc2nn(C)c(C#N)c2-c2cnc(N)c(n2)O[C@H](C)c2cc(F)ccc2C1=O] 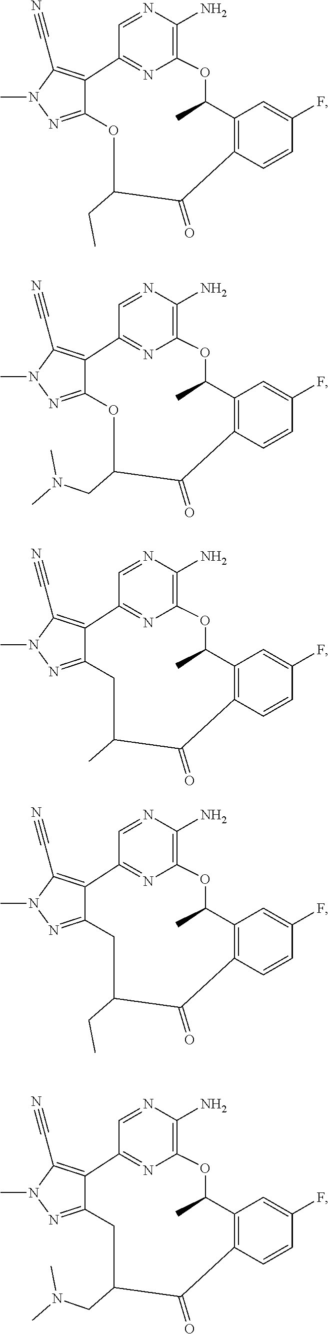 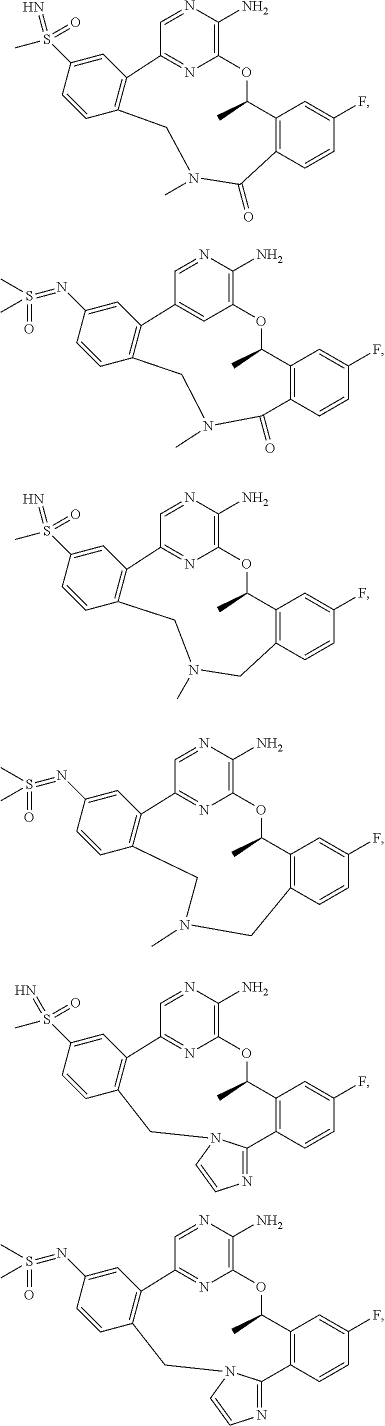 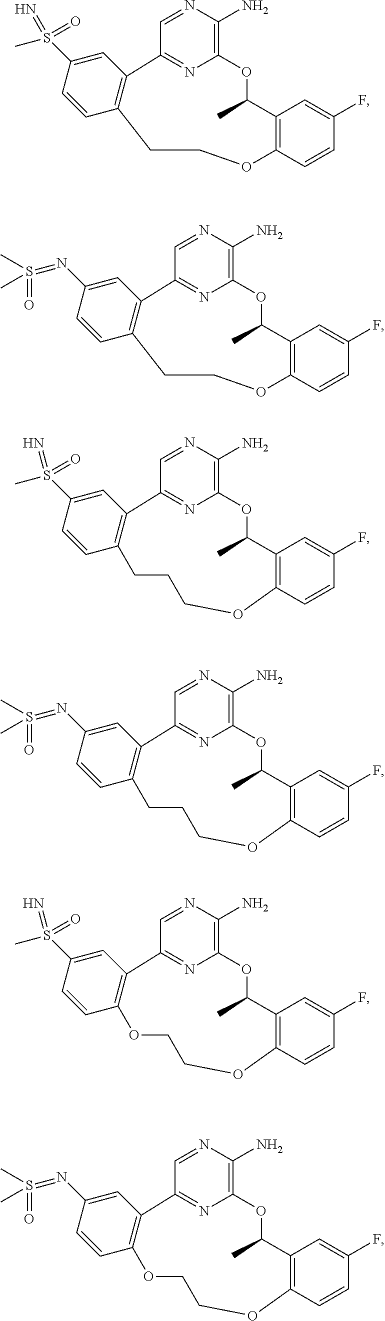 [SMILES: C[C@H]1Oc2nc(cnc2N)-c2cc(S(C)(=N)=O)ccc2CCOc2ccc(F)cc21 ; C[C@H]1Oc2nc(cnc2N)-c2cc(N=S(C)(C)=O)ccc2CCOc2ccc(F)cc21 ; C[C@H]1Oc2nc(cnc2N)-c2cc(S(C)(=N)=O)ccc2CCCOc2ccc(F)cc21 ; C[C@H]1Oc2nc(cnc2N)-c2cc(N=S(C)(C)=O)ccc2CCCOc2ccc(F)cc21 ; C[C@H]1Oc2nc(cnc2N)-c2cc(S(C)(=N)=O)ccc2OCCOc2ccc(F)cc21 ; C[C@H]1Oc2nc(cnc2N)-c2cc(N=S(C)(C)=O)ccc2OCCOc2ccc(F)cc21] 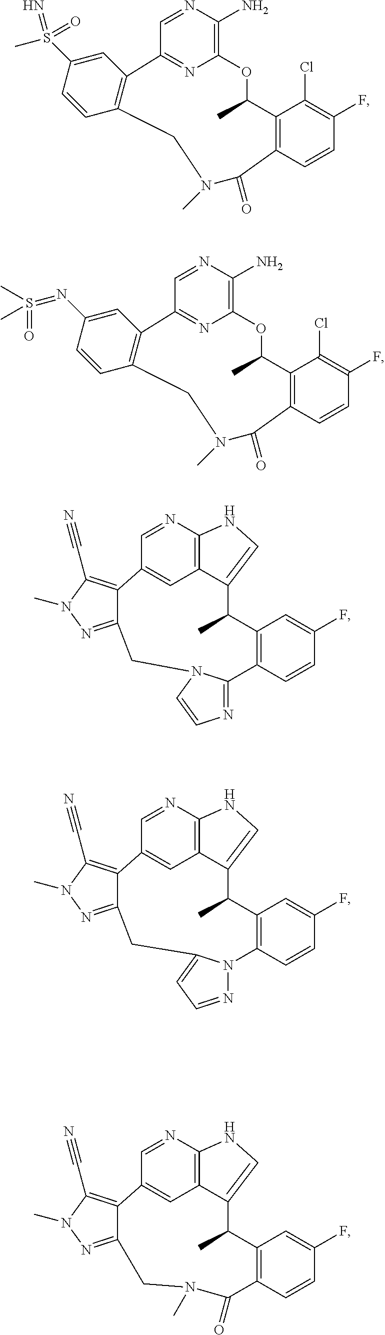 [SMILES: C[C@H]1Oc2nc(cnc2N)-c2cc(S(C)(=N)=O)ccc2CN(C)C(=O)c2ccc(F)c(Cl)c21 ; C[C@H]1Oc2nc(cnc2N)-c2cc(N=S(C)(C)=O)ccc2CN(C)C(=O)c2ccc(F)c(Cl)c21 ; C[C@@H]1c2cc(F)ccc2-c2nccn2Cc2nn(C)c(C#N)c2-c2cnc3[nH]cc1c3c2 ; C[C@@H]1c2cc(F)ccc2-n2nccc2Cc2nn(C)c(C#N)c2-c2cnc3[nH]cc1c3c2 ; C[C@@H]1c2cc(F)ccc2C(=O)N(C)Cc2nn(C)c(C#N)c2-c2cnc3[nH]cc1c3c2] 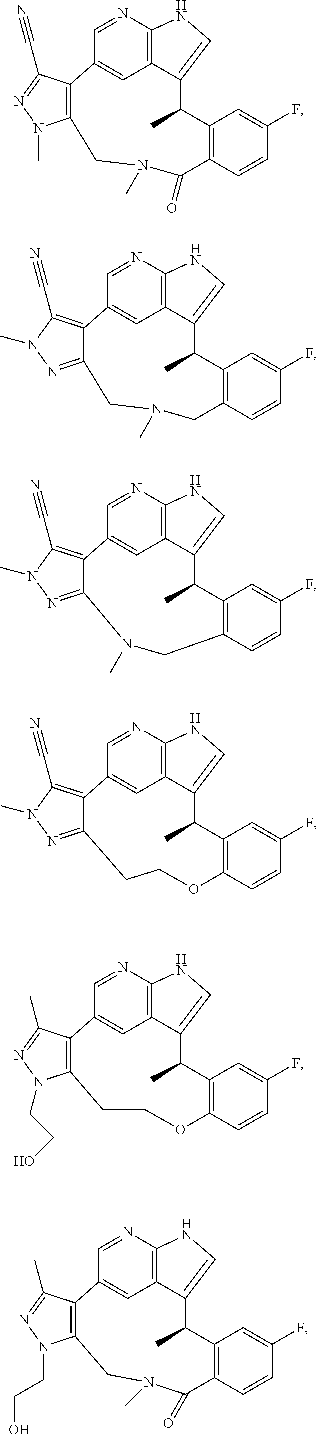 [SMILES: C[C@@H]1c2cc(F)ccc2C(=O)N(C)Cc2c(c(C#N)nn2C)-c2cnc3[nH]cc1c3c2 ; C[C@@H]1c2cc(F)ccc2CN(C)Cc2nn(C)c(C#N)c2-c2cnc3[nH]cc1c3c2 ; C[C@@H]1c2cc(F)ccc2CN(C)c2nn(C)c(C#N)c2-c2cnc3[nH]cc1c3c2 ; C[C@@H]1c2cc(F)ccc2OCCc2nn(C)c(C#N)c2-c2cnc3[nH]cc1c3c2 ; Cc1nn(CCO)c2c1-c1cnc3[nH]cc(c3c1)[C@H](C)c1cc(F)ccc1OCC2 ; Cc1nn(CCO)c2c1-c1cnc3[nH]cc(c3c1)[C@H](C)c1cc(F)ccc1C(=O)N(C)C2] 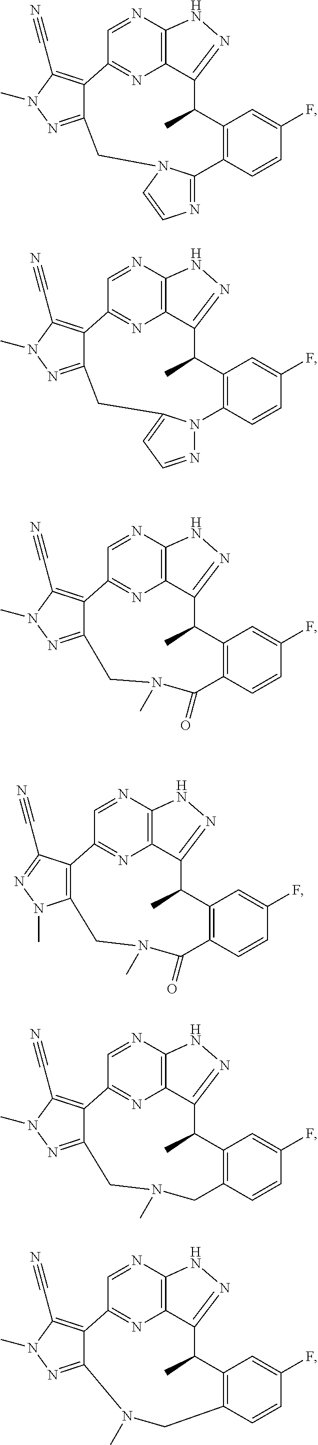 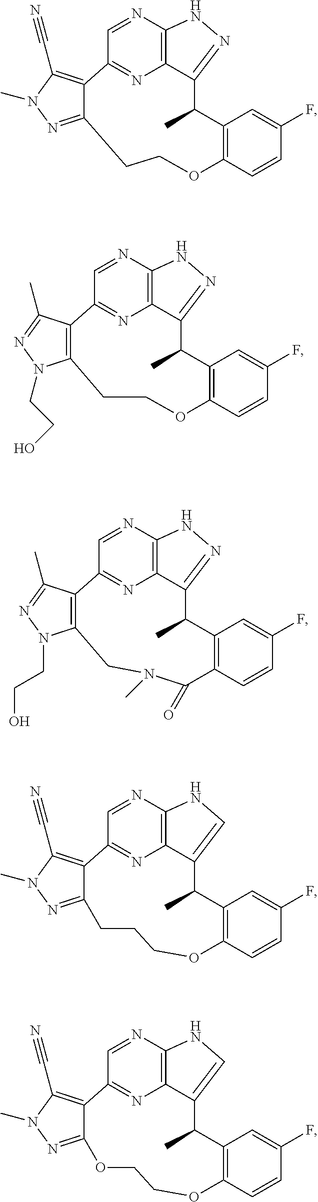 [SMILES: C[C@@H]1c2cc(F)ccc2OCCc2nn(C)c(C#N)c2-c2cnc3[nH]nc1c3n2 ; Cc1nn(CCO)c2c1-c1cnc3[nH]nc(c3n1)[C@H](C)c1cc(F)ccc1OCC2 ; Cc1nn(CCO)c2c1-c1cnc3[nH]nc(c3n1)[C@H](C)c1cc(F)ccc1C(=O)N(C)C2 ; C[C@@H]1c2cc(F)ccc2OCCCc2nn(C)c(C#N)c2-c2cnc3[nH]cc1c3n2 ; C[C@@H]1c2cc(F)ccc2OCCOc2nn(C)c(C#N)c2-c2cnc3[nH]cc1c3n2] 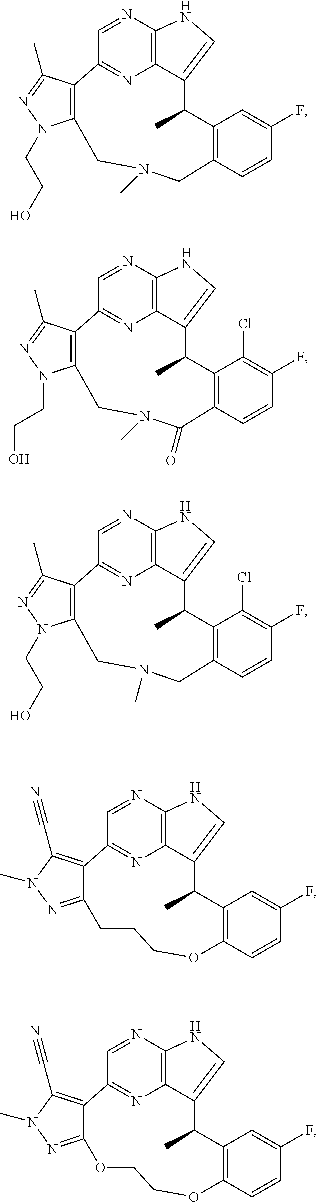 [SMILES: Cc1nn(CCO)c2c1-c1cnc3[nH]cc(c3n1)[C@H](C)c1cc(F)ccc1CN(C)C2 ; Cc1nn(CCO)c2c1-c1cnc3[nH]cc(c3n1)[C@H](C)c1c(ccc(F)c1Cl)C(=O)N(C)C2 ; Cc1nn(CCO)c2c1-c1cnc3[nH]cc(c3n1)[C@H](C)c1c(ccc(F)c1Cl)CN(C)C2 ; C[C@@H]1c2cc(F)ccc2OCCCc2nn(C)c(C#N)c2-c2cnc3[nH]cc1c3n2 ; C[C@@H]1c2cc(F)ccc2OCCOc2nn(C)c(C#N)c2-c2cnc3[nH]cc1c3n2] 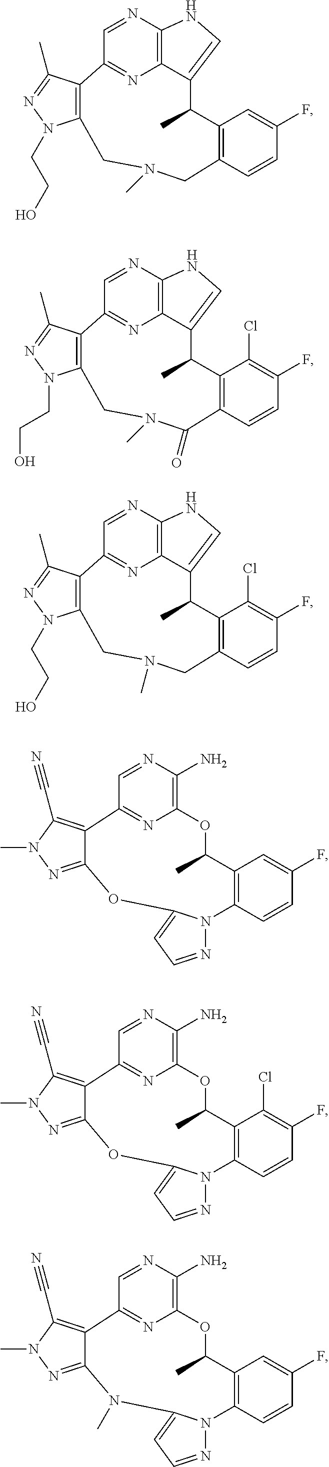 [SMILES: Cc1nn(CCO)c2c1-c1cnc3[nH]cc(c3n1)[C@H](C)c1cc(F)ccc1CN(C)C2 ; Cc1nn(CCO)c2c1-c1cnc3[nH]cc(c3n1)[C@H](C)c1c(ccc(F)c1Cl)C(=O)N(C)C2 ; Cc1nn(CCO)c2c1-c1cnc3[nH]cc(c3n1)[C@H](C)c1c(ccc(F)c1Cl)CN(C)C2 ; C[C@H]1Oc2nc(cnc2N)-c2c(nn(C)c2C#N)Oc2ccnn2-c2ccc(F)cc21 ; C[C@H]1Oc2nc(cnc2N)-c2c(nn(C)c2C#N)Oc2ccnn2-c2ccc(F)c(Cl)c21 ; C[C@H]1Oc2nc(cnc2N)-c2c(nn(C)c2C#N)N(C)c2ccnn2-c2ccc(F)cc21] 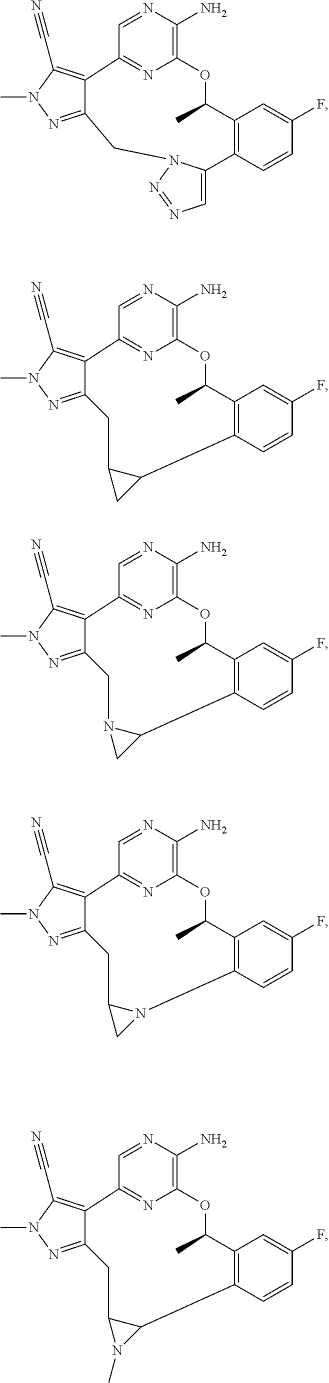 [SMILES: C[C@H]1Oc2nc(cnc2N)-c2c(nn(C)c2C#N)Cn2nncc2-c2ccc(F)cc21 ; C[C@H]1Oc2nc(cnc2N)-c2c(nn(C)c2C#N)CC2CC2c2ccc(F)cc21 ; C[C@H]1Oc2nc(cnc2N)-c2c(nn(C)c2C#N)CN2CC2c2ccc(F)cc21 ; C[C@H]1Oc2nc(cnc2N)-c2c(nn(C)c2C#N)CC2CN2c2ccc(F)cc21 ; C[C@H]1Oc2nc(cnc2N)-c2c(nn(C)c2C#N)CC2C(c3ccc(F)cc31)N2C] 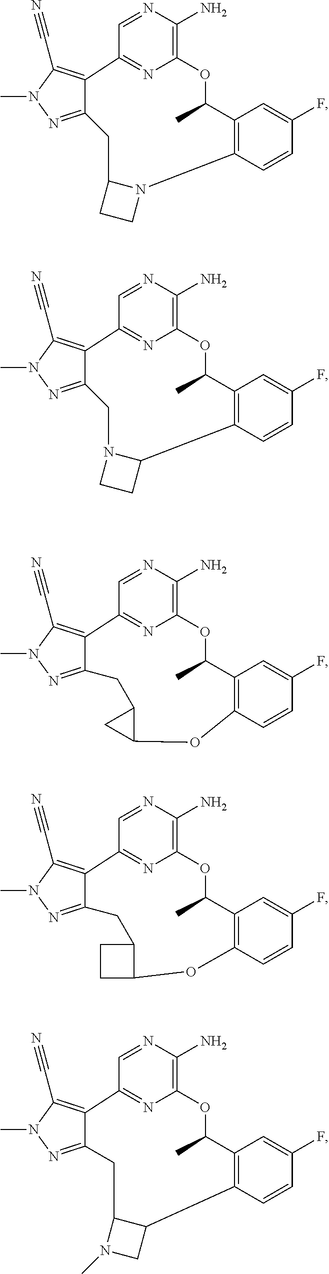 [SMILES: C[C@H]1Oc2nc(cnc2N)-c2c(nn(C)c2C#N)CC2CCN2c2ccc(F)cc21 ; C[C@H]1Oc2nc(cnc2N)-c2c(nn(C)c2C#N)CN2CCC2c2ccc(F)cc21 ; C[C@H]1Oc2nc(cnc2N)-c2c(nn(C)c2C#N)CC2CC2Oc2ccc(F)cc21 ; C[C@H]1Oc2nc(cnc2N)-c2c(nn(C)c2C#N)CC2CCC2Oc2ccc(F)cc21 ; C[C@H]1Oc2nc(cnc2N)-c2c(nn(C)c2C#N)CC2C(CN2C)c2ccc(F)cc21] 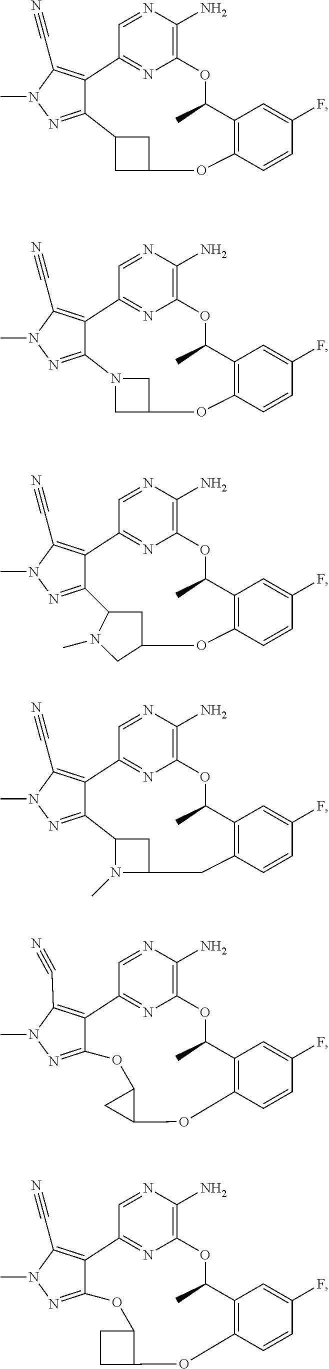 [SMILES: C[C@H]1Oc2nc(cnc2N)-c2c(nn(C)c2C#N)C2CC(C2)Oc2ccc(F)cc21 ; C[C@H]1Oc2nc(cnc2N)-c2c(nn(C)c2C#N)N2CC(C2)Oc2ccc(F)cc21 ; C[C@H]1Oc2nc(cnc2N)-c2c(nn(C)c2C#N)C2CC(CN2C)Oc2ccc(F)cc21 ; C[C@H]1Oc2nc(cnc2N)-c2c(nn(C)c2C#N)C2CC(Cc3ccc(F)cc31)N2C ; C[C@H]1Oc2nc(cnc2N)-c2c(nn(C)c2C#N)OC2CC2Oc2ccc(F)cc21 ; C[C@H]1Oc2nc(cnc2N)-c2c(nn(C)c2C#N)OC2CCC2Oc2ccc(F)cc21] 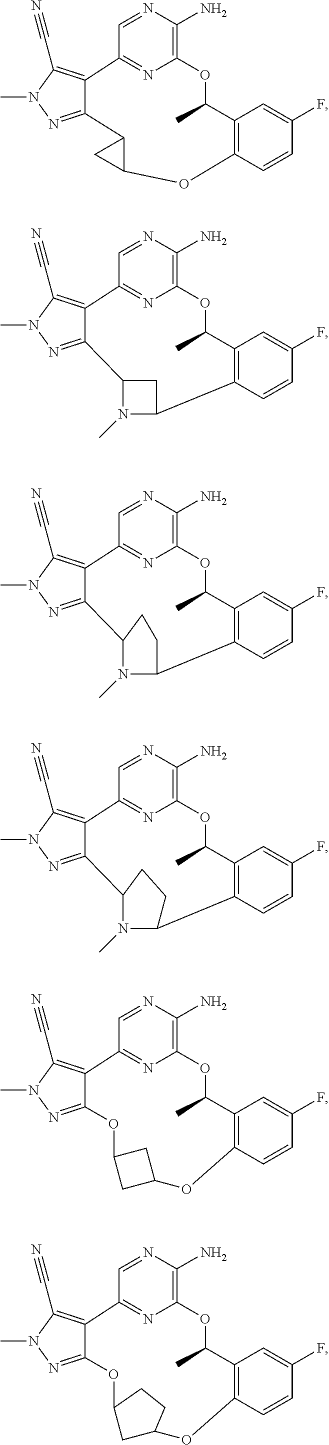 [SMILES: C[C@H]1Oc2nc(cnc2N)-c2c(nn(C)c2C#N)C2CC2Oc2ccc(F)cc21 ; C[C@H]1Oc2nc(cnc2N)-c2c(nn(C)c2C#N)C2CC(c3ccc(F)cc31)N2C ; C[C@H]1Oc2nc(cnc2N)-c2c(nn(C)c2C#N)C2CCC(c3ccc(F)cc31)N2C ; C[C@H]1Oc2nc(cnc2N)-c2c(nn(C)c2C#N)C2CCC(c3ccc(F)cc31)N2C ; C[C@H]1Oc2nc(cnc2N)-c2c(nn(C)c2C#N)OC2CC(C2)Oc2ccc(F)cc21 ; C[C@H]1Oc2nc(cnc2N)-c2c(nn(C)c2C#N)OC2CCC(C2)Oc2ccc(F)cc21] 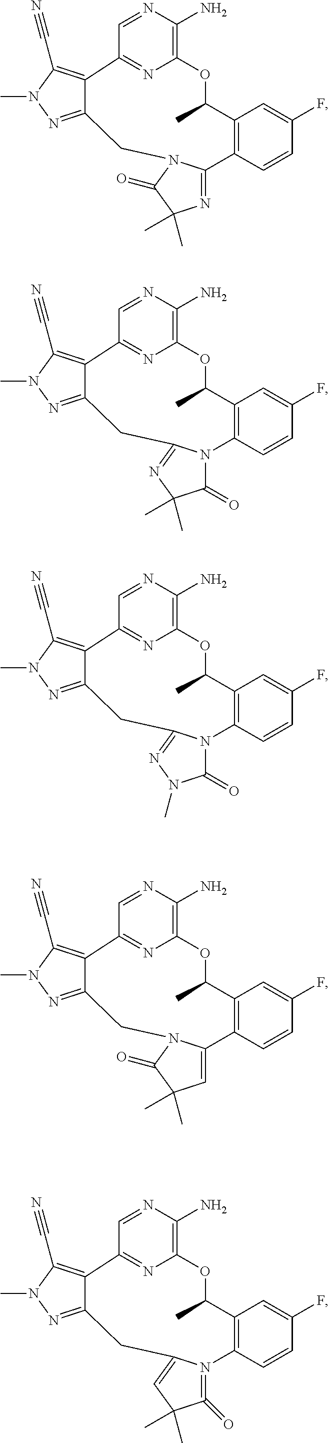 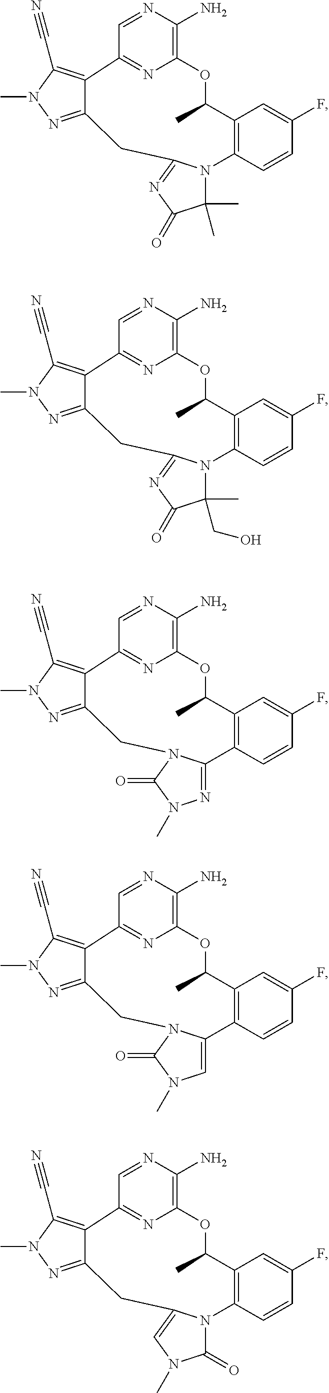 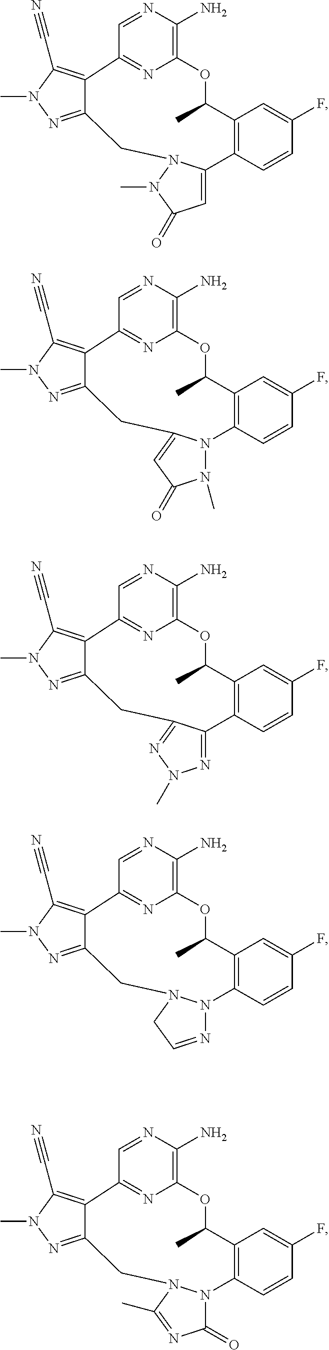 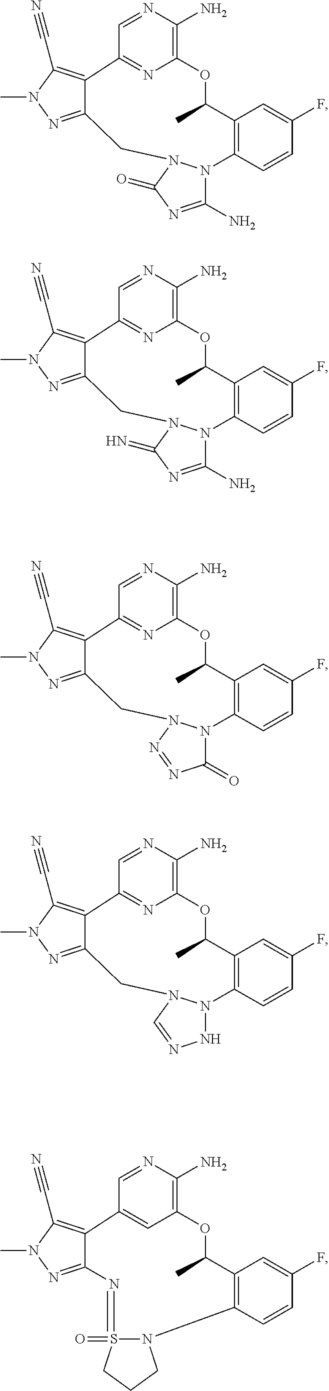 [SMILES: C[C@H]1Oc2nc(cnc2N)-c2c(nn(C)c2C#N)Cn2c(=O)nc(N)n2-c2ccc(F)cc21 ; C[C@H]1Oc2nc(cnc2N)-c2c(nn(C)c2C#N)Cn2c(=N)nc(N)n2-c2ccc(F)cc21 ; C[C@H]1Oc2nc(cnc2N)-c2c(nn(C)c2C#N)Cn2nnc(=O)n2-c2ccc(F)cc21 ; C[C@H]1Oc2nc(cnc2N)-c2c(nn(C)c2C#N)CN2C=NNN2c2ccc(F)cc21 ; C[C@H]1Oc2cc(cnc2N)-c2c(nn(C)c2C#N)N=S2(=O)CCCN2c2ccc(F)cc21] 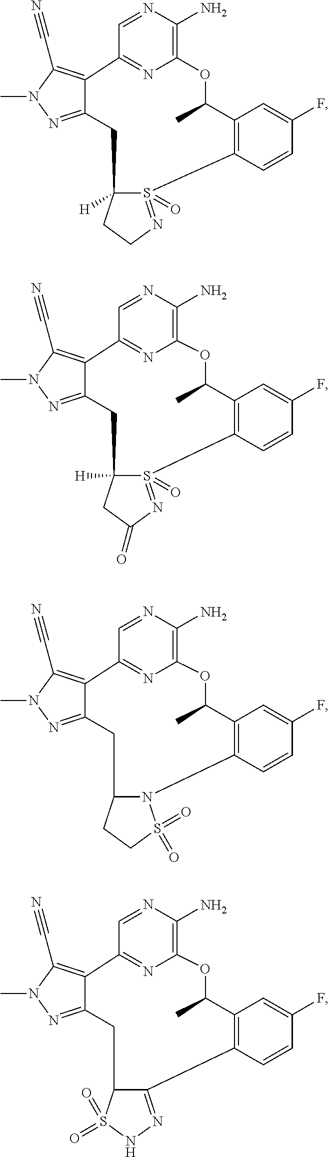 [SMILES: C[C@H]1Oc2nc(cnc2N)-c2c(nn(C)c2C#N)C[C@@H]2CCN=S2(=O)c2ccc(F)cc21 ; C[C@H]1Oc2nc(cnc2N)-c2c(nn(C)c2C#N)C[C@@H]2CC(=O)N=S2(=O)c2ccc(F)cc21 ; C[C@H]1Oc2nc(cnc2N)-c2c(nn(C)c2C#N)CC2CCS(=O)(=O)N2c2ccc(F)cc21 ; C[C@H]1Oc2nc(cnc2N)-c2c(nn(C)c2C#N)CC2C(=NNS2(=O)=O)c2ccc(F)cc21] 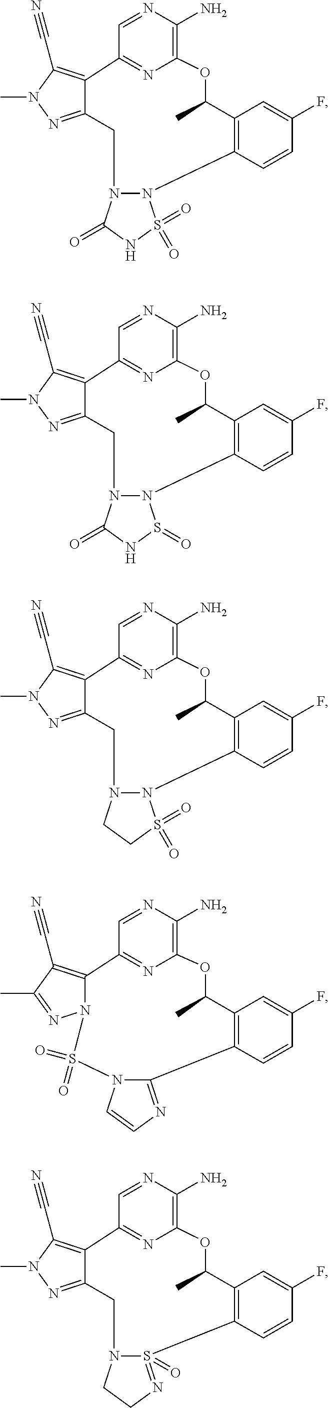 [SMILES: C[C@H]1Oc2nc(cnc2N)-c2c(nn(C)c2C#N)CN2C(=O)NS(=O)(=O)N2c2ccc(F)cc21 ; C[C@H]1Oc2nc(cnc2N)-c2c(nn(C)c2C#N)CN2C(=O)NS(=O)N2c2ccc(F)cc21 ; C[C@H]1Oc2nc(cnc2N)-c2c(nn(C)c2C#N)CN2CCS(=O)(=O)N2c2ccc(F)cc21 ; Cc1nn2c(c1C#N)-c1cnc(N)c(n1)O[C@H](C)c1cc(F)ccc1-c1nccn1S2(=O)=O ; C[C@H]1Oc2nc(cnc2N)-c2c(nn(C)c2C#N)CN2CCN=S2(=O)c2ccc(F)cc21] 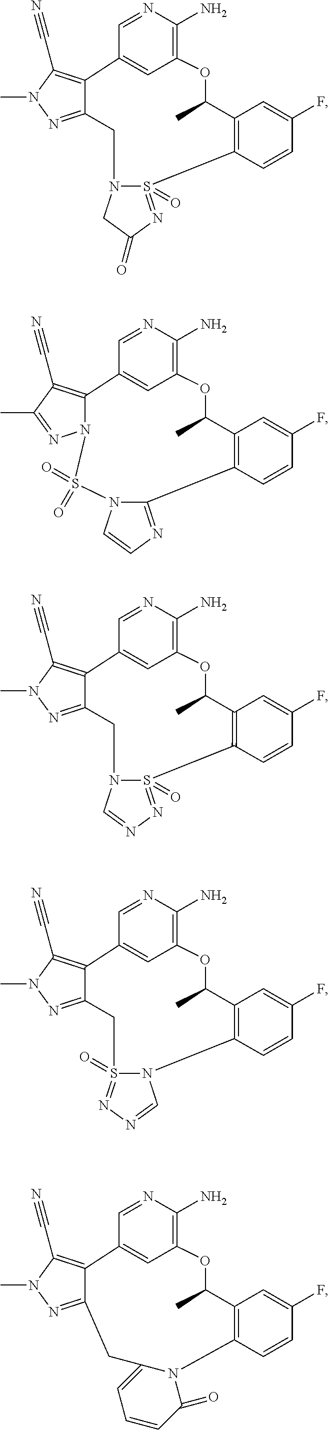 [SMILES: C[C@H]1Oc2cc(cnc2N)-c2c(nn(C)c2C#N)CN2CC(=O)N=S2(=O)c2ccc(F)cc21 ; Cc1nn2c(c1C#N)-c1cnc(N)c(c1)O[C@H](C)c1cc(F)ccc1-c1nccn1S2(=O)=O ; C[C@H]1Oc2cc(cnc2N)-c2c(nn(C)c2C#N)CN2C=NN=S2(=O)c2ccc(F)cc21 ; C[C@H]1Oc2cc(cnc2N)-c2c(nn(C)c2C#N)CS2(=O)=NN=CN2c2ccc(F)cc21 ; C[C@H]1Oc2cc(cnc2N)-c2c(nn(C)c2C#N)Cc2cccc(=O)n2-c2ccc(F)cc21] 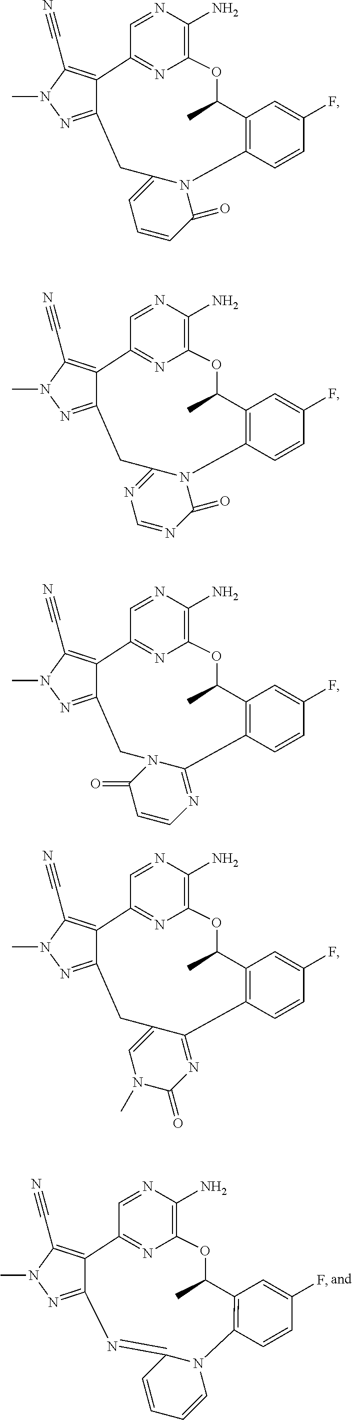 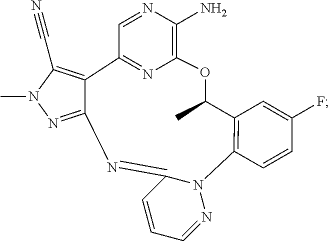 [SMILES: C[C@H]1Oc2nc(cnc2N)-c2c(nn(C)c2C#N)/N=C2\C=CC=NN2c2ccc(F)cc21]
or a pharmaceutically acceptable salt thereof.

57. A compound selected from the group consisting of 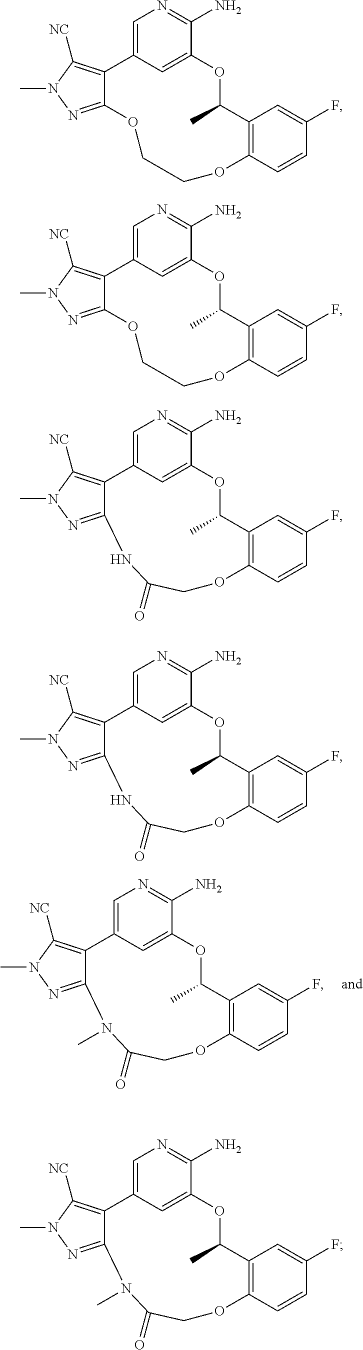
or a pharmaceutically acceptable salt thereof.

58. A pharmaceutical composition comprising the compound of one any one of claims 1-57, or a pharmaceutically acceptable salt thereof, and a pharmaceutically acceptable carrier or excipient.

59. A methods of treating or inhibiting cell proliferation, cell invasiveness, metastases, apoptosis, or angiogenesis in a mammal, comprising administering to a mammal in need thereof the compound of any one of claims 1-57, or a pharmaceutically acceptable salt thereof.
